(19) United States
(12) Patent Application Publication
(10) Pub. No.: US 2024/0066114 A1
(43) Pub. Date: Feb. 29, 2024

(54) MULTIVALENT NUCLEIC ACID BASED CORONAVIRUS VACCINES

(71) Applicant: CureVac SE, Tübingen (DE)

(72) Inventors: Susanne RAUCH, Tübingen (DE); Nicole ROTH, Tübingen (DE); Benjamin PETSCH, Tübingen (DE); Wolfgang GROSSE, Tübingen (DE)

(73) Assignee: CureVac SE, Tübingen (DE)

(21) Appl. No.: 18/043,317

(22) PCT Filed: Aug. 30, 2021
(86) PCT No.: PCT/EP2021/073885

§ 371 Date: Feb. 27, 2023

(30) Foreign Application Priority Data
Aug. 31, 2020 (WO) PCT/EP2020/000145
Aug. 31, 2020 (WO) PCT/EP2020/074251
Feb. 3, 2021 (WO) PCT/EP2021/052555

(51) Int. Cl. A61K 39/215; A61K 9/127; A61K 9/19; A61P 31/14

(52) U.S. Cl. CPC A61K 39/215; A61K 9/1272; A61K 9/19; A61P 31/14; A61K 2039/53

(57) ABSTRACT

The present invention is inter alia directed to compositions comprising at least one nucleic acid encoding at least one antigenic peptide or protein selected or derived from a Coronavirus membrane protein (M), nucleocapsid protein (N), non-structural protein, and/or accessory protein or an immunogenic fragment or immunogenic variant thereof. The composition may additionally comprise at least one nucleic acid encoding at least one antigenic peptide or protein selected or derived from a Coronavirus spike protein (S). The nucleic acid sequences of the compositions are preferably in association with a polymeric carrier, a polycationic protein or peptide, or a lipid nanoparticle (LNP). The compositions provided herein are for use in treatment or prophylaxis of an infection with at least one Coronavirus, and may therefore be comprised in a vaccine, preferably a multivalent vaccine. The invention is also directed to first and second and further medical uses and to methods of treating or preventing Coronavirus infections.

### INTRODUCTION

The present invention is inter alia directed to compositions comprising at least one nucleic acid encoding at least one antigenic peptide or protein selected or derived from a Coronavirus membrane protein (M), nucleocapsid protein (N), non-structural protein, and/or accessory protein. The composition may additionally comprise at least one nucleic acid encoding at least one antigenic peptide or protein selected or derived from a Coronavirus spike protein (S). Nucleic acid sequences of the compositions are preferably in association with a polymeric carrier, a polycationic protein or peptide, or a lipid nanoparticle (LNP). The compositions provided herein are for use in treatment or prophylaxis of an infection with at least one Coronavirus, and may therefore be comprised in a vaccine, preferably a multivalent vaccine. Also provided are medical uses and methods of treating or preventing Coronavirus infections.

Coronaviruses are highly contagious, enveloped, positive single stranded RNA viruses of the Coronaviridae family. Coronaviruses (CoV) are genetically highly variable, and individual virus species can also infect several host species by overcoming the species barrier. Such transfers have resulted in infections in humans with the SARS-associated coronavirus (SARS-CoV-1), with the Middle East respiratory syndrome coronavirus (MERS-CoV), and with SARS-CoV-2 (causing COVID-19 disease).

SARS-CoV-1, which causes severe acute respiratory syndrome (SARS), infected 8422 humans and resulted in 916 deaths in 37 countries between 2002 and 2003. MERS-CoV was first identified in the Middle East in 2012. A report confirmed 1791 MERS-CoV infection cases, including at least 640 deaths in 27 countries, as of July 2016.

The Coronavirus pandemic that presumably started in the Chinese city of Wuhan at the turn of 2019/2020 has been attributed to a previously unknown coronavirus (SARS-CoV-2) which causes a severe respiratory disease (COVID-19). By end of August 2020, there were about 25,000,000 confirmed cases of a SARS-CoV-2 infection, spreading across almost every country in the world, with more than 850,000 COVID-19 associated deaths.

These current outbreaks shows the substantial risk of a severe global pandemic that can be caused by Coronaviruses. It would be fundamentally important for the global health to provide a vaccine that provides protection against (a pandemic) Coronavirus. It would be advantageous to provide a vaccine that protects against a Coronavirus infection by inducing strong Coronavirus specific neutralizing antibody responses including T-cell responses.

Further, as the elderly population is strongly affected by such viruses, it would be advantageous to provide a vaccine that is efficient in the elderly population.

Nucleic acid based vaccination, including DNA or RNA, represents a promising technique for novel vaccines against emerging viruses. Nucleic acids can be genetically engineered and administered to a human subject. Transfected cells directly produce the encoded antigen (e.g. provided by a DNA or an RNA, in particular an mRNA), which results in protective immunological responses.

A pivotal role for virus-specific memory T-cells in broad and long-term protection against SARS-CoV infection has been elucidated (see e.g. Channappanavar, Rudragouda, et al. “Virus-specific memory CD8 T cells provide substantial protection from lethal severe acute respiratory syndrome coronavirus infection.” Journal of virology 88.19 (2014): 11034-11044). Virus-specific CD8 T cells are e.g. required for pathogen clearance and for mediating protection after viral challenge. An effective SARS-CoV-2 vaccine should therefore not only induce strong functional humoral immune responses against SARS-CoV-2, but also induce SARS-CoV-2 specific CD8+ T-cell and CD4+ T-cell responses.

During the pandemic, new SARS-CoV-2 variant strains were emerging that are often more contagious or more pathogenic then the original SARS-CoV-2 strain. Such new emerging SARS-CoV-2 strains may potentially lead to a reduced efficiency of first generation vaccines that were developed against the original SARS-CoV-2 strain.

It is the object of the underlying invention to provide a nucleic acid based vaccine that provides protection against Coronavirus infections, preferably against a SARS-CoV-2 infection.

### Definitions

For the sake of clarity and readability the following definitions are provided. Any technical feature mentioned for these definitions may be read on each and every embodiment of the invention. Additional definitions and explanations may be specifically provided in the context of these embodiments.

Percentages in the context of numbers should be understood as relative to the total number of the respective items. In other cases, and unless the context dictates otherwise, percentages should be understood as percentages by weight (wt.-%).

About: The term “about” is used when determinants or values do not need to be identical, i.e. 100% the same. Accordingly, “about” means, that a determinant or values may diverge by 0.1% to 20%, preferably by 0.1% to 10%; in particular, by 0.5%, 1%, 2%, 3%, 4%, 5%, 6%, 7%, 8%, 9%, 10%, 11%, 12%, 13%, 14%, 15%, 16%, 17%, 18%, 19%, 20%. The skilled person will know that e.g. certain parameters or determinants may slightly vary based on the method how the parameter was determined. For example, if a certain determinants or value is defined herein to have e.g. a length of “about 1000 nucleotides”, the length may diverge by 0.1% to 20%, preferably by 0.1% to 10%; in particular, by 0.5%, 1%, 2%, 3%, 4%, 5%, 6%, 7%, 8%, 9%, 10%, 11%, 12%, 13%, 14%, 15%, 16%, 17%, 18%, 19%, 20%. Accordingly, the skilled person will know that in that specific example, the length may diverge by 1 to 200 nucleotides, preferably by 1 to 200 nucleotides; in particular, by 5, 10, 20, 30, 40, 50, 60, 70, 80, 90, 100, 110, 120, 130, 140, 150, 160, 170, 180, 190, 200 nucleotides.

Adaptive immune response: The term “adaptive immune response” as used herein will be recognized and understood by the person of ordinary skill in the art, and is e.g. intended to refer to an antigen-specific response of the immune system (the adaptive immune system). Antigen specificity allows for the generation of responses that are tailored to specific pathogens or pathogen-infected cells. The ability to mount these tailored responses is usually maintained in the body by “memory cells” (B-cells). In the context of the invention, the antigen is provided by the nucleic acid (e.g. an RNA or a DNA) encoding at least one antigenic peptide or protein derived from a Coronavirus, e.g. from SARS-CoV-2

Antigen: The term “antigen” as used herein will be recognized and understood by the person of ordinary skill in the art, and is e.g. intended to refer to a substance which may be recognized by the immune system, preferably by the adaptive immune system, and is capable of triggering an antigen-specific immune response, e.g. by formation of antibodies and/or antigen-specific T cells as part of an adaptive immune response. Typically, an antigen may be or may comprise a peptide or protein which may be presented by the MHC to T-cells. Also fragments, variants and derivatives of peptides or proteins comprising at least one epitope are understood as antigens in the context of the invention. In the context of the present invention, an antigen may be the product of translation of a provided nucleic acid as specified herein.

Antigenic peptide or protein: The term “antigenic peptide or protein” or “immunogenic peptide or protein” will be recognized and understood by the person of ordinary skill in the art, and is e.g. intended to refer to a peptide, protein derived from a (antigenic or immunogenic) protein which stimulates the body's adaptive immune system to provide an adaptive immune response. Therefore an antigenic/immunogenic peptide or protein comprises at least one epitope (as defined herein) or antigen (as defined herein) of the protein it is derived from (e.g., Coronavirus M, N, S, etc.)

Cationic: Unless a different meaning is clear from the specific context, the term “cationic” means that the respective structure bears a positive charge, either permanently or not permanently, but in response to certain conditions such as pH. Thus, the term “cationic” covers both “permanently cationic” and “cationisable”.

Cationisable: The term “cationisable” as used herein means that a compound, or group or atom, is positively charged at a lower pH and uncharged at a higher pH of its environment. Also in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKa of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation which is well-known to a person skilled in the art. E.g., in some embodiments, if a compound or moiety is cationisable, it is preferred that it is positively charged at a pH value of about 1 to 9, preferably 4 to 9, 5 to 8 or even 6 to 8, more preferably of a pH value of or below 9, of or below 8, of or below 7, most preferably at physiological pH values, e.g. about 7.3 to 7.4, i.e. under physiological conditions, particularly under physiological salt conditions of the cell in vivo. In other embodiments, it is preferred that the cationisable compound or moiety is predominantly neutral at physiological pH values, e.g. about 7.0-7.4, but becomes positively charged at lower pH values. In some embodiments, the preferred range of pKa for the cationisable compound or moiety is about 5 to about 7.

Coding sequence/coding region: The terms “coding sequence” or “coding region” and the corresponding abbreviation “cds” as used herein will be recognized and understood by the person of ordinary skill in the art, and are e.g. intended to refer to a sequence of several nucleotide triplets, which may be translated into a peptide or protein. A coding sequence in the context of the present invention may be a DNA sequence, preferably an RNA sequence, consisting of a number of nucleotides that may be divided by three, which starts with a start codon and which preferably terminates with a stop codon.

Derived from: The term “derived from” as used throughout the present specification in the context of a nucleic acid, i.e. for a nucleic acid “derived from” (another) nucleic acid, means that the nucleic acid, which is derived from (another) nucleic acid, shares e.g. at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid from which it is derived. The skilled person is aware that sequence identity is typically calculated for the same types of nucleic acids, i.e. for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing the uracils (U) by thymidines (T) throughout the sequence) or, vice versa, the DNA sequence is converted into the corresponding RNA sequence (in particular by replacing the T by U throughout the sequence). Thereafter, the sequence identity of the DNA sequences or the sequence identity of the RNA sequences is determined. Preferably, a nucleic acid “derived from” a nucleic acid also refers to nucleic acid, which is modified in comparison to the nucleic acid from which it is derived, e.g. in order to increase RNA stability even further and/or to prolong and/or increase protein production. In the context of amino acid sequences (e.g. antigenic peptides or proteins) the term “derived from” means that the amino acid sequence, which is derived from (another) amino acid sequence, shares e.g. at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence from which it is derived.

Epitope: The term “epitope” (also called “antigen determinant” in the art) as used herein will be recognized and understood by the person of ordinary skill in the art, and is e.g. intended to refer to T cell epitopes and B cell epitopes. T cell epitopes or parts of the antigenic peptides or proteins and may comprise fragments preferably having a length of about 6 to about 20 or even more amino acids, e.g. fragments as processed and presented by MHC class I molecules, preferably having a length of about 8 to about 10 amino acids, e.g. 8, 9, or 10, (or even 11, or 12 amino acids), or fragments as processed and presented by MHC class II molecules, preferably having a length of about 13 to about 20 or even more amino acids. These fragments are typically recognized by T cells in form of a complex consisting of the peptide fragment and an MHC molecule, i.e. the fragments are typically not recognized in their native form. B cell epitopes are typically fragments located on the outer surface of (native) protein or peptide antigens, preferably having 5 to 15 amino acids, more preferably having 5 to 12 amino acids, even more preferably having 6 to 9 amino acids, which may be recognized by antibodies, i.e. in their native form. Such epitopes of proteins or peptides may furthermore be selected from any of the herein mentioned variants of such proteins or peptides. In this context epitopes can be conformational or discontinuous epitopes which are composed of segments of the proteins or peptides as defined herein that are discontinuous in the amino acid sequence of the proteins or peptides as defined herein but are brought together in the three-dimensional structure or continuous or linear epitopes which are composed of a single polypeptide chain.

Fragment: The term “fragment” as used throughout the present specification in the context of a nucleic acid sequence (e.g. RNA or a DNA) or an amino acid sequence may typically be a shorter portion of a full-length sequence of e.g. a nucleic acid sequence or an amino acid sequence. Accordingly, a fragment, typically, consists of a sequence that is identical to the corresponding stretch within the full-length sequence. A preferred fragment of a sequence in the context of the present invention, consists of a continuous stretch of entities, such as nucleotides or amino acids corresponding to a continuous stretch of entities in the molecule the fragment is derived from, which represents at least 40%, 50%, 60%, 70%, 80%, 90%, 95% of the total (i.e. full-length) molecule from which the fragment is derived (e.g. Coronavirus M, N, S). The term “fragment” as used throughout the present specification in the context of proteins or peptides may, typically, comprise a sequence of a protein or peptide as defined herein, which is, with regard to its amino acid sequence, N-terminally and/or C-terminally truncated compared to the amino acid sequence of the original protein. Such truncation may thus occur either on the amino acid level or correspondingly on the nucleic acid level. A sequence identity with respect to such a fragment as defined herein may therefore preferably refer to the entire protein or peptide as defined herein or to the entire (coding) nucleic acid molecule of such a protein or peptide. Fragments of proteins or peptides may comprise at least one epitope of those proteins or peptides.

Heterologous: The terms “heterologous” or “heterologous sequence” as used throughout the present specification in the context of a nucleic acid sequence or an amino acid sequence refers to a sequence (e.g. RNA, DNA, amino acid) has to be understood as a sequence that is derived from another gene, another allele, or e.g. another species or virus. Two sequences are typically understood to be “heterologous” if they are not derivable from the same gene or from the same allele. I.e., although heterologous sequences may be derivable from the same organism or virus, in nature, they do not occur in the same nucleic acid or protein.

Humoral immune response: The terms “humoral immunity” or “humoral immune response” will be recognized and understood by the person of ordinary skill in the art, and are e.g. intended to refer to B-cell mediated antibody production and optionally to accessory processes accompanying antibody production. A humoral immune response may be typically characterized, e.g. by Th2 activation and cytokine production, germinal center formation and isotype switching, affinity maturation and memory cell generation. Humoral immunity may also refer to the effector functions of antibodies, which include pathogen and toxin neutralization, classical complement activation, and opsonin promotion of phagocytosis and pathogen elimination.

Identity (of a sequence): The term “identity” as used throughout the present specification in the context of a nucleic acid sequence or an amino acid sequence will be recognized and understood by the person of ordinary skill in the art, and is e.g. intended to refer to the percentage to which two sequences are identical. To determine the percentage to which two sequences are identical, e.g. nucleic acid sequences or amino acid (aa) sequences as defined herein, preferably the aa sequences encoded by the nucleic acid sequence as defined herein or the aa sequences themselves, the sequences can be aligned in order to be subsequently compared to one another. Therefore, e.g. a position of a first sequence may be compared with the corresponding position of the second sequence. If a position in the first sequence is occupied by the same residue as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment. The percentage to which two sequences are identical is then a function of the number of identical positions divided by the total number of positions including those positions which are only occupied in one sequence. The percentage to which two sequences are identical can be determined using an algorithm, e.g. an algorithm integrated in the BLAST program.

Immunogen, immunogenic: The terms “immunogen” or “immunogenic” will be recognized and understood by the person of ordinary skill in the art, and are e.g. intended to refer to a compound that is able to stimulate/induce an immune response. Preferably, an immunogen is a peptide, polypeptide, or protein. An immunogen in the sense of the present invention is the product of translation of a provided nucleic acid, comprising at least one coding sequence encoding at least one antigenic peptide, protein derived from e.g. a coronavirus protein as defined herein. Typically, an immunogen elicits an adaptive immune response.

Immune response: The term “immune response” will be recognized and understood by the person of ordinary skill in the art, and is e.g. intended to refer to a specific reaction of the adaptive immune system to a particular antigen (so called specific or adaptive immune response) or an unspecific reaction of the innate immune system (so called unspecific or innate immune response), or a combination thereof.

Immune system: The term “immune system” will be recognized and understood by the person of ordinary skill in the art, and is e.g. intended to refer to a system of the organism that protects the organisms from infection. If a pathogen succeeds in passing a physical barrier of an organism and enters this organism, the innate immune system provides an immediate non-specific response. If pathogens evade this innate response, vertebrates possess a second layer of protection, the adaptive immune system. The immune system adapts its response during an infection to improve its recognition of the pathogen. This improved response is then retained after the pathogen has been eliminated, in the form of an immunological memory, and allows the adaptive immune system to mount faster and stronger attacks each time this pathogen is encountered. According to this, the immune system comprises the innate and the adaptive immune system. Each of these two parts typically contains so called humoral and cellular components.

Innate immune system: The term “innate immune system” (also known as non-specific or unspecific immune system) will be recognized and understood by the person of ordinary skill in the art, and is e.g. intended to refer to a system typically comprising the cells and mechanisms that defend the host from infection by other organisms in a non-specific manner. This means that the cells of the innate system may recognize and respond to pathogens in a generic way, but unlike the adaptive immune system, it does not confer long-lasting or protective immunity to the host. The innate immune system may be activated by ligands of pattern recognition receptor e.g. Toll-like receptors, NOD-like receptors, or RIG-1 like receptors etc.

Lipidoid compound: A lipidoid compound, also simply referred to as lipidoid, is a lipid-like compound, i.e. an amphiphilic compound with lipid-like physical properties. In the context of the present invention, the term lipid is considered to encompass lipidoid compounds.

Nucleic acid, nucleic acid molecule: The terms “nucleic acid” or “nucleic acid molecule” as used herein, will be recognized and understood by the person of ordinary skill in the art. The terms “nucleic acid” or “nucleic acid molecule” preferably refers to DNA (molecules) or RNA (molecules). The term is used synonymously with the term polynucleotide. Preferably, a nucleic acid or a nucleic acid molecule is a polymer comprising or consisting of nucleotide monomers that are covalently linked to each other by phosphodiester-bonds of a sugar/phosphate-backbone. The terms “nucleic acid” or “nucleic acid molecule” also encompasses modified nucleic acid (molecules), such as base-modified, sugar-modified or backbone-modified DNA or RNA (molecules) as defined herein.

Nucleic acid sequence, DNA sequence, RNA sequence: The terms “nucleic acid sequence”, “DNA sequence”, “RNA sequence” will be recognized and understood by the person of ordinary skill in the art, and e.g. refer to a particular and individual order of the succession of its nucleotides.

Nucleic acid species: In the context of the invention, the term “nucleic acid species” is not restricted to mean “one single nucleic acid molecule” but is understood to comprise an ensemble of essentially identical nucleic acid molecules. Accordingly, it may relate to a plurality of essentially identical nucleic acid molecules, e.g. DNA or RNA molecules.

Multivalent vaccine: A multivalent Coronavirus vaccine of the invention provides more than one valence (e.g. an antigen). These at least two antigen may be derived from two different Coronaviruses (e.g. one antigen derived from SARS-CoV-1, one antigen derived from SARS-CoV-2) or may be derived the same Coronavirus (e.g., two different antigens derived from SARS-CoV-2, e.g. M and N and S).

Permanently cationic: The term “permanently cationic” as used herein will be recognized and understood by the person of ordinary skill in the art, and means, e.g., that the respective compound, or group, or atom, is positively charged at any pH value or hydrogen ion activity of its environment. Typically, the positive charge results from the presence of a quaternary nitrogen atom. Where a compound carries a plurality of such positive charges, it may be referred to as permanently polycationic.

RNA sequence: The term “RNA sequence” will be recognized and understood by the person of ordinary skill in the art, and e.g. refer to a particular and individual order of the succession of its ribonucleotides.

Stabilized RNA: The term “stabilized RNA” refers to an RNA that is modified such, that it is more stable to disintegration or degradation, e.g., by environmental factors or enzymatic digest, such as by exo- or endonuclease degradation, compared to an RNA without such modification. Preferably, a stabilized RNA in the context of the present invention is stabilized in a cell, such as a prokaryotic or eukaryotic cell, preferably in a mammalian cell, such as a human cell. The stabilization effect may also be exerted outside of cells, e.g. in a buffer solution etc., e.g., for storage of a composition comprising the stabilized RNA.

T-cell responses: The terms “cellular immunity” or “cellular immune response” or “cellular T-cell responses” as used herein will be recognized and understood by the person of ordinary skill in the art, and are for example intended to refer to the activation of macrophages, natural killer cells (NK), antigen-specific cytotoxic T-lymphocytes, and the release of various cytokines in response to an antigen. In more general terms, cellular immunity is not based on antibodies, but on the activation of cells of the immune system. Typically, a cellular immune response may be characterized e.g. by activating antigen-specific cytotoxic T-lymphocytes that are able to induce apoptosis in cells, e.g. specific immune cells like dendritic cells or other cells, displaying epitopes of foreign antigens on their surface.

UTR: The term “untranslated region” or “UTR” or “UTR element” will be recognized and understood by the person of ordinary skill in the art, and are e.g. intended to refer to a part of a nucleic acid molecule typically located 5′ or 3′ located of a coding sequence. An UTR is not translated into protein. An UTR may be part of a nucleic acid, e.g. a DNA or an RNA. An UTR may comprise elements for controlling gene expression, also called regulatory elements. Such regulatory elements may be, e.g., ribosomal binding sites, miRNA binding sites etc.

3′-UTR: The term “3-untranslated region” or “3-UTR” or “3′-UTR element” will be recognized and understood by the person of ordinary skill in the art, and are e.g. intended to refer to a part of a nucleic acid molecule located 3′ (i.e. downstream) of a coding sequence and which is not translated into protein. A 3′-UTR may be part of an RNA, located between a coding sequence and an (optional) poly(A) sequence. A 3′-UTR may comprise elements for controlling gene expression, also called regulatory elements. Such regulatory elements may be, e.g., ribosomal binding sites, miRNA binding sites etc.

5′-UTR: The term “5′-untranslated region” or “5′-UTR” or “5′-UTR element” will be recognized and understood by the person of ordinary skill in the art, and are e.g. intended to refer to a part of a nucleic acid molecule located 5′ (i.e. upstream) of a coding sequence and which is not translated into protein. A 5′-UTR may be part of an RNA, located between a coding sequence and an (optional) 5′ cap. A 5′-UTR may comprise elements for controlling gene expression, also called regulatory elements. Such regulatory elements may be, e.g., ribosomal binding sites, miRNA binding sites etc.

Variant (of a sequence): The term “variant” as used throughout the present specification in the context of a nucleic acid sequence will be recognized and understood by the person of ordinary skill in the art, and is e.g. intended to refer to a variant of a nucleic acid sequence derived from another nucleic acid sequence. E.g., a variant of a nucleic acid sequence may exhibit one or more nucleotide deletions, insertions, additions and/or substitutions compared to the nucleic acid sequence from which the variant is derived. A variant of a nucleic acid sequence may at least 50%, 60%, 70%, 80%, 90%, or 95% identical to the nucleic acid sequence the variant is derived from. The variant is a functional variant in the sense that the variant has retained at least 50%, 60%, 70%, 80%, 90%, or 95% or more of the function of the sequence where it is derived from. A “variant” of a nucleic acid sequence may have at least 70%, 75%, 80%, 85%, 90%, 95%, 98% or 99% nucleotide identity over a stretch of at least 10, 20, 30, 50, 75 or 100 nucleotide of such nucleic acid sequence.

The term “variant” as used throughout the present specification in the context of proteins or peptides is e.g. intended to refer to a proteins or peptide variant having an amino acid sequence which differs from the original sequence in one or more mutation(s)/substitution(s), such as one or more substituted, inserted and/or deleted amino acid(s). Preferably, these fragments and/or variants have the same, or a comparable specific antigenic property (immunogenic variants, antigenic variants). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra). A “variant” of a protein or peptide may have at least 70%, 75%, 80%, 85%, 90%, 95%, 98% or 99% amino acid identity over a stretch of at least 10, 20, 30, 50, 75 or 100 amino acids of such protein or peptide. Preferably, a variant of a protein comprises a functional variant of the protein, which means, in the context of the invention, that the variant exerts essentially the same, or at least 40%, 50%, 60%, 70%, 80%, 90% of the immunogenicity as the protein it is derived from.

### SHORT DESCRIPTION OF THE INVENTION

As further defined in the claims and the underlying description, these objects of the invention are inter alia solved by providing a pharmaceutical composition comprising or consisting of a nucleic acid, e.g. an RNA or a DNA, comprising at least one coding sequence encoding at least one antigenic peptide or protein from a Coronavirus.

Suitably, the pharmaceutical composition or the vaccine of the invention has at least some of the following advantageous features:
Translation of the nucleic acid at the site of injection/vaccination (e.g. muscle);
Efficient induction of antigen-specific immune responses against all the encoded proteins, suitably at a very low dosage and dosing regimen;
Suitability for vaccination of infants and/or newborns or the elderly, in particular the elderly;
Suitability of the composition/vaccine for intramuscular administration;
Induction of specific and functional humoral immune response against Coronavirus, e.g. SARS-CoV-2;
Induction of specific and functional humoral immune response against Coronavirus variants, e.g. SARS-CoV-2 variants;
Induction of broad, functional cellular T-cell responses against Coronavirus, e.g. SARS-CoV-2;
Induction of broad, functional cellular T-cell responses against Coronavirus variants, e.g. SARS-CoV-2 variants;
Induction of specific B-cell memory against Coronavirus, e.g. SARS-CoV-2;
Induction of specific B-cell memory against Coronavirus variants, e.g. SARS-CoV-2 variants;
Induction of functional antibodies that can effectively neutralize the Coronavirus, e.g. SARS-CoV-2;
Induction of functional antibodies that can effectively neutralize emerging variants of a Coronavirus, e.g. SARS-CoV-2;
Eliciting of mucosal IgA immunity by inducing of mucosal IgA antibodies;
Induction of a well-balanced B cell and T cell responses;
Induction of protective immunity against coronavirus infection, e.g. against SARS-CoV-2 or emerging variants thereof;
Fast onset of immune protection against Coronavirus and Coronavirus variants, e.g. SARS-CoV-2 variants;
Longevity of the induced immune responses against Coronavirus;
Longevity of the induced immune responses against Coronavirus by induction broad, functional cellular T-cell responses against Coronavirus, targeting also variants of Coronavirus, e.g. SARS-CoV-2;
No enhancement of a virus infection due to vaccination or immunopathological effects;
No antibody dependent enhancement (ADE) caused by the nucleic acid based vaccine;
No excessive induction of systemic cytokine or chemokine response after application of the vaccine, which could lead to an undesired high reactogenicity upon vaccination;
Well tolerability, no side-effects, non-toxicity of the vaccine;
Advantageous stability characteristics of the nucleic acid-based vaccine;
Speed, adaptability, simplicity and scalability of vaccine production;
Advantageous vaccination regimen that only requires one or two vaccination for sufficient protection;
Advantageous vaccination regimen that only requires a low dose of the composition/vaccine for sufficient protection.
Boostability of an existing immunity against SARS-CoV-2, preferably inducing additional immune responses against SARS-CoV-2 variants;
Induction of different, SARS-CoV-2 strain specific immune responses in subjects that have been exposed to a different strain or that have been vaccinated with a vaccine against a different strain;
Induction of a broad immune response across various SARS-CoV-2 variants.

The present invention is based on the inventor's surprising finding that a pharmaceutical composition comprising at least one nucleic acid encoding at least one peptide or protein from a Coronavirus membrane protein (M), nucleocapsid protein (N), non-structural protein, and/or accessory protein or an immunogenic fragment or immunogenic variant thereof can efficiently be expressed in human cells. Even more surprising and unexpected, the administration of such a pharmaceutical compositions, optionally additionally comprising at least one nucleic acid sequence encoding a Coronavirus spike protein, induces antigen-specific immune responses against the encoded Coronavirus antigen, including T-cell responses.

Those findings are the basis for a nucleic acid based vaccine of the invention that provides protection against at least one Coronavirus, e.g. a pandemic Coronavirus, preferably SARS-CoV-2.

In a first aspect, the present invention provides pharmaceutical compositions comprising or consisting of at least one nucleic acid comprising at least one coding sequence encoding at least one antigenic peptide or protein from at least one Coronavirus, wherein the at least one antigenic peptide or protein is selected or derived from membrane protein (M), nucleocapsid protein (N), envelope protein (E), non-structural protein, and/or accessory protein or an immunogenic fragment or immunogenic variant thereof.

Suitably, the composition additionally comprises at least one nucleic acid comprising at least one coding sequence encoding at least one antigenic peptide or protein selected or derived from at least one Coronavirus spike protein (S), or an immunogenic fragment or immunogenic variant thereof

The at least one Coronavirus may suitably be selected from a pandemic Coronavirus, e.g. SARS-CoV-1, SARS-CoV-2, MERS-CoV. Preferably, the Coronavirus is selected from SARS-CoV-2.

In a second aspect, the present invention provides vaccines, preferably multivalent Coronavirus vaccines, wherein the vaccines comprise the pharmaceutical compositions as defined in the first aspect.

In a third aspect, the present invention provides kits or kits of parts comprising at least one pharmaceutical composition of first aspect, and/or at least one vaccine of the second aspect.

Further aspects of the invention concern methods of treating or preventing Coronavirus infections in a subject, and first and second medical uses of the pharmaceutical compositions, the vaccines, or the kits. Also provided are methods of manufacturing the pharmaceutical compositions, or the vaccines.

### DETAILED DESCRIPTION OF THE INVENTION

The present application is filed together with a sequence listing in electronic format, which is part of the description of the present application (WIPO standard ST.25). The information contained in the sequence listing is incorporated herein by reference in its entirety. Where reference is made herein to a “SEQ ID NO”, the corresponding nucleic acid sequence or amino acid (aa) sequence in the sequence listing having the respective identifier is referred to. For many sequences, the sequence listing also provides additional detailed information, e.g. regarding certain structural features, sequence optimizations, GenBank (NCBI) or GISAID (epi) identifiers, or additional detailed information regarding its coding capacity. In particular, such information is provided under numeric identifier <223> in the WIPO standard ST.25 sequence listing. Accordingly, information provided under said numeric identifier <223> is explicitly included herein in its entirety and has to be understood as integral part of the description of the underlying invention. Where reference is made to SEQ ID NOs of published patent applications, said nucleic acid or amino acid sequences are specifically included herein. Also for those referenced sequences, information provided under numeric identifier <223> in the WIPO standard ST.25 sequence listing of the referenced sequences is included herein.

Where reference is made to “SEQ ID NOs” of other published patent applications or patents, said sequences, e.g. amino acid sequences or nucleic acid sequences, are explicitly incorporated herein by reference. For “SEQ ID NOs” that are included by reference, information provided under numeric identifier <223> (of the respective sequence protocol) is also explicitly included herein in its entirety.

Pharmaceutical Composition:

In a first aspect, the invention relates to a pharmaceutical composition suitable fora Coronavirus vaccine.

It has to be noted that specific features and embodiments that are described in the context of the first aspect of the invention, that is the pharmaceutical composition of the invention, are likewise applicable to the second aspect (vaccine of the invention), the third aspect (kit or kit of parts of the invention), or further aspects including e.g. medical uses (first and second medical uses) and e.g. method of treatments.

A nucleic acid according to the invention, e.g. the DNA or the RNA, forms the basis for a nucleic acid based pharmaceutical composition or a nucleic acid based vaccine.

Such nucleic acid based pharmaceutical composition (first aspect) or nucleic acid based vaccines (second aspect) as provided herein have advantages over classical vaccine approaches: In general, protein-based vaccines, or live attenuated vaccines are suboptimal for use in developing countries due to their high production costs. In addition, protein-based vaccines, or live attenuated vaccines require long development times and are not suitable for rapid responses of pandemic virus outbreaks such as e.g. the Coronavirus SARS-CoV-2 outbreak in 2019/2020. Furthermore, using classical approaches it remains to be a challenge to provide a multivalent vaccine that is effective against a Coronavirus.

In contrast, the nucleic acid-based pharmaceutical compositions and vaccines according to the present invention allow very fast and cost-effective manufacturing. Therefore, in comparison with known vaccines, vaccine based on the inventive nucleic acid can be produced and manufactured significantly cheaper and faster, which is very advantageous particularly for use in developing countries or in the context of a global pandemic.

One further advantage of a pharmaceutical compositions or vaccines based on nucleic acid is that the nucleic acid components are temperature-stable in comparison to e.g. protein or peptide-based vaccines.

Moreover, the inventors found that respective nucleic acid sequences encoding Coronavirus antigens can be combined to improve specific immune responses, e.g. T-cell responses, B-cell responses, neutralizing immune responses.

Nucleic Acid Encoding a Coronavirus Antigenic Peptide or Protein

In various preferred embodiments, the pharmaceutical composition comprises at least one nucleic acid comprising at least one coding sequence encoding at least one antigenic peptide or protein selected or derived from at least one Coronavirus, or an immunogenic fragment or immunogenic variant thereof.

The term “antigenic peptide or protein of a Coronavirus” relates to any peptide or protein that is selected or is derived from the respective Coronavirus as defined herein, but also to fragments, variants or derivatives thereof, preferably to immunogenic fragments or immunogenic variants thereof.

The term “immunogenic fragment” or “immunogenic variant” has to be understood as any fragment/variant of the corresponding Coronavirus antigen that is capable of raising an immune response in a subject.

Coronaviruses can be classified into the genus Alphacoronavirus, Betacoronavirus, Deltacoronavirus, Gammacoronavirus, and unclassified Coronaviruses. Coronaviruses are genetically highly variable, and individual virus species can also infect several host species by overcoming the species barrier, to potentially become pandemic.

In preferred embodiments the at least one Coronavirus is selected or derived from at least one pandemic Coronavirus.

In embodiments the at least one Coronavirus, or the at least one pandemic Coronavirus, is selected from at least one Alphacoronavirus, at least one Betacoronavirus, at least one Gammacoronavirus, and/or at least one Deltacoronavirus, preferably a pandemic Alphacoronavirus, Betacoronavirus, Gammacoronavirus, Deltacoronavirus.

In embodiments, the at least one Coronavirus, or the at least one pandemic Coronavirus, is a Betacoronavirus. Suitably, the Betacoronavirus is selected from at least one Sarbecovirus, at least one Merbecovirus, at least one Embecovirus, at least one Nobecovirus, and/or at least one Hibecovirus.

In preferred embodiments, the at least one Coronavirus, or the at least one pandemic Coronavirus, is a Betacoronavirus, preferably a Sarbecovirus. In the context of the invention, a preferred Sarbecovirus may be selected from a SARS-associated Coronavirus.

Preferred SARS-associated Coronaviruses can be selected from SARS-CoV-2 and/or SARS-CoV-1.

SARS-CoV associated viruses belong to the Coronaviridae, in particular to Orthocoronaviruses, more specifically to the genus Betacoronavirus. SARS-CoV-1 (severe acute respiratory syndrome coronavirus, SARS-Coronavirus, SCV) causes a severe respiratory syndrome disease (SARS). An exemplary SARS-CoV-1 coronaviruses is identifiable by NCBI Taxonomy: 694009, NCBI Reference: DQ182595.1. Further suitable SARS-associated viruses in the context of the invention are SARS-CoV/Tor2, HCoV/OC43, HCoV/HKU1/N5, HCoV/229E/BN1/GER/2015, HCoV/NL63/RPTEC/2004, Bat SARS-like CoV/WIV1, BatCoV/HKU9-1 BF_005I, PDCoV/Swine/Thailand/S5011/2015, PEDV/NPL-PEDv/2013/P10, PEDV/NPL-PEDv/2013/P10, or MHV/S.

As used herein, the terms “SARS-CoV-2”, “Human coronavirus 2019”, “Wuhan Human coronavirus” (WHCV), “nCoV-2019 coronavirus”, “nCoV-2019”, “Wuhan seafood market pneumonia virus”, “Wuhan coronavirus”, “WHCV coronavirus”, “HCoV-19”, “SARS2”, “COVID-19 virus”, “hCoV-19”, or “coronavirus SARS-CoV-2” may be used interchangeable throughout the present invention, relating to a new pandemic coronavirus that has been emerged in the Chinese city of Wuhan at the turn of 2019/2020, causing the disease COVID-19. According to the WHO (February 2020), the virus is officially termed “SARS-CoV-2”, and the associated disease is officially termed “COVID-19”.

SARS-CoV-2 belongs to the Coronaviridae, in particular to Orthocoronaviruses, more specifically to the genus Betacoronavirus. Exemplary SARS-CoV-2 coronaviruses are isolates including but not limited to those provided in List A and List B below.

List A: Exemplary SARS-CoV-2 Coronavirus Isolates (EPI/GISAID):
EPI_ISL_402119, EPI_ISL_402120, EPI_ISL_402121, EPI_ISL_402123, EPI_ISL_402124 (hCoV-19/Wuhan_WIV04/2019), EPI_ISL_402125, EPI_ISL_402127, EPI_ISL_402128 (BetaCoV_Wuhan_WIV05_2019_EPI_ISL_402128), EPI_ISL_402129, EPI_ISL_402130, EPI_ISL_402131, EPI_ISL_402132, EPI_ISL_403928, EPI_ISL_403929, EPI_ISL_403930, EPI_ISL_403931, EPI_ISL_403932, EPI_ISL_403933, EPI_ISL_403934, EPI_ISL_403935, EPI_ISL_403936, EPI_ISL_403937, EPI_ISL_403962, EPI_ISL_403963, EPI_ISL_404227, EPI_ISL_404228, EPI_ISL_404253, EPI_ISL_404895, EPI_ISL_405839, EPI_ISL_406030, EPI_ISL_406031, EPI_ISL_406034, EPI_ISL_406036, EPI_ISL_406223, EPI_ISL_406531, EPI_ISL_406533, EPI_ISL_406534, EPI_ISL_406535, EPI_ISL_406536, EPI_ISL_406538, EPI_ISL_406592, EPI_ISL_406593, EPI_ISL_406594, EPI_ISL_406595, EPI_ISL_406596, EPI_ISL_406597, EPI_ISL_406798, EPI_ISL_406800, EPI_ISL_406801, EPI_ISL_406844, EPI_ISL_406862, EPI_ISL_406716, EPI_ISL_406717, EPI_ISL_406970, EPI_ISL_406973, EPI_ISL_407071, EPI_ISL_407073, EPI_ISL_407079, EPI_ISL_407084, EPI_ISL_407193, EPI_ISL_407214, EPI_ISL_407215, EPI_ISL_407313, EPI_ISL_407893, EPI_ISL_407894, EPI_ISL_407896, EPI_ISL_407976, EPI_ISL_407987, EPI_ISL_407988, EPI_ISL_408008, EPI_ISL_408009, EPI_ISL_408010, EPI_ISL_408430, EPI_ISL_408431, EPI_ISL_408478, EPI_ISL_408479, EPI_ISL_408480, EPI_ISL_408481, EPI_ISL_408482, EPI_ISL_408484, EPI_ISL_408486, EPI_ISL_408488, EPI_ISL_408489, EPI_ISL_408514, EPI_ISL_408515, EPI_ISL_408665, EPI_ISL_408666, EPI_ISL_408667, EPI_ISL_408668, EPI_ISL_408669, EPI_ISL_408670, EPI_ISL_408976, EPI_ISL_408977 EPI_ISL_409067, EPI_ISL_410044, EPI_ISL_410045, EPI_ISL_410218, EPI_ISL_410301, EPI_ISL_410486, EPI_ISL_410531, EPI_ISL_410532, EPI_ISL_410535, EPI_ISL_410536, EPI_ISL_410537, EPI_ISL_410538, EPI_ISL_410539, EPI_ISL_410540, EPI_ISL_410541, EPI_ISL_410542, EPI_ISL_410713, EPI_ISL_410714, EPI_ISL_410715, EPI_ISL_410716, EPI_ISL_410717, EPI_ISL_410718, EPI_ISL_410719, EPI_ISL_410720, EPI_ISL_410984, EPI_ISL_411060, EPI_ISL_411066, EPI_ISL_411218, EPI_ISL_411219, EPI_ISL_411220, EPI_ISL_411902, EPI_ISL_411915, EPI_ISL_411926, EPI_ISL_411927, EPI_ISL_411929, EPI_ISL_411950, EPI_ISL_411951, EPI_ISL_411952, EPI_ISL_411953, EPI_ISL_411954, EPI_ISL_411955, EPI_ISL_411956, EPI_ISL_411957, EPI_ISL_412026, EPI_ISL_412028, EPI_ISL_412029, EPI_ISL_412030, EPI_ISL_412459, EPI_ISL_412862, EPI_ISL_412869, EPI_ISL_412870, EPI_ISL_412871, EPI_ISL_412872, EPI_ISL_412873, EPI_ISL_412898, EPI_ISL_412899, EPI_ISL_412912, EPI_ISL_412966, EPI_ISL_412967, EPI_ISL_412968, EPI_ISL_412969, EPI_ISL_412970, EPI_ISL_412972, EPI_ISL_412973, EPI_ISL_412974, EPI_ISL_412975, EPI_ISL_412978, EPI_ISL_412979, EPI_ISL_412980, EPI_ISL_412981, EPI_ISL_412982, EPI_ISL_412983, EPI_ISL_413014, EPI_ISL_413015, EPI_ISL_413016, EPI_ISL_413017, EPI_ISL_413018, EPI_ISL_413021, EPI_ISL_413022, EPI_ISL_413023, EPI_ISL_413024, EPI_ISL_413213, EPI_ISL_413214, EPI_ISL_413455, EPI_ISL_413456, EPI_ISL_413457, EPI_ISL_413458, EPI_ISL_413459, EPI_ISL_413485, EPI_ISL_413486, EPI_ISL_413488, EPI_ISL_413489, EPI_ISL_413490, EPI_ISL_413513, EPI_ISL_413514, EPI_ISL_413515, EPI_ISL_413516, EPI_ISL_413518, EPI_ISL_413519, EPI_ISL_413520, EPI_ISL_413521, EPI_ISL_413522, EPI_ISL_413523, EPI_ISL_413555, EPI_ISL_413557, EPI_ISL_413558, EPI_ISL_413559, EPI_ISL_413560, EPI_ISL_413562, EPI_ISL_413563, EPI_ISL_413566, EPI_ISL_413572, EPI_ISL_413573, EPI_ISL_413577, EPI_ISL_413579, EPI_ISL_413580, EPI_ISL_413581, EPI_ISL_413582, EPI_ISL_413583, EPI_ISL_413584, EPI_ISL_413587, EPI_ISL_413589, EPI_ISL_413590, EPI_ISL_413591, EPI_ISL_413592, EPI_ISL_413593, EPI_ISL_413594, EPI_ISL_413595, EPI_ISL_413596, EPI_ISL_413597, EPI_ISL_413598, EPI_ISL_413599, EPI_ISL_413600, EPI_ISL_413602, EPI_ISL_413603, EPI_ISL_413604, EPI_ISL_413606, EPI_ISL_413607, EPI_ISL_413608, EPI_ISL_413609, EPI_ISL_413610, EPI_ISL_413611, EPI_ISL_413612, EPI_ISL_413613, EPI_ISL_413614, EPI_ISL_413615, EPI_ISL_413616, EPI_ISL_413617, EPI_ISL_413618, EPI_ISL_413619, EPI_ISL_413620, EPI_ISL_413621, EPI_ISL_413622, EPI_ISL_413647, EPI_ISL_413648, EPI_ISL_413691, EPI_ISL_413692, EPI_ISL_413693, EPI_ISL_413694, EPI_ISL_413697, EPI_ISL_413711, EPI_ISL_413729, EPI_ISL_413746, EPI_ISL_413748, EPI_ISL_413749, EPI_ISL_413750, EPI_ISL_413751, EPI_ISL_413761, EPI_ISL_413791, EPI_ISL_413809, EPI_ISL_413852, EPI_ISL_413853, EPI_ISL_413854, EPI_ISL_413856, EPI_ISL_413857, EPI_ISL_413858, EPI_ISL_413860, EPI_ISL_413861, EPI_ISL_413862, EPI_ISL_413863, EPI_ISL_413928, EPI_ISL_413931, EPI_ISL_413996, EPI_ISL_413997, EPI_ISL_413999, EPI_ISL_414005, EPI_ISL_414006, EPI_ISL_414007, EPI_ISL_414008, EPI_ISL_414009, EPI_ISL_414011, EPI_ISL_414012, EPI_ISL_414019, EPI_ISL_414020, EPI_ISL_414021, EPI_ISL_414022, EPI_ISL_414023, EPI_ISL_414027, EPI_ISL_414040, EPI_ISL_414041, EPI_ISL_414042, EPI_ISL_414043, EPI_ISL_414044, EPI_ISL_414045, EPI_ISL_414363, EPI_ISL_414366, EPI_ISL_414367, EPI_ISL_414368, EPI_ISL_414369, EPI_ISL_414414, EPI_ISL_414423, EPI_ISL_414428, EPI_ISL_414429, EPI_ISL_414433, EPI_ISL_414435, EPI_ISL_414439, EPI_ISL_414443, EPI_ISL_414445, EPI_ISL_414446, EPI_ISL_414451, EPI_ISL_414457, EPI_ISL_414468, EPI_ISL_414470, EPI_ISL_414476, EPI_ISL_414477, EPI_ISL_414479, EPI_ISL_414480, EPI_ISL_414481, EPI_ISL_414482, EPI_ISL_414483, EPI_ISL_414484, EPI_ISL_414485, EPI_ISL_414487, EPI_ISL_414500, EPI_ISL_414505, EPI_ISL_414509, EPI_ISL_414510, EPI_ISL_414511, EPI_ISL_414517, EPI_ISL_414519, EPI_ISL_414520, EPI_ISL_414521, EPI_ISL_414522, EPI_ISL_414523, EPI_ISL_414524, EPI_ISL_414525, EPI_ISL_414526, EPI_ISL_414527, EPI_ISL_414528, EPI_ISL_414529, EPI_ISL_414530, EPI_ISL_414531, EPI_ISL_414532, EPI_ISL_414534, EPI_ISL_414535, EPI_ISL_414545, EPI_ISL_414546, EPI_ISL_414547, EPI_ISL_414548, EPI_ISL_414549, EPI_ISL_414552, EPI_ISL_414554, EPI_ISL_414555, EPI_ISL_414556, EPI_ISL_414557, EPI_ISL_414558, EPI_ISL_414559, EPI_ISL_414560, EPI_ISL_414561, EPI_ISL_414562, EPI_ISL_414564, EPI_ISL_414565, EPI_ISL_414566, EPI_ISL_414569, EPI_ISL_414571, EPI_ISL_414574, EPI_ISL_414577, EPI_ISL_414578, EPI_ISL_414579, EPI_ISL_414580, EPI_ISL_414586, EPI_ISL_414587, EPI_ISL_414588, EPI_ISL_414589, EPI_ISL_414590, EPI_ISL_414591, EPI_ISL_414592, EPI_ISL_414593, EPI_ISL_414594, EPI_ISL_414595, EPI_ISL_414596, EPI_ISL_414597, EPI_ISL_414600, EPI_ISL_414601, EPI_ISL_414616, EPI_ISL_414617, EPI_ISL_414618, EPI_ISL_414619, EPI_ISL_414620, EPI_ISL_414621, EPI_ISL_414622, EPI_ISL_414623, EPI_ISL_414624, EPI_ISL_414625, EPI_ISL_414626, EPI_ISL_414627, EPI_ISL_414628, EPI_ISL_414629, EPI_ISL_414630, EPI_ISL_414631, EPI_ISL_414632, EPI_ISL_414633, EPI_ISL_414635, EPI_ISL_414637, EPI_ISL_414638, EPI_ISL_414641, EPI_ISL_414642, EPI_ISL_414643, EPI_ISL_414646, EPI_ISL_414648, EPI_ISL_414663, EPI_ISL_414684, EPI_ISL_414685, EPI_ISL_414686, EPI_ISL_414687, EPI_ISL_414688, EPI_ISL_414689, EPI_ISL_414690, EPI_ISL_414691, EPI_ISL_414692, EPI_ISL_414936, EPI_ISL_414937, EPI_ISL_414938, EPI_ISL_414940, EPI_ISL_414941, EPI_ISL_415105, EPI_ISL_415128, EPI_ISL_415129, EPI_ISL_415136, EPI_ISL_415141, EPI_ISL_415142, EPI_ISL_415147, EPI_ISL_415150, EPI_ISL_415151, EPI_ISL_415152, EPI_ISL_415153, EPI_ISL_415154, EPI_ISL_415155, EPI_ISL_415156, EPI_ISL_415157, EPI_ISL_415158, EPI_ISL_415159, EPI_ISL_415710, EPI_ISL_416426, EPI_ISL_416457, EPI_ISL_416481, EPI_ISL_416489, EPI_ISL_416491, EPI_ISL_416492, EPI_ISL_416514, EPI_ISL_416515, EPI_ISL_416516, EPI_ISL_416517, EPI_ISL_416518, EPI_ISL_416538, EPI_ISL_416539, EPI_ISL_416683, EPI_ISL_416685, EPI_ISL_416704, EPI_ISL_416711, EPI_ISL_416713, EPI_ISL_416715, EPI_ISL_416717, EPI_ISL_416744, EPI_ISL_416830, EPI_ISL_416831, EPI_ISL_416832, EPI_ISL_417020, EPI_ISL_417021, EPI_ISL_417022, EPI_ISL_417023, EPI_ISL_417024, EPI_ISL_417025, EPI_ISL_417026, EPI_ISL_417027, EPI_ISL_417028, EPI_ISL_417034, EPI_ISL_417200, EPI_ISL_417201, EPI_ISL_417202, EPI_ISL_417203, EPI_ISL_417204, EPI_ISL_417374, EPI_ISL_417375, EPI_ISL_417376, EPI_ISL_417377, EPI_ISL_417379, EPI_ISL_417382, EPI_ISL_417408, EPI_ISL_417409, EPI_ISL_417410, EPI_ISL_417411, EPI_ISL_417412, EPI_ISL_417413, EPI_ISL_417420, EPI_ISL_417435, EPI_ISL_417436, EPI_ISL_417437, EPI_ISL_417438, EPI_ISL_417439, EPI_ISL_417440, EPI_ISL_417441, EPI_ISL_417442, EPI_ISL_417467, EPI_ISL_417468, EPI_ISL_417504, EPI_ISL_417505, EPI_ISL_417506, EPI_ISL_417507, EPI_ISL_417508, EPI_ISL_417509, EPI_ISL_417510, EPI_ISL_417512, EPI_ISL_417513, EPI_ISL_417514, EPI_ISL_417515, EPI_ISL_417516, EPI_ISL_417517, EPI_ISL_417526, EPI_ISL_417527, EPI_ISL_417528, EPI_ISL_417529, EPI_ISL_417530, EPI_ISL_417531, EPI_ISL_417532, EPI_ISL_417533, EPI_ISL_417534, EPI_ISL_417536, EPI_ISL_417537, EPI_ISL_417538, EPI_ISL_417539, EPI_ISL_417540, EPI_ISL_417541, EPI_ISL_417542, EPI_ISL_417543, EPI_ISL_417544, EPI_ISL_417545, EPI_ISL_417546, EPI_ISL_417547, EPI_ISL_417548, EPI_ISL_417550, EPI_ISL_417551, EPI_ISL_417552, EPI_ISL_417553, EPI_ISL_417554, EPI_ISL_417555, EPI_ISL_417556, EPI_ISL_417557, EPI_ISL_417558, EPI_ISL_417559, EPI_ISL_417560, EPI_ISL_417561, EPI_ISL_417562, EPI_ISL_417563, EPI_ISL_417564, EPI_ISL_417565, EPI_ISL_417566, EPI_ISL_417567, EPI_ISL_417568, EPI_ISL_417569, EPI_ISL_417570, EPI_ISL_417571, EPI_ISL_417572, EPI_ISL_417573, EPI_ISL_417574, EPI_ISL_417575, EPI_ISL_417576, EPI_ISL_417577, EPI_ISL_417578, EPI_ISL_417579, EPI_ISL_417580, EPI_ISL_417581, EPI_ISL_417582, EPI_ISL_417583, EPI_ISL_417584, EPI_ISL_417585, EPI_ISL_417586, EPI_ISL_417587, EPI_ISL_417588, EPI_ISL_417589, EPI_ISL_417590, EPI_ISL_417591, EPI_ISL_417592, EPI_ISL_417593, EPI_ISL_417594, EPI_ISL_417595, EPI_ISL_417596, EPI_ISL_417597, EPI_ISL_417598, EPI_ISL_417599, EPI_ISL_417600, EPI_ISL_417601, EPI_ISL_417602, EPI_ISL_417603, EPI_ISL_417604, EPI_ISL_417605, EPI_ISL_417606, EPI_ISL_417607, EPI_ISL_417608, EPI_ISL_417609, EPI_ISL_417610, EPI_ISL_417611, EPI_ISL_417612, EPI_ISL_417613, EPI_ISL_417614, EPI_ISL_417615, EPI_ISL_417616, EPI_ISL_417617, EPI_ISL_417618, EPI_ISL_417619, EPI_ISL_417620, EPI_ISL_417621, EPI_ISL_417622, EPI_ISL_417623, EPI_ISL_417624, EPI_ISL_417625, EPI_ISL_417626, EPI_ISL_417627, EPI_ISL_417628, EPI_ISL_417629, EPI_ISL_417630, EPI_ISL_417631, EPI_ISL_417632, EPI_ISL_417633, EPI_ISL_417634, EPI_ISL_417635, EPI_ISL_417636, EPI_ISL_417637, EPI_ISL_417638, EPI_ISL_417639, EPI_ISL_417640, EPI_ISL_417641, EPI_ISL_417642, EPI_ISL_417643, EPI_ISL_417644, EPI_ISL_417645, EPI_ISL_417646, EPI_ISL_417647, EPI_ISL_417648, EPI_ISL_417649, EPI_ISL_417650, EPI_ISL_417651, EPI_ISL_417652, EPI_ISL_417653, EPI_ISL_417654, EPI_ISL_417666, EPI_ISL_417667, EPI_ISL_417668, EPI_ISL_417669, EPI_ISL_417670, EPI_ISL_417671, EPI_ISL_417672, EPI_ISL_417676, EPI_ISL_417678, EPI_ISL_417680, EPI_ISL_417685, EPI_ISL_417699, EPI_ISL_417700, EPI_ISL_417703, EPI_ISL_417706, EPI_ISL_417709, EPI_ISL_417712, EPI_ISL_417716, EPI_ISL_417717, EPI_ISL_417724, EPI_ISL_417733, EPI_ISL_417737, EPI_ISL_417740, EPI_ISL_417742, EPI_ISL_417743, EPI_ISL_417746, EPI_ISL_417750, EPI_ISL_417752, EPI_ISL_417753, EPI_ISL_417754, EPI_ISL_417762, EPI_ISL_417763, EPI_ISL_417764, EPI_ISL_417766, EPI_ISL_417774, EPI_ISL_417808, EPI_ISL_417809, EPI_ISL_417813, EPI_ISL_417814, EPI_ISL_417815, EPI_ISL_417816, EPI_ISL_417818, EPI_ISL_417819, EPI_ISL_417820, EPI_ISL_417821, EPI_ISL_417822, EPI_ISL_417823, EPI_ISL_417824, EPI_ISL_417825, EPI_ISL_417826, EPI_ISL_417827, EPI_ISL_417829, EPI_ISL_417830, EPI_ISL_417831, EPI_ISL_417832, EPI_ISL_417833, EPI_ISL_417834, EPI_ISL_417835, EPI_ISL_417836, EPI_ISL_417837, EPI_ISL_417838, EPI_ISL_417839, EPI_ISL_417864, EPI_ISL_417917, EPI_ISL_417918, EPI_ISL_417920, EPI_ISL_417925, EPI_ISL_417926, EPI_ISL_417931, EPI_ISL_417932, EPI_ISL_417933, EPI_ISL_417935, EPI_ISL_417936, EPI_ISL_417937, EPI_ISL_417938, EPI_ISL_417939, EPI_ISL_417940, EPI_ISL_417941, EPI_ISL_417942, EPI_ISL_417943, EPI_ISL_417944, EPI_ISL_417945, EPI_ISL_417946, EPI_ISL_417947, EPI_ISL_417948, EPI_ISL_417949, EPI_ISL_417950, EPI_ISL_417951, EPI_ISL_417953, EPI_ISL_417955, EPI_ISL_417958, EPI_ISL_417959, EPI_ISL_417960, EPI_ISL_417962, EPI_ISL_417964, EPI_ISL_417965, EPI_ISL_417966, EPI_ISL_417968, EPI_ISL_417970, EPI_ISL_417971, EPI_ISL_417973, EPI_ISL_417974, EPI_ISL_417976, EPI_ISL_417977, EPI_ISL_417982, EPI_ISL_417983, EPI_ISL_417984, EPI_ISL_417985, EPI_ISL_418009, EPI_ISL_418017, EPI_ISL_418018, EPI_ISL_418019, EPI_ISL_418020, EPI_ISL_418021, EPI_ISL_418022, EPI_ISL_418023, EPI_ISL_418024, EPI_ISL_418025, EPI_ISL_418026, EPI_ISL_418027, EPI_ISL_418029, EPI_ISL_418030, EPI_ISL_418031, EPI_ISL_418032, EPI_ISL_418033, EPI_ISL_418034, EPI_ISL_418037, EPI_ISL_418038, EPI_ISL_418040, EPI_ISL_418046, EPI_ISL_418047, EPI_ISL_418048, EPI_ISL_418050, EPI_ISL_418052, EPI_ISL_418053, EPI_ISL_418054, EPI_ISL_418063, EPI_ISL_418064, EPI_ISL_418067, EPI_ISL_418071, EPI_ISL_418072, EPI_ISL_418073, EPI_ISL_418074, EPI_ISL_418075, EPI_ISL_418076, EPI_ISL_418077, EPI_ISL_418078, EPI_ISL_418079, EPI_ISL_418080, EPI_ISL_418081, EPI_ISL_418082, EPI_ISL_418101, EPI_ISL_418102, EPI_ISL_418103, EPI_ISL_418104, EPI_ISL_418105, EPI_ISL_418126, EPI_ISL_418127, EPI_ISL_418128, EPI_ISL_418129, EPI_ISL_418130, EPI_ISL_418131, EPI_ISL_418132, EPI_ISL_418133, EPI_ISL_418134, EPI_ISL_418135, EPI_ISL_418136, EPI_ISL_418137, EPI_ISL_418138, EPI_ISL_418139, EPI_ISL_418140, EPI_ISL_418148, EPI_ISL_418149, EPI_ISL_418150, EPI_ISL_418151, EPI_ISL_418152, EPI_ISL_418153, EPI_ISL_418154, EPI_ISL_418155, EPI_ISL_418156, EPI_ISL_418157, EPI_ISL_418158, EPI_ISL_418159, EPI_ISL_418160, EPI_ISL_418161, EPI_ISL_418162, EPI_ISL_418163, EPI_ISL_418164, EPI_ISL_418165, EPI_ISL_418183, EPI_ISL_418184, EPI_ISL_418185, EPI_ISL_418186, EPI_ISL_418187, EPI_ISL_418188, EPI_ISL_418189, EPI_ISL_418190, EPI_ISL_418191, EPI_ISL_418192, EPI_ISL_418193, EPI_ISL_418194, EPI_ISL_418195, EPI_ISL_418197, EPI_ISL_418198, EPI_ISL_418199, EPI_ISL_418200, EPI_ISL_418201, EPI_ISL_418202, EPI_ISL_418203, EPI_ISL_418204, EPI_ISL_418231, EPI_ISL_418232, EPI_ISL_418233, EPI_ISL_418235, EPI_ISL_418236, EPI_ISL_418237, EPI_ISL_418238, EPI_ISL_418239, EPI_ISL_418240, EPI_ISL_418257, EPI_ISL_418260, EPI_ISL_418263, EPI_ISL_418264 or EPI_ISL_418265 or EPI_ISL_616802 (hCoV-19/Denmark/DCGC-3024/2020).

Exemplary SARS-CoV-2 coronaviruses can also be defined or identified by genetic information provided by GenBank Accession Numbers as provided in List B below.

List B: GenBank Accession Numbers of Different SARS-CoV-2 Isolates:
NC_045512, LC528232, LC528233, LC529905, MN908947, MN938384, MN938385, MN938386, MN938387, MN938388, MN938389, MN938390, MN970003, MN970004, MN975262, MN975263, MN975264, MN975265, MN975266, MN975267, MN975268, MN985325, MN988668, MN988669, MN994467, MN994468, MN996527, MN996528, MN996529, MN996530, MN996531, MN997409, MT007544, MT012098, MT019529, MT019530, MT019531, MT019532, MT019533, MT020880, MT020881, MT027062, MT027063, MT027064, MT039873, MT039887, MT039888, MT039890, MT044257, MT044258, MT049951, MT050493, MT066156, MT066175, MT066176, MT072688, MT093571, MT093631, MT106052, MT106053, MT106054, MT118835, MT121215, MT123290, MT123291, MT123292, MT123293, MT126808, MT135041, MT135042, MT135043, MT135044, MT152824, MT159705, MT159706, MT159707, MT159708, MT159709, MT159710, MT159711, MT159712, MT159713, MT159714, MT159715, MT159716, MT159717, MT159718, MT159719, MT159720, MT159721, MT159722, MT163716, MT163717, MT163718, MT163719, MT163720, MT163721, MT184907, MT184908, MT184909, MT184910, MT184911, MT184912, MT184913, MT188339, MT188340, MT188341, MT192759, MT192765, MT192772 or MT192773.

SARS-CoV-2 coronavirus has been attributed the NCBI Taxonomy ID (NCBR:txid or taxID): 2697049.

In preferred embodiments, the at least one SARS-CoV-2 is a SARS-Cov-2 SARS-CoV-2 isolate, SARS-CoV-2 variant or a SARS-CoV-2 variant strain or a SARS-CoV-2 lineage. In particularly preferred embodiments, the SARS-CoV-2 variant is selected from or is derived from the following SARS-CoV-2 lineages: B.1.351 (South Africa), B.1.1.7 (UK), P.1 (Brazil), B.1.429 (California), B.1.525 (Nigeria), B.1.258 (Czech republic), B.1.526 (New York), A.23.1 (Uganda), B.1.617.1 (India), B.1.617.2 (India), 8.1.617.3 (India), P.2 (Brazil), C37.1 (Peru).

In a preferred embodiments, the at least one Coronavirus, or the at least one pandemic Coronavirus, is a Betacoronavirus, preferably a Merbecovirus. In the context of the invention, a preferred Merbecovirus may be selected from a MERS-associated coronavirus. Preferred MERS-associated Coronaviruses can be selected from MERS-CoV.

MERS-CoV belongs to the Coronaviridae, in particular to Orthocoronaviruses, more specifically to the genus Betacoronavirus. MERS-CoV (Middle East respiratory syndrome coronavirus, MERS-Coronavirus, EMC/2012 (HCoV-EMC/2012)) causes a severe respiratory syndrome disease. An exemplary MERS-CoV coronaviruses is identifiable by NCBI Taxonomy: 1335626, NCBI Reference: NC_038294.1. Suitable MERS-CoV strains/isolates may be selected from MERS-CoV/MERS-CoV-Jeddah-human-1, MERS-CoV/AI-Hasa_4_2013, MERS-CoV/Riyadh_14_2013, MERS-CoV/Riyadh_14_2013, MERS-CoV/Riyadh_14_2013 spike protein, MERS-CoV/England 1 spike protein, MERS-CoV/England 1 spike protein (variant).

In the context of the invention, any protein, preferably any membrane protein (M), nucleocapsid protein (N), non-structural protein (NSP), accessory protein, envelope protein (E), or Spike protein (S), or an immunogenic fragment or immunogenic variant thereof selected or derived from a Coronavirus, preferably a pandemic Coronavirus, may be used in the context of the invention and may be suitably encoded by the coding sequence or the nucleic acid.

It is further in the scope of the underlying invention, that the at least one antigenic peptide or protein may comprise or consist of a synthetically engineered or an artificial Coronavirus peptide or protein. The term “synthetically engineered” Coronavirus peptide or protein, or the term “artificial Coronavirus peptide or protein” relates to a protein that does not occur in nature. Accordingly, an “artificial Coronavirus peptide or protein” or a “synthetically engineered Coronavirus peptide or protein” may for example differ in at least one amino acid compared to the naturally existing Coronavirus peptide or protein, and/or may comprise an additional peptide or protein element (e.g. a heterologous element), and/or may be N-terminally or C-terminally extended or truncated.

According to various preferred embodiments, the nucleic acid of encodes at least one antigenic peptide or protein from Coronavirus as defined herein, preferably of a pandemic Coronavirus, and, additionally, at least one heterologous peptide or protein element.

Suitably, the at least one heterologous peptide or protein element may promote or improve secretion of the encoded Coronavirus antigenic peptide or protein (e.g. via secretory signal sequences), promote or improve anchoring of the encoded antigenic peptide or protein of the invention in the plasma membrane (e.g. via transmembrane elements), promote or improve formation of antigen complexes (e.g. via multimerization domains or antigen clustering elements), or promote or improve virus-like particle formation (VLP forming sequence). In addition, the nucleic acid of additionally encode peptide linker elements, self-cleaving peptides, immunologic adjuvant sequences or dendritic cell targeting sequences.

Suitable multimerization domains may be selected from the list of amino acid sequences according to SEQ ID NOs: 1116-1167 of WO2017081082, or fragments or variants of these sequences. Suitable transmembrane elements may be selected from the list of amino acid sequences according to SEQ ID NOs: 1228-1343 of WO2017081082, or fragments or variants of these sequences. Suitable VLP forming sequences may be selected from the list of amino acid sequences according to SEQ ID NOs: 1168-1227 of the patent application WO2017081082, or fragments or variants of these sequences. Suitable peptide linkers may be selected from the list of amino acid sequences according to SEQ ID NOs: 1509-1565 of the patent application WO2017081082, or fragments or variants of these sequences. Suitable self-cleaving peptides may be selected from the list of amino acid sequences according to SEQ ID NOs: 1434-1508 of the patent application WO2017081082, or fragments or variants of these sequences. Suitable immunologic adjuvant sequences may be selected from the list of amino acid sequences according to SEQ ID NOs: 1360-1421 of the patent application WO2017081082, or fragments or variants of these sequences. Suitable dendritic cell (DCs) targeting sequences may be selected from the list of amino acid sequences according to SEQ ID NOs: 1344-1359 of the patent application WO2017081082, or fragments or variants of these sequences. Suitable secretory signal peptides may be selected from the list of amino acid sequences according to SEQ ID NOs: 1-1115 and SEQ ID NO: 1728 of published PCT patent application WO2017081082, or fragments or variants of these sequences

In preferred embodiments, the at least one coding sequence additionally encodes one or more heterologous peptide or protein elements selected from a signal peptide, a linker peptide, a helper epitope, an antigen clustering element, a trimerization or multimerization element, a transmembrane element, or a VLP forming sequence.

Nucleic Acid Encoding a Coronavirus M, N, NSP, Accessory Protein, and/or E

In preferred embodiments, the pharmaceutical composition comprises at least one nucleic acid comprising at least one coding sequence encoding at least one antigenic peptide or protein selected or derived from a Coronavirus, wherein the at least one antigenic peptide or protein is selected or derived from membrane protein (M), nucleocapsid protein (N), non-structural protein (NSP), accessory protein, and/or envelope protein (E), or an immunogenic fragment or immunogenic variant thereof.

Membrane protein M: The Coronavirus membrane (M) protein (ORF5 protein) is an integral membrane protein that plays an important role in viral assembly. In addition, the Coronavirus M protein has been shown to induce apoptosis. The M protein interacts with the nucleocapsid (N) protein to encapsulate the RNA genome. Exemplary Coronavirus membrane (M) proteins are e.g. SARS-CoV-1 M protein (NP_828855.1) and SARS-CoV-2 M protein (BCA87364.1 or YP_009724393.1, encoded by SARS-CoV-2 (NC_045512.2/MN908947.2/EPI_ISL_402128) according to reference SEQ ID NO:15235).

Nucleocapsid protein (N): The Coronavirus nucleocapsid (N) protein (ORF 9/9a protein) of coronaviruses is a structural protein that binds directly to viral RNA and providing stability. Furthermore, the Coronavirus nucleocapsid (N) has been found to antagonize antiviral RNAi. Exemplary Coronavirus nucleocapsid (N) proteins are e.g. SARS-CoV-1 N protein (ORF9a, NP_828858.1) and SARS-CoV-2 N protein (ORF9, BCA87368.1 or YP_009724397.2, expressed by SARS-CoV-2 (NC_045512.2/MN908947.2/EPI_ISL_402128) according to reference SEQ ID NO: 15310).

Non structural proteins (NSP): The first gene (ORF1) of Coronavirus expresses a polyprotein that is typically composed of 16 non-structural proteins (NSP) (NSPs). NSP1 (exemplary accession No of SARS-CoV-2: YP_009725297.1) is a protein (typically about 180 aa) that induces host mRNA (leader protein) cleavage. NSP2 (exemplary accession No of SARS-CoV-2: YP_009725298.1) is a protein (typically about 638 aa) that induces host mRNA (leader protein) cleavage. NSP3 (exemplary accession No of SARS-CoV-2: YP_009725299.1) is a protein (typically about 1945 aa) that has a Papain like proteinase function. NSP4 (exemplary accession No of SARS-CoV-2: YP_009725300.1) is a protein (typically about 500 aa) that is involved in Membrane rearrangement. NSP5 (exemplary accession No of SARS-CoV-2: YP_009725301.1) is a protein (typically about 306 aa) that cleaves at 11 sites of (3C-like proteinase) NSP polyprotein. NSP6 (exemplary accession No of SARS-CoV-2: YP_009725302.1) is a protein (typically about 290 aa) that generates autophagosomes. NSP7 (exemplary accession No of SARS-CoV-2: YP_009725303.1) is a protein (typically about 83 aa) that dimerizes with NSP8. NSP8 (exemplary accession No of SARS-CoV-2: YP_009725304.1) is a protein (typically about 198 aa) that stimulates the function of NSP12. NSP9 (exemplary accession No of SARS-CoV-2: YP_009725305.1) is a protein (typically about 113 aa) that binds to a helicase. NSP10 (exemplary accession No of SARS-CoV-2: YP_009725306.1) is a protein (typically about 139 aa) that stimulates the function of NSP16. NSP11 (exemplary accession No of SARS-CoV-2: YP_009725312.1) is a protein (typically about 13 aa) with unknown biological function. NSP12 (exemplary accession No of SARS-CoV-2: YP_009725307.1) is a protein (typically about 932 aa) that copies viral RNA (RNA polymerase) methylation (guanine). NSP13 (exemplary accession No of SARS-CoV-2: YP_009725308.1) is a protein (typically about 601 aa) that unwinds duplex RNA (Helicase). NSP14 (exemplary accession No of SARS-CoV-2: YP_009725309.1) is a protein (typically about 527 aa) that has a 5′-cap RNA (3′ to 5′ exonuclease, guanine N7-methyltransferase) activity. NSP15 (exemplary accession No of SARS-CoV-2: YP_009725310.1) is a protein (typically about 346 aa) that degrade RNA to (endoRNAse/endoribonuclease) to evade host defence. NSP16 (exemplary accession No of SARS-CoV-2: YP_009725311.1) is a protein (typically about 298 aa) that has a 5′-cap RNA (2-O-ribose-methyltransferase) methylation (adenine) function.

NSP3: Coronavirus NSP3 protein is a papain-like proteinase protein that possesses several conserved domains: ssRNA binding, ADPr binding, G-quadruplex binding, ssRNA binding, protease (papain-like protease), and NSP4 binding), and transmembrane domain. The papain like protease domain of NSP3 is responsible for the release of NSP1, NSP2, and NSP3 from the N-terminal region of polyproteins 1a and lab from Coronaviruses. Exemplary Coronavirus NSP3 proteins are e.g. SARS-CoV-1 NSP3 protein (NP_828862.2) and SARS-CoV-2 NSP3 protein (YP_009725299.1, encoded by SARS-CoV-2 (NC_045512.2/MN908947.2/EPI_ISL_402128) according to reference SEQ ID NO: 15733).

NSP4: Coronavirus NSP4 protein interacts with e.g. NSP3 and possibly host proteins to confer a role related to membrane rearrangement in Coronaviruses. Moreover, the interaction between NSP4 and NSP3 is essential for viral replication. Typically, NSP4 has a transmembrane domain. Exemplary Coronavirus NSP4 proteins are e.g. SARS-CoV-1 NSP4 protein (NP_904322.1) and SARS-CoV-2 NSP4 protein (YP_009725300.1, encoded by SARS-CoV-2 (NC_045512.2/MN908947.2/EPI_ISL_402128) according to reference SEQ ID NO: 16415).

NSP6: Coronavirus NSP6 protein is involved in autophagosome formation from the endoplasmic reticulum (ER). Autophagosomes facilitate assembly of replicase proteins. Furthermore, Coronavirus NSP6 may play a role in inducing membrane vesicles. Typically, NSP6 has a transmembrane domain. Exemplary Coronavirus NSP6 proteins are e.g. SARS-CoV-1 NSP6 protein (NP_828864.1) and SARS-CoV-2 NSP6 protein (YP_009725302.1, encoded by SARS-CoV-2 (NC_045512.2/MN908947.2/EPI_ISL_402128) according to reference SEQ ID NO: 16582).

NSP13: Coronavirus NSP13 protein is a multifunctional superfamily 1 helicase capable of using both dsDNA and dsRNA as substrates, in addition to working with NSP12 in viral genome replication, it is also involved in viral mRNA capping, it associates with nucleoprotein in membranous complexes. Exemplary Coronavirus NSP13 proteins are e.g. SARS-CoV-1 NSP13 protein (NP_828870.1) and SARS-CoV-2 NSP13 protein (YP_009725308.1, encoded by SARS-CoV-2 (NC_045512.2/MN908947.2/EPI_ISL_402128) according to reference SEQ ID NO: 27908).

NSP14: Coronavirus NSP14 protein has both 3′-5′ exoribonuclease (proofreading during RNA replication) and N7-guanine methyltransferase (viral mRNA capping) activities. Exemplary Coronavirus NSP13 proteins are e.g. SARS-CoV-1 NSP14 protein (NP_828871.1) and SARS-CoV-2 NSP14 protein (YP_009725309.1, encoded by SARS-CoV-2 (NC_045512.2/MN908947.2/EPI_ISL_402128) according to reference SEQ ID NO: 27909). Accessory proteins: Coronavirus accessory proteins can be selected from ORF3a, ORF3b, ORF6, ORF7a, ORF7b, ORF8, ORF8a, ORF8b, ORF9b, and/or ORF10. Coronavirus ORF3a (exemplary accession No of SARS-CoV-2: BCA87362.1) is a protein (typically about 275 aa) that is an ion channel protein. Coronavirus ORF3b (exemplary accession No of SARS-CoV-1: NP_828853.1) is a protein with as yet undescribed function. Coronavirus ORF6 (exemplary accession No of SARS-CoV-1: NP_828856.1; accession No of SARS-CoV-2: BCA87365.1) is a protein (typically about 63 aa) that plays a role in Coronavirus pathogenesis. Coronavirus ORF7a (exemplary accession No of SARS-CoV-1: NP_828857.1; accession No of SARS-CoV-2: BCA87366.1) is a type I transmembrane protein (typically about 122 aa). Coronavirus ORF7b (exemplary accession No of SARS-CoV-1: NP_849175.1; accession No of SARS-CoV-2: BCB15096.1) is a protein (typically about 44 aa) localized in the Golgi compartment. Coronavirus ORF8 (exemplary accession No of SARS-CoV-2: QJA17759.1) is a protein (typically about 121 aa) that is involved in interferon signalling. Coronavirus ORF8a (exemplary accession No of SARS-CoV-1: NP_849176.1) is a protein (typically about 39 aa) with unknown function.

Coronavirus ORF8b (exemplary accession No of SARS-CoV-1: NP_849177.1) is a protein (typically about 121 aa) that is involved in interferon signalling. Coronavirus ORF9b (exemplary accession No of SARS-CoV-1: NP_828859.1) is a protein (typically about 98 aa) with unknown function. Coronavirus ORF10 (exemplary accession No of SARS-CoV-2: BCA87369.1) is a protein (typically about 38 aa) with unknown function.

ORF3a accessory protein: Coronavirus ORF3a protein is an ion channel related to NLRP3 inflammasome activation. ORF3a interacts with TRAF3, which in turn activates ASC ubiquitination, and as a result, leads to activation of caspase 1 and IL-1R maturation. Exemplary Coronavirus ORF3a proteins are e.g. SARS-CoV-1 ORF3a protein (NP_828852.2) and SARS-CoV-2 ORF3a protein (BCA87362.1 or YP_009724391.1, encoded by SARS-CoV-2 (NC_045512.2/MN908947.2/EPI_ISL_402128) according to reference SEQ ID NO: 16684).

ORF8 accessory protein: Coronavirus ORF8 protein binds to the IRF association domain (IAD) region of interferon regulatory factor 3 (IRF3), which in turn inactivates interferon signalling. Some Coronaviruses have a single ORF8 protein while others have two ORF8 proteins (ORF8a and ORE8b). The term “Coronavirus ORF8” encompasses all Coronavirus ORF8 proteins including ORF8a proteins and ORF8b proteins. Exemplary Coronavirus ORF8 proteins are e.g. SARS-CoV-1 ORF8a (NP_849176.1) and ORF8b (NP_849177.1) and SARS-CoV-2 ORF8 protein (QJA17759.1 or YP_009724396.1, encoded by SARS-CoV-2 (NC_045512.2/MN908947.2/EPI_ISL_402128) according to reference SEQ ID NO: 16997).

Envelope protein (E): Coronavirus E envelope protein is a small integral membrane protein in coronaviruses, which can oligomerize and create an ion channel. Exemplary Coronavirus envelope (E) protein are e.g. SARS-CoV-1 E protein (NP_828854.1) and SARS-CoV-2 E protein (BCA87363.1 or YP_009724392.1, encoded by SARS-CoV-2 (NC_045512.2/MN908947.2/EPI_ISL_402128) according to reference SEQ ID NO: 15689).

In embodiments, the membrane protein (M), nucleocapsid protein (N), non-structural protein (NSP), accessory protein, and/or envelope protein (E), or an immunogenic fragment or immunogenic variant thereof, is selected or derived from a SARS-associated virus. In preferred embodiments, the membrane protein (M), nucleocapsid protein (N), non-structural protein (NSP), accessory protein, and/or envelope protein (E), or an immunogenic fragment or immunogenic variant thereof, is selected or derived from SARS-CoV-1 or SARS-CoV2, most preferably SARS-CoV2.

In further preferred embodiments, the membrane protein (M), nucleocapsid protein (N), non-structural protein (NSP), accessory protein, and/or envelope protein (E), or an immunogenic fragment or immunogenic variant thereof, is selected or derived from a SARS-CoV-2 variant.

Variant Membrane protein (M): The SARS-CoV-2 membrane (M) protein comprises at least one of the following amino acid variations (amino acid positions according to reference according to reference SEQ ID NO:15235): A2S; A2T; A2V; D3G; V10A; L17F; L17I; V23L; F28L; L29F; L34F; R44K; I48V; I52T; A63T; A63S; A69S; V70L; V70F; I76V; A81S; I82T; I82S; A85S; C86F; L87F; G89S; A98S; A104V; M109I; N121K; H125Y; L138I; H148Y; H155Y; R158L; K162N; T175M; K180R; S197N; I201V; or D209Y. In preferred embodiments the SARS-CoV-2 membrane (M) protein comprises the following amino acid variations (amino acid positions according to reference according to reference SEQ ID NO:15235): I82T; A2S_F28L_V70L; A2T_I201V; N121K_H125Y_H148Y_H155Y_K162N; A63T_H125Y; A2S_M109I_H125Y; A2V_M109I_H125Y: L17F_M109I; F28L; H125Y: A2T A104V_H125Y_H155Y R158L: R44K L138I_H155Y; I82S; A2V_L17F_H125Y_D209Y; D3G; D3G_I82T; L17I; F28L_V70F_T175M; F28L_I82T; I48V; I52T_L87F_H125Y_H155Y; A63S_V70L_A98S; V70F; or A85S_H125Y_D209Y; or C86F_A104V.

Variant Nucleocapsid protein (N): The SARS-CoV-2 nucleocapsid (N) protein comprises at least one of the following amino acid variations (amino acid positions according to reference according to reference SEQ ID NO: 15310): S2Y; D3L; D3del; P6L; Q9H; A12G; P13L; P13S; P13T; D63G; P67S; P80R; A90T; A119S; T135I; L139F; P151L; I157V; S187L; S194L; P199L; S201I; S202N; S202R; R203K; R203M; R203G; G204R; G204P; T205I; A208G; R209del; G212V; G214C; G215C; A220V; T325I; S327L; M234I; S235F; T325I; T362I; P365S; T366I; D371Y; A376T; D377Y; E378Q; R385K; T391I; A398V; D401Y; or Q418H.

In preferred embodiments the SARS-CoV-2 nucleocapsid (N) protein comprises the following amino acid variations (amino acid positions according to reference according to reference SEQ ID NO: 15310): D3L_R203K_G204R_S235F; T205I; P80R; P80R_R203K_G204R; D63G_R203M_G215C_D377Y; D63G_R203M_D377Y; T205I_T362I; S2Y_D3del_A12G_T205I; P13L_R203K_G204R_G214C; P67S_R203M_D377Y; I157V_R203K_G204R; A119S_R203K_G204R_M234I; R203K_G204R; R203K_G204R_G212V; R203K_G204R_A208G_R209del; R203M_D377Y; T205I_M234I; M234I; A12G_T205I; P13L_P199L_S202R_T205I_M234I; P13L_R203K_G204R_G214C_T366I; R203K_G204R_A208G_R209del_M234I; D3L_T205I; P13L_S201I_T205I; S187L_R203K_G204R_Q418H; S202N; R203M; R203G; T325I; D3L_P199L_R203K_G204R_T205I_S235F; P6L_M234I_D401Y; P67S_P199L_D377Y; P67S_P199L_E378Q; A90T_R203K_G204R_M234I; P151L_R203K_G204R; S194L; S194L_T205I_M234I; S194L_D371Y_T391I; P199L; R203K_G204R_A398V; A220V; A220V_P365S; or M234I_A376T_R385K.

Variant non-structural protein 3 (NSP3): The SARS-CoV-2 non-structural protein 3 (NSP3) comprises at least one of the following amino acid variations (amino acid positions according to reference according to reference SEQ ID NO: 15733): A42V; A86V; E96D; S127L; D136N; P142S; P154L; T184I; L199F; D219E; T238A; H296Y; G308C; D310Y; A329T; H343Y; S371L; E379V; T429I; K430N; I442V; V474F; A489S; N507S; S544P; I581V; A656V; T721I; D737G; T750I; S795L; D808E; T820I; T821I; D822N; P823L; K838N; A862S; A891D; K928I; K978Q; A995D; I1022V; T1064I; K1078R; G1129S; V1174I; T1190I; L1222F; P1229L; K1242R; H1275Y; A1280V; G1301D; A1306V; T1307I; N1330D; T1364I; I1413T; M1442I; S1444Y; P1470S; A1527V; A1528V; F1570V; N1588S; S1589L; I1684T; K1694N; N1706T; A1712V; A1737V; K1772R; N1779S; V1787A; M1789I; E1790K; S1808F; T1831I; E1847D; or Q1885H.

In preferred embodiments the SARS-CoV-2 non-structural protein 3 (NSP3) protein comprises the following amino acid variations (amino acid positions according to reference according to reference SEQ ID NO: 15733): T184I_A891D_I1413T; A329T_P823L; S371L_K978Q; A489S_P1229L_P1470S; K838N; K838N_P1229L_N1779S; K838N_N1779S; A489S_P1229L_P1470S_A1712V; S795L_K838N; A42V_T429I_D822N_P1470S; T238A_T721I_N1330D; H343Y_P823L_K928I_N1588S; E379V_K1694N; T429I_P1470S_F1570V; D737G_S1808F; T750I; T1190I; A1527V_T1831I; A86V_A656V_I1022V_E1847D; D136N_E379V_K1694N; A862S_G1129S; T1190I_K1772R; P142S; D310Y_A489S_P1229L_P1470S; N507S_T821I_S1444Y; I581V_T1064I; A995D; V1174I; A1306V_E1790K; I1684T; S1808F; A489S_P823L_P1229L_P1470S_A1712V; T820I_I1684T; D219E_Q1885H; L199F_K1078R_A1280V; H296Y_I442V; K430N; S544P_V1787A; D808E_G1301 D_N1706T; L1222F_T1307I_Q1885H; K1242R; T1364I_A1737V; S1589L; or M1789I.

Variant non-structural protein 4 (NSP4): The SARS-CoV-2 non-structural protein 4 (NSP4) comprises at least one of the following amino acid variations (amino acid positions according to reference according to reference SEQ ID NO: 16415): N6S; F18L; I24T; M34L; T115I; S138L; S164F; V168L; T174I; G179S; S185N; T190I; Y206H; D218N; D218G; S219F; S239N; D260N; L265F; D280N; T296I; F309Y; M325V; M325I; I346S; L354F; T355A; A381V; S396T; Y398H; K400E; R4n1S; L439P; L439F; T440M; Y442H; A447V; L448F; M459I; D460N; A473S; S482L; or T493I.

In preferred embodiments the SARS-CoV-2 non-structural protein 4 (NSP4) protein comprises the following amino acid variations (amino acid positions according to reference according to reference SEQ ID NO: 16415): V168L_T493I; F18L; 124T_G179S_S185N_A447V; V168L_A447V_T493I; Y206H_L265F_T296I_A473S; T174I_A447V; T190I_T493I; D218N_L439P: D218N_D460N; S396T; L439P; A447V; L439P_T493I; T115I_S396T; K400E_L439P_A447V; L448F; Y398H; T493I; D218G_M325V_A381V_L439F; N6S_S138L_S219F_L439P; S164F_I346S_L354F_S482L; V168L_M325I_L439P_A447V_T493I; T190I_D260N_T440M_T493I; D218G_Y442H: S219F_D280N_T493I; M34L; S239N; F309Y; F309Y_T493I; M325I; T355A_A381V_R401S; A381V; K400E_L439P_A447V_T493I; or M459I. Variant non-structural protein 6 (NSP6): The SARS-CoV-2 non-structural protein 6 (NSP6) comprises at least one of the following amino acid variations (amino acid positions according to reference according to reference SEQ ID NO: 16582): V27G; L38F; M48I; I50V; A55S; P78L; T78A; V85F; M87I; L99F; L106C; S107del; G108del; G108S; F109del; F109L; L126F; L143F; M144I; V150A; V150F; Q161R; I163V; S164A; M165T; V180I; T182I; M184I; L186F; E196D; Q209R; F221L; C222F; L261F; K271R; or V279I.

In preferred embodiments the SARS-CoV-2 non-structural protein 6 (NSP6) protein comprises the following amino acid variations (amino acid positions according to reference according to reference SEQ ID NO: 16582): T78A_V150A_I163V_T182I; S107del_G108del_F109del; S107del_G108del_F109del_M165T; V27G_L38F_F221L_C222F; L38F_L126F; S107del_G108del_F109del_L126F; S107del_G108del_F109del_V279I; L38F_S164A_E196D; I50V; T78A; T78A_V150A_T182I; M87I_L99F_M184I_L186F; S107del_G108del_F109del_M184I; L38F_I50V_S107del_G108del_F109del; L38F_A55S_L143F; L38F_T78A_T182I; L38F_V85F_S107del_G108del_F109del; L38F_V150F; M48I_Q161R; M87I_M144I; L106C_S107del_G108del; Q209R; or K271R.

Variant non-structural protein 13 (NSP13): The SARS-CoV-2 non-structural protein 13 (NSP13) comprises at least one of the following amino acid variations (amino acid positions according to reference according to reference SEQ ID NO: 27908): P48S; P54L; S75L; P78L; P79S; Q89H; V99F; D106Y; T128I; H165Y; V170F; K172R; P173H; V188L; I196T; G207C; K219R; M234I; T251I; D261Y; E262D; H291Y; A297S; L298F; E342D; T352I; P420S; M430I; T432I; G440R; R443Q; A447S; K461R; T482M; P492S; P505L; P530L; Y542C; M577I; R580G; L582F; T589I; R596K; or A599S. In preferred embodiments the SARS-CoV-2 non-structural protein 13 (NSP13) protein comprises the following amino acid variations (amino acid positions according to reference according to reference SEQ ID NO: 27908): P78L; P79S_K461R; E342D; T589I; P54L_D261Y; Q89H_H165Y; D106Y_P530L; P173H_E262D_P420S_P492S; D261Y; P54L_H291Y_A599S; G207C_M430I; H291Y_A599S; G440R_M577I; R580G_R596K; P48S_P78L; S75L_V170F_L298F_T352I_L582F; P78L_D261Y_K461R; V99F_M234I_T432I; T128I_G440R_K461R_T482M; K172R_T482M; V188L; I196T_T251I_R443Q; K219R; K219R_E262D_A447S; E262D; or A297S.

Variant non-structural protein 14 (NSP14): The SARS-CoV-2 non-structural protein 14 (NSP14) comprises at least one of the following amino acid variations (amino acid positions according to reference according to reference SEQ ID NO: 27909): T17I; T22A; T32I; P44L; P44S; P47L; M50I; G60S; M73I; A97V; N117D; N130D; S138I; P141L; P143S; D145E; D145G; I151T; P204L; F218Y; S219A; T251I; S256I; A275S; K305N; M316I; A321V; D325E; P328Q; D346Y; E348G; A354V; Q355H; A361V; A372T; V382L; A395V; P413H; P413S; A431T; N439S; P444S; P452S; E454D; H456Y; H487Y; or M502I.

In preferred embodiments the SARS-CoV-2 non-structural protein 14 (NSP14) protein comprises the following amino acid variations (amino acid positions according to reference according to reference SEQ ID NO: 27909): T32I_P143S_P444S; P204L_F218Y_P328Q; E348G_P452S; A395V; D145E_A275S; D145G_Q355H; S256I_A361V_A372T: T22A_M316I_A372T-P47L_E454D; M50I_D346Y_H487Y: G60S_P141L; M73I; P413H; T17I; T32I_N130D: P44L; P44L_P413H_N439S; P44S_D325E; A97V_A431T; N117D_S256I_K305N_P413S; N130D; N130D_A321V; N130D_A395V; S138I_S219A_H456Y; I151T; T251I; A354V_M502I; V382L; or M502I.

Variant ORF3a accessory protein: The SARS-CoV-2 ORF3a protein comprises at least one of the following amino acid variations (amino acid positions according to reference according to reference SEQ ID NO: 16684): L15F; E19del; I20del; K21del; D22del; A23del; T24del; P25del; S26L; S26del; D27del; F28del; Q38L; Q38R; P42L; A54V; Q57H; W69L; A72S; H78Y; L83F; L95F; G100C; A103T; P104L; A110S; R122I; W131C; W131L; T151I; D155Y; S165F; S166L; S171L; G172C; G172V; G172R; D173G; Q185H; V202L; T223I; G224C; P240S; S253P; N257del; or V259L.

In preferred embodiments the SARS-CoV-2 ORF3a protein comprises the following amino acid variations (amino acid positions according to reference according to reference SEQ ID NO: 16684): Q57H_S171L; D155Y_S253P; S253P; L15F_W131C_T151I; P42L_Q57H; Q57H_N257del; N257del; P42L_Q57H_P104L; S26L; Q57H; Q57H_N257del_V259L; E19del_l20del_K21del_D22del_A23del_T24del_P25del_S26del_D27del_F28del_G172C; Q38L_A54V_W69L_A103T_T151I_G172C A72S_H78Y_S171L; Q185H; S26L_Q57H_G172V; Q38R_G172R_V202L; Q57H_L83F_S171L; Q57H_L95F; Q57H_G172V; Q57H_T223I; Q57H_G224C; A110S; R122I; T223I; W131L_T151I_S165F; S166L; or D173G.

Variant ORF8 accessory protein: The SARS-CoV-2 ORF8 protein comprises at least one of the following amino acid variations (amino acid positions according to reference according to reference SEQ ID NO: 16997): K2Q; F3Y; L4P; I10T; T11I; T11K; H17Y; S24L; C25F; T26I; P30L; V32L; P38S; H40Y; K44R; W45L; A51S; R52I; S54L; C61F; V62L; A65V; A65S; S67F; S69L; Y73C; Y79F; L84S; T87S; F86del; E92K; V100L; L118V; D119I; F120V; F120S; F120L; F120del; I121L; I121V; I121del; Q27stop; E64stop; K68stop; or E106stop.

In preferred embodiments the SARS-CoV-2 ORF8 protein comprises the following amino acid variations (amino acid positions according to reference according to reference SEQ ID NO: 16997): R52I_Y73C; E92K; D119I_F120del_I121del; K44R_F120V_I121L; K2Q; T11I; T11K_P38S_S67F; T26I; F3Y; T11I_A51S; V32L_V100L; A65V; L84S_E92K; L4P_A65V; I10T_S69L; C25F; R52I_F120S_I121V; A65S_S67F_F86del_T87S_F120L; T11I_S24L; H17Y_W45L_A65S; S24L; S24L_S54L; P30L_H40Y; V32L; R52I_V62L_Y73C V62L_L84S_E92K; S67F_Y79F; or L84S.

Variant envelope protein (E): The SARS-CoV-2 E envelope protein comprises at least one of the following amino acid variations (amino acid positions according to reference according to reference SEQ ID NO: 15689): V5I; T9I; F20L; L21F; L21V; F23L; V24A; T30I; A32V; C43F; I46V; V49L; S55F; V58F; V58I; V62F; N64T; S68A; S68F; S68C; R69G; R69I; V70F; P71L; P71S; D72Y; D72G; L73F; L73J; or L73I.

In preferred embodiments the SARS-CoV-2 E envelope protein comprises the following amino acid variations (amino acid positions according to reference according to reference SEQ ID NO: 15689): P71L; T9I_C43F_V49L_V58F_V62F_L73F; T9I_V58F_V62F_S68F_L73F; T9I_V58F_S68A_R69G_P71S; L21F; T9I_L21F_S55F_V58F_S68F_L73F; T9I_S55F_V58F_S68F_P71L_D72Y_L73F; T9I_S55F_V58F_S68F_D72Y_L73F; F20L_T30I; T9I_L21V_T30I_S68F_V70F; T9I_L21F_V49L_N64T_S68F_P71S_L73F; V24A_S68F_L73F; V58F_V62F_L73J; L21F_S68F_P71L_L73F; L21V; L21V_P71L; L73F; P71S; T30I_V62F_S68F_P71L_L73F; T9I; T9I_F23L_T30I_A32V_S55F_V58F_V62F_S68F; T9I_L21F_V62F_S68F_R69I_P71L_L73F; V49L_S68F_D72G_L73F; V58F; V58I_S68C_L73F; V5I_I46V_S55F_V62F_S68F_L73F; or V62F_S68F_L73I_L73F.

In the context of the invention, any protein that is selected from or is derived from SARS-CoV-2 comprising at least one amino acid substitution selected from a SARS-CoV-2 variant may be used and may be suitably encoded by the coding sequence or the nucleic acid may be used in the context of the invention. It is further in the scope of the underlying invention, that the at least one antigenic peptide or protein may comprise or consist of a synthetically engineered or an artificial SARS-CoV-2 protein. The term “synthetically engineered” SARS-CoV-2 protein, or the term “artificial SARS-CoV-2 protein” relates to a protein that does not occur in nature. Accordingly, an “artificial SARS-CoV-2 protein” or a “synthetically engineered SARS-CoV-2 protein” may for example differ in at least one amino acid compared to the natural SARS-CoV-2 protein, and/or may comprise an additional heterologous peptide or protein element, and/or may be N-terminally or C-terminally extended or truncated.

In embodiments, the membrane protein (M), nucleocapsid protein (N), non-structural protein (NSP), accessory protein, and/or envelope protein (E), or an immunogenic fragment or immunogenic variant thereof, is selected or derived from a MERS-associated virus. In preferred embodiments, the membrane protein (M), nucleocapsid protein (N), non-structural protein (NSP), accessory protein, and/or envelope protein (E), or an immunogenic fragment or immunogenic variant thereof, is selected or derived from a MERS-CoV.

In embodiments, the encoded at least one antigenic peptide or protein comprises a membrane protein (M) fragment, nucleocapsid protein (N) fragment, non-structural protein (NSP) fragment, accessory protein fragment, and/or envelope protein (E) fragment, e.g. a fragment that lacks at least 20%, 30%, 40% of the membrane protein (M), nucleocapsid protein (N), non-structural protein (NSP), accessory protein, and/or envelope protein (E).

In embodiments, the encoded at least one antigenic peptide or protein comprises a Coronavirus membrane protein (M) fragment, nucleocapsid protein (N) fragment, non-structural protein (NSP) fragment, accessory protein fragment, and/or envelope protein (E) fragment is N-terminally or C-terminally truncated.

In particularly preferred embodiments, the encoded at least one antigenic peptide or protein comprises or consists of a full-length a Coronavirus membrane protein (M), nucleocapsid protein (N), non-structural protein (NSP), accessory protein, and/or envelope protein (E), or an immunogenic variant of any of these. The term “full-length protein” has to be understood as a protein having an amino acid sequence corresponding to essentially the respective full protein sequence.

Nucleic Acid Encoding a Coronavirus M:

In preferred embodiments, the pharmaceutical composition comprises at least one nucleic acid comprising at least one coding sequence encoding at least one antigenic peptide or protein selected or derived from at least one membrane protein (M), or an immunogenic fragment or immunogenic variant thereof.

Preferred antigenic peptide or proteins selected or derived from a Coronavirus membrane (M) protein as defined above are provided in Table 1 (rows 1 to 16, 119 to 126). Further provided in Table 1 are preferred nucleic acid sequences (coding sequences, mRNA sequences) encoding said antigenic peptide or proteins.

In embodiments, the Coronavirus membrane protein (M) comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 15221-15295, or 27910-27917, or an immunogenic fragment or immunogenic variant of any of these. Further information regarding respective amino acid sequences is provided under <223> identifier of the respective SEQ ID NO in the sequence listing, and in Table 1, rows 1 to 16. 119 to 126, see in particular Column A).

In preferred embodiments, the Coronavirus membrane protein (M) is selected or derived from SARS-CoV-2 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 15235-15295, or an immunogenic fragment or immunogenic variant of any of these. Particularly preferred amino acid sequences in the context of the invention are provided in Table 9.

In further embodiments, the Coronavirus membrane protein (M) selected or derived from SARS-CoV-2 is a variant membrane protein (M) and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 27910-27917, or an immunogenic fragment or immunogenic variant of any of these.

According to preferred embodiments, the at least one nucleic acid of the composition comprises at least one coding sequence encoding at least one antigenic peptide or protein derived from a Coronavirus membrane protein (M) as defined above, or fragments and variants thereof. In that context, any coding sequence encoding at least one Coronavirus membrane protein (M) as defined herein, or fragments and variants thereof may be understood as suitable coding sequence and may therefore be comprised in the nucleic acid of the composition.

In preferred embodiments, the at least one nucleic acid comprises or consists of at least one coding sequence encoding at least one antigenic peptide or protein selected or derived from Coronavirus membrane protein (M) as defined herein, preferably encoding any one of SEQ ID NOs: 15221-15295, 27910-27917, or fragments of variants thereof. It has to be understood that, on nucleic acid level, any sequence (DNA or RNA sequence) which encodes an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 15221-15295, 27910-27917, or fragments or variants thereof, may be selected and may accordingly be understood as suitable coding sequence of the invention. Further information regarding said amino acid sequences is also provided in Table 1 and under <223> identifier of the ST25 sequence listing of respective sequence SEQ ID NOs.

In preferred embodiments, the at least one coding sequence of the nucleic acid is a codon modified coding sequence as defined herein, wherein the amino acid sequence, that is Coronavirus membrane protein (M), encoded by the at least one codon modified coding sequence, is preferably not being modified compared to the amino acid sequence encoded by the corresponding wild type or reference coding sequence.

In particularly preferred embodiments, the at least one coding sequence of the nucleic acid is a codon modified coding sequence, wherein the codon modified coding sequence is selected a G/C optimized coding sequence, a human codon usage adapted coding sequence, or a G/C modified coding sequence.

In embodiments, the at least one coding sequence comprises or consists at least one nucleic acid sequence encoding a Coronavirus membrane protein (M) being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 17098-17172, 18975-19049, 20852-20926, 28271, or a fragment or variant of any of these sequences. Further information regarding respective nucleic acid sequences is provided under <223> identifier of the respective SEQ ID NO in the sequence listing, and in Table 1, rows 1 to 16, see in particular Column C).

In preferred embodiments, the at least one coding sequence comprises or consists at least one nucleic acid sequence encoding a SARS-CoV-2 membrane protein (M) being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 17112-17172, 18989-19049, 20866-20926, or a fragment or variant of any of these sequences. Particularly preferred nucleic acid sequences encoding SARS-CoV-2 M in the context of the invention are provided in Table 9.

In further embodiments, the at least one coding sequence comprises or consists at least one nucleic acid sequence encoding a SARS-CoV-2 variant membrane protein (M) being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 27910-27917, 28031-28038, 28152-28159, 28282-28289, 28403-28410, or a fragment or variant of any of these sequences. Further information regarding respective nucleic acid sequences is provided under <223> identifier of the respective SEQ ID NO in the sequence listing, and in Table 1, rows 119 to 126).

In embodiments, the at least one nucleic acid comprises or consists of a nucleic acid sequence encoding a Coronavirus membrane protein (M) which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 18975-19049, 20852-20926, 28152-28159, 28271, 28282-28289, 28403-28410, or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides. Further information regarding respective nucleic acid (mRNA) sequences is provided under <223> identifier of the respective SEQ ID NO in the sequence listing, and in Table 1, rows 1 to 16, 119 to 126 (see in particular Column D).

In embodiments, the at least one nucleic acid comprises or consists of a nucleic acid sequence encoding a SARS-CoV-2 membrane protein (M) which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 18989-19049, 20866-20926 or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides. Particularly preferred mRNA sequences in the context of the invention encoding SARS-CoV-2 M are provided in Table 9.

In further embodiments, the at least one nucleic acid comprises or consists of a nucleic acid sequence encoding a SARS-CoV-2 variant membrane protein (M) which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 28152-28159, 28282-28289, 28403-28410 or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides.

Nucleic Acid Encoding a Coronavirus N:

In preferred embodiments, the pharmaceutical composition comprises at least one nucleic acid comprising at least one coding sequence encoding at least one antigenic peptide or protein selected or derived from at least one nucleocapsid protein (N), or an immunogenic fragment or immunogenic variant thereof.

Preferred antigenic peptide or proteins selected or derived from a Coronavirus nucleocapsid protein (N) as defined herein are provided in Table 1 (rows 17-32, 127-148). Further provided in Table 1 are preferred nucleic acid sequences (coding sequences, mRNA sequences) encoding said antigenic peptide or proteins.

In embodiments, the Coronavirus nucleocapsid protein (N) comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 15296-15674, 27918-27939, or an immunogenic fragment or immunogenic variant of any of these. Further information regarding respective amino acid sequences is provided under <223> identifier of the respective SEQ ID NO in the sequence listing, and in Table 1, row 17-32, 127-148, see in particular Column B).

In preferred embodiments, the Coronavirus nucleocapsid protein (N) is selected or derived from SARS-CoV-2 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 15310-15674, or an immunogenic fragment or immunogenic variant of any of these. Particularly preferred amino acid sequences in the context of the invention are provided in Table 9.

In further embodiments, the Coronavirus nucleocapsid protein (N) selected or derived from SARS-CoV-2 is a variant nucleocapsid protein (N) and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 27918-27939, or an immunogenic fragment or immunogenic variant of any of these.

According to preferred embodiments, the at least one nucleic acid of the composition comprises at least one coding sequence encoding at least one antigenic peptide or protein derived from a Coronavirus nucleocapsid protein (N) as defined above, or fragments and variants thereof. In that context, any coding sequence encoding at least one Coronavirus nucleocapsid protein (N) as defined herein, or fragments and variants thereof may be understood as suitable coding sequence and may therefore be comprised in the nucleic acid of the composition.

In preferred embodiments, the at least one nucleic acid comprises or consists of at least one coding sequence encoding at least one antigenic peptide or protein selected or derived from Coronavirus nucleocapsid protein (N) as defined herein, preferably encoding any one of SEQ ID NOs: 15296-15674, 27918-27939, or fragments of variants thereof. It has to be understood that, on nucleic acid level, any sequence (DNA or RNA sequence) which encodes an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 15296-15674, 27918-27939, or fragments or variants thereof, may be selected and may accordingly be understood as suitable coding sequence of the invention. Further information regarding said amino acid sequences is also provided in Table 1, and under <223> identifier of the ST25 sequence listing of respective sequence SEQ ID NOs.

In preferred embodiments, the at least one coding sequence of the nucleic acid is a codon modified coding sequence as defined herein, wherein the amino acid sequence, that is Coronavirus nucleocapsid protein (N), encoded by the at least one codon modified coding sequence, is preferably not being modified compared to the amino acid sequence encoded by the corresponding wild type or reference coding sequence.

In particularly preferred embodiments, the at least one coding sequence of the nucleic acid is a codon modified coding sequence, wherein the codon modified coding sequence is selected a G/C optimized coding sequence, a human codon usage adapted coding sequence, or a G/C modified coding sequence.

In embodiments, the at least one coding sequence comprises or consists at least one nucleic acid sequence encoding a Coronavirus nucleocapsid protein (N) being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 17173-17551, 19050-19428, 20927-21305, 28039-28060, 28160-28181, 28272, 28290-28311, 28411-28432, or a fragment or variant of any of these sequences. Further information regarding respective nucleic acid sequences is provided under <223> identifier of the respective SEQ ID NO in the sequence listing, and in Table 1, row 17-32, 127-148, see in particular Column C).

In preferred embodiments, the at least one coding sequence comprises or consists at least one nucleic acid sequence encoding a SARS-CoV-2 nucleocapsid protein (N) being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 17187-17551, 19064-19428, 20941-21305, or a fragment or variant of any of these sequences. Particularly preferred nucleic acid sequences encoding SARS-CoV-2 N in the context of the invention are provided in Table 9.

In further embodiments, the at least one coding sequence comprises or consists at least one nucleic acid sequence encoding a SARS-CoV-2 variant nucleocapsid protein (N) being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 28039-28060, 28160-28181, 28272, 28290-28311, 28411-28432 or a fragment or variant of any of these sequences. Further information regarding respective nucleic acid sequences is provided under <223> identifier of the respective SEQ ID NO in the sequence listing, and in Table 1, rows 127-148).

In embodiments, the at least one nucleic acid comprises or consists of a nucleic acid sequence encoding a Coronavirus nucleocapsid protein (N) which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 19050-19428, 20927-21305, 28160-28181, 28272, 28290-28311, 28411-28432, or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides. Further information regarding respective nucleic acid sequences is provided under <223> identifier of the respective SEQ ID NO in the sequence listing, and in Table 1, row 17-32, 127-148, see in particular Column D).

In embodiments, the at least one nucleic acid comprises or consists of a nucleic acid sequence encoding a SARS-CoV-2 nucleocapsid protein (N) which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 19064-19428, 20941-21305, 28272, or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides. Particularly preferred mRNA sequences encoding SARS-CoV-2 N in the context of the invention are provided in Table 9.

In further embodiments, the at least one nucleic acid comprises or consists of a nucleic acid sequence encoding a SARS-CoV-2 variant nucleocapsid protein (N) which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 28160-28181, 28290-28311, 28411-28432, or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides.

Nucleic Acid Encoding a Coronavirus NSPs:

In embodiments, the pharmaceutical composition comprises the at least one nucleic acid comprising at least one coding sequence encoding at least one antigenic peptide or protein selected or derived from at least one non-structural protein selected from NSP1, NSP2, NSP3, NSP4, NSP5, NSP6, NSP7, NSP8, NSP9, NSP10, NSP11, NSP12, NSP13, NSP14, NSP15, and/or NSP16 or an immunogenic fragment or immunogenic variant of any of these.

In preferred embodiments, the pharmaceutical composition comprises the at least one nucleic acid comprising at least one coding sequence encoding at least one antigenic peptide or protein selected or derived from at least one non-structural protein selected from NSP3, NSP4, and/or NSP6 or an immunogenic fragment or immunogenic variant of any of these.

In preferred embodiments, the pharmaceutical composition comprises at least one nucleic acid comprising at least one coding sequence encoding at least one antigenic peptide or protein selected or derived from at least one Coronavirus NSP3 protein, or an immunogenic fragment or immunogenic variant thereof.

Preferred antigenic peptide or proteins selected or derived from a Coronavirus NSP3 as defined herein are provided in Table 1 (rows 49-64, 157-178). Further provided in Table 1 are preferred nucleic acid sequences (coding sequences, mRNA sequences) encoding said antigenic peptide or proteins.

In embodiments, the Coronavirus NSP3 protein comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 15720-16401, 27948-27969, or an immunogenic fragment or immunogenic variant of any of these. Further information regarding respective amino acid sequences is provided under <223> identifier of the respective SEQ ID NO in the sequence listing, and in Table 1, row 49-64, 157-178, see in particular Column B).

In preferred embodiments, the Coronavirus NSP3 protein is selected or derived from SARS-CoV-2 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 15734-16401 or an immunogenic fragment or immunogenic variant of any of these. Particularly preferred amino acid sequences in the context of the invention are provided in Table 9.

In further embodiments, the Coronavirus NSP3 protein selected or derived from SARS-CoV-2 is a variant membrane protein (M) and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 27948-27969, or an immunogenic fragment or immunogenic variant of any of these.

According to preferred embodiments, the at least one nucleic acid of the composition comprises at least one coding sequence encoding at least one antigenic peptide or protein derived from a Coronavirus NSP3 protein as defined above, or fragments and variants thereof. In that context, any coding sequence encoding at least one Coronavirus NSP3 protein as defined herein, or fragments and variants thereof may be understood as suitable coding sequence and may therefore be comprised in the nucleic acid of the composition.

In preferred embodiments, the at least one nucleic acid comprises or consists of at least one coding sequence encoding at least one antigenic peptide or protein selected or derived from Coronavirus NSP3 protein as defined herein, preferably encoding any one of SEQ ID NOs: 15720-16401, 27948-27969, or fragments of variants thereof. It has to be understood that, on nucleic acid level, any sequence (DNA or RNA sequence) which encodes an amino acid sequences being identical or at least 70%0, 80%, 85%, 86%, 87%, 88%, 89%, 90%, 91%, 92%, 93%, 94%, 95%, 96%, 97%, 98%, or 99% identical to any one of SEQ ID NOs: 15720-16401, 27948-27969, or fragments or variants thereof, may be selected and may accordingly be understood as suitable coding sequence of the invention. Further information regarding said amino acid sequences is also provided in Table 1, and under <223> identifier of the ST25 sequence listing of respective sequence SEQ ID NOs.

In preferred embodiments, the at least one coding sequence of the nucleic acid is a codon modified coding sequence as defined herein, wherein the amino acid sequence, that is Coronavirus NSP3 protein, encoded by the at least one codon modified coding sequence, is preferably not being modified compared to the amino acid sequence encoded by the corresponding wild type or reference coding sequence.

In particularly preferred embodiments, the at least one coding sequence of the nucleic acid is a codon modified coding sequence, wherein the codon modified coding sequence is selected a G/C optimized coding sequence, a human codon usage adapted coding sequence, or a G/C modified coding sequence.

In embodiments, the at least one coding sequence comprises or consists at least one nucleic acid sequence encoding a Coronavirus NSP3 protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 17597-18278, 19474-20155, 21351-22032, 28069-28090, 28190-28211, 28320-28341, 28441-28462, 28274, 28275, or a fragment or variant of any of these sequences. Further information regarding respective nucleic acid sequences is provided under <223> identifier of the respective SEQ ID NO in the sequence listing, and in Table 1, row 49-64, 157-178, see in particular Column C).

In preferred embodiments, the at least one coding sequence comprises or consists at least one nucleic acid sequence encoding a SARS-CoV-2 NSP3 protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 17611-18278, 19488-20155, 21365-22032, or a fragment or variant of any of these sequences. Particularly preferred nucleic acid sequences encoding SARS-CoV-2 NSP3 in the context of the invention are provided in Table 9.

In further embodiments, the at least one coding sequence comprises or consists at least one nucleic acid sequence encoding a SARS-CoV-2 variant NSP3 protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 28069-28090, 28190-28211, 28320-28341, 28441-28462, or a fragment or variant of any of these sequences. Further information regarding respective nucleic acid sequences is provided under <223> identifier of the respective SEQ ID NO in the sequence listing, and in Table 1, rows 157-178).

In embodiments, the at least one nucleic acid comprises or consists of a nucleic acid sequence encoding a Coronavirus NSP3 protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 19474-20155, 21351-22032, 28190-28211, 28320-28341, 28441-28462, 28274, 28275, or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides. Further information regarding respective nucleic acid sequences is provided under <223> identifier of the respective SEQ ID NO in the sequence listing, and in Table 1, row 49-64, 157-178, see in particular Column D).

In embodiments, the at least one nucleic acid comprises or consists of a nucleic acid sequence encoding a SARS-CoV-2 NSP3 protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 19488-20155, 21365-22032 or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides. Particularly preferred mRNA sequences encoding SARS-CoV-2 NSP3 in the context of the invention are provided in Table 9.

In further embodiments, the at least one nucleic acid comprises or consists of a nucleic acid sequence encoding a SARS-CoV-2 variant NSP3 protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 28190-28211, 28320-28341, 28441-28462 or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides.

In preferred embodiments, the pharmaceutical composition comprises at least one nucleic acid comprising at least one coding sequence encoding at least one antigenic peptide or protein selected or derived from at least one Coronavirus NSP4 protein, or an immunogenic fragment or immunogenic variant thereof.

Preferred antigenic peptide or proteins selected or derived from a Coronavirus NSP4 as defined herein are provided in Table 1 (rows 65-79, 179-194). Further provided in Table 1 are preferred nucleic acid sequences (coding sequences, mRNA sequences) encoding said antigenic peptide or proteins.

In embodiments, the Coronavirus NSP4 protein comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 16402-16568, 27970-27985, or an immunogenic fragment or immunogenic variant of any of these. Further information regarding respective amino acid sequences is provided under <223> identifier of the respective SEQ ID NO in the sequence listing, and in Table 1, row 65-79, 179-194, see in particular Column B).

In preferred embodiments, the Coronavirus NSP4 protein is selected or derived from SARS-CoV-2 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 16415-16568 or an immunogenic fragment or immunogenic variant of any of these. Particularly preferred amino acid sequences in the context of the invention are provided in Table 9.

In further embodiments, the Coronavirus NSP4 protein selected or derived from SARS-CoV-2 is a variant membrane protein (M) and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 27970-27985, or an immunogenic fragment or immunogenic variant of any of these.

According to preferred embodiments, the at least one nucleic acid of the composition comprises at least one coding sequence encoding at least one antigenic peptide or protein derived from a Coronavirus NSP4 protein as defined above, or fragments and variants thereof. In that context, any coding sequence encoding at least one Coronavirus NSP4 protein as defined herein, or fragments and variants thereof may be understood as suitable coding sequence and may therefore be comprised in the nucleic acid of the composition.

In preferred embodiments, the at least one nucleic acid comprises or consists of at least one coding sequence encoding at least one antigenic peptide or protein selected or derived from Coronavirus NSP4 protein as defined herein, preferably encoding any one of SEQ ID NOs: 16402-16568, 27970-27985, or fragments of variants thereof. It has to be understood that, on nucleic acid level, any sequence (DNA or RNA sequence) which encodes an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 16402-16568, 27970-27985, or fragments or variants thereof, may be selected and may accordingly be understood as suitable coding sequence of the invention. Further information regarding said amino acid sequences is also provided in Table 1, and under <223> identifier of the ST25 sequence listing of respective sequence SEQ ID NOs.

In preferred embodiments, the at least one coding sequence of the nucleic acid is a codon modified coding sequence as defined herein, wherein the amino acid sequence, that is Coronavirus NSP4 protein, encoded by the at least one codon modified coding sequence, is preferably not being modified compared to the amino acid sequence encoded by the corresponding wild type or reference coding sequence.

In particularly preferred embodiments, the at least one coding sequence of the nucleic acid is a codon modified coding sequence, wherein the codon modified coding sequence is selected a G/C optimized coding sequence, a human codon usage adapted coding sequence, or a G/C modified coding sequence.

In embodiments, the at least one coding sequence comprises or consists at least one nucleic acid sequence encoding a Coronavirus NSP4 protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 18279-18445, 20156-20322, 22033-22199, 28091-28106, 28212-28227, 28276, 28342-28357, 28463-28478, or a fragment or variant of any of these sequences. Further information regarding respective nucleic acid sequences is provided under <223> identifier of the respective SEQ ID NO in the sequence listing, and in Table 1, row 65-79, 179-194, see in particular Column C).

In preferred embodiments, the at least one coding sequence comprises or consists at least one nucleic acid sequence encoding a SARS-CoV-2 NSP4 protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 18292-18445, 20169-20322, 22046-22199, or a fragment or variant of any of these sequences. Particularly preferred nucleic acid sequences encoding SARS-CoV-2 NSP4 in the context of the invention are provided in Table 9.

In further embodiments, the at least one coding sequence comprises or consists at least one nucleic acid sequence encoding a SARS-CoV-2 variant NSP4 protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 28091-28106, 28212-28227, 28342-28357, 28463-28478, or a fragment or variant of any of these sequences. Further information regarding respective nucleic acid sequences is provided under <223> identifier of the respective SEQ ID NO in the sequence listing, and in Table 1, rows 179-194.

In embodiments, the at least one nucleic acid comprises or consists of a nucleic acid sequence encoding a Coronavirus NSP4 protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 20156-20322, 22033-22199, 28190-28211, 28320-28341, 28276, 28441-28462, or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides. Further information regarding respective nucleic acid sequences is provided under <223> identifier of the respective SEQ ID NO in the sequence listing, and in Table 1, row 65-79, 179-194, see in particular Column D).

In embodiments, the at least one nucleic acid comprises or consists of a nucleic acid sequence encoding a SARS-CoV-2 NSP4 protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 20169-20322, 22046-22199, or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides. Particularly preferred mRNA sequences encoding SARS-CoV-2 NSP4 in the context of the invention are provided in Table 9.

In further embodiments, the at least one nucleic acid comprises or consists of a nucleic acid sequence encoding a SARS-CoV-2 variant NSP4 protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 28190-28211, 28320-28341, 28441-28462, or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides.

In preferred embodiments, the pharmaceutical composition comprises at least one nucleic acid comprising at least one coding sequence encoding at least one antigenic peptide or protein selected or derived from at least one Coronavirus NSP6 protein, or an immunogenic fragment or immunogenic variant thereof.

Preferred antigenic peptide or proteins selected or derived from a Coronavirus NSP6 as defined herein are provided in Table 1 (rows 80-94, 195-201). Further provided in Table 1 are preferred nucleic acid sequences (coding sequences, mRNA sequences) encoding said antigenic peptide or proteins.

In embodiments, the Coronavirus NSP6 protein comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 16569-16671, 27986-27992, or an immunogenic fragment or immunogenic variant of any of these. Further information regarding respective nucleic acid sequences is provided under <223> identifier of the respective SEQ ID NO in the sequence listing, and in Table 1, row 80-94, 195-201, see in particular Column B).

In preferred embodiments, the Coronavirus NSP6 is selected or derived from SARS-CoV-2 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 16582-16671, or an immunogenic fragment or immunogenic variant of any of these. Particularly preferred amino acid sequences in the context of the invention are provided in Table 9.

In further embodiments, the Coronavirus NSP6 protein selected or derived from SARS-CoV-2 is a variant NSP6 protein and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 27986-27992, or an immunogenic fragment or immunogenic variant of any of these.

According to preferred embodiments, the at least one nucleic acid of the composition comprises at least one coding sequence encoding at least one antigenic peptide or protein derived from a Coronavirus NSP6 as defined above, or fragments and variants thereof. In that context, any coding sequence encoding at least one Coronavirus NSP6 as defined herein, or fragments and variants thereof may be understood as suitable coding sequence and may therefore be comprised in the nucleic acid of the composition.

In preferred embodiments, the at least one nucleic acid comprises or consists of at least one coding sequence encoding at least one antigenic peptide or protein selected or derived from Coronavirus NSP6 as defined herein, preferably encoding any one of SEQ ID NOs: 16569-16671, 27986-27992, or fragments of variants thereof. It has to be understood that, on nucleic acid level, any sequence (DNA or RNA sequence) which encodes an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 16569-16671, 27986-27992, or fragments or variants thereof, may be selected and may accordingly be understood as suitable coding sequence of the invention. Further information regarding said amino acid sequences is also provided in Table 1, and under <223> identifier of the ST25 sequence listing of respective sequence SEQ ID NOs.

In preferred embodiments, the at least one coding sequence of the nucleic acid is a codon modified coding sequence as defined herein, wherein the amino acid sequence, that is Coronavirus NSP6, encoded by the at least one codon modified coding sequence, is preferably not being modified compared to the amino acid sequence encoded by the corresponding wild type or reference coding sequence.

In particularly preferred embodiments, the at least one coding sequence of the nucleic acid is a codon modified coding sequence, wherein the codon modified coding sequence is selected a G/C optimized coding sequence, a human codon usage adapted coding sequence, or a G/C modified coding sequence.

In embodiments, the at least one coding sequence comprises or consists at least one nucleic acid sequence encoding a Coronavirus NSP6 protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 18446-18548, 20323-20425, 22200-22302, 28107-28113, 28228-28234, 28277, 28358-28364, 28479-28485, or a fragment or variant of any of these sequences. Further information regarding respective nucleic acid sequences is provided under <223> identifier of the respective SEQ ID NO in the sequence listing, and in Table 1, row 80-94, 195-201, see in particular Column C).

In preferred embodiments, the at least one coding sequence comprises or consists at least one nucleic acid sequence encoding a SARS-CoV-2 NSP6 protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 18459-18548, 20336-20425, 22213-22302, or a fragment or variant of any of these sequences. Particularly preferred nucleic acid sequences encoding SARS-CoV-2 NSP6 in the context of the invention are provided in Table 9.

In further embodiments, the at least one coding sequence comprises or consists at least one nucleic acid sequence encoding a SARS-CoV-2 variant NSP6 protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 28107-28113, 28228-28234, 28358-28364, 28479-28485, or a fragment or variant of any of these sequences. Further information regarding respective nucleic acid sequences is provided under <223> identifier of the respective SEQ ID NO in the sequence listing, and in Table 1, rows 195-201).

In embodiments, the at least one nucleic acid comprises or consists of a nucleic acid sequence encoding a Coronavirus NSP6 protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 20323-20425, 22200-22302, 28228-28234, 28277, 28358-28364, 28479-28485, or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides. Further information regarding respective nucleic acid sequences is provided under <223> identifier of the respective SEQ ID NO in the sequence listing, and in Table 1, row 80-94, 195-201, see in particular Column D).

In embodiments, the at least one nucleic acid comprises or consists of a nucleic acid sequence encoding a SARS-CoV-2 NSP6 protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 20336-20425, 22213-22302 or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides. Particularly preferred mRNA sequences SARS-CoV-2 NSP6 in the context of the invention are provided in Table 9.

In further embodiments, the at least one nucleic acid comprises or consists of a nucleic acid sequence encoding a SARS-CoV-2 variant NSP6 protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 28228-28234, 28358-28364, 28479-28485 or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides.

In preferred embodiments, the pharmaceutical composition comprises at least one nucleic acid comprising at least one coding sequence encoding at least one antigenic peptide or protein selected or derived from at least one Coronavirus NSP13 protein, or an immunogenic fragment or immunogenic variant thereof.

Preferred antigenic peptide or proteins selected or derived from a Coronavirus NSP13 as defined herein are provided in Table 1 (rows 117, 222-230). Further provided in Table 1 are preferred nucleic acid sequences (coding sequences, mRNA sequences) encoding said antigenic peptide or proteins.

In embodiments, the Coronavirus NSP13 protein comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 27908, 28013-28021, or an immunogenic fragment or immunogenic variant of any of these. Further information regarding respective nucleic acid sequences is provided under <223> identifier of the respective SEQ ID NO in the sequence listing, and in Table 1, rows 117, 222-230, see in particular Column B).

In preferred embodiments, the Coronavirus NSP13 is selected or derived from SARS-CoV-2 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 27908, or an immunogenic fragment or immunogenic variant thereof.

In further embodiments, the Coronavirus NSP13 selected or derived from SARS-CoV-2 is a variant NSP13 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 28013-28021, or an immunogenic fragment or immunogenic variant of any of these.

According to preferred embodiments, the at least one nucleic acid of the composition comprises at least one coding sequence encoding at least one antigenic peptide or protein derived from a Coronavirus NSP13 as defined above, or fragments and variants thereof. In that context, any coding sequence encoding at least one Coronavirus NSP13 as defined herein, or fragments and variants thereof may be understood as suitable coding sequence and may therefore be comprised in the nucleic acid of the composition.

In preferred embodiments, the at least one nucleic acid comprises or consists of at least one coding sequence encoding at least one antigenic peptide or protein selected or derived from Coronavirus NSP13 as defined herein, preferably encoding any one of SEQ ID NOs: 27908, 28013-28021, or fragments of variants thereof. It has to be understood that, on nucleic acid level, any sequence (DNA or RNA sequence) which encodes an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 27908, 28013-28021, or fragments or variants thereof, may be selected and may accordingly be understood as suitable coding sequence of the invention. Further information regarding said amino acid sequences is also provided in Table 1, and under <223> identifier of the ST25 sequence listing of respective sequence SEQ ID NOs.

In preferred embodiments, the at least one coding sequence of the nucleic acid is a codon modified coding sequence as defined herein, wherein the amino acid sequence, that is Coronavirus NSP13, encoded by the at least one codon modified coding sequence, is preferably not being modified compared to the amino acid sequence encoded by the corresponding wild type or reference coding sequence.

In particularly preferred embodiments, the at least one coding sequence of the nucleic acid is a codon modified coding sequence, wherein the codon modified coding sequence is selected a G/C optimized coding sequence, a human codon usage adapted coding sequence, or a G/C modified coding sequence.

In embodiments, the at least one coding sequence comprises or consists at least one nucleic acid sequence encoding a Coronavirus NSP13 protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 28029, 28150, 28280, 28401, 28134-28142, 28255-28263, 28385-28393, 28506-28514, or a fragment or variant of any of these sequences. Further information regarding respective nucleic acid sequences is provided under <223> identifier of the respective SEQ ID NO in the sequence listing, and in Table 1, row 117, 222-230, see in particular Column C).

In preferred embodiments, the at least one coding sequence comprises or consists at least one nucleic acid sequence encoding a SARS-CoV-2 NSP13 protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 28029, 28150, 28280, 28401, or a fragment or variant of any of these sequences.

In further embodiments, the at least one coding sequence comprises or consists at least one nucleic acid sequence encoding a SARS-CoV-2 variant NSP13 protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 28134-28142, 28255-28263, 28385-28393, 28506-28514, or a fragment or variant of any of these sequences. Further information regarding respective nucleic acid sequences is provided under <223> identifier of the respective SEQ ID NO in the sequence listing, and in Table 1, rows 222-230).

In embodiments, the at least one nucleic acid comprises or consists of a nucleic acid sequence encoding a Coronavirus NSP13 protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 28150, 28280, 28401, 28255-28263, 28385-28393, 28506-28514, or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides. Further information regarding respective nucleic acid sequences is provided under <223> identifier of the respective SEQ ID NO in the sequence listing, and in Table 1, row 117, 222-230, see in particular Column D).

In embodiments, the at least one nucleic acid comprises or consists of a nucleic acid sequence encoding a SARS-CoV-2 NSP13 protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 28150, 28280, 28401, or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides.

In further embodiments, the at least one nucleic acid comprises or consists of a nucleic acid sequence encoding a SARS-CoV-2 variant NSP13 which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 28255-28263, 28385-28393, 28506-28514, or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψp) nucleotides.

In preferred embodiments, the pharmaceutical composition comprises at least one nucleic acid comprising at least one coding sequence encoding at least one antigenic peptide or protein selected or derived from at least one Coronavirus NSP14 protein, or an immunogenic fragment or immunogenic variant thereof.

Preferred antigenic peptide or proteins selected or derived from a Coronavirus NSP14 as defined herein are provided in Table 1 (rows 118, 231-237). Further provided in Table 1 are preferred nucleic acid sequences (coding sequences, mRNA sequences) encoding said antigenic peptide or proteins.

In embodiments, the Coronavirus NSP14 protein comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 27909, 28022-28028, or an immunogenic fragment or immunogenic variant of any of these. Further information regarding respective nucleic acid sequences is provided under <223> identifier of the respective SEQ ID NO in the sequence listing, and in Table 1, row 118, 231-237, see in particular Column B).

In preferred embodiments, the Coronavirus NSP14 is selected or derived from SARS-CoV-2 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 27909, or an immunogenic fragment or immunogenic variant thereof.

In further embodiments, the Coronavirus NSP14 protein selected or derived from SARS-CoV-2 is a variant NSP14 protein and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 28022-28028, or an immunogenic fragment or immunogenic variant of any of these.

According to preferred embodiments, the at least one nucleic acid of the composition comprises at least one coding sequence encoding at least one antigenic peptide or protein derived from a Coronavirus NSP14 as defined above, or fragments and variants thereof. In that context, any coding sequence encoding at least one Coronavirus NSP14 as defined herein, or fragments and variants thereof may be understood as suitable coding sequence and may therefore be comprised in the nucleic acid of the composition.

In preferred embodiments, the at least one nucleic acid comprises or consists of at least one coding sequence encoding at least one antigenic peptide or protein selected or derived from Coronavirus NSP14 as defined herein, preferably encoding any one of SEQ ID NOs: 27909, 28022-28028, or fragments of variants thereof. It has to be understood that, on nucleic acid level, any sequence (DNA or RNA sequence) which encodes an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 27909, 28022-28028, or fragments or variants thereof, may be selected and may accordingly be understood as suitable coding sequence of the invention. Further information regarding said amino acid sequences is also provided in Table 1, and under <223> identifier of the ST25 sequence listing of respective sequence SEQ ID NOs.

In preferred embodiments, the at least one coding sequence of the nucleic acid is a codon modified coding sequence as defined herein, wherein the amino acid sequence, that is Coronavirus NSP14, encoded by the at least one codon modified coding sequence, is preferably not being modified compared to the amino acid sequence encoded by the corresponding wild type or reference coding sequence.

In particularly preferred embodiments, the at least one coding sequence of the nucleic acid is a codon modified coding sequence, wherein the codon modified coding sequence is selected a G/C optimized coding sequence, a human codon usage adapted coding sequence, or a G/C modified coding sequence.

In embodiments, the at least one coding sequence comprises or consists at least one nucleic acid sequence encoding a Coronavirus NSP14 protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 28030, 28151, 28281, 28402, 28143-28149, 28264-28270, 28394-28400, 28515-28521, or a fragment or variant of any of these sequences. Further information regarding respective nucleic acid sequences is provided under <223> identifier of the respective SEQ ID NO in the sequence listing, and in Table 1, row 118, 231-237, see in particular Column C).

In preferred embodiments, the at least one coding sequence comprises or consists at least one nucleic acid sequence encoding a SARS-CoV-2 NSP14 protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 28030, 28151, 28281, 28402, or a fragment or variant of any of these sequences.

In further embodiments, the at least one coding sequence comprises or consists at least one nucleic acid sequence encoding a SARS-CoV-2 variant NSP14 protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 28143-28149, 28264-28270, 28394-28400, 28515-28521, or a fragment or variant of any of these sequences. Further information regarding respective nucleic acid sequences is provided under <223> identifier of the respective SEQ ID NO in the sequence listing, and in Table 1, rows 231-237).

In embodiments, the at least one nucleic acid comprises or consists of a nucleic acid sequence encoding a Coronavirus NSP14 protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 28151, 28281, 28402, 28264-28270, 28394-28400, 28515-28521, or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides. Further information regarding respective nucleic acid sequences is provided under <223> identifier of the respective SEQ ID NO in the sequence listing, and in Table 1, row 118, 231-237, see in particular Column D).

In embodiments, the at least one nucleic acid comprises or consists of a nucleic acid sequence encoding a SARS-CoV-2 NSP14 protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 28151, 28281, 28402, or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides.

Nucleic Acid Encoding a Coronavirus Accessory Protein:

In embodiments, the pharmaceutical composition comprises at least one nucleic acid comprising at least one coding sequence encoding at least one antigenic peptide or protein selected or derived from at least one accessory protein selected from ORF3a, ORF3b, ORF6, ORF7a, ORF7b, ORF8, ORF8a, ORF8b, ORF9b, and/or ORF10 or an immunogenic fragment or immunogenic variant of any of these.

In preferred embodiments, the pharmaceutical composition comprises the at least one nucleic acid comprising at least one coding sequence encoding at least one antigenic peptide or protein selected or derived from at least one Coronavirus accessory protein selected from ORF3a and/or ORF8 or an immunogenic fragment or immunogenic variant of any of these.

In preferred embodiments, the pharmaceutical composition comprises at least one nucleic acid comprising at least one coding sequence encoding at least one antigenic peptide or protein selected or derived from at least one Coronavirus ORF3a protein, or an immunogenic fragment or immunogenic variant thereof.

Preferred antigenic peptide or proteins selected or derived from a Coronavirus ORF3/ORF3a protein as defined herein are provided in Table 1 (rows 95-108, 202-209). Further provided in Table 1 are preferred nucleic acid sequences (coding sequences, mRNA sequences) encoding said antigenic peptide or proteins.

In embodiments, the Coronavirus ORF3a protein comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 16672-16990, 27993-28000 or an immunogenic fragment or immunogenic variant of any of these. Further information regarding respective amino acid sequences is provided under <223> identifier of the respective SEQ ID NO in the sequence listing, and in Table 1, rows 95-108, 202-209, see in particular Column B).

In preferred embodiments, the Coronavirus ORF3a protein is selected or derived from SARS-CoV-2 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 16684-16990, or an immunogenic fragment or immunogenic variant of any of these, Particularly preferred amino acid sequences in the context of the invention are provided in Table 9.

In further embodiments, the Coronavirus ORF3a protein selected or derived from SARS-CoV-2 is a variant ORF3a protein and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 27993-28000, or an immunogenic fragment or immunogenic variant of any of these.

According to preferred embodiments, the at least one nucleic acid of the composition comprises at least one coding sequence encoding at least one antigenic peptide or protein derived from a Coronavirus ORF3a protein as defined above, or fragments and variants thereof. In that context, any coding sequence encoding at least one Coronavirus ORF3a protein as defined herein, or fragments and variants thereof may be understood as suitable coding sequence and may therefore be comprised in the nucleic acid of the composition.

In preferred embodiments, the at least one nucleic acid comprises or consists of at least one coding sequence encoding at least one antigenic peptide or protein selected or derived from Coronavirus ORF3a protein as defined herein, preferably encoding any one of SEQ ID NOs: 16672-16990, 27993-28000, or fragments of variants thereof. It has to be understood that, on nucleic acid level, any sequence (DNA or RNA sequence) which encodes an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 16672-16990, 27993-28000, or fragments or variants thereof, may be selected and may accordingly be understood as suitable coding sequence of the invention. Further information regarding said amino acid sequences is also provided in Table 1, and under <223> identifier of the ST25 sequence listing of respective sequence SEQ ID NOs.

In preferred embodiments, the at least one coding sequence of the nucleic acid is a codon modified coding sequence as defined herein, wherein the amino acid sequence, that is Coronavirus ORF3a protein, encoded by the at least one codon modified coding sequence, is preferably not being modified compared to the amino acid sequence encoded by the corresponding wild type or reference coding sequence.

In particularly preferred embodiments, the at least one coding sequence of the nucleic acid is a codon modified coding sequence, wherein the codon modified coding sequence is selected a G/C optimized coding sequence, a human codon usage adapted coding sequence, or a G/C modified coding sequence.

In embodiments, the at least one coding sequence comprises or consists at least one nucleic acid sequence encoding a Coronavirus ORF3a protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 18549-18867, 20426-20744, 22303-22621, 28114-28121, 28235-28242, 28278, 28365-28372, 28486-28493, or a fragment or variant of any of these sequences. Further information regarding respective nucleic acid sequences is provided under <223> identifier of the respective SEQ ID NO in the sequence listing, and in Table 1, rows 95-108, 202-209, see in particular Column C).

In preferred embodiments, the at least one coding sequence comprises or consists at least one nucleic acid sequence encoding a SARS-CoV-2 ORF3a protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 18561-18867, 20438-20744, 22315-22621 or a fragment or variant of any of these sequences. Particularly preferred nucleic acid sequences encoding SARS-CoV-2 ORF3a in the context of the invention are provided in Table 9.

In further embodiments, the at least one coding sequence comprises or consists at least one nucleic acid sequence encoding a SARS-CoV-2 variant ORF3a protein being identical or at least 70%, 80%, 85%, 86%, 87%, 88%, 89%, 90%, 91%, 92%, 93%, 94%, 95%, 96% 97% 98%, or 99% identical to any one of the nucleic acid sequences selected from SEQ ID NOs: 28114-28121, 28235-28242, 28365-28372, 28486-28493 or a fragment or variant of any of these sequences. Further information regarding respective nucleic acid sequences is provided under <223> identifier of the respective SEQ ID NO in the sequence listing, and in Table 1, rows 202-209).

In embodiments, the at least one nucleic acid comprises or consists of a nucleic acid sequence encoding a Coronavirus ORF3a protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 20426-20744, 22303-22621, 28235-28242, 28278, 28365-28372, 28486-28493 or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides. Further information regarding respective nucleic acid sequences is provided under <223> identifier of the respective SEQ ID NO in the sequence listing, and in Table 1, rows 95-108, 202-209, see in particular Column D).

In embodiments, the at least one nucleic acid comprises or consists of a nucleic acid sequence encoding a SARS-CoV-2 ORF3a protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 20438-20744, 22315-22621, or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides. Particularly preferred mRNA sequences encoding SARS-CoV-2 ORF3a in the context of the invention are provided in Table 9.

In further embodiments, the at least one nucleic acid comprises or consists of a nucleic acid sequence encoding a SARS-CoV-2 variant ORF3a protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 28235-28242, 28365-28372, 28486-28493 or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides.

In preferred embodiments, the pharmaceutical composition comprises at least one nucleic acid comprising at least one coding sequence encoding at least one antigenic peptide or protein selected or derived from at least one Coronavirus ORF8 protein, or an immunogenic fragment or immunogenic variant thereof.

Preferred antigenic peptide or proteins selected or derived from a Coronavirus ORF8 protein as defined herein are provided in Table 1 (rows 109-116, 210-221). Further provided in Table 1 are preferred nucleic acid sequences (coding sequences, mRNA sequences) encoding said antigenic peptide or proteins.

In embodiments, the Coronavirus ORF8 protein comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 16991-17097, 28001-28012, or an immunogenic fragment or immunogenic variant of any of these. Further information regarding respective amino acid sequences is provided under <223> identifier of the respective SEQ ID NO in the sequence listing, and in Table 1, row 109-116, 210-221, see in particular Column B).

In preferred embodiments, the Coronavirus ORF8 protein is selected or derived from SARS-CoV-2 and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 16997-17097, or an immunogenic fragment or immunogenic variant of any of these, Particularly preferred amino acid sequences in the context of the invention are provided in Table 9.

In further embodiments, the Coronavirus ORF8 protein selected or derived from SARS-CoV-2 is a variant ORF8 protein and comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 28001-28012, or an immunogenic fragment or immunogenic variant of any of these.

According to preferred embodiments, the at least one nucleic acid of the composition comprises at least one coding sequence encoding at least one antigenic peptide or protein derived from a Coronavirus ORF8 protein as defined above, or fragments and variants thereof. In that context, any coding sequence encoding at least one Coronavirus ORF8 protein as defined herein, or fragments and variants thereof may be understood as suitable coding sequence and may therefore be comprised in the nucleic acid of the composition.

In preferred embodiments, the at least one nucleic acid comprises or consists of at least one coding sequence encoding at least one antigenic peptide or protein selected or derived from Coronavirus ORF8 protein as defined herein, preferably encoding any one of SEQ ID NOs: 16991-17097, 28001-28012, or fragments of variants thereof. It has to be understood that, on nucleic acid level, any sequence (DNA or RNA sequence) which encodes an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 16991-17097, 28001-28012, or fragments or variants thereof, may be selected and may accordingly be understood as suitable coding sequence of the invention. Further information regarding said amino acid sequences is also provided in Table 1, and under <223> identifier of the ST25 sequence listing of respective sequence SEQ ID NOs.

In preferred embodiments, the at least one coding sequence of the nucleic acid is a codon modified coding sequence as defined herein, wherein the amino acid sequence, that is Coronavirus ORF8 protein, encoded by the at least one codon modified coding sequence, is preferably not being modified compared to the amino acid sequence encoded by the corresponding wild type or reference coding sequence.

In particularly preferred embodiments, the at least one coding sequence of the nucleic acid is a codon modified coding sequence, wherein the codon modified coding sequence is selected a G/C optimized coding sequence, a human codon usage adapted coding sequence, or a G/C modified coding sequence.

In embodiments, the at least one coding sequence comprises or consists at least one nucleic acid sequence encoding a Coronavirus ORF8 protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 18868-18974, 20745-20851, 22622-22728, 28122-28133, 28243-28254, 28373-28384, 28494-28505, or a fragment or variant of any of these sequences. Further information regarding respective nucleic acid sequences is provided under <223> identifier of the respective SEQ ID NO in the sequence listing, and in Table 1, row 109-116, 210-221, see in particular Column C).

In preferred embodiments, the at least one coding sequence comprises or consists at least one nucleic acid sequence encoding a SARS-CoV-2 ORF8 protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 18874-18974, 20751-20851, 22628-22728, or a fragment or variant of any of these sequences. Particularly preferred nucleic acid sequences encoding SARS-CoV-2 ORF8 in the context of the invention are provided in Table 9.

In further embodiments, the at least one coding sequence comprises or consists at least one nucleic acid sequence encoding a SARS-CoV-2 variant ORF8 protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 28122-28133, 28243-28254, 28373-28384, 28494-28505, or a fragment or variant of any of these sequences. Further information regarding respective nucleic acid sequences is provided under <223> identifier of the respective SEQ ID NO in the sequence listing, and in Table 1, rows 210-221).

In embodiments, the at least one nucleic acid comprises or consists of a nucleic acid sequence encoding a Coronavirus ORF8 protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 20745-20851, 22622-22728, 28243-28254, 28373-28384, 28494-28505, or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides. Further information regarding respective nucleic acid sequences is provided under <223> identifier of the respective SEQ ID NO in the sequence listing, and in Table 1, row 109-116, 210-221, see in particular Column D).

In embodiments, the at least one nucleic acid comprises or consists of a nucleic acid sequence encoding a SARS-CoV-2 ORF8 protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 20751-20851, 22628-22728, or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides. Particularly preferred mRNA sequences encoding SARS-CoV-2 ORF8 in the context of the invention are provided in Table 9.

In further embodiments, the at least one nucleic acid comprises or consists of a nucleic acid sequence encoding a SARS-CoV-2 variant ORF8 protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 28254, 28373-28384, 28494-28505, or a fragment or variant of any of these sequences. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides.

Nucleic Acid Encoding a Coronavirus E Protein:

In embodiments, the pharmaceutical composition comprises at least one nucleic acid comprising at least one coding sequence encoding at least one antigenic peptide or protein selected or derived from at least one Coronavirus envelope (E) protein, or an immunogenic fragment or immunogenic variant thereof.

In preferred embodiments, the Coronavirus E protein comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 15675-15719, 27940-27947, or an immunogenic fragment or immunogenic variant of any of these. Further information regarding respective amino acid sequences is provided under <223> identifier of the respective SEQ ID NO in the sequence listing, and in Table 1, row 33-48, 149-156, see in particular Column B).

In preferred embodiments, the at least one coding sequence comprises or consists at least one nucleic acid sequence encoding a Coronavirus E protein being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 17552-17596, 19429-19473, 21306-21350, 28061-28068, 28182-28189, 28312-28319, 28433-28440, or a fragmentor variant ofany of these sequences. Further information regarding respective amino acid sequences is provided under <223> identifier of the respective SEQ ID NO in the sequence listing, and in Table 1, row 33-48, 149-156, see in particular Column C and D).

Preferred antigenic peptide or proteins selected or derived from a Coronavirus antigen constructs as defined above are provided in Table U (rows 1 to 237). Therein, each row 1 to 237 corresponds to a suitable Coronavirus constructs. Column A of Table provides a description of the Coronavirus antigen constructs. Column B of Table provides protein (amino acid) SEQ ID NOs of respective Coronavirus antigen constructs encoded by the nucleic acids. Column C of Table 1 provides SEQ ID NO of the corresponding G/C optimized nucleic acid coding sequences (opt1, gc). Column D of Table 1 provided respective suitable mRNA sequences. Further information for each sequence ID is provided under <223> identifier of the respective SEQ ID NOs in the sequence listing. Particularly preferred sequences in the context of the invention are provided in Table 9.

TABLE 1
Coronavirus constructs (amino acid sequences, nucleic acid coding sequences, mRNA):

| | | B | C | |
|---|---|---|---|---|
| | | SEQ ID | SEQ ID | D |
| | A | NO: | NO: CDS | SEQ ID NO: |
| Row | Name | Protein | opt1 (gc) | mRNA |
| 1 | MERS-COV/MERS-COV-Jeddah-human-1 membrane protein | 15221 | 17098 | 18975, 20852 |
| 2 | MERS-COV/AI-Hasa_4_2013 membrane protein | 15222 | 17099 | 18976, 20853 |
| 3 | MERS-COV/Riyadh_14_2013 membrane protein | 15223 | 17100 | 18977, 20854 |
| 4 | MERS-COV/England 1 membrane protein | 15224 | 17101 | 18978, 20855 |
| 5 | SARS-COV/Tor2 membrane protein | 15225 | 17102 | 18979, 20856 |
| 6 | HCoV/OC43 membrane protein | 15226 | 17103 | 18980, 20857 |
| 7 | HCoV/HKU1 membrane protein | 15227 | 17104 | 18981, 20858 |
| 8 | HCoV/229E/BN1/GER/2015 membrane protein | 15228 | 17105 | 18982, 20859 |
| 9 | HCoV/NL63/RPTEC/2004 membrane protein | 15229 | 17106 | 18983, 20860 |
| 10 | Bat SARS-like CoV/WIV1 membrane protein | 15230 | 17107 | 18984, 20861 |
| 11 | BatCoV/HKU9-1 BF_0051 membrane protein | 15231 | 17108 | 18985, 20862 |
| 12 | PDCoV/Swine/Thailand/S5011/2015 membrane protein | 15232 | 17109 | 18986, 20863 |
| 13 | PEDV/NPL-PEDv/2013/P10 membrane protein | 15233 | 17110 | 18987, 20864 |
| 14 | MHV/A59 membrane protein | 15234 | 17111 | 18988, 20865 |
| 15 | SARS-COV-2/Wuhan-Hu-1 membrane protein | 15235 | 17112 | 18989, 20866, |
| | | | | 28271 |
| 16 | SARS-COV-2 “divers strains/isolates” membrane protein | 15236- | 17113- | 18990-19049, |
| | | 15295 | 17172 | 20867-20926 |
| 17 | MERS-COV/MERS-COV-Jeddah-human-1 nucleocapsid protein | 15296 | 17173 | 19050, 20927 |
| 18 | MERS-COV/Al-Hasa_4_2013 nucleocapsid protein | 15297 | 17174 | 19051, 20928 |
| 19 | MERS-COV/Riyadh_14_2013 nucleocapsid protein | 15298 | 17175 | 19052, 20929 |
| 20 | MERS-COV/England 1 nucleocapsid protein | 15299 | 17176 | 19053, 20930 |
| 21 | SARS-COV/Tor2 nucleocapsid protein | 15300 | 17177 | 19054, 20931 |
| 22 | HCoV/OC43 nucleocapsid protein | 15301 | 17178 | 19055, 20932 |
| 23 | HCoV/HKU1 nucleocapsid protein | 15302 | 17179 | 19056, 20933 |
| 24 | HCoV/229E/BN1/GER/2015 nucleocapsid protein | 15303 | 17180 | 19057, 20934 |
| 25 | HCoV/NL63/RPTEC/2004 nucleocapsid protein | 15304 | 17181 | 19058, 20935 |
| 26 | Bat SARS-like CoV/WIV1 nucleocapsid protein | 15305 | 17182 | 19059, 20936 |
| 27 | BatCoV/HKU9-1 BF_0051 nucleocapsid protein | 15306 | 17183 | 19060, 20937 |
| 28 | PDCoV/Swine/Thailand/S5011/2015 nucleocapsid protein | 15307 | 17184 | 19061, 20938 |
| 29 | PEDV/NPL-PEDv/2013/P10 nucleocapsid protein | 15308 | 17185 | 19062, 20939 |
| 30 | MHV/A59 nucleocapsid protein | 15309 | 17186 | 19063, 20940 |
| 31 | SARS-COV-2/Wuhan-Hu-1 nucleocapsid protein | 15310 | 17187 | 19064, 20941, |
| | | | | 28272 |
| 32 | SARS-COV-2 “divers strains/isolates” nucleocapsid protein | 15311- | 17188- | 19065-19428, |
| | | 15674 | 17551 | 20942-21305 |
| 33 | MERS-COV/MERS-COV-Jeddah-human-1 envelope protein | 15675 | 17552 | 19429, 21306 |
| 34 | MERS-COV/Al-Hasa_4_2013 envelope protein | 15676 | 17553 | 19430, 21307 |
| 35 | MERS-COV/Riyadh_14_2013 envelope protein | 15677 | 17554 | 19431, 21308 |
| 36 | MERS-COV/England 1 envelope protein | 15678 | 17555 | 19432, 21309 |
| 37 | SARS-COV/Tor2 envelope protein | 15679 | 17556 | 19433, 21310 |
| 38 | HCoV/OC43 envelope protein | 15680 | 17557 | 19434, 21311 |
| 39 | HCoV/HKU1 envelope protein | 15681 | 17558 | 19435, 21312 |
| 40 | HCoV/229E/BN1/GER/2015 envelope protein | 15682 | 17559 | 19436, 21313 |
| 41 | HCoV/NL63/RPTEC/2004 envelope protein | 15683 | 17560 | 19437, 21314 |
| 42 | Bat SARS-like CoV/WIV1 envelope protein | 15684 | 17561 | 19438, 21315 |
| 43 | BatCoV/HKU9-1 BF_0051 envelope protein | 15685 | 17562 | 19439, 21316 |
| 44 | PDCoV/Swine/Thailand/S5011/2015 envelope protein | 15686 | 17563 | 19440, 21317 |
| 45 | PEDV/NPL-PEDv/2013/P10 envelope protein | 15687 | 17564 | 19441, 21318 |
| 46 | MHV/A59 envelope protein | 15688 | 17565 | 19442, 21319 |
| 47 | SARS-CoV-2/Wuhan-Hu-1 envelope protein | 15689 | 17566 | 19443, 21320, |
| | | | | 28273 |
| 48 | SARS-COV-2 “divers strains/isolates” envelope protein | 15690- | 17567- | 19444-19473, |
| | | 15719 | 17596 | 21321-21350 |
| 49 | MERS-COV/MERS-COV-Jeddah-human-1 non-structural protein 3 | 15720 | 17597 | 19474, 21351 |
| 50 | MERS-COV/AI-Hasa_4_2013 non-structural protein 3 | 15721 | 17598 | 19475, 21352 |
| 51 | MERS-COV/Riyadh_14_2013 non-structural protein 3 | 15722 | 17599 | 19476, 21353 |
| 52 | MERS-COV/England 1 non-structural protein 3 | 15723 | 17600 | 19477, 21354 |
| 53 | SARS-COV/Tor2 non-structural protein 3 | 15724 | 17601 | 19478, 21355 |
| 54 | HCoV/OC43 non-structural protein 3 | 15725 | 17602 | 19479, 21356 |
| 55 | HCoV/HKU1 non-structural protein 3 | 15726 | 17603 | 19480, 21357 |
| 56 | HCoV/229E/BN1/GER/2015 non-structural protein 3 | 15727 | 17604 | 19481, 21358 |
| 57 | HCoV/NL63/RPTEC/2004 non-structural protein 3 | 15728 | 17605 | 19482, 21359 |
| 58 | Bat SARS-like CoV/WIV1 non-structural protein 3 | 15729 | 17606 | 19483, 21360 |
| 59 | BatCoV/HKU9-1 BF_0051 non-structural protein 3 | 15730 | 17607 | 19484, 21361 |
| 60 | PEDV/NPL-PEDv/2013/P10 non-structural protein 3 | 15731 | 17608 | 19485, 21362 |
| 61 | MHV/A59 non-structural protein 3 | 15732 | 17609 | 19486, 21363 |
| 62 | SARS-COV-2/Wuhan-Hu-1 non-structural protein 3 | 15733 | 17610 | 19487, 21364, |
| | | | | 28274 |
| 63 | SARS-COV-2/Wuhan/WIV05/2019 non-structural protein 3 | 15734 | 17611 | 19488, 21365, |
| | | | | 28275 |
| 64 | SARS-COV-2 “divers strains/isolates” non-structural protein 3 | 15735- | 17612- | 19489-20155, |
| | | 16401 | 18278 | 21366-22032 |
| 65 | MERS-COV/MERS-COV-Jeddah-human-1 non-structural protein 4 | 16402 | 18279 | 20156, 22033 |
| 66 | MERS-COV/AI-Hasa_4_2013 non-structural protein 4 | 16403 | 18280 | 20157, 22034 |
| 67 | MERS-COV/Riyadh_14_2013 non-structural protein 4 | 16404 | 18281 | 20158, 22035 |
| 68 | MERS-COV/England 1 non-structural protein 4 | 16405 | 18282 | 20159, 22036 |
| 69 | SARS-COV/Tor2 non-structural protein 4 | 16406 | 18283 | 20160, 22037 |
| 70 | HCoV/OC43 non-structural protein 4 | 16407 | 18284 | 20161, 22038 |
| 71 | HCoV/HKU1 non-structural protein 4 | 16408 | 18285 | 20162, 22039 |
| 72 | HCoV/229E/BN1/GER/2015 non-structural protein 4 | 16409 | 18286 | 20163, 22040 |
| 73 | HCoV/NL63/RPTEC/2004 non-structural protein 4 | 16410 | 18287 | 20164, 22041 |
| 74 | Bat SARS-like CoV/WIV1 non-structural protein 4 | 16411 | 18288 | 20165, 22042 |
| 75 | BatCoV/HKU9-1 BF_0051 non-structural protein 4 | 16412 | 18289 | 20166, 22043 |
| 76 | PEDV/NPL-PEDv/2013/P10 non-structural protein 4 | 16413 | 18290 | 20167, 22044 |
| 77 | MHV/A59 non-structural protein 4 | 16414 | 18291 | 20168, 22045 |
| 78 | SARS-COV-2/Wuhan-Hu-1 non-structural protein 4 | 16415 | 18292 | 20169, 22046, |
| | | | | 28276 |
| 79 | SARS-COV-2 “divers strains/isolates” non-structural protein 4 | 16416- | 18293- | 20170-20322, |
| | | 16568 | 18445 | 22047-22199 |
| 80 | MERS-COV/MERS-COV-Jeddah-human-1 non-structural protein 6 | 16569 | 18446 | 20323, 22200 |
| 81 | MERS-COV/Al-Hasa_4_2013 non-structural protein 6 | 16570 | 18447 | 20324, 22201 |
| 82 | MERS-COV/Riyadh_14_2013 non-structural protein 6 | 16571 | 18448 | 20325, 22202 |
| 83 | MERS-COV/England 1 non-structural protein 6 | 16572 | 18449 | 20326, 22203 |
| 84 | SARS-COV/Tor2 non-structural protein 6 | 16573 | 18450 | 20327, 22204 |
| 85 | HCoV/OC43 non-structural protein 6 | 16574 | 18451 | 20328, 22205 |
| 86 | HCoV/HKU1 non-structural protein 6 | 16575 | 18452 | 20329, 22206 |
| 87 | HCoV/229E/BN1/GER/2015 non-structural protein 6 | 16576 | 18453 | 20330, 22207 |
| 88 | HCoV/NL63/RPTEC/2004 non-structural protein 6 | 16577 | 18454 | 20331, 22208 |
| 89 | Bat SARS-like CoV/WIV1 non-structural protein 6 | 16578 | 18455 | 20332, 22209 |
| 90 | BatCoV/HKU9-1 BF_0051 non-structural protein 6 | 16579 | 18456 | 20333, 22210 |
| 91 | PEDV/NPL-PEDv/2013/P10 non-structural protein 6 | 16580 | 18457 | 20334, 22211 |
| 92 | MHV/A59 non-structural protein 6 | 16581 | 18458 | 20335, 22212 |
| 93 | SARS-COV-2/Wuhan-Hu-1 non-structural protein 6 | 16582 | 18459 | 20336, 22213, |
| | | | | 28277 |
| 94 | SARS-COV-2 “divers strains/isolates” non-structural protein 6 | 16583- | 18460- | 20337-20425, |
| | | 16671 | 18548 | 22214-22302 |
| 95 | MERS-COV/MERS-COV-Jeddah-human-1 ORF3 protein | 16672 | 18549 | 20426, 22303 |
| 96 | MERS-COV/Al-Hasa_4_2013 ORF3 protein | 16673 | 18550 | 20427, 22304 |
| 97 | MERS-COV/Riyadh_14_2013 ORF3 protein | 16674 | 18551 | 20428, 22305 |
| 98 | MERS-COV/England 1 ORF3 protein | 16675 | 18552 | 20429, 22306 |
| 99 | SARS-COV/Tor2 ORF3 protein | 16676 | 18553 | 20430, 22307 |
| 100 | HCoV/OC43 ORF3 protein | 16677 | 18554 | 20431, 22308 |
| 101 | HCoV/HKU1 ORF3 protein | 16678 | 18555 | 20432, 22309 |
| 102 | HCoV/229E/BN1/GER/2015 ORF3 protein | 16679 | 18556 | 20433, 22310 |
| 103 | HCoV/NL63/RPTEC/2004 ORF3 protein | 16680 | 18557 | 20434, 22311 |
| 104 | Bat SARS-like CoV/WIV1 ORF3 protein | 16681 | 18558 | 20435, 22312 |
| 105 | BatCoV/HKU9-1 BF_0051 ORF3 protein | 16682 | 18559 | 20436, 22313 |
| 106 | PEDV/NPL-PEDv/2013/P10 ORF3 protein | 16683 | 18560 | 20437, 22314 |
| 107 | SARS-COV-2/Vuhan-Hu-1 ORF3a protein | 16684 | 18561 | 20438, 22315, |
| | | | | 28278 |
| 108 | SARS-COV-2 “divers strains/isolates” ORF3a protein | 16685- | 18562- | 20439-20744, |
| | | 16990 | 18867 | 22316-22621 |
| 109 | MERS-COV/MERS-COV-Jeddah-human-1 ORF8b protein | 16991 | 18868 | 20745, 22622 |
| 110 | MERS-COV/Al-Hasa_4_2013 ORF8b protein | 16992 | 18869 | 20746, 22623 |
| 111 | MERS-COV/Riyadh_14_2013 ORF8b protein | 16993 | 18870 | 20747, 22624 |
| 112 | SARS-COV/Tor2 ORF8a protein | 16994 | 18871 | 20748, 22625 |
| 113 | SARS-COV/Tor2 ORF8b protein | 16995 | 18872 | 20749, 22626 |
| 114 | Bat SARS-like CoV/WIV1 ORF8 protein | 16996 | 18873 | 20750, 22627 |
| 115 | SARS-COV-2/Wuhan-Hu-1 ORF8 protein | 16997 | 18874 | 20751, 22628, |
| | | | | 28279 |
| 116 | SARS-COV-2 “divers strains/isolates” ORF8 protein | 16998- | 18875- | 20752-20851, |
| | | 17097 | 18974 | 22629-22728 |
| 117 | SARS-COV-2/Wuhan-Hu-1 non-structural protein 13 | 27908 | 28029 | 28150, 28280, |
| | | | | 28401 |
| 118 | SARS-COV-2/Wuhan-Hu-1 non-structural protein 14 | 27909 | 28030 | 28151, 28281, |
| | | | | 28402 |
| 119 | SARS-COV-2/Wuhan-Hu-1 membrane protein (M) (1-222)(182T) | 27910 | 28031 | 28152, 28282, |
| | | | | 28403 |
| 120 | SARS-COV-2/Wuhan-Hu-1 membrane protein (M) (1- | 27911 | 28032 | 28153, 28283, |
| | 222)(A2S_F28L_V70L) | | | 28404 |
| 121 | SARS-COV-2/Wuhan-Hu-1 membrane protein (M) (1- | 27912 | 28033 | 28154, 28284, |
| | 222)(A2T_1201V) | | | 28405 |
| 122 | SARS-COV-2/Wuhan-Hu-1 membrane protein (M) (1- | 27913 | 28034 | 28155, 28285, |
| | 222)(N121K_H125Y_H148Y_H155Y_K162N) | | | 28406 |
| 123 | SARS-COV-2/Wuhan-Hu-1 membrane protein (M) (1- | 27914 | 28035 | 28156, 28286, |
| | 222)(A63T_H125Y) | | | 28407 |
| 124 | SARS-COV-2/Wuhan-Hu-1 membrane protein (M) (1- | 27915 | 28036 | 28157, 28287, |
| | 222)(A2S_M1091_H125Y) | | | 28408 |
| 125 | SARS-COV-2/Wuhan-Hu-1 membrane protein (M) (1- | 27916 | 28037 | 28158, 28288, |
| | 222)(A2V_M1091_H125Y) | | | 28409 |
| 126 | SARS-COV-2/Wuhan-Hu-1 membrane protein (M) (1- | 27917 | 28038 | 28159, 28289, |
| | 222)(L17F_M1091) | | | 28410 |
| 127 | SARS-COV-2/Wuhan-Hu-1 nucleocapsid protein (N) (1- | 27918 | 28039 | 28160, 28290, |
| | 419)(D3L_R203K_G204R_S235F) | | | 28411 |
| 128 | SARS-COV-2/Wuhan-Hu-1 nucleocapsid protein (N) (1- | 27919 | 28040 | 28161, 28291, |
| | 419)(T2051) | | | 28412 |
| 129 | SARS-COV-2/Wuhan-Hu-1 nucleocapsid protein (N) (1- | 27920 | 28041 | 28162, 28292, |
| | 419)(P80R) | | | 28413 |
| 130 | SARS-COV-2/Wuhan-Hu-1 nucleocapsid protein (N) (1- | 27921 | 28042 | 28163, 28293, |
| | 419)(P80R_R203K_G204R) | | | 28414 |
| 131 | SARS-COV-2/Wuhan-Hu-1 nucleocapsid protein (N) (1- | 27922 | 28043 | 28164, 28294, |
| | 419)(D63G_R203M_G215C_D377Y) | | | 28415 |
| 132 | SARS-COV-2/Wuhan-Hu-1 nucleocapsid protein (N) (1- | 27923 | 28044 | 28165, 28295, |
| | 419)(D63G_R203M_D377Y) | | | 28416 |
| 133 | SARS-COV-2/Vuhan-Hu-1 nucleocapsid protein (N) (1- | 27924 | 28045 | 28166, 28296, |
| | 419)(T205\|_T3621) | | | 28417 |
| 134 | SARS-COV-2/Wuhan-Hu-1 nucleocapsid protein (N) (1- | 27925 | 28046 | 28167, 28297, |
| | 419)(S2Y_D3del_A12G_T2051) | | | 28418 |
| 135 | SARS-COV-2/Wuhan-Hu-1 nucleocapsid protein (N) (1- | 27926 | 28047 | 28168, 28298, |
| | 419)(P13L_R203K_G204R_G214C) | | | 28419 |
| 136 | SARS-COV-2/Wuhan-Hu-1 nucleocapsid protein (N) (1- | 27927 | 28048 | 28169, 28299, |
| | 419)(P67S_R203M_D377Y) | | | 28420 |
| 137 | SARS-COV-2/Wuhan-Hu-1 nucleocapsid protein (N) (1- | 27928 | 28049 | 28170, 28300, |
| | 419)(1157V_R203K_G204R) | | | 28421 |
| 138 | SARS-COV-2/Wuhan-Hu-1 nucleocapsid protein (N) (1- | 27929 | 28050 | 28171, 28301, |
| | 419)(A119S_R203K_G204R_M234I) | | | 28422 |
| 139 | SARS-COV-2/Wuhan-Hu-1 nucleocapsid protein (N) (1- | 27930 | 28051 | 28172, 28302, |
| | 419)(R203K_G204R) | | | 28423 |
| 140 | SARS-COV-2/Wuhan-Hu-1 nucleocapsid protein (N) (1- | 27931 | 28052 | 28173, 28303, |
| | 419)(R203K_G204R_G212V) | | | 28424 |
| 141 | SARS-COV-2/Wuhan-Hu-1 nucleocapsid protein (N) (1- | 27932 | 28053 | 28174, 28304, |
| | 419)(R203K_G204R_A208G_R209del) | | | 28425 |
| 142 | SARS-COV-2/Wuhan-Hu-1 nucleocapsid protein (N) (1- | 27933 | 28054 | 28175, 28305, |
| | 419)(R203M_D377Y) | | | 28426 |
| 143 | SARS-COV-2/Wuhan-Hu-1 nucleocapsid protein (N) (1- | 27934 | 28055 | 28176, 28306, |
| | 419)(T2051_M2341) | | | 28427 |
| 144 | SARS-COV-2/Wuhan-Hu-1 nucleocapsid protein (N) (1- | 27935 | 28056 | 28177, 28307, |
| | 419)(M2341) | | | 28428 |
| 145 | SARS-COV-2/Wuhan-Hu-1 nucleocapsid protein (N) (1- | 27936 | 28057 | 28178, 28308, |
| | 419)(A12G_T2051) | | | 28429 |
| 146 | SARS-COV-2/Wuhan-Hu-1 nucleocapsid protein (N) (1- | 27937 | 28058 | 28179, 28309, |
| | 419)(P13L_P199L_S202R_T2051_M2341) | | | 28430 |
| 147 | SARS-COV-2/Wuhan-Hu-1 nucleocapsid protein (N) (1- | 27938 | 28059 | 28180, 28310, |
| | 419)(P13L_R203K_G204R_G214C_T3661) | | | 28431 |
| 148 | SARS-COV-2/Wuhan-Hu-1 nucleocapsid protein (N) (1- | 27939 | 28060 | 28181, 28311, |
| | 419)(R203K_G204R_A208G_R209del_M2341) | | | 28432 |
| 149 | SARS-COV-2/Wuhan-Hu-1 envelope protein (E) (1-75)(P71L) | 27940 | 28061 | 28182, 28312, |
| | | | | 28433 |
| 150 | SARS-COV-2/Wuhan-Hu-1 envelope protein (E) (1- | 27941 | 28062 | 28183, 28313, |
| | 75)(T91_C43F_V49L_V58F_V62F_L73F) | | | 28434 |
| 151 | SARS-COV-2/Wuhan-Hu-1 envelope protein (E) (1- | 27942 | 28063 | 28184, 28314, |
| | 75)(T91_V58F_V62F_S68F_L73F) | | | 28435 |
| 152 | SARS-COV-2/Wuhan-Hu-1 envelope protein (E) (1- | 27943 | 28064 | 28185, 28315, |
| | 75)(T91_V58F_S68A_R69G_P71S) | | | 28436 |
| 153 | SARS-COV-2/Wuhan-Hu-1 envelope protein (E) (1-75)(L21F) | 27944 | 28065 | 28186, 28316, |
| | | | | 28437 |
| 154 | SARS-COV-2/Wuhan-Hu-1 envelope protein (E) (1- | 27945 | 28066 | 28187, 28317, |
| | 75)(T91_L21F_S55F_V58F_S68F_L73F) | | | 28438 |
| 155 | SARS-COV-2/Wuhan-Hu-1 envelope protein (E) (1- | 27946 | 28067 | 28188, 28318, |
| | 75)(T91_S55F_V58F_S68F_P71L_D72Y_L73F) | | | 28439 |
| 156 | SARS-COV-2/Wuhan-Hu-1 envelope protein (E) (1- | 27947 | 28068 | 28189, 28319, |
| | 75)(T91_S55F_V58F_S68F_D72Y_L73F) | | | 28440 |
| 157 | SARS-COV-2/Wuhan-Hu-1 non-structural protein 3 (NSP3) (1- | 27948 | 28069 | 28190, 28320, |
| | 1946)(T1841_A891D_11413T) | | | 28441 |
| 158 | SARS-COV-2/Wuhan-Hu-1 non-structural protein 3 (NSP3) (1- | 27949 | 28070 | 28191, 28321, |
| | 1946)(A329T_P823L) | | | 28442 |
| 159 | SARS-COV-2/Wuhan-Hu-1 non-structural protein 3 (NSP3) (1- | 27950 | 28071 | 28192, 28322, |
| | 1946)(S371L_K978Q) | | | 28443 |
| 160 | SARS-COV-2/Wuhan-Hu-1 non-structural protein 3 (NSP3) (1- | 27951 | 28072 | 28193, 28323, |
| | 1946)(A489S_P1229L_P1470S) | | | 28444 |
| 161 | SARS-COV-2/Wuhan-Hu-1 non-structural protein 3 (NSP3) (1- | 27952 | 28073 | 28194, 28324, |
| | 1946)(K838N) | | | 28445 |
| 162 | SARS-COV-2/Wuhan-Hu-1 non-structural protein 3 (NSP3) (1- | 27953 | 28074 | 28195, 28325, |
| | 1946)(K838N_P1229L_N1779S) | | | 28446 |
| 163 | SARS-COV-2/Wuhan-Hu-1 non-structural protein 3 (NSP3) (1- | 27954 | 28075 | 28196, 28326, |
| | 1946)(K838N_N1779S) | | | 28447 |
| 164 | SARS-COV-2/Wuhan-Hu-1 non-structural protein 3 (NSP3) (1- | 27955 | 28076 | 28197, 28327, |
| | 1946)(A489S_P1229L_P1470S_A1712V) | | | 28448 |
| 165 | SARS-COV-2/Wuhan-Hu-1 non-structural protein 3 (NSP3) (1- | 27956 | 28077 | 28198, 28328, |
| | 1946)(S795L_K838N) | | | 28449 |
| 166 | SARS-COV-2/Wuhan-Hu-1 non-structural protein 3 (NSP3) (1- | 27957 | 28078 | 28199, 28329, |
| | 1946)(A42V_T4291_D822N_P1470S) | | | 28450 |
| 167 | SARS-COV-2/Wuhan-Hu-1 non-structural protein 3 (NSP3) (1- | 27958 | 28079 | 28200, 28330, |
| | 1946)(T238A_T721I_N1330D) | | | 28451 |
| 168 | SARS-COV-2/Wuhan-Hu-1 non-structural protein 3 (NSP3) (1- | 27959 | 28080 | 28201, 28331, |
| | 1946)(H343Y_P823L_K928I_N1588S) | | | 28452 |
| 169 | SARS-COV-2/Wuhan-Hu-1 non-structural protein 3 (NSP3) (1- | 27960 | 28081 | 28202, 28332, |
| | 1946)(E379V_K1694N) | | | 28453 |
| 170 | SARS-COV-2/Wuhan-Hu-1 non-structural protein 3 (NSP3) (1- | 27961 | 28082 | 28203, 28333, |
| | 1946)(T4291_P1470S_F1570V) | | | 28454 |
| 171 | SARS-COV-2/Wuhan-Hu-1 non-structural protein 3 (NSP3) (1- | 27962 | 28083 | 28204, 28334, |
| | 1946)(D737G_S1808F) | | | 28455 |
| 172 | SARS-COV-2/Wuhan-Hu-1 non-structural protein 3 (NSP3) (1- | 27963 | 28084 | 28205, 28335, |
| | 1946)(T7501) | | | 28456 |
| 173 | SARS-COV-2/Wuhan-Hu-1 non-structural protein 3 (NSP3) (1- | 27964 | 28085 | 28206, 28336, |
| | 1946)(T11901) | | | 28457 |
| 174 | SARS-COV-2/Wuhan-Hu-1 non-structural protein 3 (NSP3) (1- | 27965 | 28086 | 28207, 28337, |
| | 1946)(A1527V_T18311) | | | 28458 |
| 175 | SARS-COV-2/Wuhan-Hu-1 non-structural protein 3 (NSP3) (1- | 27966 | 28087 | 28208, 28338, |
| | 1946)(A86V_A656V_11022V_E1847D) | | | 28459 |
| 176 | SARS-COV-2/Wuhan-Hu-1 non-structural protein 3 (NSP3) (1- | 27967 | 28088 | 28209, 28339, |
| | 1946)(D136N_E379V_K1694N) | | | 28460 |
| 177 | SARS-COV-2/Wuhan-Hu-1 non-structural protein 3 (NSP3) (1- | 27968 | 28089 | 28210, 28340, |
| | 1946)(A862S_G1129S) | | | 28461 |
| 178 | SARS-COV-2/Wuhan-Hu-1 non-structural protein 3 (NSP3) (1- | 27969 | 28090 | 28211, 28341, |
| | 1946)(T1190L_K1772R) | | | 28462 |
| 179 | SARS-COV-2/Wuhan-Hu-1 non-structural protein 4 (NSP4) (1- | 27970 | 28091 | 28212, 28342, |
| | 501)(V168L_T4931) | | | 28463 |
| 180 | SARS-COV-2/Wuhan-Hu-1 non-structural protein 4 (NSP4) (1- | 27971 | 28092 | 28213, 28343, |
| | 501)(F18L) | | | 28464 |
| 181 | SARS-COV-2/Wuhan-Hu-1 non-structural protein 4 (NSP4) (1- | 27972 | 28093 | 28214, 28344, |
| | 501)(124T_G179S_S185N_A447V) | | | 28465 |
| 182 | SARS-COV-2/Wuhan-Hu-1 non-structural protein 4 (NSP4) (1- | 27973 | 28094 | 28215, 28345, |
| | 501)(V168L_A447V_T4931) | | | 28466 |
| 183 | SARS-COV-2/Wuhan-Hu-1 non-structural protein 4 (NSP4) (1- | 27974 | 28095 | 28216, 28346, |
| | 501)(Y206H_L265F_T2961_A473S) | | | 28467 |
| 184 | SARS-COV-2/Wuhan-Hu-1 non-structural protein 4 (NSP4) (1- | 27975 | 28096 | 28217, 28347, |
| | 501)(T174\|_A447V) | | | 28468 |
| 185 | SARS-COV-2/Wuhan-Hu-1 non-structural protein 4 (NSP4) (1- | 27976 | 28097 | 28218, 28348, |
| | 501)(T190\|_T4931) | | | 28469 |
| 186 | SARS-COV-2/Wuhan-Hu-1 non-structural protein 4 (NSP4) (1- | 27977 | 28098 | 28219, 28349, |
| | 501)(D218N_L439P) | | | 28470 |
| 187 | SARS-COV-2/Wuhan-Hu-1 non-structural protein 4 (NSP4) (1- | 27978 | 28099 | 28220, 28350, |
| | 501)(D218N_D460N) | | | 28471 |
| 188 | SARS-COV-2/Wuhan-Hu-1 non-structural protein 4 (NSP4) (1- | 27979 | 28100 | 28221, 28351, |
| | 501)(S396T) | | | 28472 |
| 189 | SARS-COV-2/Wuhan-Hu-1 non-structural protein 4 (NSP4) (1- | 27980 | 28101 | 28222, 28352, |
| | 501)(L439P) | | | 28473 |
| 190 | SARS-COV-2/Wuhan-Hu-1 non-structural protein 4 (NSP4) (1- | 27981 | 28102 | 28223, 28353, |
| | 501)(A447V) | | | 28474 |
| 191 | SARS-COV-2/Wuhan-Hu-1 non-structural protein 4 (NSP4) (1- | 27982 | 28103 | 28224, 28354, |
| | 501)(L439P_T4931) | | | 28475 |
| 192 | SARS-COV-2/Wuhan-Hu-1 non-structural protein 4 (NSP4) (1- | 27983 | 28104 | 28225, 28355, |
| | 501)(T115l_S396T) | | | 28476 |
| 193 | SARS-COV-2/Wuhan-Hu-1 non-structural protein 4 (NSP4) (1- | 27984 | 28105 | 28226, 28356, |
| | 501)(K400E_L439P_A447V) | | | 28477 |
| 194 | SARS-COV-2/Wuhan-Hu-1 non-structural protein 4 (NSP4) (1- | 27985 | 28106 | 28227, 28357, |
| | 501)(L448F) | | | 28478 |
| 195 | SARS-COV-2/Wuhan-Hu-1 non-structural protein 6 (NSP6) (1- | 27986 | 28107 | 28228, 28358, |
| | 291)(T78A_V150A_1163V_T1821) | | | 28479 |
| 196 | SARS-COV-2/Wuhan-Hu-1 non-structural protein 6 (NSP6) (1- | 27987 | 28108 | 28229, 28359, |
| | 291)(S107del_G108del_F109del) | | | 28480 |
| 197 | SARS-COV-2/Wuhan-Hu-1 non-structural protein 6 (NSP6) (1- | 27988 | 28109 | 28230, 28360, |
| | 291)(S107del_G108del_F109del_M165T) | | | 28481 |
| 198 | SARS-COV-2/Wuhan-Hu-1 non-structural protein 6 (NSP6) (1- | 27989 | 28110 | 28231, 28361, |
| | 291)(V27G_L38F_F221L_C222F) | | | 28482 |
| 199 | SARS-COV-2/Wuhan-Hu-1 non-structural protein 6 (NSP6) (1- | 27990 | 28111 | 28232, 28362, |
| | 291)(L38F_L126F) | | | 28483 |
| 200 | SARS-COV-2/Wuhan-Hu-1 non-structural protein 6 (NSP6) (1- | 27991 | 28112 | 28233, 28363, |
| | 291)(S107del_G108del_F109del_L126F) | | | 28484 |
| 201 | SARS-COV-2/Wuhan-Hu-1 non-structural protein 6 (NSP6) (1- | 27992 | 28113 | 28234, 28364, |
| | 291)(S107del_G108del_F109del_V2791) | | | 28485 |
| 202 | SARS-COV-2/Wuhan-Hu-1 ORF3a protein (ORF3a) (1- | 27993 | 28114 | 28235, 28365, |
| | 275)(Q57H_S171L) | | | 28486 |
| 203 | SARS-COV-2/Wuhan-Hu-1 ORF3a protein (ORF3a) (1- | 27994 | 28115 | 28236, 28366, |
| | 275)(D155Y_S253P) | | | 28487 |
| 204 | SARS-COV-2/Wuhan-Hu-1 ORF3a protein (ORF3a) (1- | 27995 | 28116 | 28237, 28367, |
| | 275)(S253P) | | | 28488 |
| 205 | SARS-COV-2/Wuhan-Hu-1 ORF3a protein (ORF3a) (1- | 27996 | 28117 | 28238, 28368, |
| | 275)(L15F_W131C_T1511) | | | 28489 |
| 206 | SARS-COV-2/Wuhan-Hu-1 ORF3a protein (ORF3a) (1- | 27997 | 28118 | 28239, 28369, |
| | 275)(P42L_Q57H) | | | 28490 |
| 207 | SARS-COV-2/Wuhan-Hu-1 ORF3a protein (ORF3a) (1- | 27998 | 28119 | 28240, 28370, |
| | 275)(Q57H_N257del) | | | 28491 |
| 208 | SARS-COV-2/Wuhan-Hu-1 ORF3a protein (ORF3a) (1- | 27999 | 28120 | 28241, 28371, |
| | 275)(N257del) | | | 28492 |
| 209 | SARS-COV-2/Wuhan-Hu-1 ORF3a protein (ORF3a) (1- | 28000 | 28121 | 28242, 28372, |
| | 275)(P42L_Q57H_P104L) | | | 28493 |
| 210 | SARS-COV-2/Wuhan-Hu-1 ORF8 protein (ORF8) (1- | 28001 | 28122 | 28243, 28373, |
| | 121)(R521_Y73C) | | | 28494 |
| 211 | SARS-COV-2/Wuhan-Hu-1 ORF8 protein (ORF8) (1-121)(E92K) | 28002 | 28123 | 28244, 28374, |
| | | | | 28495 |
| 212 | SARS-COV-2/Wuhan-Hu-1 ORF8 protein (ORF8) (1- | 28003 | 28124 | 28245, 28375, |
| | 121)(D119I_F120del_\|121del) | | | 28496 |
| 213 | SARS-COV-2/Wuhan-Hu-1 ORF8 protein (ORF8) (1- | 28004 | 28125 | 28246, 28376, |
| | 121)(K44R_F120V_1121L) | | | 28497 |
| 214 | SARS-COV-2/Wuhan-Hu-1 ORF8 protein (ORF8) (1-121)(K2Q) | 28005 | 28126 | 28247, 28377, |
| | | | | 28498 |
| 215 | SARS-COV-2/Wuhan-Hu-1 ORF8 protein (ORF8) (1-121)(T111) | 28006 | 28127 | 28248, 28378, |
| | | | | 28499 |
| 216 | SARS-COV-2/Wuhan-Hu-1 ORF8 protein (ORF8) (1- | 28007 | 28128 | 28249, 28379, |
| | 121)(T11K_P38S_S67F) | | | 28500 |
| 217 | SARS-COV-2/Vuhan-Hu-1 ORF8 protein (ORF8) (1-121)(T261) | 28008 | 28129 | 28250, 28380, |
| | | | | 28501 |
| 218 | SARS-COV-2/Wuhan-Hu-1 ORF8 protein (ORF8) (1-121)(F3Y) | 28009 | 28130 | 28251, 28381, |
| | | | | 28502 |
| 219 | SARS-COV-2/Wuhan-Hu-1 ORF8 protein (ORF8) (1- | 28010 | 28131 | 28252, 28382, |
| | 121)(T11\|_A51S) | | | 28503 |
| 220 | SARS-COV-2/Wuhan-Hu-1 ORF8 protein (ORF8) (1- | 28011 | 28132 | 28253, 28383, |
| | 121)(V32L_V100L) | | | 28504 |
| 221 | SARS-COV-2/Wuhan-Hu-1 ORF8 protein (ORF8) (1-121)(A65V) | 28012 | 28133 | 28254, 28384, |
| | | | | 28505 |
| 222 | SARS-COV-2/Wuhan-Hu-1 non-structural protein 13 (NSP13) (1- | 28013 | 28134 | 28255, 28385, |
| | 602)(P78L) | | | 28506 |
| 223 | SARS-COV-2/Wuhan-Hu-1 non-structural protein 13 (NSP13) (1- | 28014 | 28135 | 28256, 28386, |
| | 602)(P79S_K461R) | | | 28507 |
| 224 | SARS-COV-2/Wuhan-Hu-1 non-structural protein 13 (NSP13) (1- | 28015 | 28136 | 28257, 28387, |
| | 602)(E342D) | | | 28508 |
| 225 | SARS-COV-2/Wuhan-Hu-1 non-structural protein 13 (NSP13) (1- | 28016 | 28137 | 28258, 28388, |
| | 602)(T5891) | | | 28509 |
| 226 | SARS-COV-2/Wuhan-Hu-1 non-structural protein 13 (NSP13) (1- | 28017 | 28138 | 28259, 28389, |
| | 602)(P54L_D261Y) | | | 28510 |
| 227 | SARS-COV-2/Wuhan-Hu-1 non-structural protein 13 (NSP13) (1- | 28018 | 28139 | 28260, 28390, |
| | 602)(Q89H_H165Y) | | | 28511 |
| 228 | SARS-COV-2/Wuhan-Hu-1 non-structural protein 13 (NSP13) (1- | 28019 | 28140 | 28261, 28391, |
| | 602)(D106Y_P530L) | | | 28512 |
| 229 | SARS-COV-2/WVuhan-Hu-1 non-structural protein 13 (NSP13) (1- | 28020 | 28141 | 28262, 28392, |
| | 602)(P173H_E262D_P420S_P492S) | | | 28513 |
| 230 | SARS-COV-2/Wuhan-Hu-1 non-structural protein 13 (NSP13) (1- | 28021 | 28142 | 28263, 28393, |
| | 602)(D261Y) | | | 28514 |
| 231 | SARS-COV-2/Wuhan-Hu-1 non-structural protein 14 (NSP14) (1- | 28022 | 28143 | 28264, 28394, |
| | 528)(T321_P143S_P444S) | | | 28515 |
| 232 | SARS-COV-2/Wuhan-Hu-1 non-structural protein 14 (NSP14) (1- | 28023 | 28144 | 28265, 28395, |
| | 528)(P204L_F218Y_P328Q) | | | 28516 |
| 233 | SARS-COV-2/Wuhan-Hu-1 non-structural protein 14 (NSP14) (1- | 28024 | 28145 | 28266, 28396, |
| | 528)(E348G_P452S) | | | 28517 |
| 234 | SARS-COV-2/Wuhan-Hu-1 non-structural protein 14 (NSP14) (1- | 28025 | 28146 | 28267, 28397, |
| | 528)(A395V) | | | 28518 |
| 235 | SARS-COV-2/Wuhan-Hu-1 non-structural protein 14 (NSP14) (1- | 28026 | 28147 | 28268, 28398, |
| | 528)(D145E_A275S) | | | 28519 |
| 236 | SARS-COV-2/Wuhan-Hu-1 non-structural protein 14 (NSP14) (1- | 28027 | 28148 | 28269, 28399, |
| | 528)(D145G_Q355H) | | | 28520 |
| 237 | SARS-COV-2/Wuhan-Hu-1 non-structural protein 14 (NSP14) (1- | 28028 | 28149 | 28270, 28400, |
| | 528)(S2561_A361V_A372T) | | | 28521 |

Suitable features and embodiments that apply to the at least one nucleic acid encoding Coronavirus M, N, NSP3, NSP4, NSP6, NSP13, NSP14, ORF3A, ORF8, E are provided in paragraph “Nucleic acid features and embodiments” below.

Suitably, the nucleic acids is formulated and/or complexed. Suitable features and embodiments that apply to nucleic acids complexation or formulation of nucleic acid encoding Coronavirus M, N, NSP3, NSP4, NSP6, NSP13, NSP14, ORF3A, ORF8, E are provided in paragraph “Formulation and Complexation” below.

In preferred embodiments, intramuscular or intradermal administration of the composition comprising at least one nucleic acid encoding Coronavirus M, N, NSP3, NSP4, NSP6, NSP13, NSP14, ORF3A, ORF8, and/or E as defined herein results in expression of the encoded Coronavirus M, N, NSP3, NSP4, NSP6, NSP13, NSP14, ORF3A, ORF8, and/or E in a subject. In embodiments where the nucleic acid is an RNA, administration results in translation of the RNA and to a production of the encoded Coronavirus M, N, NSP3, NSP4, NSP6, NSP13, NSP14, ORF3A, ORF8, and/or E. In embodiments where the nucleic acid is a DNA (e.g. plasmid DNA, adenovirus DNA), administration of said composition results in transcription of the DNA into RNA, and to a subsequent translation of the RNA into the encoded Coronavirus M, N, NSP3, NSP4, NSP6, NSP13, NSP14, ORF3A, ORF8, and/or E in a subject.

In embodiments, administration of the pharmaceutical composition comprising at least one nucleic acid encoding Coronavirus M, N, NSP3, NSP4, NSP6, NSP13, NSP14, ORF3A, ORF8, and/or E as defined herein to a subject elicits neutralizing antibodies against Coronavirus and does not elicit disease enhancing antibodies.

In preferred embodiments, administration of the pharmaceutical composition comprising at least one nucleic acid encoding Coronavirus M, N, NSP3, NSP4, NSP6, NSP13, NSP14, ORF3A, ORF8, and/or E as defined herein to a subject elicits antigen-specific immune responses comprising T-cell responses and/or B-cell responses against the encoded Coronavirus M, N, NSP3, NSP4, NSP6, NSP13, NSP14, ORF3A, ORF8, and/or E antigen.

Combinations of M, N, NSP3, NSP4, NSP6, NSP13, NSP14, ORF3A, ORF8, and/or E (Multivalent Composition)

In preferred embodiments, the pharmaceutical composition comprises the at least one, preferably at least two or a plurality of the following nucleic acid sequences encoding at least one antigenic peptide or protein is selected or derived from membrane protein (M) as defined herein, nucleocapsid protein (N) as defined herein, non-structural protein as defined herein, and/or accessory protein as defined herein:
M nucleic acid comprising at least one coding sequence encoding at least one antigenic peptide or protein selected or derived from at least one membrane (M) protein as defined herein, or an immunogenic fragment or immunogenic variant thereof, and/or
N nucleic acid comprising at least one coding sequence encoding at least one antigenic peptide or protein selected or derived from at least one nucleocapsid (N) protein as defined herein, or an immunogenic fragment or immunogenic variant thereof, and/or
NSP3 nucleic acid comprising at least one coding sequence encoding at least one antigenic peptide or protein selected or derived from at least one NSP3 protein as defined herein, or an immunogenic fragment or immunogenic variant thereof, and/or
NSP4 nucleic acid comprising at least one coding sequence encoding at least one antigenic peptide or protein selected or derived from at least one NSP4 protein as defined herein, or an immunogenic fragment or immunogenic variant thereof, and/or
NSP6 nucleic acid comprising at least one coding sequence encoding at least one antigenic peptide or protein selected or derived from at least one NSP6 protein as defined herein, or an immunogenic fragment or immunogenic variant thereof, and/or
NSP13 nucleic acid comprising at least one coding sequence encoding at least one antigenic peptide or protein selected or derived from at least one NSP6 protein as defined herein, or an immunogenic fragment or immunogenic variant thereof, and/or
NSP14 nucleic acid comprising at least one coding sequence encoding at least one antigenic peptide or protein selected or derived from at least one NSP6 protein as defined herein, or an immunogenic fragment or immunogenic variant thereof, and/or
ORF3A nucleic acid comprising at least one coding sequence encoding at least one antigenic peptide or protein selected or derived from at least one ORF3a protein, or an immunogenic fragment or immunogenic variant thereof, and/or
ORF8 nucleic acid comprising at least one coding sequence encoding at least one antigenic peptide or protein selected or derived from at least one ORF8 protein, or an immunogenic fragment or immunogenic variant thereof.
E nucleic acid comprising at least one coding sequence encoding at least one antigenic peptide or protein selected or derived from at least one E protein, or an immunogenic fragment or immunogenic variant thereof.

In embodiments, the pharmaceutical composition comprises 2, 3, 4, 5, 6, 7, or more of M, N, NSP3, NSP4, NSP6, NSP13, NSP14, ORF3A, ORF8, E wherein the antigenic peptide or proteins are selected or derived from the same Coronavirus. Suitably, the 2, 3, 4, 5, 6, 7 or more antigenic peptide or proteins are selected or derived from SARS-CoV-2 or SARS-CoV-2 variants. In embodiments, the 2, 3, 4, 5, 6, 7 or more antigenic peptide or proteins are selected or derived from different Coronaviruses, preferably different pandemic Coronaviruses, e.g. SARS-CoV-2, SARS-CoV-1, and/or MERS-CoV or from different pandemic SARS-CoV-2 variants.

In embodiments, the pharmaceutical composition of the invention comprises at least two nucleic acid sequences according to the following combinations:
M and N; M and NSP3; M and NSP4; M and NSP6; M and NSP13; M and NSP14; M and ORF3A; M and ORF8; N and NSP3; N and NSP4; N and NSP6; N and NSP13; N and NSP 14, N and ORF3A; N and ORF8: NSP3 and NSP4; NSP3 and NSP6; NSP3 and NSP13; NSP3 and NSP14; NSP3 and ORF3A; NSP3 and ORF8; NSP4 and NSP6; NSP4 and NSP13; NSP4 and NSP14; NSP4 and ORF3A; NSP4 and ORF8; NSP6 and NSP13; NSP6 and NSP14; NSP6 and ORF3A; NSP6 and ORF8; NSP13 and NSP14; NSP13 and ORF3A; NSP13 and ORF8; NSP14 and OFR3A; NSP14 and ORF8; or ORF3A and ORF8,
optionally, wherein the antigenic peptide or proteins are selected or derived from the same Coronavirus, preferably SARS-CoV-2 or SARS-CoV-2 variants or wherein the antigenic peptide or proteins are selected or derived from different Coronaviruses, preferably different pandemic Coronaviruses or Coronavirus variants, e.g. SARS-CoV-2, SARS-CoV-1, and/or MERS-CoV or from different pandemic SARS-CoV-2 variants.

In embodiments, the pharmaceutical composition of the invention comprises at least two nucleic acid sequences according to the following combinations:
M and N; M and NSP3; M and NSP4; M and NSP6; M and ORF3A; M and ORF8; M and NSP13; M and NSP14;
N and NSP3; N and NSP4; N and NSP6; N and ORF3A; N and ORF8; N and NSP13; N and NSP14;
optionally, wherein the antigenic peptide or proteins are selected or derived from the same Coronavirus, preferably SARS-CoV-2 or SARS-CoV-2 variants or wherein the antigenic peptide or proteins are selected or derived from different Coronaviruses, preferably different pandemic Coronaviruses, e.g. SARS-CoV-2, SARS-CoV-1, and/or MERS-CoV or from different pandemic SARS-CoV-2 variants.

In preferred embodiments, the pharmaceutical composition of the invention comprises at least two nucleic acid sequences according to the following combinations:
N and NSP3; N and NSP4; N and ORF8; NSP3 and NSP4; NSP3 and ORF8; NSP4 and ORF8;
optionally wherein the antigenic peptide or proteins are selected or derived from the same SARS-CoV-2 virus or SARS-CoV-2 variant or wherein the antigenic peptide or proteins are selected or derived from different Coronaviruses, preferably different pandemic Coronaviruses, e.g. SARS-CoV-2, SARS-CoV-1, and/or MERS-CoV or from different pandemic SARS-CoV-2 variants.

In embodiments, the pharmaceutical composition of the invention comprises at least three nucleic acid sequences according to the following combinations:
M and N and NSP3; M and N and NSP4; M and N and NSP6; M and N and NSP13; M and N and NSP14; M and N and ORF3A; M and N and ORF8; M and NSP3 and NSP4; M and NSP3 and NSP6; M and NSP3 and NSP13; M and NSP3 and NSP14; M and NSP3 and ORF3A; M and NSP3 and ORF8; M and NSP4 and NSP6; M and NSP4 and NSP13; M and NSP4 and NSP14; M and NSP4 and ORF3A; M and NSP4 and ORF8; M and NSP6 and NSP13; M and NSP6 and NSP14; M and NSP6 and ORF3A; M and NSP6 and ORF8; M and NSP13 and NSP14; M and NSP13 and ORF3A; M and NSP13 and ORF8; M and NSP14 and ORF 3A; M and NSP14 and ORF; M and ORF3A and ORF8; N and NSP3 and NSP4; N and NSP3 and NSP6; N and NSP3 and NSP13; N and NSP3 and NSP14; N and NSP3 and ORF3A; N and NSP3 and ORF8; N and NSP4 and NSP6; N and NSP4 and NSP13; N and NSP4 and SNP14; N and NSP4 and ORF3A; N and NSP4 and ORF8; N and NSP6 and ORF3A; N and NSP6 and NSP13; N and NSP6 and NSP14; N and NSP6 and ORF8; N and NSP13 and NSP14; N and NSP13 and ORF3A; N and NSP13 and ORF8; N and NSP14 and ORF3A; N and NSP14 and ORF8; N and ORF3A and ORF8; NSP3 and NSP4 and NSP6; NSP3 and NSP4 and NSP13; NSP3 and NSP4 and NSP14; NSP3 and NSP4 and ORF3A; NSP3 and NSP4 and ORF8; NSP3 and NSP6 and ORF3A; NSP3 and NSP6 and ORF8; NSP3 and NSP13 and ORF3A; NSP3 and NSP13 and ORF8; NSP3 and ORF3A and ORF8; NSP4 and NSP6 and NSP13; NSP4 and NSP6 and NSP14; NSP4 and NSP6 and ORF3A; NSP4 and NSP6 and ORF8; NSP13 and NSP14 and ORF3A; NSP13 and NSP14 and ORF8; NSP4 and ORF3A and ORF8; NSP6 and ORF3A and ORF8, NSP13 and ORF3A and ORF8; or NSP14 and ORF3A and ORF8,
optionally, wherein the antigenic peptide or proteins are selected or derived from the same Coronavirus, preferably SARS-CoV-2 or SARS-CoV-2 variants or wherein the antigenic peptide or proteins are selected or derived from different Coronaviruses, preferably different pandemic Coronaviruses, e.g. SARS-CoV-2, SARS-CoV-1, and/or MERS-CoV or from different pandemic SARS-CoV-2 variants.

In embodiments, the pharmaceutical composition of the invention comprises at least three nucleic acid sequences according to the following combinations:
M and N and NSP3; M and N and NSP4; M and N and NSP6; M and N and ORF3A; M and N and ORF8;
M and NSP3 and NSP4; M and NSP3 and NSP6; M and NSP3 and ORF3A;
M and NSP3 and ORF8; M and NSP4 and NSP6; M and NSP4 and ORF3A; M and NSP4 and ORF8;
M and NSP6 and ORF3A; M and NSP6 and ORF8; M and ORF3A and ORF8; N and NSP3 and NSP4;
N and NSP3 and NSP6; N and NSP3 and ORF3A; N and NSP3 and ORF8; N and NSP4 and NSP6;
N and NSP4 and ORF3A; N and NSP4 and ORF8; N and NSP6 and ORF3A; N and NSP6 and ORF8;
N and ORF3A and ORF8,
optionally, wherein the antigenic peptide or proteins are selected or derived from the same Coronavirus, preferably SARS-CoV-2 or SARS-CoV-2 variants or wherein the antigenic peptide or proteins are selected or derived from different Coronaviruses, preferably different pandemic Coronaviruses, e.g. SARS-CoV-2, SARS-CoV-1, and/or MERS-CoV or from different pandemic SARS-CoV-2 variants.

In preferred embodiments, the pharmaceutical composition of the invention comprises at least three nucleic acid sequences according to the following combinations:
N and NSP3 and NSP4; N and NSP3 and ORF8; N and NSP4 and ORF8; N and ORF3A and ORF8, NSP3 and NSP4 and ORF8,
optionally, wherein the antigenic peptide or proteins are selected or derived from the same Coronavirus, preferably SARS-CoV-2 or SARS-CoV-2 variants or wherein the antigenic peptide or proteins are selected or derived from different Coronaviruses, preferably different pandemic Coronaviruses, e.g. SARS-CoV-2, SARS-CoV-1, and/or MERS-CoV or from different pandemic SARS-CoV-2 variants.

In embodiments, the pharmaceutical composition of the invention comprises at least four nucleic acid sequences according to the following combinations:
NSP3 and NSP4 and NSP6 and M; NSP3 and NSP4 and ORF3A and M;
NSP3 and NSP4 and ORF8 and M; NSP3 and NSP6 and ORF3A and M;
NSP3 and NSP6 and ORF8 and M; NSP3 and ORF3A and ORF8 and M;
NSP4 and NSP6 and ORF3A and M; NSP4 and NSP6 and ORF8 and M;
NSP4 and ORF3A and ORF8 and M; NSP6 and ORF3A and ORF8 and M;
NSP3 and NSP4 and NSP6 and N; NSP3 and NSP4 and ORF3A and N;
NSP3 and NSP4 and ORF8 and N; NSP3 and NSP6 and ORF3A and N;
NSP3 and NSP6 and ORF8 and N; NSP3 and ORF3A and ORF8 and N;
NSP4 and NSP6 and ORF3A and N; NSP4 and NSP6 and ORF8 and N; or
NSP4 and ORF3A and ORF8 and N; NSP6 and ORF3A and ORF8 and N,
optionally, wherein the antigenic peptide or proteins are selected or derived from the same Coronavirus, preferably SARS-CoV-2 or SARS-CoV-2 variants or wherein the antigenic peptide or proteins are selected or derived from different Coronaviruses, preferably different pandemic Coronaviruses, e.g. SARS-CoV-2, SARS-CoV-1, and/or MERS-CoV or from different pandemic SARS-CoV-2 variants.

In particularly preferred embodiments, the pharmaceutical composition of the invention comprises at least four nucleic acid sequences according to the following combination:
NSP3 and NSP4 and ORF8 and N;
optionally, wherein the antigenic peptide or proteins are selected or derived from the same Coronavirus, preferably SARS-CoV-2 or SARS-CoV-2 variants or wherein the antigenic peptide or proteins are selected or derived from different Coronaviruses, preferably different pandemic Coronaviruses or Coronavirus variants, e.g. SARS-CoV-2, SARS-CoV-1, and/or MERS-CoV or from different pandemic SARS-CoV-2 variants.

In more preferred embodiments, the pharmaceutical composition of the invention comprises at least four nucleic acid sequences according to the following combination: NSP3 and NSP4 and ORF8 and N;
wherein the antigenic peptide or proteins are selected or derived from the same SARS-CoV-2 or a SARS-CoV-2 variant or wherein the antigenic peptide or proteins are selected or derived from different SARS-CoV-2 or SARS-CoV-2 variants.

In embodiments, the pharmaceutical composition of the invention comprises at least five nucleic acid sequences according to the following combinations:
NSP3 and NSP4 and NSP6 and M and N; NSP3 and NSP4 and ORF3A and M and N;
NSP3 and NSP4 and ORF8 and M and N; NSP3 and NSP6 and ORF3A and M and N;
NSP3 and NSP6 and ORF8 and M and N; NSP3 and ORF3A and ORF8 and M and N;
NSP4 and NSP6 and ORF3A and M and N; NSP4 and NSP6 and ORF8 and M and N; or
NSP4 and ORF3A and ORF8 and M and N; NSP6 and ORF3A and ORF8 and M and N,
optionally, wherein the antigenic peptide or proteins are selected or derived from the same Coronavirus, preferably SARS-CoV-2 SARS-CoV-2 variants or wherein the antigenic peptide or proteins are selected or derived from different Coronaviruses, preferably different pandemic Coronaviruses, e.g. SARS-CoV-2, SARS-CoV-1, and/or MERS-CoV or from different pandemic SARS-CoV-2 variants.

Nucleic Acid Encoding Coronavirus Spike Protein

In various embodiments, the pharmaceutical composition of the first aspect further comprises at least one (additional) nucleic acid comprising at least one coding sequence encoding at least one antigenic peptide or protein selected or derived from at least one Coronavirus spike protein (S), or an immunogenic fragment or immunogenic variant thereof.

Accordingly, in preferred embodiments, the pharmaceutical composition comprises at least two nucleic acids,
(i) at least one nucleic acid comprising at least one coding sequence encoding at least one antigenic peptide or protein selected or derived from at least one Coronavirus spike protein (S), or an immunogenic fragment or immunogenic variant thereof, and
(ii) at least one nucleic acid comprising at least one coding sequence encoding at least one antigenic peptide or protein from at least one Coronavirus, wherein the at least one antigenic peptide or protein is selected or derived from membrane protein (M), nucleocapsid protein (N), non-structural protein, and/or accessory protein or an immunogenic fragment or immunogenic variant thereof.

The Coronavirus spike protein of the composition that is provided by the at least one (additional) nucleic acid can be selected or derived from any Coronavirus.

In preferred embodiments the at least one Coronavirus spike protein (S) is selected or derived from at least one pandemic Coronavirus.

In preferred embodiments the at least one Coronavirus spike protein (S) is selected or derived from at least one Alphacoronavirus, at least one Betacoronavirus, at least one Gammacoronavirus, and/or at least one Deltacoronavirus, preferably a pandemic Alphacoronavirus, Betacoronavirus, Gammacoronavirus, Deltacoronavirus.

In embodiments, the at least one Coronavirus spike protein (S) is selected or derived from at least one Betacoronavirus. Suitably, the Betacoronavirus is selected from at least one Sarbecovirus, at least one Merbecovirus, at least one Embecovirus, at least one Nobecovirus, and/or at least one Hibecovirus.

In a preferred embodiments, the at least one Coronavirus spike protein (S) is selected or derived from a Betacoronavirus, preferably a Sarbecovirus. In the context of the invention, a preferred Sarbecovirus may be selected from a SARS-associated Coronavirus. Preferred SARS-associated viruses can be selected from SARS-CoV-1 and/or SARS-CoV-2 or variants thereof.

In a preferred embodiments, the at least one Coronavirus spike protein (S) is selected or derived from a Betacoronavirus, preferably a Merbecovirus. In the context of the invention, a preferred Merbecovirus may be selected from a MERS-associated Coronavirus. Preferred MERS-associated Coronaviruses can be selected from MERS-CoV.

The term “antigenic peptide or protein derived from at least one Coronavirus spike protein (S)” relates to any peptide or protein that is selected or is derived from the respective Coronavirus S protein as defined herein, but also to fragments, variants or derivatives thereof, preferably to immunogenic fragments or immunogenic variants thereof.

The term “immunogenic fragment of Coronavirus spike protein (S)” or “immunogenic variant of Coronavirus spike protein (S)” has to be understood as any fragment/variant of the corresponding Coronavirus spike protein (S) that is capable of raising an immune response in a subject.

In the context of the invention, any protein selected or derived from a Coronavirus spike protein (S), preferably a pandemic Coronavirus spike protein (S), may be used in the context of the invention and may be suitably encoded by the coding sequence of the additional nucleic acid. It is further in the scope of the underlying invention, that the at least one antigenic peptide or protein may comprise or consist of a synthetically engineered or an artificial Coronavirus S peptide or protein. The term “synthetically engineered” Coronavirus S peptide or protein, or the term “artificial Coronavirus S peptide or protein” relates to a protein that does not occur in nature. Accordingly, an “artificial Coronavirus S peptide or protein” or a “synthetically engineered Coronavirus S peptide or protein” may for example differ in at least one amino acid compared to the naturally existing Coronavirus peptide or protein, and/or may comprise an additional peptide or protein element (e.g. a heterologous element), and/or may be N-terminally or C-terminally extended or truncated.

In preferred embodiments, the encoded at least one antigenic peptide or protein comprises or consists at least one peptide or protein selected or derived from a Coronavirus spike protein (S, S1, S2, or S1 and S2), or an immunogenic fragment or immunogenic variant of any of these.

The term spike protein (S) as used herein refers to a Coronavirus protein. Spike protein (S) is a typical type I viral fusion protein that exists as trimer on the viral surface with each monomer consisting of a Head (S1) and stem (S2). Individual precursor S polypeptides form a homotrimer and undergo glycosylation within the Golgi apparatus as well as processing to remove the signal peptide, and cleavage by a cellular protease to generate separate S1 and S2 polypeptide chains, which remain associated as S1/S2 protomers within the homotrimer and is therefore a trimer of heterodimers. The S1 domain of the spike glycoprotein includes the receptor binding domain (RBD) that engages (most likely) with the angiotensin-converting enzyme 2 receptors and mediates viral fusion into the host cell, an N-terminal domain that may make initial contact with target cells, and 2 subdomains, all of which are susceptible to neutralizing antibodies. S2 domain consists of a six helix bundle fusion core involved in membrane fusion with the host endosomal membrane and is also a target for neutralization. The S2 subunit further comprises two heptad-repeat sequences (HR1 and HR2) and a central helix typical of fusion glycoproteins, a transmembrane domain, and the cytosolic tail domain.

Without wishing to being bound to theory, RBD and CND domains may be crucial for immunogenicity of the Coronavirus spike protein (S). Both regions are located at the S1 fragment of the Coronavirus spike protein. Accordingly, it may be suitable in the context of the invention that the antigenic peptide or protein comprises or consists of an S1 fragment of the spike protein of a Coronavirus or an immunogenic fragment or immunogenic variant thereof. Suitably, such an S1 fragment may comprise at least an RBD and/or a CND domain as defined above.

In particularly preferred embodiments, the encoded at least one antigenic peptide or protein comprises or consists of a Coronavirus spike protein (S), wherein spike protein (S) comprises or consists of a spike protein fragment S1, or an immunogenic fragment or immunogenic variant thereof.

Without wishing to being bound to theory, it may be suitable that the antigenic peptide or protein comprises or consists of Coronavirus spike protein fragment S1 and (at least a fragment of) Coronavirus spike protein fragment S2, because the formation of an immunogenic Coronavirus spike protein may be promoted.

In particularly preferred embodiments, the encoded at least one antigenic peptide or protein comprises or consists of a full-length Coronavirus spike protein or an immunogenic fragment or immunogenic variant of any of these. The term “full-length Coronavirus spike protein” has to be understood as a Coronavirus spike protein, preferably derived from a pandemic Coronavirus, having an amino acid sequence corresponding to essentially the full spike protein.

In particularly preferred embodiments, the Coronavirus spike protein (S) that is provided by the (additional) nucleic is designed or adapted to stabilize the S antigen in pre-fusion conformation. A pre-fusion conformation is particularly advantageous in the context of an efficient vaccine, as several potential epitopes for neutralizing antibodies may merely be accessible in said pre-fusion protein conformation. Furthermore, remaining of the S protein in the pre-fusion conformation is aimed to avoid immunopathological effects, like e.g. enhanced disease and/or antibody dependent enhancement (ADE).

Accordingly, in preferred embodiments, the (additional) nucleic acid comprises at least one coding sequence encoding at least one antigenic peptide or protein that is selected or derived from an Coronavirus S, preferably a pandemic Coronavirus S, wherein the spike protein (S) is a pre-fusion stabilized spike protein (S_stab). Suitably, said pre-fusion stabilized spike protein comprises at least one pre-fusion stabilizing mutation.

The term “pre-fusion conformation” as used herein relates to a structural conformation adopted by the ectodomain of the coronavirus S protein following processing into a mature coronavirus S protein in the secretory system, and prior to triggering of the fusogenic event that leads to transition of coronavirus S to the postfusion conformation.

A “pre-fusion stabilized spike protein (S_stab)” as described herein comprises one or more amino acid substitutions, deletions, or insertions compared to a native coronavirus S sequence that provide for increased retention of the prefusion conformation compared to coronavirus S ectodomain trimers formed from a corresponding native coronavirus S sequence. The “stabilization” of the prefusion conformation by the one or more amino acid substitutions, deletions, or insertions can be, for example, energetic stabilization (for example, reducing the energy of the prefusion conformation relative to the post-fusion open conformation) and/or kinetic stabilization (for example, reducing the rate of transition from the prefusion conformation to the postfusion conformation). Additionally, stabilization of the coronavirus S ectodomain trimer in the prefusion conformation can include an increase in resistance to denaturation compared to a corresponding native coronavirus S sequence.

Accordingly, in preferred embodiments, the Coronavirus spike protein includes one or more amino acid substitutions that stabilize the S protein in the pre-fusion conformation, for example, substitutions that stabilize the membrane distal portion of the S protein (including the N-terminal region) in the pre-fusion conformation.

In preferred embodiments, the at least one pre-fusion stabilizing mutation comprises a cavity filling mutation that further stabilizes the pre-fusion state of the Coronavirus S protein. The term “cavity filling mutation” or “cavity filling amino acid substitution” relates to an amino acid substitution that fills a cavity within the protein core of a protein, such as a coronavirus S protein ectodomain. Cavities are essentially voids within a folded protein where amino acids or amino acid side chains are not present. In several embodiments, a cavity-filling amino acid substitution is introduced to fill a cavity present in the prefusion conformation of a Coronavirus S ectodomain core that collapses (e.g., has reduced volume) after transition to the postfusion conformation.

In preferred embodiments, the at least one pre-fusion stabilizing mutation comprises a mutated protonation site that further stabilizes the pre-fusion state.

In preferred embodiments, the at least one pre-fusion stabilizing mutation comprises an artificial intramolecular disulfide bond. Such an artificial intramolecular disulfide bond can be introduced to further stabilize the membrane distal portion of the S protein (including the N-terminal region) in the pre-fusion conformation; that is, in a conformation that specifically binds to one or more pre-fusion specification antibodies, and/or presents a suitable antigenic site that is present on the pre-fusion conformation but not in the post fusion conformation of the S protein. In embodiments, the at least one pre-fusion stabilizing mutation comprises 2, 3, 4, 5, 6, 7, or 8 different artificial intramolecular disulfide bonds.

It has to be emphasized that in the context of the invention any Coronavirus S protein, preferably any pandemic Coronavirus S protein may be mutated as described above to stabilize the spike protein in the pre-fusion conformation. Accordingly, a spike protein may be selected from any Coronavirus, preferably from any Alphacoronavirus, Betacoronavirus, Gammacoronavirus, Deltacoronavirus, more preferably Betacoronavirus.

According to various preferred embodiments, the (additional) nucleic acid encodes at least one antigenic peptide or protein from Coronavirus S as defined herein, preferably of a pandemic Coronavirus, and, additionally, at least one heterologous peptide or protein element.

Suitably, the at least one heterologous peptide or protein element may promote or improve secretion of the encoded Coronavirus S antigenic peptide or protein (e.g. via secretory signal sequences), promote or improve anchoring of the encoded antigenic peptide or protein of the invention in the plasma membrane (e.g. via transmembrane elements), promote or improve formation of antigen complexes (e.g. via multimerization domains or antigen clustering elements), or promote or improve virus-like particle formation (VLP forming sequence). In addition, the nucleic acid of may additionally encode peptide linker elements, self-cleaving peptides, immunologic adjuvant sequences or dendritic cell targeting sequences.

Suitable multimerization domains may be selected from the list of amino acid sequences according to SEQ ID NOs: 1116-1167 of WO2017081082, or fragments or variants of these sequences. Suitable transmembrane elements may be selected from the list of amino acid sequences according to SEQ ID NOs: 1228-1343 of WO2017081082, or fragments or variants of these sequences. Suitable VLP forming sequences may be selected from the list of amino acid sequences according to SEQ ID NOs: 1168-1227 of the patent application WO2017081082, or fragments or variants of these sequences. Suitable peptide linkers may be selected from the list of amino acid sequences according to SEQ ID NOs: 1509-1565 of the patent application WO2017081082, or fragments or variants of these sequences. Suitable self-cleaving peptides may be selected from the list of amino acid sequences according to SEQ ID NOs: 1434-1508 of the patent application WO2017081082, or fragments or variants of these sequences. Suitable immunologic adjuvant sequences may be selected from the list of amino acid sequences according to SEQ ID NOs: 1360-1421 of the patent application WO2017081082, or fragments or variants of these sequences. Suitable dendritic cell (DCs) targeting sequences may be selected from the list of amino acid sequences according to SEQ ID NOs: 1344-1359 of the patent application WO2017081082, or fragments or variants of these sequences. Suitable secretory signal peptides may be selected from the list of amino acid sequences according to SEQ ID NOs: 1-1115 and SEQ ID NO: 1728 of published PCT patent application WO2017081082, or fragments or variants of these sequences.

In preferred embodiments, the at least one coding sequence additionally encodes one or more heterologous peptide or protein elements selected from a signal peptide, a linker peptide, a helper epitope, an antigen clustering element, a trimerization or multimerization element, a transmembrane element, or a VLP forming sequence.

In preferred embodiments, the (additional) nucleic acid encoding at least one antigenic protein derived from a Coronavirus S additionally encodes at least one heterologous trimerization element, an antigen clustering element, or a VLP forming sequence.

In preferred embodiments, the antigen clustering elements may be selected from a ferritin element, or a lumazine synthase element, surface antigen of Hepatitis B virus (HBsAg), or encapsulin. Expressing a stably clustered Coronavirus spike protein, preferably in in its prefusion conformation, may increases the magnitude and breadth of neutralizing activity against the encoded Coronavirus S antigen.

Lumazine synthase (Lumazine, LS, LumSynth) is an enzyme with particle-forming properties, present in a broad variety of organisms, and involved in riboflavin biosynthesis. In particularly preferred embodiments, lumazine synthase is used to promote antigen clustering and may therefore promote or enhance immune responses of the encoded Coronavirus S antigen.

In particularly preferred embodiments, the antigen clustering element (multimerization element) is or is derived from lumazine synthase, wherein the amino acid sequences of said antigen clustering domain is preferably identical or at least 70%, 80%, 85%, 86%, 87%, 88%, 89%, 90%, 91%, 92%, 93%, 94%, 95%, 96%, 97%, 98%, or 99% identical to any one of amino acid sequences SEQ ID NO: 112, a fragment or variant thereof.

Ferritin is a protein whose main function is intracellular iron storage. Almost all living organisms produce ferritin which is made of 24 subunits, each composed of a four-alpha-helix bundle, that self-assemble in a quaternary structure with octahedral symmetry. Its properties to self-assemble into nanoparticles are well-suited to carry and expose antigens.

In particularly preferred embodiments, ferritin is used to promote the antigen clustering and may therefore promote immune responses of the encoded Coronavirus S protein.

In particularly preferred embodiments, the antigen clustering element (multimerization element) is selected or derived from ferritin wherein the amino acid sequences of said antigen clustering domain is preferably identical or at least 70%, 80%, 85%, 86%, 87%, 88%, 89%, 90%, 91%, 92%, 93%, 94%, 95%, 96%, 97%, 98%, or 99% identical to any one of amino acid sequence SEQ ID NO: 113, a fragment or variant thereof.

In some embodiments, the antigen clustering domain is a Hepatitis B surface antigen (HBsAg). HBsAg forms spherical particles. The addition of a fragment of the surface antigen of Hepatitis B virus (HBsAg) sequence may be particularly effective in enhancing the immune response of the nucleic-acid-based vaccine against Coronavirus.

In particularly preferred embodiments, HBsAg is used to promote the antigen clustering and may therefore promote immune responses of the encoded Coronavirus S antigen, preferably a spike protein as defined herein.

In some embodiments, the antigen clustering element is an encapsulin element. The addition of an encapsulin sequence may be particularly effective in enhancing the immune response of the nucleic-acid-based vaccine against Coronavirus. In particularly preferred embodiments, encapsulin is used to promote the antigen clustering and may therefore promote immune responses of the encoded Coronavirus S protein as defined herein.

Encapsulin is a protein isolated from thermophile Thermotoga maritima and may be used as an element to allow self-assembly of antigens to form antigen (nano)particles. Encapsulin is assembled from 60 copies of identical 31 kDa monomers having a thin and icosahedral T=1 symmetric cage structure with interior and exterior diameters of 20 and 24 nm, respectively.

In embodiments where the coding sequence of the (additional) nucleic acid additionally encodes heterologous antigen clustering element, it is particularly preferred and suitable to generate a fusion protein comprising an antigen clustering element and an antigenic peptide or protein derived from a Coronavirus S. Suitably, said antigenic peptide or protein, preferably the spike protein, is lacking the C-terminal transmembrane domain (TM) or is lacking a part of the C-terminal transmembrane domain (TM).

In other embodiments, where the coding sequence of the (additional) nucleic acid of additionally encodes heterologous antigen clustering element as defined above, it is particularly preferred and suitable to generate a fusion protein comprising an antigen clustering element and an antigenic peptide or protein derived from a Coronavirus spike protein fragment S1 (lacking S2 and/or TM and/or TMflex). Further, it may be suitable to use linker elements for separating the heterologous antigen clustering element from the antigenic peptide or protein (e.g. a linker according to SEQ ID NO: 115, 13148, 13152).

Further suitable multimerization elements may be selected from the list of amino acid sequences according to SEQ ID NOs: 1116-1167 of WO2017081082, or fragments or variants of these sequences. SEQ ID NOs: 1116-1167 of WO2017081082 are herewith incorporated by reference.

In preferred embodiments, the trimerization element may be selected from a foldon element. In preferred embodiments, the foldon element is a fibritin foldon element. Expressing a stable trimeric spike protein, preferably in its prefusion conformation, may increases the magnitude and breadth of neutralizing activity against a Coronavirus S.

In particularly preferred embodiments, a fibritin foldon element is used to promote the antigen trimerization and may therefore promote immune responses of the encoded coronavirus antigen, preferably spike protein. Preferably, the foldon element is or is derived from a bacteriophage, preferably from bacteriophage T4, most preferably from fibritin of bacteriophage T4.

In particularly preferred embodiments, the trimerization element is selected or derived from foldon wherein the amino acid sequences of said trimerization element is preferably identical or at least 70%, 80%, 85%, 86%, 87%, 88%, 89%, 90%, 91%, 92%, 93%, 94%, 95%, 96%, 97%, 98%, or 99% identical to any one of amino acid sequence SEQ ID NO: 114, a fragment or variant of any of these.

In embodiments where the coding sequence of the (additional) nucleic acid encodes heterologous trimerization element, it is particularly preferred and suitable to generate a fusion protein comprising a trimerization element and an antigenic peptide or protein derived from a Coronavirus S. Suitably, said antigenic peptide or protein, preferably the spike protein derived from Coronavirus that is lacking the C-terminal transmembrane domain, or is lacking a part of the C-terminal transmembrane domain (TMflex).

In other embodiments, where the coding sequence of the (additional) nucleic acid encodes heterologous trimerization element as defined above, it is particularly preferred and suitable to generate a fusion protein comprising an trimerization element and an antigenic peptide or protein derived from Coronavirus spike protein fragment S1 (lacking S2 and/or TM and/or TMflex). Further, it may be suitable to use linker elements for separating the heterologous antigen clustering element from the antigenic peptide or protein (e.g. a linker according to SEQ ID NO: 115, 13148, 13152).

Further suitable trimerization elements may be selected from the list of amino acid sequences according to SEQ ID NOs: 1116-1167 of WO2017081082, or fragments or variants of these sequences. SEQ ID NOs: 1116-1167 of WO2017081082 are herewith incorporated by reference.

In preferred embodiments, the VLP forming sequence may be selected and fused to the Coronavirus S antigen as defined herein. Expressing a stably clustered spike protein in VLP form may increases the magnitude and breadth of neutralizing activity against Coronavirus. VLPs structurally mimic infectious viruses and they can induce potent cellular and humoral immune responses.

Suitable VLP forming sequences may be selected from elements derived from Hepatitis B virus core antigen, HIV-1 Gag protein, or Woodchuck hepatitis core antigen element (WhcAg).

In particularly preferred embodiments, the at least one VLP-forming sequence is a Woodchuck hepatitis core antigen element (WhcAg). The WhcAg element is used to promote VLP formation and may therefore promote immune responses of the encoded Coronavirus S antigen, preferably spike protein.

In particularly preferred embodiments, the VLP forming sequence is selected or derived from foldon wherein the amino acid sequences of said VLP forming sequences is preferably identical or at least 70%, 80%, 85%, 86%, 87%, 88%, 89%, 90%, 91%, 92%, 93%, 94%, 95%, 96%, 97%, 98%, or 99% identical to any one of amino acid sequence SEQ ID NO: 13171, a fragment or variant of any of these.

In embodiments where the coding sequence of the (additional) nucleic acid encodes heterologous VLP forming sequence, it is particularly preferred and suitable to generate a fusion protein comprising a VLP forming sequence and an antigenic peptide or protein derived from Coronavirus S. Suitably, said antigenic peptide or protein, preferably the spike protein derived from a Coronavirus that is lacking the C-terminal transmembrane domain, or is lacking a part of the C-terminal transmembrane domain.

In other embodiments, where the coding sequence of the (additional) nucleic acid encodes heterologous VLP-forming sequence as defined above, it is particularly preferred and suitable to generate a fusion protein comprising a VLP-forming sequence and an antigenic peptide or protein derived from a Coronavirus spike protein fragment S1 (lacking S2 and/or TM and/or TMflex). Further, it may be suitable to use linker elements for separating the heterologous antigen clustering element from the antigenic peptide or protein (e.g. a linker according to SEQ ID NO: 115, 13148, 13152).

Further suitable VLP forming sequences in that context may be selected from the list of amino acid sequences according to SEQ ID NOs: 1168-1227 of the patent application WO2017081082, or fragments or variants of these sequences. SEQ ID NOs: 1168-1227 of WO2017081082 are herewith incorporated by reference.

In embodiments, the antigenic peptide or protein comprises a heterologous signal peptide. A heterologous signal peptide may be used to improve the secretion of the encoded Coronavirus S antigen.

Suitable secretory signal peptides may be selected from the list of amino acid sequences according to SEQ ID NOs: 1-1115 and SEQ ID NO: 1728 of published PCT patent application WO2017081082, or fragments or variants of these sequences. 1-1115 and SEQ ID NO: 1728 of WO2017081082 are herewith incorporated by reference.

In embodiments where the coding sequence of the (additional) nucleic acid encodes heterologous secretory signal peptide, it is particularly preferred and suitable to generate a fusion protein comprising a heterologous secretory signal peptide and an antigenic peptide or protein derived from a Coronavirus S. Suitably, said antigenic peptide or protein, preferably the spike protein derived from Coronavirus is lacking the N-terminal endogenous secretory signal peptide (lacking aa 1 to aa 15).

According to preferred embodiments, the (additional) nucleic acid comprises at least one coding sequence encoding at least one antigenic peptide or protein selected or derived from a Coronavirus S as defined herein, or fragments and variants thereof. In preferred embodiments, the nucleic acid comprises at least one coding sequence encoding at least one antigenic peptide or protein selected or derived from at least one Coronavirus S as defined herein, or fragments and variants thereof, wherein said at least one Coronavirus is selected from at least one (pandemic) Alphacoronavirus, at least one (pandemic) Betacoronavirus, at least one (pandemic) Gammacoronavirus, and/or at least one (pandemic) Deltacoronavirus.

In that context, any coding sequence encoding at least one antigenic protein of a Coronavirus S as defined herein, or fragments and variants thereof may be understood as suitable coding sequence and may therefore be comprised in the (additional) nucleic acid of the pharmaceutical composition.

Suitable features and embodiments that apply to the nucleic acids as defined herein encoding spike proteins are provided in paragraph “Nucleic acid features and embodiments” below.

Suitably, the (additional) nucleic acids encoding spike proteins is formulated and/or complexed. Suitable features and embodiments that apply to nucleic acid complexation or formulation are provided in paragraph “Formulation and Complexation” below.

Preferably, intramuscular or intradermal administration of a composition comprising at least one nucleic acid encoding Coronavirus spike protein (S) as defined herein results in expression of the encoded Coronavirus spike protein (S) construct in a subject. In embodiments where the nucleic acid is an RNA, administration of the composition results in translation of the RNA and to a production of the encoded Coronavirus spike protein (S) antigen in a subject. In embodiments where the nucleic acid is a DNA (e.g. plasmid DNA, adenovirus DNA), administration of said composition results in transcription of the DNA into RNA, and to a subsequent translation of the RNA into the encoded Coronavirus spike protein (S) antigen in a subject.

In embodiments, administration of the pharmaceutical composition comprising at least one nucleic acid encoding Coronavirus spike protein (S) to a subject elicits neutralizing antibodies against Coronavirus spike protein (S) and does not elicit disease enhancing antibodies. In particular, administration of a pharmaceutical composition comprising at least one nucleic acid encoding Coronavirus spike protein (S) pre-fusion stabilized spike protein to a subject does not elicit immunopathological effects, like e.g. enhanced disease and/or antibody dependent enhancement (ADE).

In preferred embodiments, administration of the pharmaceutical composition comprising at least one nucleic acid encoding Coronavirus spike protein (S) to a subject elicits antigen-specific immune responses comprising T-cell responses and/or B-cell responses against the encoded Coronavirus spike protein (S) antigen.

Nucleic Acid Encoding SARS-CoV-2 Spike Protein

In a particularly preferred embodiment, the Coronavirus spike protein (S) is selected or derived from at least one SARS-CoV-2 spike protein (S1, S2, or S1 and S2), or an immunogenic fragment or immunogenic variant thereof.

In a further particularly preferred embodiment, the Coronavirus spike protein (S) is selected or derived from at least one SARS-CoV-2 variant spike protein (S1, S2, or S1 and S2), or an immunogenic fragment or immunogenic variant thereof.

Accordingly, in preferred embodiments of the first aspect, the (additional) nucleic acid comprises at least one coding sequence encoding at least one antigenic peptide or protein selected or derived from at least one SARS-CoV-2, or an immunogenic fragment or immunogenic variant thereof.

It has to be understood that generic embodiments and features that have been described paragraph “Nucleic acid encoding a Coronavirus spike protein” may also apply to a nucleic acid encoding a SARS-CoV-2 spike protein.

In the context of the invention, any S protein selected or derived from a SARS-CoV-2 may be used in the context of the invention and may be suitably encoded by the coding sequence or the nucleic acid. It is further in the scope of the underlying invention, that the at least one antigenic peptide or protein may comprise or consist of a synthetically engineered or an artificial SARS-CoV-2 S peptide or protein. The term “synthetically engineered” SARS-CoV-2 S peptide or protein, or the term “artificial SARS-CoV-2 S peptide or protein” relates to an S protein that does not occur in nature. Accordingly, an “artificial SARS-CoV-2 S peptide or protein” or a “synthetically engineered SARS-CoV-2 S peptide or protein” may for example differ in at least one amino acid compared to the natural SARS-CoV-2 S peptide or protein, and/or may comprise an additional heterologous peptide or protein element, and/or may be N-terminally or C-terminally extended or truncated.

In the context of the invention, any Spike protein that is selected from or is derived from a SARS-CoV-2 comprising at least one amino acid substitution selected from a SARS-CoV-2 variant may be used and may be suitably encoded by the coding sequence or the nucleic acid may be used in the context of the invention. It is further in the scope of the underlying invention, that the at least one antigenic peptide or protein may comprise or consist of a synthetically engineered or an artificial SARS-CoV-2 spike protein. The term “synthetically engineered” SARS-CoV-2 spike protein, or the term “artificial SARS-CoV-2 spike protein” relates to a protein that does not occur in nature. Accordingly, an “artificial SARS-CoV-2 spike protein” or a “synthetically engineered SARS-CoV-2 spike protein” may for example differ in at least one amino acid compared to the natural SARS-CoV-2 spike protein, and/or may comprise an additional heterologous peptide or protein element, and/or may be N-terminally or C-terminally extended or truncated.

In preferred embodiments, the (additional) nucleic acid comprises at least one coding sequence encoding at least one antigenic peptide or protein selected or derived from SARS-CoV-2 spike protein (S), or an immunogenic fragment or immunogenic variant thereof.

In particularly preferred embodiments of the pharmaceutical composition, the encoded at least one antigenic peptide or protein of the (additional) nucleic acid comprises or consists at least one peptide or protein selected or derived from a SARS-CoV-2 spike protein (S, S1, S2, or S1 and S2), or an immunogenic fragment or immunogenic variant of any of these.

Suitable SARS-CoV-2 spike antigenic peptide or proteins sequences that are provided by the (additional) nucleic acid are disclosed in Table 2, rows 1 to 41, Column A and B. In addition, further information regarding said suitable antigenic peptide or protein sequences selected or derived from SARS-CoV-2 spike protein are provided under <223> identifier of the ST25 sequence listing.

It has to be noted that where reference is made to amino acid (aa) residues and their position in a SARS-CoV-2 spike protein, any numbering used herein—unless stated otherwise—relates to the position of the respective amino acid residue in a corresponding spike protein (S) of the original or ancestral SARS-CoV-2 coronavirus isolate EPI_ISL_402128 (BetaCoV_Wuhan_WIV05_2019_EPI_ISL_402128) according to SEQ ID NO: 1. Respective amino acid positions are, if referring to SARS-CoV-2 Spike protein, exemplarily indicated for spike protein (S) of SARS-CoV-2 coronavirus isolate EPI_ISL_402128 (SEQ ID NO: 1). The person skilled in the art will of course be able to adapt the teaching provided in the present specification exemplified for SARS-CoV-2 EPI_ISL_402128 (SEQ ID NO: 1) to other antigenic peptides or proteins in other SARS-CoV-2 coronavirus isolates, e.g. to isolates including but not limited to EPI_ISL_404227, EPI_ISL_403963, EPI_ISL_403962, EPI_ISL_403931, EPI_ISL_403930, EPI_ISL_403929, EPI_ISL_402130, EPI_ISL_402129, EPI_ISL_402128, EPI_ISL_402126, EPI_ISL_402125, EPI_ISL_402124, EPI_ISL_402123, EPI_ISL_402120, EPI_ISL_402119 (further SARS-CoV-2 isolates are provided in List A and/or List B).

Protein annotation for SARS-CoV-2 spike protein (S) was performed using SEQ ID NO: 1 as a reference protein. The full-length S of SARS-CoV-2 reference protein has 1273 amino acid residues, and comprises the following elements:
secretory signal peptide: amino acid position aa 1 to aa 15 (see SEQ ID NO: 28)
spike protein fragment S1: amino acid position aa 1 to aa 681 (see SEQ ID NO: 27)
S1-N-Terminal Domain (S1-NTD) amino acid position aa 1 to aa 681 (see SEQ ID NO: 26992)
receptor binding domain (RBD): amino acid position aa 319 to aa 541 (see SEQ ID NO: 13243)
critical neutralisation domain (CND): amino acid position aa 329 to aa 529 (see SEQ ID NO: 13310)
spike protein fragment S2: amino acid position aa 682 to aa 1273 (see SEQ ID NO: 30)
transmembrane domain (TM) amino acid position aa 1212 to aa 1273 (see SEQ ID NO: 49)
transmembrane domain (TMflex) amino acid position aa 1148 to aa 1273 (see SEQ ID NO: 13176)
Furine cleavage site region (S1/S2) amino acid position aa 681 to aa 685 (see SEQ ID NO: 26994)

It has to be noted that variation on amino acid level naturally occurs between spike proteins derived from different SARS-CoV-2 isolates or SARS-CoV-2 variants. In the context of the invention, such amino acid variations can be applied to each antigenic peptide or protein derived from a SARS-CoV-2 spike protein as described herein. Suitably, the amino acid variations or mutations are selected in a way to induce an immune response against the SARS-CoV-2 virus variant the substitution/mutation is derived from.

Accordingly, in particularly preferred embodiments, the nucleic acid of the invention comprises at least one coding sequence encoding at least one SARS-CoV-2 spike protein, or an immunogenic fragment or immunogenic variant thereof, wherein the SARS-CoV-2 spike protein comprises at least one amino acid substitution, deletion, or insertion selected from a SARS-CoV-2 variant.

In that context, the term “at least one amino acid substitution, deletion, or insertion selected from a SARS-CoV-2 variant” has to be understood as at least one amino acid position in the SARS-CoV-2 spike protein (or fragment thereof) that is different to the original SARS-CoV-2 spike protein (according to the SEQ ID NO: 1 reference strain).

In preferred embodiments, the SARS-CoV-2 variant is selected from or is derived from the following SARS-CoV-2 lineages: B.1.351 (South Africa), B.1.1.7 (UK), P.1 (Brazil), B.1.429 (California), B.1.525 (Nigeria), B.1.258 (Czech republic), B.1.526 (New York), A.23.1 (Uganda), B.1.617.1 (India), B.1.617.2 (India), B.1.617.3 (India), P.2 (Brazil), C37.1 (Peru).

In particularly preferred embodiments, the SARS-CoV-2 variant is selected from or derived from the following SARS-CoV-2 lineages: B.1.351 (South Africa), P.1 (Brazil), B.1.617.1 (India), B.1.617.2 (India), B.1.617.3 (India).

Accordingly, each spike protein of SARS-CoV-2 provided herein and contemplated as suitable antigen in the context of the invention may have one or more of the following amino acid variations, substitutions or mutations (amino acid positions according to reference SEQ ID NO: 1):
D614G or G614D; H49Y or Y49H; V367F or F367V; P1263L or L1263P; V483A or A483V; S939F or F939S; S943P or P943S; L5F or F5L; L8V or V8L; S940F or F940S; C1254F or F1254C; Q239K or K239Q; M153T or T153M; V1040F or F1040V; A845S or S845A; Y145H or H145Y; A831V or V831A; M1229I or I1229M; H69 or H69del/aa deleted; V70 or H70del/aa deleted; H69_V70 or H69del and H70del/aa deleted; A222V or V222A; Y453F or F453Y; S477N or N477S; I692V or V692I; R403K or K403R; K417N or N417K; N437S or S437N; N439K or K439N; V445A or A445V; V445I or I445V; V445F or F445V; G446V or V446G; G446S or S446G; G446A or A446G; L455F or F455L; F456L or L456F; K458N or N458K; A475V or V475A; G476S or S476G; G476A or A476G; S477I or I477S; S477R or R477S; S477G or G477S; S477T or T477S; T478I or I478T; T478K or K478T; T478R or R478T; T478A or A478T; E484Q or Q484E; E484K or K484E; E484A or A484E; E484D or D484E; G485R or R485G; G485S or S485G, F486L or L486F; N487I or I487N; Y489H or H489Y; F490S or S490F; F490L or L490F; Q493L or L493Q; Q493K or K493Q; S494P or P494S; S494L or L494S; P499L or L499P; T500I or I500T; N501Y or Y501N; N501T or T501N; N501S or S501N; V503F or F503V; V503I or I503V; G504D or D504G; Y505W or W505Y; Q506K or K506Q; Q506H or H506Q; H69 or H69del/aa deleted; V70 or H70del/aa deleted; H69_V70 or H69del and H70del/aa deleted; A222V or V222A; Y453F or F453Y; S477N or N477S; I692V or V692I; R403K or K403R; K417N or N417K; N437S or S437N; N439K or K439N; V445A or A445V; V445I or I445V; V445F or F445V; G446V or V446G; G446S or S446G; G446A or A446G; L455F or F455L; F456L or L456F; K458N or N458K; A475V or V475A; G476S or S476G; G476A or A476G; S477I or I477S; S477R or R477S; S477G or G477S; S477T or T477S; T478I or I478T; T478K or K478T; T478R or R478T; T478A or A478T; E484Q or Q484E; E484K or K484E; E484A or A484E; E484D or D484E; G485R or R485G; G485S or S485G; F486L or L486F; N487I or I487N; Y489H or H489Y; F490S or S490F; F490L or L490F; Q493L or L493Q; Q493K or K493Q; S494P or P494S; S494L or L494S; P499L or L499P; T500I or I500T: N501Y or Y501N; N501T or T501N; N501S or S501N; V503F or F503V; V503I or I503V; G504D or D504G; Y505W or W505Y; Q506K or K506Q; Q506H or H506Q; Y144 or Y144del/aa del; A570D or D570A; P681H or H681P; T716I or I716T; S982A or A982S; D1118H or H1118D; L18F or F18L; D80A or A80D; D215G or G215D; L242 or L242del/aa deleted; A243 or A243del/aa deleted; L244 or L244del/aa deleted; L242_A243_L244 or L242del and A243del and L244del/aa deleted; R246I or I246R; A701V or V701A; T20N or N20T; P26S or S26P; D138Y or Y138D; R190S or S190R; H655Y or Y655H; T1027I or I1027T; S13I or I13S; W152C or C152W; L452R or R452L; R346T or T346R; P384L or L384P; L452M or M452L; F456A or A456F; F456K or K456F; F456V or V456F; E484P or P484E; K417T or T417K; G447V or V447G; L452Q or Q452L; A475S or S475A; F486I or I486F; F490Y or Y490F; Q493R or R493Q; S494A or A494S; P499H or H499P; P499S or S499P; G502V or V502G; T748K or K748T; A522S or S522A; and/or V1176F or F1176V.

The variations or mutations provided below are derived from novel emerging SARS-CoV-2 virus variants, and may be integrated into the spike protein that is provided by the nucleic acid of the invention:

List 1A: Amino Acid Positions for Substitutions Deletions and/or Insertions
H69; V70; A222; Y453; S477; I692; R403; K417; N437; N439; V445; G446; L455; F456; K458; A475; G476; T478; E484; G485, F486; N487; Y489; F490; Q493; S494; P499; T500; N50I; V503; G504; Y505; 0506; Y144; A570; P68I; T716; S982; D1118; L18; D80; D215; L242; A243; L244; R246; A70I; T20; P26; D138; R190; H655; T1027; S13; W152; L452; R346; P384; G447; G502; T748; A522; V1176; T859; S247; Y248; L249; T250; P25I; G252; G75; T76; D950; E154; G769; S254; Q613; F157; R158; Q957; D253; T95; F888; Q677; A67; Q414; N450; V483; G669; T732; Q949; Q107I; E1092; H110I; N1187; W258; T19; V126; H245; S12; A899; G142; E156; K558; or Q52 (relative to the sequence of SEQ ID NO: 1).

List 1B: Amino Acid Substitutions Deletions or Insertions
H69del; V70del; A222V; Y453F; S477N; I692V; R403K; K417N; N437S; N439K; V445A; V445I; V445F; G446V; G446S; G446A; L455F; F456L; K458N; A475V; G476S; G476A; S477I; S477R; S477G; S477T; T478I; T478K; T478R; T478A; E484Q; E484K; E484A; E484D; G485R; G485S, F486L; N487I; Y489H; F490S; F490L; Q493L; Q493K; S494P; S494L; P499L; T500I; N501Y; N501T; N501S; V503F; V503I; G504D; Y505W; Q506K; Q506H; Y144del; A570D; P681H; T716I; S982A; D1118H; L18F; D80A; D215G; L242del; A243del; L244del; L242del; A243del; L244del; R246I; A701V; T20N; P26S; D138Y; R190S; H655Y; T1027I; S13I; W152C; L452R; R346T; P384L; L452M; F456A; F456K; F456V; E484P; K417T; G447V; L452Q; A475S; F486I; F490Y; Q493R; S494A; P499H; P499S; G502V; T748K; A522S; V1176F; T859N; S247del; Y248del; L249del; T250del; P251del; G252del; R246del; S247del; Y248del; L249del; T250del; P251del; G252del; G75V; T76I; G75V; T76I; D950N; P681R; E154K; G769V; S254F; Q613H; F157L; F157del; R158del; Q957R; D253G; T95I; F888L; Q677H; A67V; Q414K; N450K; V483A; G669S; T732A; Q949R; Q1071H; E1092K; H1101Y; N1187D; W258L; V70F; T19R; Y144T; Y145S; ins145N; R346K; R346S; V126A; H245Y; ins214TDR; S12F; W152R; A899S; G142D; E156G; K558N; or Q52R (relative to the sequence of SEQ ID NO: 1).

In a preferred embodiment there is provided a RNA comprising at least one coding sequence encoding at least one SARS-CoV-2 spike protein or an immunogenic fragment or immunogenic variant thereof, wherein the SARS-CoV-2 spike protein comprises at least one amino acid substitution, deletion or insertion at a position corresponding to H69; V70; A222; Y453; S477; I692; R403; K417; N437; N439; V445; G446; L455; F456; K458; A475; G476; T478; E484; G485, F486; N487; Y489; F490; Q493; S494; P499; T500; N50I; V503; G504; Y505; 0506; Y144; A570; P68I; T716; S982; D1118; L18; D80; D215; L242; A243; L244; R246; A70I; T20; P26; D138; R190; H655; T1027; S13; W152; L452; R346; P384; G447; G502; T748; A522; V1176; T859; S247; Y248; L249; T250; P25I; G252; G75; T76; D950; E154: G769; S254; 0613; F157; R158; Q957; D253; T95; F888; Q677; A67; Q414; N450; V483; G669; T732; Q949; Q107I; E1092; H110I; N1187; W258; T19; V126; H245; S12; A899; G142; E156; K558; and/or Q52 relative to the sequence of SEQ ID NO: 1.

In particularly preferred embodiments, the SARS-CoV-2 spike protein comprises at least one amino acid substitution, deletion or insertion at a position corresponding to H69del; V70del; A222V; Y453F; S477N; I692V; R403K; K417N; N437S; N439K; V445A; V445I; V445F; G446V; G446S; G446A; L455F; F456L; K458N; A475V; G476S; G476A; S477I; S477R; S477G; S477T; T478I; T478K; T478R; T478A; E484Q; E484K; E484A; E484D; G485R; G485S, F486L; N487I; Y489H; F490S; F490L; Q493L; Q493K; S494P; S494L; P499L; T500I; N501Y; N501T; N501S; V503F; V503I; G504D; Y505W; Q506K; Q506H; Y144del; A570D; P681H; T716I; S982A; D1118H; L18F; D80A; D215G; L242del; A243del; L244del; L242del; A243del; L244del; R246I; A701V; T20N; P26S; D138Y; R190S; H655Y; T1027I; S13I; W152C; L452R; R346T; P384L; L452M; F456A; F456K; F456V; E484P; K417T; G447V; L452Q; A475S; F486I; F490Y; Q493R; S494A; P499H; P499S; G502V; T748K; A522S; V1176F; T859N; S247del; Y248del; L249del; T250del; P251del; G252del; R246del; S247del; Y248del; L249del; T250del; P251del; G252del; G75V; T76I; G75V; T76I; D950N; P681R; E154K; G769V; S254F; Q613H; F157L; F157del; R158del; Q957R; D253G; T95I; F888L; Q677H; A67V; Q414K; N450K; V483A; G669S; T732A; Q949R; Q1071H; E1092K; H1101Y; N1187D; W258L; V70F; T19R; Y144T; Y145S; ins145N; R346K; R346S; V126A; H245Y; ins214TDR; S12F; W152R; A899S; G142D; E156G; K558N; and/or Q52R relative to the sequence of SEQ ID NO: 1.

In certain embodiments there is provided a nucleic acid comprising at least one coding sequence encoding at least one SARS-CoV-2 spike protein or an immunogenic fragment or immunogenic variant thereof, wherein the SARS-CoV-2 spike protein comprises at least one amino acid substitution, deletion or insertion at a position corresponding to H69; V70; A222; Y453; S477; I692; R403; K417; N437; N439; V445; G446; L455; F456; K458; A475; G476; T478; E484; G485, F486; N487; Y489; F490; Q493; S494; P499; T500; N50I; V503; G504; Y505; and/or Q506 relative to the sequence of SEQ ID NO: 1. Thus, in some embodiments the SARS-CoV-2 spike protein comprises at least one amino acid substitution, deletion or insertion at a position corresponding to H69del; V70del; A222V; Y453F; S477N; I692V; R403K; K417N; N437S; N439K; V445A; V445I; V445F; G446V; G446S; G446A; L455F; F456L; K458N; A475V; G476S; G476A; S477I; S477R; S477G; S477T; T478I; T478K; T478R; T478A; E484Q; E484K; E484A; E484D; G485R; G485S, F486L; N487I; Y489H; F490S; F490L; Q493L; Q493K; S494P; S494L; P499L; T500I; N501Y; N501T; N501S; V503F; V503I; G504D; Y505W; Q506K; and/or Q506H relative to the sequence of SEQ ID NO: 1.

In a further embodiment there is provided a nucleic acid comprising at least one coding sequence encoding at least one SARS-CoV-2 spike protein or an immunogenic fragment or immunogenic variant thereof, wherein the SARS-CoV-2 spike protein comprises at least one amino acid substitution, deletion or insertion at a position corresponding to H69; V70; A222; Y453; S477; I692; R403; K417; N437; N439; V445; G446; L455; F456; K458; A475; G476; T478; E484; G485, F486; N487; Y489; F490; Q493; S494; P499; T500; N50I; V503; G504; Y505; Q506; Y144; A570; P68I; T716; S982; D1118; L18; D80; D215; L242; A243; L244; R246; A70I; T20; P26; D138; R190; H655; T1027; S13; W152; L452; R346; P384; G447; G502; T748; A522; or V1176 relative to the sequence of SEQ ID NO: 1. Thus, in some embodiments the SARS-CoV-2 spike protein comprises at least one amino acid substitution, deletion or insertion at a position corresponding to H69del; V70del; A222V; Y453F; S477N; I692V; R403K; K417N; N437S; N439K; V445A; V445I; V445F; G446V; G446S; G446A; L455F; F456L; K458N; A475V; G476S; G476A; S477I; S477R; S477G; S477T; T478I; T478K; T478R; T478A; E484Q; E484K; E484A; E484D; G485R; G485S, F486L; N487I; Y489H; F490S; F490L; Q493L; Q493K; S494P; S494L; P499L; T500I; N501Y; N501T; N501S; V503F; V503I; G504D; Y505W; Q506K; Q506H; Y144del; A570D; P681H; T716I; S982A; D1118H; L18F; D80A; D215G; L242del; A243del; L244del; L242del; A243del; L244del; R246I; A701V; T20N; P26S; D138Y; R190S; H655Y; T1027I; S13I; W152C; L452R; R346T; P384L; L452M; F456A; F456K; F456V; E484P; K417T; G447V; L452Q; A475S; F486I; F490Y; Q493R; S494A; P499H; P499S; G502V; T748K; A522S; and/or V1176F relative to the sequence of SEQ ID NO: 1.

In a further preferred embodiment there is provided a nucleic acid comprising at least one coding sequence encoding at least one SARS-CoV-2 spike protein or an immunogenic fragment or immunogenic variant thereof, wherein the SARS-CoV-2 spike protein comprises at least one amino acid substitution, deletion or insertion at a position corresponding to T859; R246; S247; Y248; L249; T250; P251; G252; G75; T76; D950; E154; G769; S254; Q613; F157; Q957; D253; T95; F888; Q677; A67; Q414; N450; V483; G669; T732; Q949; Q1071; E1092; H1101; N1187; F157; R158; W258; T19; H245; S12; A899; G142; E156; K558 and/or Q52 relative to the sequence of SEQ ID NO: 1. Thus, in some embodiments the SARS-CoV-2 spike protein comprises at least one amino acid substitution, deletion or insertion at a position corresponding to T859N; S247del; Y248del; L249del; T250del; P251del; G252del; R246del; S247del; Y248del; L249del; T250del; P251del; G252del; G75V; T76I; G75V; T76I; D950N; P681R; E154K; G769V; S254F; Q613H; F157L; Q957R; D253G; T95I; F888L; Q677H; A67V; Q414K; N450K; V483A; G669S; T732A; Q949R; Q1071H; E1092K; H1101Y; N1187D; F157del; R158del; W258L; V70F; T19R; Y144T; Y145S; ins145N; R346K; R346S; V126A; H245Y; ins214TDR; S12F; W152R; A899S; G142D; E156G; K558N and/or Q52R relative to the sequence of SEQ ID NO: 1.

In still a further embodiment there is provided a nucleic acid comprising at least one coding sequence encoding at least one SARS-CoV-2 spike protein or an immunogenic fragment or immunogenic variant thereof, wherein the SARS-CoV-2 spike protein comprises at least one amino acid substitution, deletion or insertion at a position corresponding to D614; H49; V367; P1263; V483; S939; S943; L5; L8; S940; C1254; Q239; M153; V1040; A845; Y145; A831; and/or M1229 relative to the sequence of SEQ ID NO: 1. Thus, in some embodiments the SARS-CoV-2 spike protein comprises at least one amino acid substitution, deletion or insertion at a position corresponding to D614G; H49Y; V367F; P1263L; V483A; S939F; S943P; L5F; L8V; S940F; C1254F; Q239K; M153T; V1040F; A845S; Y145H; A831V; and/or M1229I relative to the sequence of SEQ ID NO: 1.

In still a further embodiment there is provided a nucleic acid comprising at least one coding sequence encoding at least one SARS-CoV-2 spike protein or an immunogenic fragment or immunogenic variant thereof, wherein the SARS-CoV-2 spike protein comprises at least one amino acid substitution or deletion at a position corresponding to H69; V70; A222; Y453; S477; I692; R403; K417; N437; N439; V445; G446; L455; F456; K458; A475; G476; T478; E484; G485, F486; N487; Y489; F490; Q493; S494; P499; T500; N501; V503; G504; Y505; Q506; Y144; A570; P681; T716; S982; D1118; L18; D80; D215; L242; A243; L244; R246; A701; T20; P26; D138; R190; H655; T1027; S13; W152; L452; R346; P384; G447; G502; T748; A522; V1176; T859; S247; Y248; L249; T250; P251; G252; G75; T76; D950; E154; G769; S254; Q613; F157; Q957; D253; T95; F888; Q677; A67; Q414; N450; V483; G669; T732; Q949; Q1071; E1092; H1101; N1187 and/or Q52 relative to the sequence of SEQ ID NO: 1. Thus, in some embodiments the SARS-CoV-2 spike protein comprises at least one amino acid substitution or deletion at a position corresponding to H69del; V70del; A222V; Y453F; S477N; I692V; R403K; K417N; N437S; N439K; V445A; V445I; V445F; G446V; G446S; G446A; L455F; F456L; K458N; A475V; G476S; G476A; S477I; S477R; S477G; S477T; T478I; T478K; T478R; T478A; E484Q; E484K; E484A; E484D; G485R; G485S, F486L; N487I; Y489H; F490S; F490L; Q493L; Q493K; S494P; S494L; P499L; T500I; N501Y; N501T; N501S; V503F; V503I; G504D; Y505W; Q506K; Q506H; Y144del; A570D; P681H; T716I; S982A; D1118H; L18F; D80A; D215G; L242del; A243del; L244del; L242del; A243del; L244del; R246I; A701V; T20N; P26S; D138Y; R190S; H655Y; T1027I; S13I; W152C; L452R; R346T; P384L; L452M; F456A; F456K; F456V; E484P; K417T; G447V; L452Q; A475S; F486I; F490Y; Q493R; S494A; P499H; P499S; G502V; T748K; A522S; V176F; T859N; S247del; Y248del; L249del; T250del; P251del; G252del; R246del; S247del; Y248del; L249del; T250del; P251del; G252del; G75V; T76I; G75V; T76I; D950N; P681R; E154K; G769V; S254F; Q613H; F157L: F157del; R158del; Q957R; D253G; T95I; F888L; Q677H; A67V; Q414K; N450K; V483A; G669S; T732A; Q949R; Q1071H; E1092K; H1101Y; N1187D; W258L; V70F; T19R; Y144T; Y145S; R346K; R346S; V126A; H245Y; S12F; W152R; A899S; G142D; E156G; K558N; and/or Q52R.

In preferred embodiments, the SARS-CoV-2 spike protein comprises an amino acid substitution at a position located in the RBD domain (amino acid position aa 319 to aa 541; amino acid positions according to reference SEQ ID NO: 1) or the CND domain (amino acid position aa 329 to aa 529; amino acid positions according to reference SEQ ID NO: 1). Without wishing to be bound to theory, amino acid substitutions or mutations in the CND domain may help novel emerging SARS-CoV-2 variants to evade antibody detection of some types of antibodies induced in subjects vaccinated with first generation vaccines (designed against the original SARS-CoV-2 strain) or induced in subjects after infection with the original SARS-CoV-2 strain.

Accordingly, in preferred embodiments, the first aspect of the invention relates to a nucleic acid comprising at least one coding sequence encoding at least one antigenic peptide or protein from a SARS-CoV-2 spike protein or an immunogenic fragment or immunogenic variant thereof, wherein the SARS-CoV-2 spike protein comprises at least one amino acid substitution at position located in the RBD domain (amino acid position aa 319 to aa 541; amino acid positions according to reference SEQ ID NO: 1) or the CND domain (amino acid position aa 329 to aa 529 amino acid positions according to reference SEQ ID NO: 1).

In certain preferred embodiments, the SARS-CoV-2 spike protein comprises an amino acid substitution, insertion or deletion in at least one of the following positions: R346; V367, P384; R403; K417; N437; N439; V445; G446; G447; N450; L452; Y453; L455; F456; A475; G476; S477; T478; E484; G485; F486; N487; Y489; F490; Q493; S494; P499; T500; N501; G502; V503; G504; Y505; Q506; A522 (amino acid positions according to reference SEQ ID NO: 1).

Accordingly, in certain preferred embodiments, the first aspect of the invention relates to an nucleic acid comprising at least one coding sequence encoding at least one antigenic peptide or protein from a SARS-CoV-2 spike protein or an immunogenic fragment or immunogenic variant thereof, wherein the SARS-CoV-2 spike protein comprises at least one amino acid substitution at positions selected from K417; L452; E484; N501 and/or P681 (amino acid positions according to reference SEQ ID NO: 1).

Without wishing to be bound to theory, an amino acid substitution at position E484 may help SARS-CoV-2 virus variants to evade antibody detection of some types of antibodies induced in subjects vaccinated with first generation vaccines (designed against the original SARS-CoV-2 strain) or induced in subjects after infection with the original SARS-CoV-2 strain. A mutation/substitution in N501 occurs near the top of the coronavirus spike, where it may alter the shape of the protein, which may help to evade some types of coronavirus antibodies. Such SARS-CoV-2 are called SARS-CoV-2 E484 variants throughout the present invention and include e.g. SARS-CoV-2 B.1.351 (South Africa), SARS-CoV-2 B.1.617 (India), or P.1 (Brazil).

Accordingly, in some embodiments, it may be advantageous that the nucleic acid of the invention provides a SARS-CoV-2 spike protein comprising a substitution in position E484 to allow the induction of efficient immune responses against virus SARS-CoV-2 E484 variants.

In preferred embodiments, the SARS-CoV-2 spike protein comprises an amino acid substitution at position E484, wherein the amino acids E484 is substituted with K, P, Q, A, or D (amino acid positions according to reference SEQ ID NO: 1). Accordingly, the antigenic peptide or protein selected from or derived from SARS-CoV-2 spike protein comprises a E484K, E484P, E484Q, E484A, E484D amino acid substitution.

In particularly preferred embodiments, the SARS-CoV-2 spike protein comprises an amino acid substitution at position E484, wherein the amino acids E484 is substituted with K or Q (amino acid positions according to reference SEQ ID NO: 1). Accordingly, the antigenic peptide or protein selected from or derived from SARS-CoV-2 spike protein comprises a E484K or E484Q amino acid substitution. In certain preferred embodiments a SARS-CoV-2 spike protein comprises a E484K amino acid substitution.

In preferred embodiments, the SARS-CoV-2 spike protein comprises an amino acid substitution at position N501, wherein the amino acids N501 is substituted with a different amino acid (amino acid positions according to reference SEQ ID NO: 1).

Without wishing to be bound to theory, an amino acid substitution at position N501 may help SARS-CoV-2 virus variants to evade antibody detection of some types of antibodies induced in subjects vaccinated with first generation vaccines (designed against the original SARS-CoV-2 strain) or induced in subjects after infection with the original SARS-CoV-2 strain. A mutation/substitution in N501 occurs near the top of the coronavirus spike, where it may alter the shape of the protein, which may help to evade some types of coronavirus antibodies. Such SARS-CoV-2 are called SARS-CoV-2 N501 variants throughout the present invention and include e.g. SARS-CoV-2 B.1.351 (South Africa), SARS-CoV-2 B.1.1.7 (UK), or P.1 (Brazil).

Accordingly it may be advantageous that the nucleic acid of the invention provides a SARS-CoV-2 spike protein comprising a substitution in position N501 to allow the induction of efficient immune responses against virus SARS-CoV-2 N501 variants.

In preferred embodiments, the SARS-CoV-2 spike protein comprises an amino acid substitution at position N501, wherein the amino acids N501 is substituted with Y, T, S (amino acid positions according to reference SEQ ID NO: 1). Accordingly, the antigenic peptide or protein selected from or derived from SARS-CoV-2 spike protein comprises a N501Y, N501T, N501S amino acid substitution.

In particularly preferred embodiments, the SARS-CoV-2 spike protein comprises an amino acid substitution at position N501, wherein the amino acids N501 is substituted with Y (amino acid positions according to reference SEQ ID NO: 1). Accordingly, the antigenic peptide or protein selected from or derived from SARS-CoV-2 spike protein comprises a N501Y amino acid substitution.

In preferred embodiments, the SARS-CoV-2 spike protein comprises an amino acid substitution at position K417, wherein the amino acids K417 is substituted with a different amino acid (amino acid positions according to reference SEQ ID NO: 1).

Without wishing to be bound to theory, an amino acid substitution at position K417 may help SARS-CoV-2 virus variants to evade antibody detection of some types of antibodies induced in subjects vaccinated with first generation vaccines (designed against the original SARS-CoV-2 strain) or induced in subjects after infection with the original SARS-CoV-2 strain. A mutation/substitution in K417 occurs near the top of the coronavirus spike, where it may alter the shape of the protein, which may help to evade some types of coronavirus antibodies. Such SARS-CoV-2 are called SARS-CoV-2 K417 variants throughout the present invention and include e.g. SARS-CoV-2 B.1.351 (South Africa), SARS-CoV-2 B.1.1.7 (UK), P.1 (Brazil) or AY.1/AY.2.

Accordingly it may be advantageous that the nucleic acid of the invention provides a SARS-CoV-2 spike protein comprising a substitution in position K417 to allow the induction of efficient immune responses against virus SARS-CoV-2 K417 variants.

In preferred embodiments, the SARS-CoV-2 spike protein comprises an amino acid substitution at position K417, wherein the amino acids N501 is substituted with S, T, Q or N (amino acid positions according to reference SEQ ID NO: 1). Accordingly, the antigenic peptide or protein selected from or derived from SARS-CoV-2 spike protein comprises a K417S, K417T, K417Q or K417N amino acid substitution.

In particularly preferred embodiments, the SARS-CoV-2 spike protein comprises an amino acid substitution at position N501, wherein the amino acids K417 is substituted with T or N (amino acid positions according to reference SEQ ID NO: 1). Accordingly, the antigenic peptide or protein selected from or derived from SARS-CoV-2 spike protein comprises a K417T or K417N amino acid substitution. In certain preferred embodiments the antigenic peptide or protein selected from or derived from SARS-CoV-2 spike protein comprises a K417N amino acid substitution.

In preferred embodiments, the SARS-CoV-2 spike protein comprises an amino acid substitution at position L452, wherein the amino acids L452 is substituted with a different amino acid (amino acid positions according to reference SEQ ID NO: 1).

Without wishing to be bound to theory, an amino acid substitution at position L452 may help SARS-CoV-2 virus variants to evade antibody detection of some types of antibodies induced in subjects vaccinated with first generation vaccines (designed against the original SARS-CoV-2 strain) or induced in subjects after infection with the original SARS-CoV-2 strain. A mutation/substitution in L452 occurs near the top of the coronavirus spike, where it may alter the shape of the protein, which may help to evade some types of coronavirus antibodies. Such SARS-CoV-2 are called SARS-CoV-2 L452 variants throughout the present invention and include e.g. SARS-CoV-2 B.1.617.1 (India), SARS-CoV-2 B.1.617.2 (India), or SARS-CoV-2 B.1.617.3 (India).

Accordingly it may be advantageous that the nucleic acid of the invention provides a SARS-CoV-2 spike protein comprising a substitution in position L452 to allow the induction of efficient immune responses against virus SARS-CoV-2 L452 variants.

In preferred embodiments, the SARS-CoV-2 spike protein comprises an amino acid substitution at position L452, wherein the amino acids L452 is substituted with R or Q (amino acid positions according to reference SEQ ID NO: 1). Accordingly, the antigenic peptide or protein selected from or derived from SARS-CoV-2 spike protein comprises an L452R or L452Q amino acid substitution.

In particularly preferred embodiments, the SARS-CoV-2 spike protein comprises an amino acid substitution at position L452, wherein the amino acids L452 is substituted with R (amino acid positions according to reference SEQ ID NO: 1). Accordingly, the antigenic peptide or protein selected from or derived from SARS-CoV-2 spike protein comprises a L452R amino acid substitution.

In preferred embodiments, the SARS-CoV-2 spike protein comprises an amino acid substitution at a position located in the furin cleavage site (amino acid position aa 681 to 685; amino acid positions according to reference SEQ ID NO: 1). That sequence stretch (PRRAR in SEQ ID NO: 1) is believed to serve as a recognition site for furin cleavage.

Without wishing to be bound to theory, amino acid substitutions or mutations in the furin cleavage site may help novel emerging SARS-CoV-2 variants to increased membrane fusion and thus cause increased transmissibility.

In preferred embodiments, the SARS-CoV-2 spike protein comprises an amino acid substitution at position P681 in the furin cleavage site. Suitably, the amino acids P681 is substituted with a different amino acid (amino acid positions according to reference SEQ ID NO: 1), preferably an amino acid that improves furin cleavage. Such SARS-CoV-2 are called SARS-CoV-2 P681 variants throughout the present invention and include e.g. SARS-CoV-2 B.1.617.1 (India), SARS-CoV-2 B.1.617.2 (India), or SARS-CoV-2 B.1.617.3 (India), SARS-CoV-2 B.1.1.7 (UK), SARS-CoV-2 A.23.1 (Uganda).

Accordingly it may be advantageous that the nucleic acid of the invention provides a SARS-CoV-2 spike protein comprising a substitution in position P681 to allow the induction of efficient immune responses against virus SARS-CoV-2 P681 variants.

In preferred embodiments, the SARS-CoV-2 spike protein comprises an amino acid substitution at position P681, wherein the amino acids P681 is substituted with R or H (amino acid positions according to reference SEQ ID NO: 1). Accordingly, the antigenic peptide or protein selected from or derived from SARS-CoV-2 spike protein comprises an P681R or P681H amino acid substitution.

In particularly preferred embodiments, the SARS-CoV-2 spike protein comprises an amino acid substitution at position P681, wherein the amino acids P681 is substituted with R (amino acid positions according to reference SEQ ID NO: 1). Accordingly, the antigenic peptide or protein selected from or derived from SARS-CoV-2 spike protein comprises a P681R amino acid substitution.

In particularly preferred embodiments, the SARS-CoV-2 spike protein that is provide by the nucleic acid of the invention comprises an amino acid substitution at position L452 as defined herein, preferably L452R, and an amino acid substitution at position P681 as defined herein, preferably P681R (amino acid positions according to reference SEQ ID NO: 1).

In another particularly preferred embodiment, the SARS-CoV-2 spike protein that is provided by the nucleic acid of the invention comprises an amino acid substitution at position L452 as defined herein, preferably L452R, and an amino acid substitution at position P681 as defined herein, preferably P681R (amino acid positions according to reference SEQ ID NO: 1). In further preferred embodiment, the SARS-CoV-2 spike protein that is provided by the nucleic acid of the invention comprises an amino acid substitution at position L452 as defined herein, preferably L452R, an amino acid substitution at position P681 as defined herein, preferably P681R and D614 as defined herein, preferably D614G, (amino acid positions according to reference SEQ ID NO: 1).

In particularly preferred embodiments, the SARS-CoV-2 spike protein that is provided by the nucleic acid of the invention comprises an amino acid substitution at position N501 as defined herein, preferably N501Y, and an amino acid substitution at position E484 as defined herein, preferably E484K (amino acid positions according to reference SEQ ID NO: 1).

In particularly preferred embodiments, the SARS-CoV-2 spike protein that is provided by the nucleic acid of the invention comprises an amino acid substitution at position L452 as defined herein, preferably L452R, and an amino acid substitution at position E484 as defined herein, preferably E484Q (amino acid positions according to reference SEQ ID NO: 1)

In preferred embodiments, the SARS-CoV-2 spike protein comprises, in addition to the substitutions defined above (at positions E484, N501, L452 and optionally P681), at least one, in particular 1, 2, 3, 4, 5, 6, 7, 8, 9, or 10 additional amino acid substitution, insertion or deletion selected from List 1A or List 1B.

In particularly preferred embodiments, the SARS-CoV-2 spike protein that is provided by the nucleic acid of the invention comprises an amino acid substitution or deletion at position H69 as defined herein, preferably H69del, and an amino acid substitution or deletion at position V70 as defined herein, preferably V70del (amino acid positions according to reference SEQ ID NO: 1). In further preferred embodiment, the SARS-CoV-2 spike protein that is provide by the RNA of the invention comprises a deletion at both H69 and V70.

In preferred embodiments, the SARS-CoV-2 spike protein that is provided by the nucleic acid of the invention comprises at least one further amino acid substitution or deletion selected from the following SARS-CoV-2 isolates B.1.351 (South Africa), B.1.1.7 (UK), P.1 (Brazil), B.1.429 (California), B.1.525 (Nigeria), B.1.258 (Czech republic), B.1.526 (New York), A.23.1 (Uganda), B.1.617.1 (India), B.1.617.2 (India), B.1.617.3 (India), P.2 (Brazil), C37.1 (Peru).

In preferred embodiments, the SARS-CoV-2 spike protein that is provided by the nucleic acid of the invention comprises amino acid substitutions or deletions selected from (amino acid positions according to reference SEQ ID NO: 1):
E484K, N501Y, L18F, D80A, D215G, L242del, A243del, L244del, R246I, K417N, D614G, and A701V; (SA; B.1.351)
E484K, N501Y, L18F, D80A, D215G, L242del, A243del, L244del, K417N, D614G, and A701V; (SA; B.1.351)
E484K, N501Y, L18F, T20N, P26S, D138Y, R190S, K417T, D614G, H655Y, and T1027I; (Brazil; P1)
E484K, N501Y, L18F, T20N, P26S, D138Y, R190S, K417T, D614G, H655Y, T1027I, and V1176F; (Brazil P1)
L452R, P681R, and D614G; (B.1.617.1; India)
L452R, E484Q, P681R, E154K, D614G, and Q1071H; (B.1.617.2; India)
L452R, P681R, T19R, F157del, R158del, T478K, D614G, and D950N; (B.1.617.2; India)
T19R, L452R, E484Q, D614G, P681R and D950N; (B.1.617.3; India)
G75V, T76I, S247del, Y248del, L249del, T250del, P251del, G252del, D253del, L452Q, F490S, D614G, and T859N; (C.37.1; Peru)
T95I, Y145N, R346K, E484K, N501Y, D614G, P681H, and D950N; (B.1.1.621)
T95I, Y144T, Y145S, ins145N, R346K, E484K, N501Y, D614G, P681H, and D950N; (B.1.1.621)
H69del, V70del, Y144del, E484K, N501Y, A570D, D614G, P681H, T716I, S982A, and D1118H; (B.1.1.7-E484K)
S13I, W152C, L452R, and D614G; (B.1.429)
L452R; and D614G; (B.1.429)
H69del; V70del; N439K; D614G; (B.1.258)
T95I; E484K; D614G; and A701V; (B.1.526)
L5F, T95I, D253G, E484K, D614G, and A701V; (B.1.526)
L5F, T95I, D253G, S477N, D614G, and Q957R; (B.1.526)
F157L; V367F; Q613H; and P681R (A.23.1)
S254F; D614G; P681R; and G769V (A.23.1)
T478K; D614G; P681H; and T732A (B.1.1.519; Mexico)
P26S, H69del, V70del, V126A, Y144del, L242del, A243del, L244del, H245Y, S477N, E484K, D614G, P681H, T1027I and D1118H; (B.1.620; Africa)
ins214TDR, Q414K, N450K, D614G, and T716I; (B.1.214.2)
S12F, H69del, V70del, W152R, R346S, L452R, D614G, Q677H and A899S; (C.36.3; Thailand)
E484K, D614G and V1176F; (P2)
Q52R; A67V; H69del; V70del; F157del; R158del; E484K; D614G; Q677H and F888L; (B.1.525)
Q52R; A67V; H69del; V70del; Y144del; E484K; D614G; Q677H and F888L; (B.1.525)
A67V; H69del; V70del; Y144del; E484K; D614G; Q677H and F888L; (B.1.525)
T19R; T95I; G142D, E156G, F157del; R158del; W258L; K417N; L452R; T478K; K558N, D614G; P681R; and D950N; (AY.1)
T19R; V70F; G142D, E156G, F157del; R158del; A222V, K417N; L452R; T478K; D614G; P681R; and D950N; (AY.2)
T19R; T95I; F157del; R158del; W258L; K417N; L452R; T478K; D614G; P681R; and D950N; or (AY.1)
T19R; V70F; F157del; R158del; A222V; K417N; L452R; T478K; D614G; P681R; and D950N; (AY.2)
H69del, V70del and D614G;
D614G and M1229I;
A222V and D614G;
S477N and D614G;
N439K and D614G;
H69del, V70del, Y453F, D614G and 1692I;
Y453F and D614G;
D614G and I692V;
H69del, V70del, A222V, Y453F, D614G and 1692I;
N501Y and D614G;
K417N; E484K; N501Y and D614G; or
E484K and D614G.

In particularly preferred embodiments, the SARS-CoV-2 spike protein that is provided by the nucleic acid of the invention comprises the following amino acid substitutions or deletions (relative to SEQ ID NO: 1):
E484K, N501Y, L18F, D80A, D215G, L242del, A243del, L244del, R246I, K417N, D614G, and A701V;
E484K, N501Y, L18F, D80A, D215G, L242del, A243del, L244del, K417N, D614G, and A701V;
E484K, N501Y, L18F, T20N, P26S, D138Y, R190S, K417T, D614G, H655Y, and T1027I;
E484K, N501Y, L18F, T20N, P26S, D138Y, R190S, K417T, D614G, H655Y, T1027I, and V1176F;
L452R, P681R, and D614G;
L452R, E484Q, P681R, E154K, D614G, and Q1071H; or
L452R, P681R, T19R, F157del, R158del, T478K, D614G, and D950N.

In even more preferred embodiments, the SARS-CoV-2 spike protein that is provided by the nucleic acid of the invention comprises the following amino acid substitutions or deletions (relative to SEQ ID NO: 1):
E484K, N501Y, L18F, D80A, D215G, L242del, A243del, L244del, R246I, K417N, D614G, and A701V; or
E484K, N501Y, L18F, D80A, D215G, L242del, A243del, L244del, K417N, D614G, and A701V.

In further particularly preferred embodiments, the SARS-CoV-2 spike protein that is provided by the nucleic acid of the invention comprises the following amino acid substitutions or deletions (relative to SEQ ID NO: 1):
L452R, P681R, and D614G;
L452R, E484Q, P681R, E154K, D614G, and Q1071H;
L452R, P681R, T19R, F157del, R158del, T478K, D614G, and D950N; or
T19R, L452R, E484Q, D614G, P681R and D950N.

The following amino acid variations (amino acid positions according to reference SEQ ID NO: 1) are particularly preferred:
H69del, V70del, Y144del, N501Y, A570D, D614G, P681H, T716I, S982A, and D1118H
L18F, D80A, D215G, L242del, A243del, L244del, R246I, K417N, E484K, N501Y, D614G, and A701V
K417N, E484K, N501Y, and D614G
E484K and D614G
L18F, T20N, P26S, D138Y, R190S, K417T, E484K, N501Y, D614G, H655Y, and T1027I
S13I, W152C, L452R, and D614G
delH69, delV70, Y453F, D614G, I692V, and M1229I
E484K, E484P, or E484Q
G446V
G485R

In further preferred embodiments, the SARS-CoV-2 spike proteins (S) comprises the following amino acid variations (amino acid positions according to reference SEQ ID NO: 1): L18F, D80A, D215G, delL242, delA243, delL244, R246I, K417N, E484K, N501Y, D614G, A701V.

Suitable SARS-CoV-2 spike proteins (S) may be selected or derived from emerging SARS-CoV-2 variants according to the following List 1C (only examples of SARS-CoV-2 variants are provided, not limited to those):

List 1C: List of Emerging SARS-CoV-2 Variants

| Variant | Amino acid changes in spike protein |
|---|---|
| Mink Cluster 5 variant GISAID: EPI_ISL_616802 (hCoV- | delH69, delV70, Y453F, D614G, I692V, M1229I |
| 19/Denmark/DCGC-3024/2020) | |
| B.1.1.7 (a.k.a., 20B/501Y.V1, 501Y.V1, Variant of Concern- | delH69, delV70, delY144, N501Y, A570D, D614G, |
| 202012/01, VOC-202012/01, VUI-202012/01, B117, “UK | P681H, T716I, S982A, D1118H |
| variant”) | |
| B.1.351 (a.k.a., 20C/501Y.V2, 501Y.V2, N501Y.V2, “SA | L18F, D80A, D215G, delL242, delA243, delL244, |
| variant”, “South Africa variant”) | R246I, K417N, E484K, N501Y, D614G, A701V |
| P.1 (a.k.a., “Brazil variant” = “Japan variant”) | L18F, T20N, P26S, D138Y, R190S, K417T, E484K, |
| | N501Y, D614G, H655Y, T1027I |
| CAL.20C (a.k.a., “California variant”) | S13I, W152C, L452R, D614G |
| NCBI: QQN00429.1 (CAL.20.C example: SARS-COV- | |
| 2/human/USA/CA-LACPHL-AF00114/2021) | |

In some embodiments, a fragment of a SARS-CoV-2 spike protein as defined herein may be encoded by the nucleic acid, wherein said fragment may be N-terminally truncated, lacking the N-terminal amino acids 1 to up to 100 of the full-length SARS-CoV-2 spike reference protein (SEQ ID NO: 1) or of a SARS-CoV-2 spike variant protein and/or wherein said fragment may be C-terminally truncated, lacking the C-terminal amino acids (aa) 531 to up to aa 1273 of the full-length SARS-CoV-2 coronavirus reference protein (SEQ ID NO: 1) or of a SARS-CoV-2 spike variant protein. Such “fragment of a spike protein (S)” may additionally comprise amino acid substitutions (as described below) and may additionally comprise at least one heterologous peptide or protein element (as described below). In preferred embodiments, a fragment of a SARS-CoV-2 spike protein (S) may be C-terminally truncated, thereby lacking the C-terminal transmembrane domain (that is, lacking aa 1212 to aa 1273 or lacking aa 1148 to aa 1273))amino acid positions according to reference SEQ ID NO: 1)

In other embodiments, the encoded spike protein (S) derived from SARS-CoV-2 lacks the transmembrane domain (TM) (amino acid position aa 1212 to aa 1273 according to reference SEQ ID NO: 1). In embodiments, the encoded SARS-CoV-2 spike protein (S) lacks an extended part of the transmembrane domain (TMflex) (amino acid position aa 1148 to aa 1273, according to reference SEQ ID NO: 1). Without wishing to being bound to theory, a SARS-CoV-2 spike protein (S) lacking the transmembrane domain (TM or TMflex) as defined herein could be suitable for a SARS-CoV-2 vaccine, as such a protein would be soluble and not anchored in the cell membrane. A soluble protein may therefore be produced (that is translated) in higher concentrations upon administration to a subject, leading to improved immune responses.

Without wishing to being bound to theory, RBD (aa 319 to aa 541) and CND (aa 29 to aa 529) domains may be crucial for immunogenicity of SARS-CoV-2 spike protein (S). Both regions are located at the S1 fragment of the spike protein. Accordingly, it may be suitable in the context of the invention that the antigenic peptide or protein comprises or consists of an S1 fragment of the spike protein or an immunogenic fragment or immunogenic variant thereof.

Suitably, a S1 fragment of SARS-CoV-2 may comprise at least an RBD and/or a CND domain as defined above.

In preferred embodiments, the encoded at least one antigenic peptide or protein comprises or consists of a receptor-binding domain (RBD; aa 319 to aa 541), wherein the RBD comprises or consists of a spike protein fragment, or an immunogenic fragment or immunogenic variant thereof.

In further preferred embodiments, the encoded at least one antigenic peptide or protein comprises or consists of a truncated receptor-binding domain (truncRBD; aa 334 to aa 528), wherein the RBD comprises or consists of a spike protein fragment, or an immunogenic fragment or immunogenic variant thereof.

Such “fragment of a spike protein (S)” (RBD; aa 319 to aa 541 or truncRBD, aa 334 to aa 528), may additionally comprise amino acid substitutions (as described herein) and may additionally comprise at least one heterologous peptide or protein element (as described herein).

In particularly preferred embodiments, the encoded SARS-CoV-2 spike protein (S) comprises or consists of a spike protein fragment S1, or an immunogenic fragment or immunogenic variant thereof.

In preferred embodiments, the SARS-CoV-2 spike protein fragment S1 lacks at least 70%, 80%, 90%, preferably 100% of spike protein fragment S2 (aa 682 to aa 1273). Such embodiments may be beneficial as the SARS-CoV-2 S1 fragment comprises neutralizing epitopes without potential problems of full-length protein comprising S1 and S2.

Without wishing to being bound to theory, it may be suitable that the antigenic peptide or protein comprises or consists of SARS-CoV-2 spike protein fragment S1 and (at least a fragment of) SARS-CoV-2 spike protein fragment S2, because the formation of an immunogenic SARS-CoV-2 spike protein may be promoted.

Accordingly, in particularly preferred embodiments, the encoded SARS-CoV-2 spike protein (S) comprises or consists of a SARS-CoV-2 spike protein fragment S1 or an immunogenic fragment or immunogenic variant thereof, and SARS-CoV-2 spike protein fragment S2 or an immunogenic fragment or immunogenic variant thereof.

In particularly preferred embodiments, the encoded at least one antigenic peptide or protein comprises or consists of a full-length SARS-CoV-2 spike protein or an immunogenic fragment or immunogenic variant of any of these.

The term “full-length SARS-CoV-2 spike protein” has to be understood as a spike protein derived from SARS-CoV-2 or a variant SARS-CoV-2 having an amino acid sequence corresponding to essentially the full spike protein. Accordingly, a “full-length spike protein” may comprise aa 1 to aa 1273 (reference protein: SEQ ID NO: 1). Accordingly, a full-length SARS-CoV-2 spike protein may typically comprise a secretory signal peptide, a spike protein fragment S1, a spike protein fragment S2, a receptor binding domain (RBD), and a critical neutralisation domain CND, and a transmembrane domain. Notably, also variants that comprise certain amino acid substitutions (e.g. for allowing pre-fusion stabilization of the S protein) or natural occurring amino acid deletions are encompassed by the term “full-length SARS-CoV-2 spike protein”.

In particularly preferred embodiments, the SARS-CoV-2 spike protein (S) that is provided by the nucleic acid is designed or adapted to stabilize the antigen in pre-fusion conformation. A pre-fusion conformation is particularly advantageous in the context of an efficient SARS-CoV-2 vaccine, as several potential epitopes for neutralizing antibodies may merely be accessible in said pre-fusion protein conformation. Furthermore, remaining of the protein in the pre-fusion conformation is aimed to avoid immunopathological effects, like e.g. enhanced disease and/or antibody dependent enhancement (ADE).

In preferred embodiments, administration of a nucleic acid (or a composition or vaccine) encoding pre-fusion stabilized spike protein to a subject elicits spike protein neutralizing antibodies and does not elicit disease-enhancing antibodies. In particular, administration of a nucleic acid (or a composition or vaccine) encoding pre-fusion stabilized spike protein to a subject does not elicit immunopathological effects, like e.g. enhanced disease and/or antibody dependent enhancement (ADE).

Accordingly, in preferred embodiments, the (additional) nucleic acid comprises at least one coding sequence encoding at least one antigenic peptide or protein that is selected or derived from a SARS-CoV-2 spike protein (S), wherein the spike protein (S) is a pre-fusion stabilized spike protein (S_stab). Suitably, said pre-fusion stabilized spike protein comprises at least one pre-fusion stabilizing mutation.

Stabilization of the SARS-CoV-2 spike protein may be obtained by substituting at least one amino acids at position K986 and/or V987 with amino acids that stabilize the spike protein in a perfusion conformation (amino acid positions according to reference SEQ ID NO: 1).

In embodiments, the pre-fusion stabilizing mutation of SARS-CoV-2 spike protein comprises an amino acid substitution at position K986, wherein the amino acids K986 is substituted with one selected from A, I, L, M, F, V, G, or P (amino acid positions according to reference SEQ ID NO: 1), preferably wherein the amino acids K986 is substituted with P. In embodiments, the pre-fusion stabilizing mutation comprises an amino acid substitution at position V987, wherein the amino acids V987 is substituted with one selected from A, I, L, M, F, V, G, or P (amino acid positions according to reference SEQ ID NO: 1), preferably wherein the amino acids V987 is substituted with P.

Suitably, stabilization of the SARS-CoV-2 spike protein may be obtained by substituting two consecutive amino acids at position K986 and V987 with amino acids that stabilize the spike protein in a perfusion conformation (Amino acid positions according to reference SEQ ID NO: 1).

In preferred embodiments, the pre-fusion stabilizing mutation of the SARS-CoV-2 spike protein comprises an amino acid substitution at position K986 and V987, wherein the amino acids K986 and/or V987 are substituted with one selected from A, I, L, M, F, V, G, or P (amino acid positions according to reference SEQ ID NO: 1).

Preferably, stabilization of the perfusion conformation is obtained by introducing two consecutive proline substitutions at residues K986 and V987 in the SARS-CoV-2 spike protein (Amino acid positions according to reference SEQ ID NO: 1).

Accordingly, in preferred embodiments, the pre-fusion stabilized spike protein (S_stab) of SARS-CoV-2 comprises at least one pre-fusion stabilizing mutation, wherein the at least one pre-fusion stabilizing mutation comprises the following amino acid substitutions: K986P and V987P (amino acid positions according to reference SEQ ID NO: 1).

Accordingly, any NCBI Protein Accession numbers provided above, or any protein selected from SEQ ID NOs: 1-9, 274-340, 22737, 22739, 22741, 22743, 22745, 22747, 22749, 22751, 22753, 22755, 22757, 22929-22946 or fragments or variants thereof can be chosen by the skilled person to introduce such amino acid changes into SARS-CoV-2 spike proteins, preferably amino acid substitutions: K986P and V987P (amino acid positions according to reference SEQ ID NO: 1).

In particularly preferred embodiments, the SARS-CoV-2 spike protein that is encoded by the nucleic acid of the invention is a pre-fusion stabilized spike protein (S_stab) comprising at least one pre-fusion stabilizing K986P and V987P mutation and additionally comprising the following amino acid substitutions or deletions (amino acid positions according to reference SEQ ID NO: 1):
E484K, N501Y, and optionally L18F, D80A, D215G, L242del, A243del, L244del, R246I, K417N, D614G, A701V;
E484K, N501Y, and optionally L18F, D80A, D215G, L242del, A243del, L244del, K417N, D614G, A701V;
N501Y, P681H, and optionally H69del, V70del, Y144del, A570D, D614G, T716I, S982A, D1118H;
N501Y, P681H, E484K, and optionally H69del, V70del, Y144del, A570D, D614G, T716I, S982A, D1118H;
E484K, N501Y, and optionally L18F, T20N, P26S, D138Y, R190S, K417T, D614G, H655Y, T1027I;
E484K, N501Y, and optionally L18F, T20N, P26S, D138Y, R190S, K417T, D614G, H655Y, T1027I, V1176F;
N501Y, P681H, E484K, and optionally H69del, V70del, Y144del, A570D, D614G, T716I, S982A, D1118H;
L452R, and optionally S13I, W152C, D614G;
L452R, D614D and optionally P681R;
L452R, D614D, P681R and optionally E484Q, E154K, Q1071H;
L452R, D614D, P681R and optionally T19R, L452R, D950N;
L452R, D614D, P681R and optionally T19R, F157del, T478K, D950N;
E484K, and optionally Q52R, A67V, H69del, V70del, delY144, D614G, Q677H, F888L;
E484K, and optionally A67V, H69del, V70del, Y144del, D614G, Q677H, F888L;
E484K, and optionally L5F, T95I, D253G, D614G, A701V;
P681R, and optionally F157L, V367F, Q613H;
P681R, and optionally S254F, D614G, G769V;
L452R, P681R, and optionally D614G;
L452R, E484Q, P681R, and optionally E154K, D614G, Q1071H;
L452R, P681R, and optionally T19R, F157del, R158del, T478K, D614G, D950N;
E484K, and optionally D614G, V1176F;
L452Q, and optionally G75V, T76I, R246del, S247del, Y248del, L249del, T250del, P251del, G252del, F490S, D614G, T859N;
K417N, and optionally P681R;
K417N, P681R, and optionally D614G;
K417N, L452R, P681R, and optionally D614G;
K417N, T478K, P681R, and optionally D614G;
K417N, D950N, P681R, and optionally D614G.
K417N, D614G, P681R, and optionally T478K;
K417N, D614G, P681R, and optionally L452R;
K417N, D614G, P681R, L452R and optionally T478K;
S247del, Y248del, L249del, T250del, P251del, G252del, D253del and optionally D614G;
S247del, Y248del, L249del, T250del, P251del, G252del, D253del and optionally L452Q, D614G;
H69del, V70del and optionally D614G;
H69del, V70del, E484K and optionally D614G;
H69del, V70del, N501Y and optionally D614G; or
H69del, V70del, N501Y, E484K and optionally P681H.

In particularly preferred embodiments, the SARS-CoV-2 spike protein that is encoded by the nucleic acid of the invention is a pre-fusion stabilized spike protein (S_stab) (or a fragment or variant thereof) comprising at least one pre-fusion stabilizing K986P and V987P mutation and additionally comprises the following amino acid substitutions or deletions (amino acid positions according to reference SEQ ID NO: 1):
E484K, N501Y, L18F, D80A, D215G, L242del, A243del, L244del, R246I, K417N, D614G, and A701V;
E484K, N501Y, L18F, D80A, D215G, L242del, A243del, L244del, K417N, D614G, and A701V;
E484K, N501Y, L18F, T20N, P26S, D138Y, R190S, K417T, D614G, H655Y, and T1027I;
E484K, N501Y, L18F, T20N, P26S, D138Y, R190S, K417T, D614G, H655Y, T1027I, V1176F;
L452R, P681R, and D614G;
L452R, E484Q, P681R, E154K, D614G, and Q1071H; or
L452R, P681R, T19R, F157del, R158del, T478K, D614G, and D950N.

In particularly preferred embodiments, the SARS-CoV-2 spike protein that is encoded by the nucleic acid of the invention is a pre-fusion stabilized spike protein (S_stab) (or a fragment or variant thereof) comprising amino acid substitutions or deletions selected from (amino acid positions according to reference SEQ ID NO: 1):
K986P, V987P, E484K, N501Y, L18F, D80A, D215G, L242del, A243del, L244del, R246I, K417N, D614G, and A701V; or
K986P, V987P, E484K, N501Y, L18F, D80A, D215G, L242del, A243del, L244del, K417N, D614G, and A701V.

It has to be emphasized that in the context embodiments of the invention any SARS-CoV-2 coronavirus spike protein as defined herein may be mutated as described above (exemplified for reference protein SEQ ID NO: 1) to stabilize the spike protein in the pre-fusion conformation.

In preferred embodiments, the at least one pre-fusion stabilizing mutation of SARS-CoV-2 spike protein comprises a cavity filling mutation that further stabilizes the pre-fusion state, wherein said mutation/amino acid substitution is selected from the list comprising T887W; A1020W; T887W and A1020W; or P1069F (amino acid positions according to reference SEQ ID NO: 1).

In embodiments, at least one of the following amino acid substitutions T887W; A1020W; T887W and A1020W; or P1069F may be combined with a (K986P and V987P) substitution in the SARS-CoV-2 spike protein (amino acid positions according to reference SEQ ID NO: 1).

In particularly preferred embodiments, the SARS-CoV-2 spike protein comprises at least one of the following amino acid substitutions (amino acid positions according to reference SEQ ID NO: 1):
T887W; K986P and V987P
A1020W; K986P and V987P
T887W and A1020W; K986P and V987P
P1069F; K986P and V987P

Accordingly, any NCBI protein accession numbers of SARS-CoV-2 S provided above, or any protein selected from SEQ ID NOs: 1-9, 274-340, 22737, 22739, 22741, 22743, 22745, 22747, 22749, 22751, 22753, 22755, 22757, 22929-22946 or fragments or variants thereof, can be chosen by the skilled person to introduce such amino acid changes, suitably amino acid substitutions selected from T887W; A1020W; T887W and A1020W; or P1069F; or amino acid substitutions selected from (T887W; K986P and V987P); (A1020W; K986P and V987P); (T887W and A1020W; K986P and V987P); (P1069F; K986P and V987P) (amino acid positions according to reference SEQ ID NO: 1).

In embodiments, at least one of the following amino acid substitutions F817P, A892P, A899P and A942P may be combined with a (K986P and V987P) substitution (amino acid positions according to reference SEQ ID NO: 1).

In embodiments, the SARS-CoV-2 coronavirus spike protein comprises at least one of the following amino acid substitutions (Amino acid positions according to reference SEQ ID NO: 1):
F817P; K986P and V987P
A892P; K986P and V987P
A899P; K986P and V987P
A942P; K986P and V987P

In particularly preferred embodiments, the SARS-CoV-2 coronavirus spike protein comprises the following amino acid substitutions (Amino acid positions according to reference SEQ ID NO: 1):
F817P, A892P, A899P, A942P, K986P and V987P (S_stab_PP_hex)

Accordingly, any NCBI protein accession numbers provided above, or any protein selected from SEQ ID NOs: 1-9, 274-340, 22737, 22739, 22741, 22743, 22745, 22747, 22749, 22751, 22753, 22755, 22757, 22929-22946 or fragments or variants thereof can be chosen by the skilled person to introduce such amino acid changes, suitably amino acid substitutions selected from F817P, A892P, A899P, A942P; or amino acid substitutions selected from (F817P; K986P and V987P); (A892P; K986P and V987P); (A899P; K986P and V987P); (A942P; K986P and V987P); (F817P, A892P, A899P, A942P, K986P and V987P) (amino acid positions according to reference SEQ ID NO: 1).

In preferred embodiments, the at least one pre-fusion stabilizing mutation of SARS-CoV-2 spike protein comprises a mutated protonation site that further stabilizes the pre-fusion state, wherein said mutation/amino acid substitution is selected from H1048Q and H1064N; H1083N and H1101N; or H1048Q and H1064N and H1083N and H1101N (amino acid positions according to reference SEQ ID NO: 1).

In embodiments, at least one of the following amino acid substitutions H1048Q and H1064N; H1083N and H1101N; or H1048Q and H1064N and H1083N and H1101N may be combined with a (K986P and V987P) substitution (amino acid positions according to reference SEQ ID NO: 1) into a SARS-CoV-2 spike protein.

In particularly preferred embodiments, the SARS-CoV-2 spike protein comprises at least one of the following amino acid substitutions (Amino acid positions according to reference SEQ ID NO: 1):
H1048Q and H1064N; K986P and V987P
H1083N and H1101N; K986P and V987P
H1048Q and H1064N and H1083N and H1101N; K986P and V987P

Accordingly, any SARS-CoV-2 NCBI protein accession numbers provided above, or any protein selected from SEQ ID NOs: 1-9, 274-340, 22737, 22739, 22741, 22743, 22745, 22747, 22749, 22751, 22753, 22755, 22757, 22929-22946 or fragments or variants thereof can be chosen by the skilled person to introduce such amino acid changes into a SARS-CoV-2 spike protein, suitably amino acid substitutions selected from H1048Q and H1064N; H1083N and H1101N; or H1048Q and H1064N and H1083N and H1101N; or amino acid substitutions selected from (H1048Q and H1064N; K986P and V987P); (H1083N and H1101N; K986P and V987P); (H1048Q and H1064N and H1083N and H1101N; K986P and V987P); (amino acid positions according to reference SEQ ID NO: 1).

In preferred embodiments, the at least one pre-fusion stabilizing mutation of the SARS-CoV-2 spike protein comprises an artificial intramolecular disulfide bond. Such an artificial intramolecular disulfide bond can be introduced to further stabilize the membrane distal portion of the SARS-CoV-2 S protein (including the N-terminal region) in the pre-fusion conformation; that is, in a conformation that specifically binds to one or more pre-fusion specification antibodies, and/or presents a suitable antigenic site that is present on the pre-fusion conformation but not in the post fusion conformation of the SARS-CoV-2 S protein.

In preferred embodiments, the at least one pre-fusion stabilizing mutation of the SARS-CoV-2 spike protein comprises an artificial intramolecular disulfide bond, preferably wherein the at least one artificial intramolecular disulfide bond comprises at least two of the following amino acid substitutions selected from the list comprising I712C, I714C, P715C, T874C, G889C, A890C, I909C, N914C, Q965C, F970C, A972C, R995C, G999C, S1003C, L1034C, V1040C, Y1047C, S1055C, P1069C, T1077C, Y1110C, or S1123C (amino acid positions according to reference SEQ ID NO: 1).

In preferred embodiments, the at least one pre-fusion stabilizing mutation of the SARS-CoV-2 spike protein comprises an artificial intramolecular disulfide bond, wherein the at least one artificial intramolecular disulfide bond comprises at least one of the following amino acid substitutions: I712C and T1077C; I714C and Y1110C; P715C and P1069C; G889C and L1034C; I909C and Y1047C; Q965C and S1003C; F970C and G999C; A972C and R995C; A890C and V1040C; T874C and S1055C; or N914C and S1123C (amino acid positions according to reference SEQ ID NO: 1).

In embodiments, the at least one pre-fusion stabilizing mutation of the SARS-CoV-2 spike protein comprises 2, 3, 4, 5, 6, 7, or 8 different artificial intramolecular disulfide bonds, wherein each may be selected from the following amino acid substitutions: I712C and T1077C; I714C and Y1110C; P715C and P1069C; G889C and L1034C; I909C and Y1047C; Q965C and S1003C; F970C and G999C; A972C and R995C; A890C and V1040C; N914C and S11230; T874C and S1055C; or N914C and S1123C (amino acid positions according to reference SEQ ID NO: 1).

In embodiments, at least one, preferably 2, 3, 4, 5 or more of the following amino acid substitutions I712C and T1077C; I714C and Y1110C; P715C and P1069C; G889C and L1034C; I909C and Y1047C; Q965C and S1003C; F970C and G999C; A972C and R995C; A890C and V1040C; T874C and S1055C; or N914C and S1123C may be combined with a (K986P and V987P) substitution. For example, a pre-fusion stabilized SARS-CoV-2 S protein may comprise two different artificial intramolecular disulfide bonds, e.g. I712C and T1077C; P715C and P10690; and additionally a K986P and V987P substitution, etc. (amino acid positions according to reference SEQ ID NO: 1).

In particularly preferred embodiments, the SARS-CoV-2 spike protein comprises at least one of the following amino acid substitutions (amino acid positions according to reference SEQ ID NO: 1):
I712C and T1077C; K986P and V987P
I714C and Y1110C; K986P and V987P
P715C and P1069C; K986P and V987P
G889C and L1034C; K986P and V987P
I909C and Y1047C; K986P and V987P
Q965C and S1003C; K986P and V987P
F970C and G999C; K986P and V987P
A972C and R995C; K986P and V987P
A890C and V10400; K986P and V987P
T874C and S1055C; K986P and V987P
N914C and S1123C; K986P and V987P

Accordingly, any SASR-CoV-2 NCBI protein accession numbers provided above, or any protein selected from SEQ ID NOs: 1-9, 274-340, 22737, 22739, 22741, 22743, 22745, 22747, 22749, 22751, 22753, 22755, 22757, 22929-22946 or fragments or variants thereof can be chosen by the skilled person to introduce such amino acid changes into a SARS-CoV-2 spike protein, suitably amino acid substitutions selected from I712C and T1077C; I714C and Y1110C; P715C and P1069C; G889C and L1034C; I909C and Y1047C; Q965C and S1003C; F970C and G999C; A972C and R995C; A890C and V1040C; T874C and S1055C; or N914C and S1123C; or amino acid substitutions selected from (I712C; T1077C; K986P; V987P) or (I714C; Y1110C; K986P; V987P) or (P715C; P1069C; K986P; V987P) or (G889C; L1034C; K986P; V987P) or (I909C; Y1047C; K986P; V987P) or (Q965C; S1003C; K986P; V987P) or (F970C; G999C; K986P; V987P) or (A972C; R995C; K986P; V987P) or (A890C and V1040C; K986P and V987P) or (T874C and S1055C; K986P and V987P) or (N914C and S1123C; K986P and V987P) (amino acid positions according to reference SEQ ID NO: 1).

It has to be emphasized that in the context of the invention any SARS-CoV-2 spike protein may be mutated or modified as described above (exemplified for reference protein SEQ ID NO: 1) to stabilize the spike protein in the pre-fusion conformation.

According to various preferred embodiments, the (additional) nucleic acid encodes at least one antigenic peptide or protein selected or derived from SARS-CoV-2 spike protein as defined herein and, additionally, at least one heterologous peptide or protein element, preferably selected or derived from a signal peptide, a linker, a helper epitope, an antigen clustering element, a trimerization element, a transmembrane element, and/or a VLP-forming sequence.

Suitably, the at least one heterologous peptide or protein element may promote or improve secretion of the encoded SARS-CoV-2 spike protein (e.g. via secretory signal sequences), promote or improve anchoring of the encoded SARS-CoV-2 spike protein in the plasma membrane (e.g. via transmembrane elements), promote or improve formation of antigen complexes (e.g. via multimerization domains or antigen clustering elements), or promote or improve virus-like particle formation (VLP forming sequence). In addition, the nucleic acid may additionally encode peptide linker elements, self-cleaving peptides, immunologic adjuvant sequences or dendritic cell targeting sequences.

In preferred embodiments, the nucleic acid additionally encodes at least one heterologous trimerization element, an antigen clustering element, or a VLP forming sequence.

In preferred embodiments, the antigen clustering elements may be selected from a ferritin element, or a lumazine synthase element, surface antigen of Hepatitis B virus (HBsAg), or encapsulin. Expressing a stably clustered SARS-CoV-2 spike protein, preferably in in its prefusion conformation may increases the magnitude and breadth of neutralizing activity against the encoded SARS-CoV-2 peptide/protein.

In preferred embodiments, lumazine synthase is used to promote antigen clustering of the SARS-CoV-2 spike protein and may therefore promote or enhance immune responses of the encoded SARS-CoV-2 spike antigen. In preferred embodiments, ferritin is used to promote the antigen clustering of the SARS-CoV-2 spike protein and may therefore promote immune responses of the encoded SARS-CoV-2 antigen. In preferred embodiments, HBsAg is used to promote the antigen clustering of the SARS-CoV-2 spike protein and may therefore promote immune responses of the encoded SARS-CoV-2 antigen. In preferred embodiments, encapsulin is used to promote the antigen clustering of the SARS-CoV-2 spike protein and may therefore promote immune responses of the encoded SARS-CoV-2 antigen.

In embodiments where the coding sequence additionally encodes heterologous antigen clustering element, it is particularly preferred and suitable to generate a fusion protein comprising an antigen clustering element and an antigenic peptide or protein derived from SARS-CoV-2 spike protein. Suitably, said spike protein is lacking the C-terminal transmembrane domain (TM) (lacking aa 1212 to aa 1273) or is lacking a part of the C-terminal transmembrane domain (TMflex), e.g. lacking aa 1148 to aa 1273.

Accordingly, any amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 1-26, 274-1278,13521-13587, 22732, 22737-22758, 22929-22964 can be modified to remove the endogenous transmembrane domain (TM) at position aa 1212 to aa 1273 and may therefore be used as “C-terminally truncated” SARS-CoV-2 spike proteins in the context of the invention (amino acid positions according to reference SEQ ID NO: 1). Furthermore, any amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 1-26, 274-1278, 13521-13587, 22732, 22737-22758, 22929-22964 can be modified to remove a part the endogenous transmembrane domain (TMflex) at position aa 1148 to aa 1273 and may therefore be used as “C-terminally truncated” SARS-CoV-2 spike proteins in the context of the invention (Amino acid positions according to reference SEQ ID NO: 1). Suitable spike proteins lacking the C-terminal transmembrane domain (TM or TMflex) may be selected from SEQ ID NOs: 31-39, 1614-3623, 13377-13510.

In other embodiments, where the coding sequence additionally encodes heterologous antigen clustering element as defined above, it is particularly preferred and suitable to generate a fusion protein comprising an antigen clustering element and an antigenic peptide or protein selected or derived from SARS-CoV-2 spike protein fragment S1 (lacking S2 and/or (TM and/or TMflex). Further, it may be suitable to use linker elements for separating the heterologous antigen clustering element from the antigenic peptide or protein (e.g. a linker according to SEQ ID NO: 115, 13148, 13152).

In preferred embodiments, the trimerization element may be selected from a foldon element. In preferred embodiments, the foldon element is a fibritin foldon element. Expressing a stable trimeric spike protein, preferably in its prefusion conformation, may increases the magnitude and breadth of neutralizing activity against SARS-CoV-2 spike.

In particularly preferred embodiments, a fibritin foldon element is used to promote the antigen trimerization and may therefore promote immune responses of the encoded SARS-CoV-2 spike protein. Preferably, the foldon element is or is derived from a bacteriophage, preferably from bacteriophage T4, most preferably from fibritin of bacteriophage T4.

In embodiments where the coding sequence of the nucleic acid additionally encodes heterologous trimerization element, it is particularly preferred and suitable to generate a fusion protein comprising a trimerization element and an antigenic peptide or protein derived from SARS-CoV-2 spike. Suitably, said spike protein derived from SARS-CoV-2 is lacking the C-terminal transmembrane domain (lacking aa 1212 to aa 1273), or is lacking a part of the C-terminal transmembrane domain (TMflex), e.g. lacking aa 1148 to aa 1273.

In other embodiments, where the coding sequence of the nucleic acid additionally encodes heterologous trimerization element as defined above, it is particularly preferred and suitable to generate a fusion protein comprising an trimerization element and SARS-CoV-2 spike protein fragment S1 (lacking S2 and/or (TM and/or TMflex). Further, it may be suitable to use linker elements for separating the heterologous antigen clustering element from the antigenic peptide or protein (e.g. a linker according to SEQ ID NO: 115, 13148, 13152).

In preferred embodiments, a VLP forming sequence may be selected and fused to the SARS-CoV-2 spike as defined herein. Expressing a stably clustered SARS-CoV-2 spike protein in VLP form may increases the magnitude and breadth of neutralizing activity against SARS-CoV-2. VLPs structurally mimic infectious viruses and they can induce potent cellular and humoral immune responses.

Suitable VLP forming sequences may be selected from elements derived from Hepatitis B virus core antigen, HIV-1 Gag protein, or Woodchuck hepatitis core antigen element (WhcAg).

In particularly preferred embodiments, the at least one VLP-forming sequence is a Woodchuck hepatitis core antigen element (WhcAg). The WhcAg element is used to promote VLP formation and may therefore promote immune responses of the encoded SARS-CoV-2 spike protein.

In embodiments where the coding sequence of the nucleic acid additionally encodes heterologous VLP forming sequence, it is particularly preferred and suitable to generate a fusion protein comprising a VLP forming sequence and an antigenic peptide or protein derived from SARS-CoV-2 spike. Suitably, said SARS-CoV-2 spike protein is lacking the C-terminal transmembrane domain (lacking aa 1212 to aa 1273), or is lacking a part of the C-terminal transmembrane domain (TMflex), e.g. lacking aa 1148 to aa 1273.

Accordingly, any amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 1-26, 274-1278,13521-13587, 22732, 22737-22758, 22929-22964 can be modified to lack the endogenous transmembrane element at position aa 1212 to aa 1273 and may therefore be used as “C-terminally truncated” SARS-CoV-2 spike proteins in the context of the invention. Furthermore, any amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 1-26, 274-1278, 13521-13587, 22732, 22737-22758, 22929-22964 can be modified to remove a part the endogenous transmembrane domain (TMflex) at position aa 1148 to aa 1273 and may therefore be used as “C-terminally truncated” SARS-CoV-2 spike proteins in the context of the invention (amino acid positions according to reference SEQ ID NO: 1). Suitable SARS-CoV-2 spike proteins lacking the C-terminal transmembrane domain (TM or TMflex) may be selected from SEQ ID NOs: 31-39, 1614-3623, 13377-13510.

In other embodiments, where the coding sequence of the nucleic acid additionally encodes heterologous VLP-forming sequence as defined above, it is particularly preferred and suitable to generate a fusion protein comprising a VLP-forming sequence and SARS-CoV-2 spike protein fragment S1 (lacking S2 and/or (TM and/or TMflex). Further, it may be suitable to use linker elements for separating the heterologous antigen clustering element from the antigenic spike protein (e.g. a linker according to SEQ ID NO: 115, 13148, 13152).

In embodiments, the SARS-CoV-2 spike protein comprises a heterologous signal peptide as defined above. A heterologous signal peptide may be used to improve the secretion of the encoded SARS-CoV-2 spike antigen.

In embodiments where the coding sequence of the nucleic acid additionally encodes heterologous secretory signal peptide, it is particularly preferred and suitable to generate a fusion protein comprising a heterologous secretory signal peptide and an SARS-CoV-2 spike protein. Suitably, said SARS-CoV-2 spike protein is lacking the N-terminal endogenous secretory signal peptide (lacking aa 1 to aa 15). Accordingly, any amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 1-26, 274-1278,13521-13587, 22732 or 22737-22758, 22929-22964 can be modified to lack the endogenous secretory signal peptide at position aa 1 to aa 15 and may therefore be used as “N-terminally truncated” SARS-CoV-2 spike proteins.

In the following List 1, suitable SARS-CoV-2 spike protein constructs as defined above are further specified in detail (e.g. nomenclature, protein elements, etc.).

List 1: Exemplary Suitable SARS-CoV-2 Antigen Designs:
Full-length spike protein (S) comprising aa 1 to aa 1273;
   see for example SEQ ID NO: 1, 274.
Stabilized S protein comprising aa1-aa1273 and K986P, V987P substitutions (S_stab_PP);
   see for example SEQ ID NO: 10, 341.
Stabilized S protein comprising aa1-aa1273 and K986P, V987P substitutions (S_stab_PP);
   see for example SEQ ID NO: 22961.
Stabilized S protein comprising aa1-aa1273 and K986P, V987P substitutions (S_stab_PP);
   see for example SEQ ID NO: 22960.
Stabilized S protein comprising aa1-aa1273 and K986P, V987P, F817P, A892P, A899P, A942P proline substitutions; S_stab_PP_hex
   see for example SEQ ID NO: 22732.
Stabilized S protein comprising aa1-aa1273 and K986P, V987P substitutions and a cavity filling mutation (T887W, A1020W); S_stab_PP_cav
   see for example SEQ ID NO: 408.
Stabilized S protein comprising aa1-aa1273 and K986P, V987P substitutions and a cavity filling mutation (P1069F); S_stab_PP_cav
   see for example SEQ ID NO: 475.
Stabilized S protein comprising aa1-aa1273 and K986P, V987P substitutions and a cavity filling mutation (H1048Q, H1064N, H1083N, H1101N); S_stab_PP_prot
   see for example SEQ ID NO: 542.
Stabilized S protein comprising aa1-aa1273 and an artificial disulfide bond (S_stab_disul) I712C, T1077C;
   see for example SEQ ID NO: 19, 609.
S without transmembrane domain comprising aa1-aa1211 (S_woTM);
   see for example SEQ ID NO: 31, 1614.
S without transmembrane domain flex comprising aa1-aa1147 (S_woTMflex);
   see for example SEQ ID NO: 2619.
S_woTM comprising K986P, V987P substitutions (S_stab_PP_woTM)
   see for example SEQ ID NO: 40, 1681.
S_woTMflex comprising K986P, V987P substitutions (S_stab_PP_woTMflex)
   see for example SEQ ID NO: 2686.
Spike protein fragment S1 comprising aa 1 to aa 681 (S1);
   see for example SEQ ID NO: 27, 1279.
S_woTM comprising a lumazine synthase;
   see for example SEQ ID NO: 58, 3624.
S_woTMflex comprising a lumazine synthase;
   see for example SEQ ID NO: 7644.
S_stab_PP_woTM comprising a lumazine synthase;
   see for example SEQ ID NO: 85, 3691.
S_stab_PP_woTMflex comprising a lumazine synthase;
   see for example SEQ ID NO: 7711.
S_woTM comprising a ferritin element;
   see for example SEQ ID NO: 67, 4629.
S_woTMflex comprising a ferritin element;
   see for example SEQ ID NO: 8649.
S_stab_PP_woTM comprising a ferritin element;
   see for example SEQ ID NO: 94, 4696.
S_stab_PP_woTMflex comprising a ferritin element;
   see for example SEQ ID NO: 8716.
S_woTM comprising a foldon element;
   see for example SEQ ID NO: 76, 5634.
S_woTMflex comprising a foldon element;
   see for example SEQ ID NO: 9654.
S_stab_PP_woTM comprising a foldon element;
   see for example SEQ ID NO: 103, 5701.
S_stab_PP_woTMflex comprising a foldon element;
   see for example SEQ ID NO: 9721.
S_woTM comprising a VLP-sequence (WhcAg);
   see for example SEQ ID NO: 6639.
S_woTMflex comprising a VLP-sequence (WhcAg);
   see for example SEQ ID NO 10659.
S_stab_PP_woTM comprising a VLP-sequence (WhcAg);
   see for example SEQ ID NO: 6706.
S_stab_PP_woTMflex comprising a VLP-sequence (WhcAg);
   see for example SEQ ID NO: 10726.
truncRBD comprising a foldon element:
   see for example SEQ ID NO: 22734.
truncRBD comprising a lumazine synthase (C-terminal)
   see for example SEQ ID NO: 22735.
truncRBD comprising a lumazine synthase (N-terminal)
   see for example SEQ ID NO: 22736
truncRBD comprising a ferritin element:
   see for example SEQ ID NO: 22733.

Amino acid positions provided in List 1 are according to reference SEQ ID NO: 1.

Preferred SARS-CoV-2 spike protein as defined above are provided in Table 2 (rows 1 to 41). Therein, each row 1 to 41 corresponds to a suitable SARS-CoV-2 spike protein constructs. Column A of Table 2 provides a short description of suitable SARS-CoV-2 spike constructs. Column B of Table 2 provides protein (amino acid) SEQ ID NOs of respective SARS-CoV-2 spike constructs. Column C of Table 2 provides SEQ ID NO of the corresponding wild type or reference nucleic acid coding sequences. Column D of Table 2 provides SEQ ID NO of the corresponding G/C optimized nucleic acid coding sequences (opt1, gc). Column E of Table 2 provides SEQ ID NO of the corresponding human codon usage adapted nucleic acid coding sequences (opt 3, human). Column F of Table 2 provides SEQ ID NO of the corresponding G/C content modified nucleic acid coding sequences (opt10, gc mod).

Notably, the description of the invention explicitly includes the information provided under <223> identifier of the ST25 sequence listing of the present application. Preferred nucleic acid constructs comprising coding sequences of Table 2, e.g. mRNA sequences comprising the coding sequences of Table 2 are provided in Table 3A and B.

TABLE 2
SARS-CoV-2 spike constructs (amino acid sequences and nucleic acid coding sequences):

| row | A | B | C | D | E | F |
|---|---|---|---|---|---|---|
| 1 | Full-length spike protein; S | 1-9, 274-340, | 116-131, | 136, 11731- | 11967- | 12034, |
| | | 22737, 22739, | 11664-11730 | 11797, 22764, | 12033 | 23041-23076 |
| | | 22741, 22743, | | 22766, 22768, | | |
| | | 22745, 22747, | | 22770, 22772, | | |
| | | 22749, 22751, | | 22774, 22776, | | |
| | | 22753, 22755, | | 22778, 22780, | | |
| | | 22757, | | 22782, 22784, | | |
| | | 22929-22946 | | 22969-23040 | | |
| 2 | Stabilized spike protein; | 10-18, 341-407, | | 137, 11798, | 142 | 146, 12035, |
| | S_stab_PP | 22738, 22740, | | 22765, 22767, | | 23149-23184 |
| | | 22742, 22744, | | 22769, 22771, | | |
| | | 22746, 22748, | | 22773, 22775, | | |
| | | 22750, 22752, | | 22777, 22779, | | |
| | | 22754, 22756, | | 22781, 22783, | | |
| | | 22758, | | 22785, 23077- | | |
| | | 22947-22964 | | 23148 | | |
| 3 | Stabilized spike protein; | 408-541 | | 11799, 11800 | | 12036, 12037 |
| | S_stab_PP_cav | | | | | |
| 4 | Stabilized spike protein; | 542-608 | | 11801 | | 12038 |
| | S_stab_PP_prot | | | | | |
| 5 | Stabilized spike protein; | 19-26, 609-1278, | | 11802-11811, | | 12039- |
| | S_stab_disul | 13521-13587 | | 14124 | | 12048, 14133 |
| 6 | Spike protein fragment S1 | 27, 1279-1345 | 132 | 138, 11812 | 143 | 147, 12049 |
| 7 | Spike protein fragment S2 | 30, 1346-1412 | 135 | 141, 11813 | | 12050 |
| 8 | Signal peptide of spike | 28, 1413-1479 | 133 | 139, 11814 | 144 | 12051 |
| | protein; SP | | | | | |
| 9 | S1 without signal peptide; | 29, 1480-1546 | 134 | 140, 11815 | 145 | 12052 |
| | S1_woSP | | | | | |
| 10 | Transmembrane domain | 49-57, 1547- | | 11816, 13511 | | 12053, 13516 |
| | of spike protein; TM/ | 1613, | | | | |
| | TMflex | 13176-13242 | | | | |
| 11 | S without transmembrane | 31-39, 1614- | | 11817, 11832 | | 12054, 12069 |
| | domain; S_woTM/ | 1680, 2619-2685 | | | | |
| | woTMflex | | | | | |
| 12 | Stabilized S without | 40-48, 1681- | | 11818, 11833 | | 12055, 12070 |
| | transmembrane domain; | 1747, 2686-2752 | | | | |
| | S_stab_PP_woTM/ | | | | | |
| | woTMflex | | | | | |
| 13 | Stabilized S without | 1748-1881, | | 11819, 11820, | | 12056, 12057, |
| | transmembrane domain; | 2753-2886 | | 11834, 11835 | | 12071, 12072 |
| | S_stab_PP_cav_woTM/ | | | | | |
| | woTMflex | | | | | |
| 14 | Stabilized S without | 1882-1948, | | 11821, 11836 | | 12058, 12073 |
| | transmembrane domain; | 2887-2953 | | | | |
| | S_stab_PP_prot_woTM/ | | | | | |
| | woTMflex | | | | | |
| 15 | Stabilized S without | 1949-2618, | | 11822-11831, | | 12059-12068, |
| | transmembrane domain; | 2954-3623, | | 11837-11846, | | 12074-12083, |
| | S_stab_disul_woTM/ | 13377-13510 | | 13514, 13515 | | 13519, 13520 |
| | woTMflex | | | | | |
| 16 | S_woTM/woTMflex | 58-66, 3624- | | 11847, 11907 | | 12084, 12144 |
| | comprising a lumazine | 3690, 7644-7710 | | | | |
| | synthase | | | | | |
| 17 | S_stab_PP_woTM/ | 85-93, 3691- | | 11848, 11908 | | 12085, 12145 |
| | woTMflex comprising a | 3757, 7711-7777 | | | | |
| | lumazine synthase | | | | | |
| 18 | S_stab_PP_cav_woTM/ | 3758-3891, | | 11849, 11850, | | 12086, 12087, |
| | woTMflex comprising a | 7778-7911 | | 11909, 11910 | | 12146, 12147 |
| | lumazine synthase | | | | | |
| 19 | S_stab_PP_prot_woTM/ | 3892-3958, | | 11851, 11911 | | 12088, 12148 |
| | woTMflex comprising a | 7912-7978 | | | | |
| | lumazine synthase | | | | | |
| 20 | S_stab_disul_woTM/ | 3959-4628, | | 11852-11861, | | 12089-12098, |
| | woTMflex comprising a | 7979-8648, | | 11912-11921, | | 12149-12158, |
| | lumazine synthase | 13588-13654, | | 14125, 14129 | | 14134, 14138 |
| | | 13856-13922 | | | | |
| 21 | S_woTM/woTMflex | 67-75, 4629- | | 11862, 11922 | | 12099, 12159 |
| | comprising a ferritin | 4695, 8649-8715 | | | | |
| 22 | S_stab_PP_woTM/ | 94-102, 4696- | | 11863, 11923 | | 12100, 12160 |
| | woTMflex comprising a | 4762, 8716-8782 | | | | |
| | ferritin | | | | | |
| 23 | S_stab_PP_cav_woTM/ | 4763-4896, | | 11864, 11865, | | 12101, 12102, |
| | woTMflex comprising a | 8783-8916 | | 11924, 11925 | | 12161, 12162 |
| | ferritin | | | | | |
| 24 | S_stab_PP_prot_woTM/ | 4897-4963, | | 11866, 11926 | | 12103, 12163 |
| | woTMflex comprising a | 8917-8983 | | | | |
| | ferritin | | | | | |
| 25 | S_stab_disul_woTM/ | 4964-5633, | | 11867-11876, | | 12104-12113, |
| | woTMflex comprising a | 8984-9653, | | 11927-11936, | | 12164-12173, |
| | ferritin | 13655-13721, | | 14126, 14130 | | 14135, 14139 |
| | | 13923-13989 | | | | |
| 26 | S_woTM/woTMflex | 76-84, 5634- | | 11877, 11937 | | 12114, 12174 |
| | comprising a foldon | 5700, 9654-9720 | | | | |
| 27 | S_stab_PP_woTM/ | 103-111, 5701- | | 11878, 11938 | | 12115, 12175 |
| | woTMflex comprising a | 5767, 9721-9787 | | | | |
| | foldon | | | | | |
| 28 | S_stab_PP_cav_woTM/ | 5768-5901, | | 11879, 11880, | | 12116, 12117, |
| | woTMflex comprising a | 9788-9921 | | 11939, 11940 | | 12176, 12177 |
| | foldon | | | | | |
| 29 | S_stab_PP_prot_woTM/ | 5902-5968, | | 11881, 11941 | | 12118, 12178 |
| | woTMflex comprising a | 9922-9988 | | | | |
| | foldon | | | | | |
| 30 | S_stab_disul_woTM/ | 5969-6638, | | 11882-11891, | | 12119-12128, |
| | woTMflex comprising a | 9989-10658, | | 11942-11951, | | 12179-12188, |
| | foldon | 13722-13788, | | 14127, 14131 | | 14136, 14140 |
| | | 13990-14056 | | | | |
| 31 | S_woTM/woTMflex | 6639-6705, | | 11892, 11952 | | 12129, 12189 |
| | comprising a WhcAg | 10659-10725 | | | | |
| | (VLP) | | | | | |
| 32 | S_stab_PP_woTM/ | 6706-6772, | | 11893, 11953 | | 12130, 12190 |
| | woTMflex comprising a | 10726-10792 | | | | |
| | WhcAg (VLP) | | | | | |
| 33 | S_stab_PP_cav_woTM/ | 6773-6906, | | 11894, 11895, | | 12131, 12132, |
| | woTMflex comprising a | 10793-10926 | | 11954, 11955 | | 12191, 12192 |
| | WhcAg (VLP) | | | | | |
| 34 | S_stab_PP_prot_woTM/ | 6907-6973, | | 11896, 11956 | | 12133, 12193 |
| | woTMflex comprising a | 10927-10993 | | | | |
| | WhcAg (VLP) | | | | | |
| 35 | S_stab_disul_woTM/ | 6974-7643, | | 11897-11906, | | 12134-12143, |
| | woTMflex comprising a | 10994-11663, | | 11957-11966, | | 12194-12203, |
| | WhcAg (VLP) | 13789-13855, | | 14128, 14132 | | 14137, 14141 |
| | | 14057-14123 | | | | |
| 36 | Receptor binding domain; | 13243-13309, | | 13512, 22914, | | 13517, 22915, |
| | RBD | 22917, 22923 | | 22918, 22919, | | 22916, 22920- |
| | | | | 22924, 22925 | | 22922, |
| | | | | | | 22926-22928 |
| 37 | Critical neutralisation | 13310-13376 | | 13513 | | 13518 |
| | domain; CND | | | | | |
| 38 | Stabilized spike protein; | 22732 | | 22759 | | |
| | S_stab_PP_hex | | | | | |
| 39 | RBD comprising a | 22735, 22736 | | 22762, 22763, | | |
| | lumazyne synthase | | | 22967, 22968 | | |
| 40 | RBD comprising a ferritin | 22733 | | 22760, 22965 | | |
| 41 | RBD comprising a foldon | 22734, | | 22761, 22966, | | |
| | | 26938, 26939 | | 26940, 26941 | | |

In preferred embodiments, the at least one antigenic peptide or protein selected or derived from SARS-CoV-2 S comprises or consists of at least one of the amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 1-111, 274-11663, 13176-13510, 13521-14123, 22732-22758, 22732-22758, 22917, 22923, 22929-22964, 26938, 26939 or an immunogenic fragment or immunogenic variant of any of these. Further information regarding said amino acid sequences is also provided in Table 2 (see rows 1 to 41 of Column A and B), and under <223> identifier of the ST25 sequence listing of respective sequence SEQ ID NOs.

In preferred embodiments, the at least one antigenic peptide or protein (pre-fusion stabilized spike protein (S_stab)) selected or derived from SARS-CoV-2 S encoded by the at least one nucleic acid comprises or consists of at least one of the amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 10-26, 40-48, 85-111, 341-1278, 1681-2618, 2686-3623, 3691-4628, 4696-5633, 5701-6638, 6706-7643, 7711-8648, 8716-9653, 9721-10658, 10726-11663, 13377-13510, 13521-14123, 22732, 22738, 22740, 22742, 22744, 22746, 22748, 22750, 22752, 22754, 22756, 22758, 22947-22964 or an immunogenic fragment or immunogenic variant of any of these. Further information regarding said amino acid sequences is also provided in Table 2 (see rows 2 to 5, 12-15,17-20, 22-25, 27-30, and 32-35, 38 of Column A and B), and under <223> identifier of the ST25 sequence listing of respective sequence SEQ ID NOs.

In further preferred embodiments, the at least one antigenic peptide or protein (pre-fusion stabilized spike protein (S_stab)) selected or derived from SARS-CoV-2 S encoded by the at least one nucleic acid comprises or consists of at least one of the amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 26992-26995, 27007-27086, 27087-27109 of PCT patent application PCT/EP2021/069632 or an immunogenic fragment or immunogenic variant of any of these. SEQ ID NOs: 26992-26995, 27007-27086, 27087-27109 of PCT patent application PCT/EP2021/069632, and the corresponding disclosure relating thereto are herewith incorporated by reference.

In particularly preferred embodiments, the at least one antigenic peptide or protein selected or derived from SARS-CoV-2 S comprises or consists of at least one of the amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 10, 21, 22, 25, 27, 274, 341, 408, 475, 542, 743, 810, 1011, 1145, 1212, 1279, 8716, 10726, 22732-22758, 22929-22942, 22947-22964 or an immunogenic fragment or immunogenic variant of any of these.

In particularly preferred embodiments, the at least one antigenic peptide or protein selected or derived from SARS-CoV-2 comprises or consists of at least one of the amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 10-18, 341-407, 22738, 22740, 22742, 22744, 22746, 22748, 22750, 22752, 22754, 22756, 22758, 22947-22964 or an immunogenic fragment or immunogenic variant of any of these. Further information regarding said amino acid sequences is also provided in Table 3A (see row 2 of Column A and B), and under <223> identifier of the ST25 sequence listing of respective sequence SEQ ID NOs.

In further preferred embodiments, the at least one antigenic peptide or protein (pre-fusion stabilized spike protein (S_stab)) selected or derived from SARS-CoV-2 S encoded by the at least one nucleic acid comprises or consists of at least one of the amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 27087-27109 of PCT patent application PCT/EP2021/069632 or an immunogenic fragment or immunogenic variant of any of these. SEQ ID NOs: 27087-27109 of PCT patent application PCT/EP2021/069632, and the corresponding disclosure relating thereto are herewith incorporated by reference.

In a further, more preferred embodiment, the pre-fusion stabilized spike protein (S_stab) comprises or consists of at least one of the amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 22960, 22961, 22963 or an immunogenic fragment or immunogenic variant of any of these.

In a particularly preferred embodiment, the pre-fusion stabilized spike protein (S_stab) comprises or consists of at least one of the amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 22961, or an immunogenic fragment or immunogenic variant of any of these.

In a further particularly preferred embodiment, the pre-fusion stabilized spike protein (S_stab) comprises or consists of at least one of the amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 22959, or an immunogenic fragment or immunogenic variant of any of these.

In further particularly preferred embodiments, the pre-fusion stabilized spike protein (S_stab) comprises or consists of at least one of the amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 27093-27095 of PCT patent application PCT/EP20211069632 or an immunogenic fragment or immunogenic variant of any of these. SEQ ID NOs: 27093-27095 of PCT patent application PCT/EP2021/069632, and the corresponding disclosure relating thereto are herewith incorporated by reference.

In further particularly preferred embodiments, the pre-fusion stabilized spike protein (S_stab) comprises or consists of at least one of the amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NO: 27096 of PCT patent application PCT/EP2021/069632 or an immunogenic fragment or immunogenic variant of any of these. SEQ ID NO: 27096 of PCT patent application PCT/EP2021/069632, and the corresponding disclosure relating thereto are herewith incorporated by reference.

In an even more preferred embodiment, the at least one antigenic peptide or protein selected or derived from SARS-CoV-2 comprises or consists of at least one of the amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 10 or 341 or an immunogenic fragment or immunogenic variant of any of these.

In preferred embodiments, the nucleic acid comprises or consists of at least one coding sequence encoding at least one antigenic peptide or protein selected or derived from a SARS-CoV-2 S as defined herein, preferably encoding any one of SEQ ID NOs: 1-111, 274-11663,13176-13510, 13521-14123, 22732-22758, 22732-22758, 22917, 22923, 22929-22964, 26938, 26939 or fragments of variants thereof. It has to be understood that, on nucleic acid level, any sequence (DNA or RNA sequence) which encodes an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-111, 274-11663, 13176-13510,13521-14123, 22732-22758, 22732-22758, 22917, 22923, 22929-22964, 26938, 26939 or fragments or variants thereof, may be selected and may accordingly be understood as suitable coding sequence of the invention. Further information regarding said amino acid sequences is also provided in Table 2 (see rows 1 to 41 of Column A and B), Table 3A and B, and under <223> identifier of the ST25 sequence listing of respective sequence SEQ ID NOs.

In preferred embodiments, the nucleic acid comprises a coding sequence that comprises at least one of the nucleic acid sequences encoding a SARS-CoV-2 S antigen being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 116-132, 134-138, 140-143, 145-147, 148-175, 11664-11813, 11815, 11817-12050, 12052, 12054-13147, 13514, 13515, 13519, 13520, 14124-14177, 22759, 22764-22786, 22791-22813, 22818-22839, 22969-23184, 23189-23404, 23409-23624, 23629-23844, 23849-24064, 24069-24284, 24289-24504, 24509-24724, 24729-24944, 24949-25164, 25169-25384, 25389-25604, 25609-25824, 25829-26044, 26049-26264, 26269-26484, 26489-26704, 26709-26937 or a fragment or a fragment or variant of any of these sequences. Further information regarding said nucleic acid sequences is also provided in Table 2 (see rows 1 to 7, 9, 11-41 of Column C-F), Table 3A and B, and under <223> identifier of the ST25 sequence listing of respective sequence SEQ ID NOs.

In preferred embodiments, the at least one coding sequence is a codon modified coding sequence as defined herein, wherein the amino acid sequence, that is the SARS-CoV-2 S peptide or protein, encoded by the at least one codon modified coding sequence is preferably not being modified compared to the amino acid sequence encoded by the corresponding wild type or reference coding sequence.

The term “reference coding sequence” relates to the coding sequence, which was the origin sequence to be modified and/or optimized.

In particularly preferred embodiments, the at least one coding sequence of the (additional) nucleic acid is a codon modified coding sequence, wherein the codon modified coding sequence is selected a G/C optimized coding sequence, a human codon usage adapted coding sequence, or a G/C modified coding sequence

In preferred embodiments, the (additional) nucleic acid comprises at least one coding sequence comprising or consisting a codon modified nucleic acid sequence encoding a SARS-CoV-2 S antigen which is identical or at least 70%, 80%, 85%, 86%, 87%, 88%, 89%, 90%, 91%, 92%, 93%, 94%, 95%, 96%, 97%, 98%, or 99% identical to a codon modified nucleic acid sequence selected from the group consisting of SEQ ID NOs: 136-138, 140-143, 145-147,148-175, 11731-11813, 11815, 11817-12050, 12052, 12054-13147, 14142-14177, 22759, 22764-22786, 22791-22813, 22818-22839, 22969-23184, 23189-23404, 23409-23624, 23629-23844, 23849-24064, 24069-24284, 24289-24504, 24509-24724, 24729-24944, 24949-25164, 25169-25384, 25389-25604, 25609-25824, 25829-26044, 26049-26264, 26269-26484, 26489-26704, 26709-26937 or a fragment or variant of any of these sequences. Additional information regarding each of these suitable nucleic acid sequences encoding may also be derived from the sequence listing, in particular from the details provided therein under identifier <223>. Further information regarding said nucleic acid sequences is also provided in Table 2 (see rows 1 to 7, 9, 11-41 of Column D-F), Table 3A and B, and under <223> identifier of the ST25 sequence listing of respective sequence SEQ ID NOs.

In particularly preferred embodiments, the (additional) nucleic acid comprises at least one coding sequence comprising or consisting a G/C optimized coding sequence encoding a SARS-CoV-2 S antigen which is identical or at least 70%, 80%, 85%, 86%, 87%, 88%, 89%, 90%, 91%, 92%, 93%, 94%, 95%, 96%, 97%, 98%, or 99% identical to a codon modified nucleic acid sequence selected from the group consisting of SEQ ID NOs: 136-138, 140, 141, 148, 149, 152, 155, 156, 159, 162, 163, 166, 169, 170, 173, 11731-11813, 11815, 11817-11966, 12271-12472, 12743-12944, 13514, 13515, 14124-14132, 14142-14150, 14160-14168, 22759, 22764-22786, 22791-22813, 22818-22839, 22969-23040, 23077-23148, 23189-23260, 23297-23368, 23409-23480, 23517-23588, 23629-23700, 23737-23808, 23849-23920, 23957-24028, 24069-24140, 24177-24248, 24289-24360, 24397-24468, 24509-24580, 24617-24688, 24729-24800, 24837-24908, 24949-25020, 25057-25128, 25169-25240, 25277-25348, 25389-25460, 25497-25568, 25609-25680, 25717-25788, 25829-25900, 25937-26008, 26049-26120, 26157-26228, 26269-26340, 26377-26448, 26489-26560, 26597-26668, 26709-26780, 26817-26888, 26925-26937 or a fragment or variant of any of these sequences. Additional information regarding each of these suitable nucleic acid sequences encoding may also be derived from the sequence listing, in particular from the details provided therein under identifier <223>. Further information regarding said nucleic acid sequences is also provided in Table 2 (see rows 1 to 7, 9, 11-35 of Column D), Table 3A and B, and under <223> identifier of the ST25 sequence listing of respective sequence SEQ ID NOs.

In particularly preferred embodiments, the (additional) nucleic acid comprises at least one coding sequence comprising or consisting a human codon usage adapted coding sequence encoding a SARS-CoV-2 S which is identical or at least 70%, 80%, 85%, 86%, 87%, 88%, 89%, 90%, 91%, 92%, 93%, 94%, 95%, 96%, 97%, 98%, or 99% identical to a codon modified nucleic acid sequence selected from the group consisting of SEQ ID NOs: 142, 143, 145, 150, 153, 157, 160, 164, 167, 171, 174, 11967-12033, 12473-12539, 12945-13011 or a fragment or variant of any of these sequences. Additional information regarding each of these suitable nucleic acid sequences encoding may also be derived from the sequence listing, in particular from the details provided therein under identifier <223>. Further information regarding said nucleic acid sequences is also provided in Table 2 (see rows 1 to 7, 9, 11-41 of Column E), Table 3A and B, and under <223> identifier of the ST25 sequence listing of respective sequence SEQ ID NOs.

In particularly preferred embodiments, the (additional) nucleic acid comprises at least one coding sequence comprising or consisting a G/C modified coding sequence encoding a SARS-CoV-2 S antigen which is identical or at least 70%, 80%, 85%, 86%, 87%, 88%, 89%, 90%, 91%, 92%, 93%, 94%, 95%, 96%, 97%, 98%, or 99% identical to a codon modified nucleic acid sequence selected from the group consisting of SEQ ID NOs: 146, 147, 151, 154, 158, 161,165, 168, 172, 175, 12034-12050, 12052, 12054-12203,12540-12675, 13012-13147, 13519, 13520, 14133-14141, 14151-14159, 14169-14177, 23041-23076, 23149-23184, 23261-23296, 23369-23404, 23481-23516, 23589-23624, 23701-23736, 23809-23844, 23921-23956, 24029-24064, 24141-24176, 24249-24284, 24361-24396, 24469-24504, 24581-24616, 24689-24724, 24801-24836, 24909-24944, 25021-25056, 25129-25164, 25241-25276, 25349-25384, 25461-25496, 25569-25604, 25681-25716, 25789-25824, 25901-25936, 26009-26044, 26121-26156, 26229-26264, 26341-26376, 26449-26484, 26561-26596, 26669-26704, 26781-26816, 26889-26924 or a fragment or variant of any of these sequences. Additional information regarding each of these suitable nucleic acid sequences encoding may also be derived from the sequence listing, in particular from the details provided therein under identifier <223>. Further information regarding said nucleic acid sequences is also provided in Table 2 (see rows 1 to 7, 9, 11-35 of Column F), Table 3A and B, and under <223> identifier of the ST25 sequence listing of respective sequence SEQ ID NOs.

In even more preferred embodiments, the (additional) nucleic acid comprises at least one coding sequence comprising or consisting a G/C modified coding sequence encoding a SARS-CoV-2 S antigen which is identical or at least 70%, 80%, 85%, 86%, 87%, 88%, 89%, 90%, 91%, 92%, 93%, 94%, 95%, 96%, 97%, 98%, or 99% identical to a codon modified nucleic acid sequence selected from the group consisting of SEQ ID NOs: 136-138, 142, 143, 146, 147, 11731, 11798, 11804, 11805, 11808, 11810, 11811, 11812, 12035, 12049, 22759-22785, 22965-22982, 23077-23094, 23149 or a fragment or variant of any of these sequences. Additional information regarding each of these suitable nucleic acid sequences encoding may also be derived from the sequence listing, in particular from the details provided therein under identifier <223>. Further information regarding said nucleic acid sequences is also provided in Table 2 (see rows 1 to 7, 9, 11-41 of Column F), Table 3A and B, and under <223> identifier of the ST25 sequence listing of respective sequence SEQ ID NOs.

In further preferred embodiments, the (additional) nucleic acid comprises at least one coding sequence comprising or consisting a G/C modified coding sequence encoding a SARS-CoV-2 S antigen which is identical or at least 70%, 80%, 85%, 86%, 87%, 88%, 89%, 90%, 91%, 92%, 93%, 94%, 95%, 96%, 97%, 98%, or 99% identical to a codon modified nucleic acid sequence selected from the group consisting of SEQ ID NOs: 27110-27247 of PCT patent application PCT/EP2021/069632 or a fragment or variant of any of these sequences. SEQ ID NOs: 27110-27247 of PCT patent application PCT/EP2021/069632, and the corresponding disclosure relating thereto are herewith incorporated by reference.

In an particularly preferred embodiment, the (additional) nucleic acid comprises at least one coding sequence comprising or consisting a G/C modified coding sequence encoding a SARS-CoV-2 antigen which is identical or at least 70%, 80%, 85%, 86%, 87%, 88%, 89%, 90%, 91%, 92%, 93%, 94%, 95%, 96%, 97%, 98%, or 99% identical to a codon modified nucleic acid sequence according to SEQ ID NOs: 137 or a fragment or variant thereof.

In further particularly preferred embodiments, the (additional) nucleic acid comprises at least one coding sequence comprising or consisting a G/C modified coding sequence encoding a SARS-CoV-2 antigen which is identical or at least 70%, 80%, 85%, 86%, 87%, 88%, 89%, 90%, 91%, 92%, 93%, 94%, 95%, 96%, 97%, 98%, or 99% identical to a codon modified nucleic acid sequence according to SEQ ID NOs: 23090, 23091, 23093, 23094 or a fragment or variant thereof.

In further particularly preferred embodiments, the (additional) nucleic acid comprises at least one coding sequence comprising or consisting a G/C modified coding sequence encoding a SARS-CoV-2 antigen which is identical or at least 70%, 80%, 85%, 86%, 87%, 88%, 89%, 90%, 91%, 92%, 93%, 94%, 95%, 96%, 97%, 98%, or 99% identical to a codon modified nucleic acid sequence according to SEQ ID NOs: 23091, or a fragment or variant thereof.

In further particularly preferred embodiments, the (additional) nucleic acid comprises at least one coding sequence comprising or consisting a G/C modified coding sequence encoding a SARS-CoV-2 antigen which is identical or at least 70%, 80%, 85%, 86%, 87%, 88%, 89%, 90%, 91%, 92%, 93%, 94%, 95%, 96%, 97%, 98%, or 99% identical to a codon modified nucleic acid sequence according to SEQ ID NOs: 23089, or a fragment or variant thereof.

In further particularly preferred embodiments, the (additional) nucleic acid comprises at least one coding sequence comprising or consisting a G/C modified coding sequence encoding a SARS-CoV-2 antigen which is identical or at least 70%, 80%, 85%, 86%, 87%, 88%, 89%, 90%, 91%, 92%, 93%, 94%, 95%, 96%, 97%, 98%, or 99% identical to a codon modified nucleic acid sequence according to SEQ ID NOs: 27116-27118 of PCT patent application PCT/EP20211069632, or a fragment or variant thereof. SEQ ID NOs: 27116-27118 of PCT patent application PCT/EP2021/069632, and the corresponding disclosure relating thereto are herewith incorporated by reference.

In further particularly preferred embodiments, the (additional) nucleic acid comprises at least one coding sequence comprising or consisting a G/C modified coding sequence encoding a SARS-CoV-2 antigen which is identical or at least 70%, 80%, 85%, 86%, 87%, 88%, 89%, 90%, 91%, 92%, 93%, 94%, 95%, 96%, 97%, 98%, or 99% identical to a codon modified nucleic acid sequence according to SEQ ID NO: 27119 of PCT patent application PCT/EP20211069632, or a fragment or variant thereof. SEQ ID NO: 27119 of PCT patent application PCT/EP2021/069632, and the corresponding disclosure relating thereto are herewith incorporated by reference.

In further embodiments, the (additional) nucleic acid comprises at least one coding sequence comprising or consisting a G/C modified coding sequence encoding a SARS-CoV-2 antigen which is identical or at least 70%, 80%, 85%, 86%, 87%, 88%, 89%, 90%, 91%, 92%, 93%, 94%, 95%, 96%, 97%, 98%, or 99% identical to a codon modified nucleic acid sequence according to SEQ ID NOs: 23113, 23167 or a fragment or variant thereof.

Preferred nucleic acid sequences in that context, including particularly preferred mRNA sequences, are provided in Table 3A (column C and D). Therein, each row represents a specific suitable SARS-CoV-2 spike construct (compare with Table 2), wherein the description of the SARS-CoV-2 spike construct is indicated in column A of Table 3A and the SEQ ID NOs of the amino acid sequence of the respective SARS-CoV-2 spike construct is provided in column B. The corresponding SEQ ID NOs of the coding sequences encoding the respective SARS-CoV-2 spike constructs are provided in Table 2. Further information is provided under <223> identifier of the respective SEQ ID NOs in the sequence listing.

The corresponding nucleic acid, preferably coding RNA sequences, in particular mRNA sequences comprising preferred coding sequences are provided in columns C and D, wherein column C provides nucleic acid sequences with an UTR combination “HSD17B4/PSMB3” as defined herein, wherein column D provides nucleic acid sequences with an “alpha-globin” 3′ UTR as defined herein.

TABLE 3A
Nucleic acid, preferably mRNA constructs encoding SARS-COV-2 spike (S)

| row | A | B | C | D |
|---|---|---|---|---|
| 1 | Full-length spike protein; S | 1-9, 274-340, | 148, 155,12204-12337, | 162, 169, 12676-12809, |
| | | 22737, 22739, | 12473-12540, 22791, | 12945-13012, 22818, |
| | | 22741, 22743, | 22793, 22795, 22797, | 22820, 22822, 22824, |
| | | 22745, 22747, | 22799, 22801, 22803, | 22826, 22828, 22830, |
| | | 22749, 22751, | 22805, 22807, 22809, | 22832, 22834, 22836, |
| | | 22753, 22755, | | |
| | | 22757, 22929- | 22811, 23409-23516 | 22838, 23189-23296 |
| | | 22946 | | |
| 2 | Stabilized spike protein; S_stab_PP | 10-18, 341-407, | 149-151, 156-158, 12338, | 163-165, 170-172, |
| | | 22738, 22740, | 12541, 22792, 22794, | 12810, 13013, 22819, |
| | | 22742, 22744, | 22796, 22798, 22800, | 22821, 22823, 22825, |
| | | 22746, 22748, | 22802, 22804, 22806, | 22827, 22829, 22831, |
| | | 22750, 22752, | 22808, 22810, 22812, | 22833, 22835, 22837, |
| | | 22754, 22756, | | |
| | | 22758, 22947- | 23517-23624 | 22839, 23297-23404 |
| | | 22964 | | |
| 3 | Stabilized spike protein; S_stab_PP_cav | 408-541 | 12339, 12340, 12542, | 12811, 12812, 13014, |
| | | | 12543 | 13015 |
| 4 | Stabilized spike protein; S_stab_PP_prot | 542-608 | 12341, 12544 | 12813, 13016 |
| 5 | Stabilized spike protein; S_stab_disul | 19-26, 609- | 12342-12351, 12545- | 12814-12823, 13017- |
| | | 1278, 13521- | 12554, 14142, 14151 | 13026, 14160, 14169 |
| | | 13587 | | |
| 6 | Spike protein fragment S1 | 27, 1279-1345 | 152-154, 159-161, 12352, | 166-168, 173-175, |
| | | | 12555 | 12824, 13027 |
| 7 | S_woTM comprising a lumazine synthase | 58-66, 3624- | 12353, 12556 | 12825, 13028 |
| | | 3690 | | |
| 8 | S_stab_PP_woTM comprising a | 85-93, 3691- | 12354, 12557 | 12826, 13029 |
| | lumazine synthase | 3757 | | |
| 9 | S_stab_PP_cav_woTM comprising a | 3758-3891 | 12355, 12356, 12558, | 12827, 12828, 13030, |
| | lumazine synthase | | 12559 | 13031 |
| 10 | S_stab_PP_prot_woTM comprising a | 3892-3958 | 12357, 12560 | 12829, 13032 |
| | lumazine synthase | | | |
| 11 | S_stab_disul_woTM comprising a | 3959-4628, | 12358-12367, 12561- | 12830-12839, 13033- |
| | lumazine synthase | 13588-13654 | 12570, 14143, 14152 | 13042, 14161, 14170 |
| 12 | S_woTM comprising a ferritin | 67-75, 4629- | 12368, 12571 | 12840, 13043 |
| | | 4695 | | |
| 13 | S_stab_ PP_woTM comprising a ferritin | 94-102, 4696- | 12369, 12572 | 12841, 13044 |
| | | 4762 | | |
| 14 | S_stab_PP_cav_woTM comprising a | 4763-4896 | 12370, 12371, 12573, | 12842, 12843, 13045, |
| | ferritin | | 12574 | 13046 |
| 15 | S_stab_PP_prot_woTM comprising a | 4897-4963 | 12372, 12575 | 12844, 13047 |
| | ferritin | | | |
| 16 | S_stab_disul_woTM comprising a ferritin | 4964-5633, | 12373-12382, 12576- | 12845-12854, 13048- |
| | | 13655-13721 | 12585, 14144, 14153 | 13057, 14162, 14171 |
| 17 | S_woTM comprising a foldon | 76-84, 5634- | 12383, 12586 | 12855, 13058 |
| | | 5700 | | |
| 18 | S_stab_PP_woTM comprising a foldon | 103-111, 5701- | 12384, 12587 | 12856, 13059 |
| | | 5767 | | |
| 19 | S_stab_PP_cav_woTM comprising a | 5768-5901 | 12385, 12386, 12588, | 12857, 12858, 13060, |
| | foldon | | 12589 | 13061 |
| 20 | S_stab_PP_prot_woTM comprising a | 5902-5968 | 12387, 12590 | 12859, 13062 |
| | foldon | | | |
| 21 | S_stab_disul_woTM comprising a foldon | 5969-6638, | 12388-12397, 12591- | 12860-12869, 13063- |
| | | 13722-13788 | 12600, 14145, 14154 | 13072, 14163, 14172 |
| 22 | S_woTM comprising a WhcAg (VLP) | 6639-6705 | 12398, 12601 | 12870, 13073 |
| 23 | S_stab_ PP_woTM comprising a WhcAg | 6706-6772 | 12399, 12602 | 12871, 13074 |
| | (VLP) | | | |
| 24 | S_stab_PP_cav_woTM comprising a | 6773-6906 | 12400, 12401, 12603, | 12872, 12873, 13075, |
| | WhcAg (VLP) | | 12604 | 13076 |
| 25 | S_stab_PP_prot_woTM comprising a | 6907-6973 | 12402, 12605 | 12874, 13077 |
| | WhcAg (VLP) | | | |
| 26 | S_stab_disul_woTM comprising a WhcAg | 6974-7643, | 12403-12412, 12606- | 12875-12884, 13078- |
| | (VLP) | 13789-13855 | 12615, 14146, 14155 | 13087, 14164, 14173 |
| 27 | S_woTMflex comprising a lumazine | 7644-7710 | 12413, 12616 | 12885, 13088 |
| | synthase | | | |
| 28 | S_stab_ PP_woTMflex comprising a | 7711-7777 | 12414, 12617 | 12886, 13089 |
| | lumazine synthase | | | |
| 29 | S_stab_PP_cav_woTMflex comprising a | 7778-7911 | 12415, 12416, 12618, | 12887, 12888, 13090, |
| | lumazine synthase | | 12619 | 13091 |
| 30 | S_stab_PP_prot_woTMflex comprising a | 7912-7978 | 12417, 12620 | 12889, 13092 |
| | lumazine synthase | | | |
| 31 | S_stab_disul_woTMflex comprising a | 7979-8648, | 12418-12427, 12621- | 12890-12899, 13093- |
| | lumazine synthase | 13856-13922 | 12630, 14147, 14156 | 13102, 14165, 14174 |
| 32 | S_woTMflex comprising a ferritin | 8649-8715 | 12428, 12631 | 12900, 13103 |
| 33 | S_stab_PP_woTMflex comprising a | 8716-8782 | 12429, 12632 | 12901, 13104 |
| | ferritin | | | |
| 34 | S_stab_PP_cav_woTMflex comprising a | 8783-8916 | 12430, 12431, 12633, | 12902, 12903, 13105, |
| | ferritin | | 12634 | 13106 |
| 35 | S_stab_PP_prot_woTMflex comprising a | 8917-8983 | 12432, 12635 | 12904, 13107 |
| | ferritin | | | |
| 36 | S_stab_disul_woTMflex comprising a | 8984-9653, | 12433-12442, 12636- | 12905-12914, 13108- |
| | ferritin | 13923-13989 | 12645, 14148, 14157 | 13117, 14166, 14175 |
| 37 | S_woTMflex comprising a foldon | 9654-9720 | 12443, 12646 | 12915, 13118 |
| 38 | S_stab_ PP_woTMflex comprising a | 9721-9787 | 12444, 12647 | 12916, 13119 |
| | foldon | | | |
| 39 | S_stab_PP_cav_woTMflex comprising a | 9788-9921 | 12445, 12446, 12648, | 12917, 12918, 13120, |
| | foldon | | 12649 | 13121 |
| 40 | S_stab_PP_prot_woTMflex comprising a | 9922-9988 | 12447, 12650 | 12919, 13122 |
| | foldon | | | |
| 41 | S_stab_disul_woTMflex comprising a | 9989-10658, | 12448-12457, 12651- | 12920-12929, 13123- |
| | foldon | 13990-14056 | 12660, 14149, 14158 | 13132, 14167, 14176 |
| 42 | S_woTMflex comprising a WhcAg (VLP) | 10659-10725 | 12458, 12661 | 12930, 13133 |
| 43 | S_stab_ PP_woTMflex comprising a | 10726-10792 | 12459, 12662 | 12931, 13134 |
| | WhcAg (VLP) | | | |
| 44 | S_stab_PP_cav_woTMflex comprising a | 10793-10926 | 12460, 12461, 12663, | 12932, 12933, 13135, |
| | WhcAg (VLP) | | 12664 | 13136 |
| 45 | S_stab_PP_prot_woTMflex comprising a | 10927-10993 | 12462, 12665 | 12934, 13137 |
| | WhcAg (VLP) | | | |
| 46 | S_stab_disul_woTMflex comprising a | 10994-11663, | 12463-12472, 12666- | 12935-12944, 13138- |
| | WhcAg (VLP) | 14057-14123 | 12675, 14150, 14159 | 13147, 14168, 14177 |
| 47 | Stabilized spike protein; S_stab_PP_hex | 22732 | 22786 | 22813 |
| 48 | RBD comprising a lumazyne synthase | 22735, 22736 | 22789, 22790 | 22816, 22817 |
| 49 | RBD comprising a ferritin | 22733 | 22787 | 22814 |
| 50 | RBD comprising a foldon | 22734 | 22788 | 22815 |

Further preferred nucleic acid sequences, preferably mRNA sequences of the invention are provided in Table 3B.

In Table 31B, each column represents a specific suitable SARS-CoV-2 (nCoV-2019) construct of the invention: column B represents “Full-length spike protein; 2” (compare with Table 2 and Table 3A row 1), and column C represents the “Stabilized spike protein; S_stab_PP” (compare with Table 2 and Table 3A row 2). The SEQ ID NOs of the amino acid sequence of the respective SARS-CoV-2 construct are provided in row 1. The corresponding SEQ ID NOs of the coding sequences encoding the respective SARS-CoV-2 constructs are provided in in Table 2. Further information is provided under <223> identifier of the respective SEQ ID NOs in the sequence listing.

The corresponding nucleic acid, preferably coding RNA sequences, in particular mRNA sequences comprising preferred coding sequences are provided in rows 2-16, wherein each row provides nucleic acid sequences with UTR combinations and suitable 3′ ends.

TABLE 3B
Nucleic acid, preferably mRNA constructs encoding SARS-COV-2 spike (S)

| row | A | B | C |
|---|---|---|---|
| 1 | Protein | 1-9, 274-340, 22737, 22739, 22741, | 10-18, 341-407, 22738, 22740, 22742, |
| | | 22743, 22745, 22747, 22749, 22751, | 22744, 22746, 22748, 22750, 22752, |
| | | 22753, 22755, 22757, 22929-22946 | 22754, 22756, 22758, 22947-22964 |
| 2 | RNA i-3 | 162, 169, 12676-12809, 12945-13012, | 163-165, 170-172, 12810, 13013, 22819, |
| | (A64-N5-C30-hSL-N5) | 22818, 22820, 22822, 22824, 22826, | 22821, 22823, 22825, 22827, 22829, |
| | | 22828, 22830, 22832, 22834, 22836, | 22831, 22833, 22835, 22837, 22839, |
| | | 22838, 23189-23296 | 23297-23404 |
| 3 | RNA a-1 | 148, 155, 12204-12337, 12473-12540, | 149-151, 156-158, 12338, 12541, 22792, |
| | (hSL-A100) | 22791, 22793, 22795, 22797, 22799, | 22794, 22796, 22798, 22800, 22802, |
| | | 22801, 22803, 22805, 22807, 22809, | 22804, 22806, 22808, 22810, 22812, |
| | | 22811, 23409-23516 | 23517-23624 |
| 4 | RNA a-3 | 23629-23736 | 23737-23844 |
| | (hSL-A100) | | |
| 5 | RNA e-2 | 23849-23956 | 23957-24064 |
| | (hSL-A100) | | |
| 6 | RNA i-3 | 24069-24176, 24289-24396, | 24177-24284, 24397-24504, |
| | (hSL-A100) | 24509-24616 | 24617-24724 |
| 7 | RNA a-1 | 24729-24836 | 24837-24944 |
| | (A100) | | |
| 8 | RNA a-3 | 24949-25056 | 25057-25164 |
| | (A100) | | |
| 9 | RNA e-2 | 25169-25276 | 25277-25384 |
| | (A100) | | |
| 10 | RNA i-3 | 25389-25496, 25609-25716, | 25497-25604, 25717-25824, |
| | (A100) | 25829-25936 | 25937-26044 |
| 13 | RNA x-1 | 26049-26156 | 26157-26264 |
| | (A100) | | |
| 14 | RNA x-2 | 26269-26376 | 26377-26484 |
| | (A100) | | |
| 15 | RNA x-1 | 26489-26596 | 26597-26704 |
| | (A100-N5) | | |
| 16 | RNA x-2 | 26709-26816 | 26817-26924 |
| | (A30-N10-A70) | | |

In preferred embodiments, the (additional) nucleic acid, preferably the RNA, comprises or consists of a nucleic acid sequence encoding a SARS-CoV-2 S antigen which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected SEQ ID NOs: 148-175, 12204-13147,14142-14177, 22786-22839, 23189-23404, 23409-23624, 23629-23844, 23849-24064, 24069-24284, 24289-24504, 24509-24724, 24729-24944, 24949-25164, 25169-25384, 25389-25604, 25609-25824, 25829-26044, 26049-26264, 26269-26484, 26489-26704, 26709-26937 or a fragment or variant of any of these sequences. Further information regarding respective nucleic acid sequences is provided under <223> identifier of the respective SEQ ID NO in the sequence listing, and in Table 3A (see in particular Column C and D) and 3B (see in particular rows 2-16).

In particularly preferred embodiments, the (additional) nucleic acid, preferably the RNA, comprises or consists of a nucleic acid sequence encoding a SARS-CoV-2 S antigen which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 162-175, 12676-13147, 14160-14177, 22813-22839, 23189-23404 or a fragment or variant of any of these sequences. Further information regarding respective nucleic acid sequences is provided under <223> identifier of the respective SEQ ID NO in the sequence listing, and in Table 3A (see in particular Column D) and 3B (row 2)

In particularly preferred embodiments, the (additional) nucleic acid, preferably the RNA, comprises or consists of a nucleic acid sequence encoding a SARS-CoV-2 S antigen which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 148-161, 12204-12675, 14142-14159, 22786-22812, 23409-23624, 24729-24944 or a fragment or variant of any of these sequences. Further information regarding respective nucleic acid sequences is provided under <223> identifier of the respective SEQ ID NO in the sequence listing, and in Table 3A (see in particular Column C) and Table 3B (see rows 3, 7).

In particularly preferred embodiments, the (additional) nucleic acid, preferably the RNA, comprises or consists of a nucleic acid sequence encoding a SARS-CoV-2 S antigen which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 149-154, 156-161, 163-168, 170-175, 12338, 12352, 12541, 12555, 12810, 12824, 13013, 13027, 22786, 22792, 22794, 22796, 22798, 22800, 22802, 22804, 22806, 22808, 22810, 22812, 22813, 22819, 22821, 22823, 22825, 22827, 22829, 22831, 22833, 22835, 22837, 22839, 23517-23624, 23297-23404, 24837-24944 or a fragment or variant of any of these sequences. Further information regarding respective nucleic acid sequences is provided under <223> identifier of the respective SEQ ID NO in the sequence listing and in Table 3A (see Column C and D, rows 2 and 6) and Table 3B (see Column C)

In even more preferred embodiments, the (additional) nucleic acid, preferably the RNA, comprises or consists of a nucleic acid sequence encoding a SARS-CoV-2 S antigen which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 149, 156, 12338, 150, 157, 151, 158, 12541, 163, 170, 12810, 164,171, 165, 172, 13013, 12342-12351,12545-12554, 12814-12823,13017-13026, 14133, or a fragment or variant of any of these sequences. Further information regarding respective nucleic acid sequences is provided under <223> identifier of the respective SEQ ID NO in the sequence listing and in Table 3A and 38.

In even more preferred embodiments, the (additional) nucleic acid, preferably the RNA, comprises or consists of a nucleic acid sequence encoding a SARS-CoV-2 S antigen selected from SEQ ID NOs: 149, 150, 163, 164, 165, 24837, 23311, 23531, 24851, 23310, 23530, 24850, 23313, 23533, 24853, 23314, 23534, 24854, or a fragment or variant of any of these sequences. Further information regarding respective nucleic acid sequences is provided under <223> identifier of the respective SEQ ID NO in the sequence listing and in Table 3A and B.

In further preferred embodiments, the (additional) nucleic acid, preferably the RNA, comprises or consists of a nucleic acid sequence encoding a SARS-CoV-2 S antigen selected from SEQ ID NOs: 27248-27385, 27662-27907 of PCT patent application PCT/EP2021/069632, or a fragment or variant of any of these sequences. SEQ ID NOs: 27248-27385, 27662-27907 of PCT patent application PCT/EP2021/069632, and the corresponding disclosure relating thereto are herewith incorporated by reference.

In further preferred embodiments, the (additional) nucleic acid, preferably the RNA, comprises or consists of a nucleic acid sequence encoding a SARS-CoV-2 S antigen selected from SEQ ID NOs: 27386-27661 of PCT patent application PCT/EP20211069632, or a fragment or variant of any of these sequences. SEQ ID NOs: 27386-27661 of PCT patent application PCT/EP2021/069632, and the corresponding disclosure relating thereto are herewith incorporated by reference.

In a particularly preferred embodiment, the (additional) nucleic acid, preferably the RNA, comprises or consists of a nucleic acid sequence encoding a SARS-CoV-2 antigen selected from SEQ ID NOs: 163 or a fragment or variant of that sequence.

In a particularly preferred embodiment, the (additional) nucleic acid, preferably the RNA, comprises or consists of a nucleic acid sequence encoding a SARS-CoV-2 antigen selected from SEQ ID NOs: 149 or a fragment or variant of that sequence.

In a further particularly preferred embodiment, the (additional) nucleic acid, preferably the RNA, comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence of SEQ ID NO: 24837.

In a further preferred embodiment, the (additional nucleic acid, preferably the RNA, comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence of SEQ ID NO: 23311, 23531, 24851.

In a further preferred embodiment, the (additional) nucleic acid, preferably the RNA, comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence of SEQ ID NO: 23310, 23530, 24850.

In a further preferred embodiment, the (additional) nucleic acid, preferably the RNA, comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence of SEQ ID NO: 23309, 23529, 24849.

In further preferred embodiments, the (additional) nucleic acid, preferably the RNA, comprises or consists of a nucleic acid sequence encoding a SARS-CoV-2 S antigen selected from SEQ ID NOs: 27254, 27255, 27256, 27277, 27278, 27279, 27300, 27301, 27302, 27323, 27324, 27325, 27346, 27347, 27348, 27369, 27370, 27371, 27392, 27393, 27394, 27415, 27416, 27417, 27438, 27439, 27440, 27461, 27462, 27463, 27484, 27485, 27486, 27507, 27508, 27509, 27530, 27531, 27532, 27553, 27554, 27555, 27576, 27577, 27578, 27599, 27600, 27601, 27622, 27623, 27624, 27645, 27646, 27647, 27686, 27687, 27688, 27727, 27728, 27729, 27768, 27769, 27770, 27809, 27810, 27811, 27850, 27851, 27852, 27891, 27892, 27893 of PCT patent application PCT/EP2021/069632, or a fragment or variant of any of these sequences, wherein SEQ ID NOs: 27254-27256, 27392-27394, 27530-27532, or a fragment or variant of these sequences are particularly preferred. SEQ ID NOs: 27254, 27255, 27256, 27277, 27278, 27279, 27300, 27301, 27302, 27323, 27324, 27325, 27346, 27347, 27348, 27369, 27370, 27371, 27392, 27393, 27394, 27415, 27416, 27417, 27438, 27439, 27440, 27461, 27462, 27463, 27484, 27485, 27486, 27507, 27508, 27509, 27530, 27531, 27532, 27553, 27554, 27555, 27576, 27577, 27578, 27599, 27600, 27601, 27622, 27623, 27624, 27645, 27646, 27647, 27686, 27687, 27688, 27727, 27728, 27729, 27768, 27769, 27770, 27809, 27810, 27811, 27850, 27851, 27852, 27891, 27892, 27893 of PCT patent application PCT/EP2021/069632, and the corresponding disclosure relating thereto are herewith incorporated by reference.

In further preferred embodiments, the (additional) nucleic acid, preferably the RNA, comprises or consists of a nucleic acid sequence encoding a SARS-CoV-2 S antigen selected from SEQ ID NOs: 27257, 27280, 27303, 27326, 27349, 27372, 27689, 27730, 27771, 27812, 27853, 27894, 27395, 27418, 27441, 27464, 27487, 27510, 27533, 27556, 27579, 27602, 27625, 27648 of PCT patent application PCT/EP2021/069632 or a fragment or variant of any of these sequences. SEQ ID NOs: 27257, 27280, 27303, 27326, 27349, 27372, 27689, 27730, 27771, 27812, 27853, 27894, 27395, 27418, 27441, 27464, 27487, 27510, 27533, 27556, 27579, 27602, 27625, 27648 of PCT patent application PCT/EP2021/069632, and the corresponding disclosure relating thereto are herewith incorporated by reference.

In a further preferred embodiment, the (additional) nucleic acid, preferably the RNA, comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence of SEQ ID NO: 23313, 23533, 24853, 23314, 23534, 24854.

In a further preferred embodiment, the (additional) nucleic acid, preferably the RNA, comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence of SEQ ID NO: 26633.

In a further preferred embodiment, the (additional) nucleic acid, preferably the RNA, comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence of SEQ ID NO: 26907.

In further preferred embodiments, the (additional) nucleic acid, preferably the RNA, comprises or consists of a nucleic acid sequence encoding a SARS-CoV-2 S antigen which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 148-175,12204-13147, 14142-14177, 22786-22839, 23189-23404, 23409-23624, 23629-23844, 23849-24064, 24069-24284, 24289-24504, 24509-24724, 24729-24944, 24949-25164, 25169-25384, 25389-25604, 25609-25824, 25829-26044, 26049-26264, 26269-26484, 26489-26704, 26709-26937 optionally, wherein said RNA sequences comprise a cap1 structure as defined herein, and, optionally, wherein at least one, preferably all uracil nucleotides in said RNA sequences are replaced by pseudouridine (ψ) nucleotides and/or N1-methylpseudouridine (m1ψ) nucleotides.

In further preferred embodiments, the (additional) nucleic acid, preferably the RNA, comprises or consists of a nucleic acid sequence encoding a SARS-CoV-2 S antigen selected from SEQ ID NOs: 27248-27385, 27662-27907, 27386-27661 of PCT patent application PCT/EP2021/069632 or a fragment or variant of any of these sequences, wherein said RNA sequences comprise a cap1 structure as defined herein, and, optionally, wherein at least one, preferably all uracil nucleotides in said RNA sequences are replaced by pseudouridine (ψ) nucleotides and/or N1-methylpseudouridine (m1ψ) nucleotides.

In specific embodiments, the pharmaceutical composition comprises a plurality or at least 2, 3, 4, 5, 6, 7, 8, 9, 10 or even more of the nucleic acid species, e.g. RNA species encoding SARS-CoV-2 S.

In embodiments, the pharmaceutical composition comprises 2, 3, 4 or 5 nucleic acid species (e.g. DNA or RNA), preferably RNA species, wherein said nucleic acid species comprise or consist of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 116-132, 134-138, 140-143, 145-175, 11664-11813, 11815, 11817-12050, 12052, 12054-13147, 13514, 13515, 13519, 13520, 14124-14177, 22759, 22764-22786, 22791-22813, 22818-22839, 22969-23184, 23189-23404, 23409-23624, 23629-23844, 23849-24064, 24069-24284, 24289-24504, 24509-24724, 24729-24944, 24949-25164, 25169-25384, 25389-25604, 25609-25824, 25829-26044, 26049-26264, 26269-26484, 26489-26704, 26709-26937 and, optionally, at least one pharmaceutically acceptable carrier or excipient, wherein each of the 2, 3, 4 or 5 nucleic acid species encode a different antigenic peptide or protein of a SARS-CoV-2 coronavirus.

Accordingly, in embodiments, the pharmaceutical composition comprises two nucleic acid species (e.g. DNA or RNA), preferably RNA species, wherein the nucleic acid species comprise or consist of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 148-175, 12204-13147, 14142-14177, 22786-22839, 23189-23404, 23409-23624, 23629-23844, 23849-24064, 24069-24284, 24289-24504, 24509-24724, 24729-24944, 24949-25164, 25169-25384, 25389-25604, 25609-25824, 25829-26044, 26049-26264, 26269-26484, 26489-26704, 26709-26937, and, optionally, at least one pharmaceutically acceptable carrier or excipient, wherein each of the two nucleic acid species encode a different antigenic peptide or protein of a SARS-CoV-2 coronavirus.

In embodiments, the pharmaceutical composition comprises three nucleic acid species (e.g. DNA or RNA), preferably RNA species, wherein the nucleic acid comprises or consists of a nucleic acid sequence which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting 148-175, 12204-13147, 14142-14177, 22786-22839, 23189-23404, 23409-23624, 23629-23844, 23849-24064, 24069-24284, 24289-24504, 24509-24724, 24729-24944, 24949-25164, 25169-25384, 25389-25604, 25609-25824, 25829-26044, 26049-26264, 26269-26484, 26489-26704, 26709-26937, and, optionally, at least one pharmaceutically acceptable carrier or excipient, wherein each of the 2, 3, 4 or 5 nucleic acid species encode a different antigenic peptide or protein of a SARS-CoV-2 coronavirus.

Preferably, the at least 2, 3, 4, 5, 6, 7, 8, 9, 10 or even more different nucleic acid species encoding SARS-CoV-2 S of the composition each encode a different prefusion stabilized spike protein (as defined in the first aspect). Preferably, stabilization of the perfusion conformation is obtained by introducing two consecutive proline substitutions at residues K986 and V987 in the spike protein (Amino acid positions according to reference SEQ ID NO: 1). Accordingly, in preferred embodiments, the at least 2, 3, 4, 5, 6, 7, 8, 9, 10 pre-fusion stabilized spike proteins (S_stab) each comprises at least one pre-fusion stabilizing mutation, wherein the at least one pre-fusion stabilizing mutation comprises the following amino acid substitutions: K986P and V987P (amino acid positions according to reference SEQ ID NO: 1).

Suitably, the different spike proteins or prefusion stabilized spike proteins are derived from at least B.1.1.7, B.1.351, P.1, or CAL.20C.

Suitably, the different spike proteins or prefusion stabilized spike proteins have amino acid changes in the S protein comprising:
(i) delH69, delV70, Y453F, D614G, I692V and M1229I;
(ii) delH69, delV70, delY144, N501Y, A570D, D614G, P681H, T716I, S982A and D1118H;
(iii) L18F, D80A, D215G, delL242, delA243, delL244, R246I, K417N, E484K, N501Y, D614G and A701V;
(iv) L18F, T20N, P26S, D138Y, R190S, K417T, E484K, N501Y, D614G, H655Y and T1027I; and/or
(v) S13I, W152C, L452R, and D614G.

Accordingly, the at least 2, 3, 4, 5, 6, 7, 8, 9, 10 or even more different nucleic acid species encoding SARS-CoV-2 S of the composition each encode a different prefusion stabilized spike protein, wherein the at least 2, 3, 4, 5, 6, 7, 8, 9, 10 or even more stabilized spike proteins are selected from amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 10-26, 341-407, 609-1278,13521-13587, 22738, 22740, 22742, 22744, 22746, 22748, 22750, 22752, 22754, 22756, 22758, 22947-22964, or an immunogenic fragment or immunogenic variant of any of these.

In preferred embodiments, the composition comprises at 2, 3, 4, or 5 nucleic acid species comprising a coding sequence encoding an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 10, 22961; 22960, 22963, 22941, 22964.

In preferred embodiments, the composition comprises one nucleic acid species comprising a coding sequence encoding an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 10, wherein the multivalent composition additionally comprises at least 2, 3, 4 further RNA species selected from
i) one nucleic acid species comprises a coding sequence encoding an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 22961; and/or
ii) one nucleic acid species comprises a coding sequence encoding an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 22960; and/or
iii) one nucleic acid species comprises a coding sequence encoding an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 22963; and/or
iv) one nucleic acid species comprises a coding sequence encoding an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 22941; and/or
v) one nucleic acid species comprises a coding sequence encoding an amino acid sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 22964.

Preferably, the at least 2, 3, 4, 5, 6, 7, 8, 9, 10 or even more different nucleic acid species encoding SARS-CoV-2 S of the composition comprise nucleic acid coding sequences each encoding a different prefusion stabilized spike protein, wherein the at least 2, 3, 4, 5, 6, 7, 8, 9, 10 or even more nucleic acid coding sequences are selected from nucleic acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 136-138, 140-143, 145-175, 11731-11813, 11815, 11817-12050, 12052, 12054-12203, 13514, 13515, 13519, 13520, 14124-14141, 22759, 22764-22785, 22969-23184 or fragments or variants of any of these.

In preferred embodiments, the composition comprises one nucleic acid species comprising a coding sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 137, wherein the multivalent composition additionally comprises at least 2, 3, 4 further RNA species selected from
i) one nucleic acid species comprises a coding sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 23091; and/or
ii) one nucleic acid species comprises a coding sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 23090; and/or
iii) one nucleic acid species comprises a coding sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 23093; and/or
iv) one nucleic acid species comprises a coding sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 22999; and/or
v) one nucleic acid species comprises a coding sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 23094.

Preferably, the at least 2, 3, 4, 5, 6, 7, 8, 9, 10 or even more different nucleic acid species encoding SARS-CoV-2 S of the composition comprise nucleic acid coding sequences each encoding a different prefusion stabilized spike protein, wherein the at least 2, 3, 4, 5, 6, 7, 8, 9, 10 or even more nucleic acid coding sequences are selected from RNA sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 149-151, 163-165, 12338, 12541, 12810-12813, 12901, 12931, 13013, 22792, 22794, 22796, 22798, 22802, 22804, 22806, 22810, 22813, 22819, 22821, 22823, 22825, 22827, 22829, 22831, 22833, 22835, 22837, 22839, 23297-23314, 23369, 23517-23520, 23523-23525, 23527, 23529, 23530, 23589, 23737, 23957, 24397, 24837, 25057, 25277, 25717, 26925-26937 or fragments or variants of any of these.

In preferred embodiments, the composition comprises one RNA species comprising or consisting of an RNA sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 163, wherein the composition additionally comprises at least 2, 3, 4 further RNA species selected from
i) one RNA species comprising or consisting of an RNA sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 23311; and/or
ii) one RNA species comprises a coding sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 23310; and/or
iii) one RNA species comprises a coding sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 23313; and/or
iv) one RNA species comprises a coding sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 23219; and/or
v) one RNA species comprises a coding sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 23314;
   wherein, preferably, each of the mRNA species comprise a Cap1 structure, and, optionally, each of the mRNA species do not comprise modified nucleotides.

In preferred embodiments, the composition comprises one RNA species comprising or consisting of an RNA sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 149 or 24837, wherein the multivalent composition additionally comprises at least 2, 3, 4 further RNA species selected from
i) one RNA species comprising or consisting of an RNA sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 23531 or 24851; and/or
ii) one RNA species comprises a coding sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 23530 or 24850; and/or
iii) one RNA species comprises a coding sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 23533 or 24853; and/or
iv) one RNA species comprises a coding sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 23439 or 24759; and/or
v) one RNA species comprises a coding sequence being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 23534 or 24854;
   wherein, preferably, each of the mRNA species comprise a Cap1 structure, and, optionally, each of the mRNA species do not comprise modified nucleotides.

In further preferred embodiments, the composition comprises at least two RNA species being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 149 or 24837, 23531 or 24851, 23530 or 24850, 23533 or 24853, 23439 or 24759 or 23534 or 24854.

Nucleic Acid Encoding SARS-CoV-1 Spike Protein

In a particularly preferred embodiment, the Coronavirus spike protein (S) is selected or derived from at least one SARS-associated virus spike protein, preferably a SARS-CoV-1 spike protein (S1, S2, or S1 and S2), or an immunogenic fragment or immunogenic variant thereof.

In a particularly preferred embodiment, the Coronavirus spike protein (S) is selected from a SARS-CoV-1 virus.

It has to be understood that generic embodiments and features that have been described paragraph “Nucleic acid encoding a Coronavirus spike protein” may also apply to a nucleic acid encoding a SARS-CoV-1 spike protein.

Suitable antigenic peptide or protein sequences that are provided by the (additional) nucleic acid are disclosed in Table 4, rows 1 to 45, Column A and B. In addition, further information regarding said suitable antigenic peptide or protein sequences selected or derived from SARS-associated virus, preferably a SARS-CoV-1 are provided under <223> identifier of the ST25 sequence listing.

In particularly preferred embodiments, the encoded at least one antigenic peptide or protein comprises or consists of a SARS-associated virus spike protein (S), preferably a SARS-CoV-1 spike protein (S), wherein the spike protein (S) comprises or consists of a spike protein fragment S1, or an immunogenic fragment or immunogenic variant thereof.

In particularly preferred embodiments, the encoded at least one antigenic peptide or protein comprises or consists of a full-length SARS-associated virus spike protein (S), preferably a SARS-CoV-1 spike protein (S) or an immunogenic fragment or immunogenic variant of any of these.

In particularly preferred embodiments, the SARS-associated virus spike protein (S), preferably a SARS-CoV-1 spike protein (S) that is provided by the nucleic acid is designed or adapted to stabilize the antigen in pre-fusion conformation. A pre-fusion conformation is particularly advantageous in the context of an efficient Coronavirus vaccine, as several potential epitopes for neutralizing antibodies may merely be accessible in said pre-fusion protein conformation. Furthermore, remaining of the protein in the pre-fusion conformation is aimed to avoid immunopathological effects, like e.g. enhanced disease and/or antibody dependent enhancement (ADE).

Accordingly, in preferred embodiments, the (additional) nucleic acid of comprises at least one coding sequence encoding at least one antigenic peptide or protein that is selected or derived from an SARS-associated virus spike protein (S), preferably a SARS-CoV-1 spike protein (S), wherein the spike protein (S) is a pre-fusion stabilized spike protein (S_stab). Suitably, said pre-fusion stabilized spike protein comprises at least one pre-fusion stabilizing mutation.

Stabilization of the SARS-CoV-1 spike protein may be obtained by substituting at least one amino acids at position K968 and/or V969 with amino acids that stabilize the spike protein in a perfusion conformation (amino acid positions according to reference SEQ ID NO: 14906).

In embodiments, the pre-fusion stabilizing mutation of SARS-CoV-1 spike protein comprises an amino acid substitution at position K968, wherein the amino acids K968 is substituted with one selected from A, I, L, M, F, V, G, or P (amino acid positions according to reference SEQ ID NO: 14906), preferably wherein the amino acids K968 is substituted with P. In embodiments, the pre-fusion stabilizing mutation comprises an amino acid substitution at position V969, wherein the amino acids V969 is substituted with one selected from A, I, L, M, F, V, G, or P (amino acid positions according to reference SEQ ID NO: 14906), preferably wherein the amino acids V969 is substituted with P.

Accordingly, in preferred embodiments, the pre-fusion stabilized spike protein (S_stab) of SARS-CoV-1 comprises at least one pre-fusion stabilizing mutation, wherein the at least one pre-fusion stabilizing mutation comprises the following amino acid substitutions: K968P and V969P (amino acid positions according to reference SEQ ID NO: 14906).

Any SARS-associated virus spike protein or fragments or variants thereof can be chosen by the skilled person to introduce such amino acid changes (e.g. such a double Proline mutation). Examples comprise HCoV/OC43 spike protein (1-1353)(A1070P_L1071P), HCoV/OC43/1783A_10 spike protein (1-1362)(A1079P_L1080P), HCoV/HKU1/N5 spike protein (1-1351)(N1067P_L1068P), HCoV/229E/BN1/GER/2015 spike protein (1-1171)(I869P_I870P), HCoV/NL63/RPTEC/2004 spike protein (1-1356)(S1052P_I1053P), Bat SARS-like CoV/WIV1 spike protein (1-1256)(K969P_V970P), BatCoV/HKU9-1 BF_005I spike protein (1-1274)(G983P_L984P), PDCoV/Swine/Thailand/S5011/2015 spike protein (1-1160)((E855P_V856P), PEDV/NPL-PEDv/2013/P10 spike protein (1-1386)(I1076P_L1077P), MHV/S spike protein (1-1361)(A1073P_L1074P).

In embodiments, the at least one pre-fusion stabilizing mutation of SARS-associated virus spike protein, preferably a SARS-CoV-1 spike protein comprises a cavity filling mutation.

In embodiments, the at least one pre-fusion stabilizing mutation of SARS-associated virus spike protein, preferably a SARS-CoV-1 spike protein comprises a mutated protonation site.

In embodiments, the at least one pre-fusion stabilizing mutation of the SARS-associated virus spike protein, preferably a SARS-CoV-1 spike protein comprises an artificial intramolecular disulfide bond. Such an artificial intramolecular disulfide bond can be introduced to further stabilize the membrane distal portion of the S protein (including the N-terminal region) in the pre-fusion conformation; that is, in a conformation that specifically binds to one or more pre-fusion specification antibodies, and/or presents a suitable antigenic site that is present on the pre-fusion conformation but not in the post fusion conformation of the S protein.

Preferred antigenic peptide or proteins selected or derived from a SARS-associated virus spike protein, preferably a SARS-CoV-1 spike protein as defined above are provided in Table 4 (rows 1 to 45). Therein, each row to 45 corresponds to a suitable SARS-CoV-1 constructs or a SARS-associated virus construct. Column A of Table 4 provides a short description of suitable antigen constructs. Column B of Table 4 provides protein (amino acid) SEQ ID NOs of respective antigen constructs. Column D of Table 4 provides SEQ ID NO of the corresponding G1P optimized nucleic acid coding sequences (opt1, gc). Column E of Table 4 provides SEQ ID NO of the corresponding human codon usage adapted nucleic acid coding sequences (opt 3, human).

Notably, the description of the invention explicitly includes the information provided under <223> identifier of the ST25 sequence listing of the present application. Preferred nucleic acid constructs comprising coding sequences of Table 4, e.g. mRNA sequences comprising the coding sequences of Table 4 are provided in Table 5.

TABLE 4
SARS-COV-1 and SARS-associated spike protein constructs (amino acid
sequences and nucleic acid coding sequences):

| Row | A | B | D | E |
|---|---|---|---|---|
| 1 | SARS-COV spike protein (1-1255) | 14906 | 14951 | 14996 |
| 2 | SARS-COV spike protein (1-1255)(K968P_V969P) | 14907 | 14952 | 14997 |
| 3 | SARS-COV spike protein (1-17_747-1255) | 14908 | 14953 | 14998 |
| 4 | SARS-COV/Tor2 spike protein (1-1255) | 14909 | 14954 | 14999 |
| 5 | SARS-COV/Tor2 spike protein (1-1255)(K968P_V969P) | 14910 | 14955 | 15000 |
| 6 | SARS-COV/Tor2 spike protein (variant) | 14911 | 14956 | 15001 |
| 7 | SARS-COV/Tor2 spike protein (variant) | 14912 | 14957 | 15002 |
| 8 | HCoV/OC43 spike protein (1-1353) | 14913 | 14958 | 15003 |
| 9 | HCoV/OC43 spike protein (1-1353)(A1070P_L1071P) | 14914 | 14959 | 15004 |
| 10 | HCoV/OC43/1783A_10 spike protein (1-1362) | 14915 | 14960 | 15005 |
| 11 | HCoV/OC43/1783A_10 spike protein (1-1362)(A1079P_L1080P) | 14916 | 14961 | 15006 |
| 12 | HCoV/OC43/1783A_10 spike protein (variant) | 14917 | 14962 | 15007 |
| 13 | HCoV/OC43/1783A_10 spike protein (variant) | 14918 | 14963 | 15008 |
| 14 | HCoV/HKU1/N5 spike protein (1-1351) | 14919 | 14964 | 15009 |
| 15 | HCoV/HKU1/N5 spike protein (1-1351)(N1067P_L1068P) | 14920 | 14965 | 15010 |
| 16 | HCoV/HKU1/N5 spike protein (variant) | 14921 | 14966 | 15011 |
| 17 | HCoV/HKU1/N5 spike protein (variant) | 14922 | 14967 | 15012 |
| 18 | HCoV/229E/BN1/GER/2015 spike protein (1-1171) | 14923 | 14968 | 15013 |
| 19 | HCoV/229E/BN1/GER/2015 spike protein (1-1171)(1869P_I870P) | 14924 | 14969 | 15014 |
| 20 | HCoV/229E/BN1/GER/2015 spike protein (variant) | 14925 | 14970 | 15015 |
| 21 | HCoV/229E/BN1/GER/2015 spike protein (variant) | 14926 | 14971 | 15016 |
| 22 | HCoV/NL63/RPTEC/2004 spike protein (1-1356) | 14927 | 14972 | 15017 |
| 23 | HCoV/NL63/RPTEC/2004 spike protein (1-1356)(S1052P_I1053P) | 14928 | 14973 | 15018 |
| 24 | HCoV/NL63/RPTEC/2004 spike protein (variant) | 14929 | 14974 | 15019 |
| 25 | HCoV/NL63/RPTEC/2004 spike protein (variant) | 14930 | 14975 | 15020 |
| 26 | Bat SARS-like CoV/WIV1 spike protein (1-1256) | 14931 | 14976 | 15021 |
| 27 | Bat SARS-like CoV/WVIV1 spike protein (1-1256)(K969P_V970P) | 14932 | 14977 | 15022 |
| 28 | Bat SARS-like CoV/WIV1 spike protein (variant) | 14933 | 14978 | 15023 |
| 29 | Bat SARS-like CoV/WIV1 spike protein (variant) | 14934 | 14979 | 15024 |
| 30 | BatCoV/HKU9-1 BF_0051 spike protein (1-1274) | 14935 | 14980 | 15025 |
| 31 | BatCoV/HKU9-1 BF_0051 spike protein (1-1274)(G983P_L984P) | 14936 | 14981 | 15026 |
| 32 | BatCoV/HKU9-1 BF_005l spike protein (variant) | 14937 | 14982 | 15027 |
| 33 | BatCoV/HKU9-1 BF_005l spike protein (variant) | 14938 | 14983 | 15028 |
| 34 | PDCoV/Swine/Thailand/S5011/2015 spike protein (1-1160) | 14939 | 14984 | 15029 |
| 35 | PDCoV/Swine/Thailand/S5011/2015 spike protein (1-1160)((E855P_V856P) | 14940 | 14985 | 15030 |
| 36 | PDCoV/Swine/Thailand/S5011/2015 spike protein (variant) | 14941 | 14986 | 15031 |
| 37 | PDCoV/Swine/Thailand/S5011/2015 spike protein (variant) | 14942 | 14987 | 15032 |
| 38 | PEDV/NPL-PEDv/2013/P10 spike protein (1-1386) | 14943 | 14988 | 15033 |
| 39 | PEDV/NPL-PEDv/2013/P10 spike protein (1-1386)(I1076P_L1077P) | 14944 | 14989 | 15034 |
| 40 | PEDV/NPL-PEDv/2013/P10 spike protein (variant) | 14945 | 14990 | 15035 |
| 41 | PEDV/NPL-PEDv/2013/P10 spike protein (variant) | 14946 | 14991 | 15036 |
| 42 | MHV/S spike protein (1-1361) | 14947 | 14992 | 15037 |
| 43 | MHV/S spike protein (1-1361)(A1073P_L1074P) | 14948 | 14993 | 15038 |
| 44 | MHV/S spike protein (variant) | 14949 | 14994 | 15039 |
| 45 | MHV/S spike protein (variant) | 14950 | 14995 | 15040 |

In preferred embodiments, the at least one antigenic peptide or protein selected or derived from a SARS associated virus S, preferably SARS-CoV-1 S encoded by the at least one (additional) nucleic acid comprises or consists of at least one of the amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 14906-14950 or an immunogenic fragment or immunogenic variant of any of these. Further information regarding said amino acid sequences is also provided in Table 4 (see rows 1 to 45 of Column A and B), and under <223> identifier of the ST25 sequence listing of respective sequence SEQ ID NOs.

In further embodiments, the at least one antigenic peptide or protein selected or derived from SARS-CoV-1 comprises or consists of at least one of the amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 29, 32 or 34 of published PCT patent application WO2017070626 or an immunogenic fragment or immunogenic variant of any of these. SEQ ID NOs: 29, 32 or 34 of WO2017070626, and the corresponding disclosure relating thereto are herewith incorporated by reference. In further embodiments, the at least one antigenic peptide or protein selected or derived from SARS-CoV-1 comprises or consists of at least one of the amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 7 or 30 of published PCT patent application WO2018081318 or an immunogenic fragment or immunogenic variant of any of these. SEQ ID NOs: 7 or 30 of WO2018081318, and the corresponding disclosure relating thereto are herewith incorporated by reference. Further suitable antigenic peptide or proteins selected or derived from SARS-CoV-1 can be selected or derived from Table 12 of WO2017070626. Accordingly, the full content of Table 12 of WO2017070626 herewith incorporated by reference.

In preferred embodiments, the at least one antigenic peptide or protein (pre-fusion stabilized spike protein (S_stab)) selected or derived from a SARS associated virus, preferably a SARS-CoV-1 encoded by the at least one (additional) nucleic acid comprises or consists of at least one of the amino add sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 14907, 14910, 14914, 14916, 14920, 14924, 14928, 14932, 14936, 14940, 14944, 14948, or an immunogenic fragment or immunogenic variant of any of these. Further information regarding said amino acid sequences is also provided in Table 4, and under <223> identifier of the ST25 sequence listing of respective sequence SEQ ID NOs.

According to preferred embodiments, the (additional) nucleic acid comprises at least one coding sequence encoding at least one antigenic peptide or protein derived from SARS associated virus S, preferably a SARS-CoV-1 S as defined above, or fragments and variants thereof. In that context, any coding sequence encoding at least one SARS associated virus S, preferably a SARS-CoV-1 S antigenic protein as defined herein, or fragments and variants thereof may be understood as suitable coding sequence and may therefore be comprised in the nucleic acid.

In preferred embodiments, the (additional) nucleic acid comprises or consists of at least one coding sequence encoding at least one antigenic peptide or protein selected or derived from a SARS-associated virus as defined herein, preferably encoding any one of SEQ ID NOs: 14906-14950; SEQ ID NOs: 1-152, 1448-1548 of WO2018115527; SEQ ID NOs: 29, 32 or 34 of WO2017070626; SEQ ID NOs: 7 or 30 of WO2018081318, or fragments of variants thereof. It has to be understood that, on nucleic acid level, any sequence (DNA or RNA sequence) which encodes an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 14906-14950; SEQ ID NOs: 1-152, 1448-1548 of WO2018115527; SEQ ID NOs: 29, 32 or 34 of WO2017070626; SEQ ID NOs: 7 or 30 of WO2018081318, or fragments or variants thereof, may be selected and may accordingly be understood as suitable coding sequence of the invention. Further information regarding said amino acid sequences is also provided in Table 4 (see rows 1 to 45 of Column A and B), Table 5, and under <223> identifier of the ST25 sequence listing of respective sequence SEQ ID NOs.

In preferred embodiments, the (additional) nucleic acid comprises a coding sequence that comprises at least one of the nucleic acid sequences encoding a SARS-associated virus S antigen, preferably a SARS-CoV-1 S antigen, being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences selected from SEQ ID NOs: 14951-15220, or a fragment or a fragment or variant of any of these sequences. Further information regarding said nucleic acid sequences is also provided in Table 4 (see rows 1 to 45), Table 5, and under <223> identifier of the ST25 sequence listing of respective sequence SEQ ID NOs.

In preferred embodiments, the at least one coding sequence of the (additional) nucleic acid is a codon modified coding sequence as defined herein, wherein the amino acid sequence, that is the SARS-associated virus S, encoded by the at least one codon modified coding sequence, is preferably not being modified compared to the amino acid sequence encoded by the corresponding wild type or reference coding sequence.

In particularly preferred embodiments, the at least one coding sequence of the nucleic is a codon modified coding sequence, wherein the codon modified coding sequence is selected a G/C optimized coding sequence, a human codon usage adapted coding sequence, or a G/C modified coding sequence.

Preferred nucleic acid sequences, including particularly preferred mRNA sequences, are provided in Table 5 (column C and D). Therein, each row represents a specific suitable SARS-associated virus spike protein construct of the invention (compare with Table 4), wherein the description of the SARS-associated virus spike protein construct is indicated in column A of Table 5 and the SEQ ID NOs of the amino acid sequence of the respective SARS-associated virus construct is provided in column B. The corresponding SEQ ID NOs of the coding sequences encoding the respective SARS-associated virus constructs are provided in Table 4. Further information is provided under <223> identifier of the respective SEQ ID NOs in the sequence listing.

TABLE 5
Nucleic acid, preferably mRNA constructs encoding SARS-associated or SARS-COV-1 spike

| A | B | C | D |
|---|---|---|---|
| SARS-COV spike protein (1-1255) | 14906 | 15041, 15086 | 15131, 15176 |
| SARS-COV spike protein (1-1255)(K968P_V969P) | 14907 | 15042, 15087 | 15132, 15177 |
| SARS-COV spike protein (1-17_747-1255) | 14908 | 15043, 15088 | 15133, 15178 |
| SARS-COV/Tor2 spike protein (1-1255) | 14909 | 15044, 15089 | 15134, 15179 |
| SARS-COV/Tor2 spike protein (1-1255)(K968P_V969P) | 14910 | 15045, 15090 | 15135, 15180 |
| SARS-COV/Tor2 spike protein (variant) | 14911 | 15046, 15091 | 15136, 15181 |
| SARS-COV/Tor2 spike protein (variant) | 14912 | 15047, 15092 | 15137, 15182 |
| HCoV/OC43 spike protein (1-1353) | 14913 | 15048, 15093 | 15138, 15183 |
| HCoV/OC43 spike protein (1-1353)(A1070P_L1071P) | 14914 | 15049, 15094 | 15139, 15184 |
| HCoV/OC43/1783A_10 spike protein (1-1362) | 14915 | 15050, 15095 | 15140, 15185 |
| HCoV/OC43/1783A_10 spike protein (1-1362)(A1079P_L1080P) | 14916 | 15051, 15096 | 15141, 15186 |
| HCoV/OC43/1783A_10 spike protein (variant) | 14917 | 15052, 15097 | 15142, 15187 |
| HCoV/OC43/1783A_10 spike protein (variant) | 14918 | 15053, 15098 | 15143, 15188 |
| HCoV/HKU1/N5 spike protein (1-1351) | 14919 | 15054, 15099 | 15144, 15189 |
| HCoV/HKU1/N5 spike protein (1-1351)(N1067P_L1068P) | 14920 | 15055, 15100 | 15145, 15190 |
| HCOV/HKU1/N5 spike protein (variant) | 14921 | 15056, 15101 | 15146, 15191 |
| HCoV/HKU1/N5 spike protein (variant) | 14922 | 15057, 15102 | 15147, 15192 |
| HCoV/229E/BN1/GER/2015 spike protein (1-1171) | 14923 | 15058, 15103 | 15148, 15193 |
| HCoV/229E/BN1/GER/2015 spike protein (1-1171)(I869P_I870P) | 14924 | 15059, 15104 | 15149, 15194 |
| HCoV/229E/BN1/GER/2015 spike protein (variant) | 14925 | 15060, 15105 | 15150, 15195 |
| HCoV/229E/BN1/GER/2015 spike protein (variant) | 14926 | 15061, 15106 | 15151, 15196 |
| HCoV/NL63/RPTEC/2004 spike protein (1-1356) | 14927 | 15062, 15107 | 15152, 15197 |
| HCoV/NL63/RPTEC/2004 spike protein (1-1356)(S1052P_I1053P) | 14928 | 15063, 15108 | 15153, 15198 |
| HCoV/NL63/RPTEC/2004 spike protein (variant) | 14929 | 15064, 15109 | 15154, 15199 |
| HCoV/NL63/RPTEC/2004 spike protein (variant) | 14930 | 15065, 15110 | 15155, 15200 |
| Bat SARS-like CoV/WIV1 spike protein (1-1256) | 14931 | 15066, 15111 | 15156, 15201 |
| Bat SARS-like CoV/WIV1 spike protein (1-1256)(K969P_V970P) | 14932 | 15067, 15112 | 15157, 15202 |
| Bat SARS-like CoV/WIV1 spike protein (variant) | 14933 | 15068, 15113 | 15158, 15203 |
| Bat SARS-like CoV/WVIV1 spike protein (variant) | 14934 | 15069, 15114 | 15159, 15204 |
| BatCoV/HKU9-1 BF_0051 spike protein (1-1274) | 14935 | 15070, 15115 | 15160, 15205 |
| BatCoV/HKU9-1 BF_0051 spike protein (1-1274)(G983P_L984P) | 14936 | 15071, 15116 | 15161, 15206 |
| BatCoV/HKU9-1 BF_0051 spike protein (variant) | 14937 | 15072, 15117 | 15162, 15207 |
| BatCoV/HKU9-1 BF_0051 spike protein (variant) | 14938 | 15073, 15118 | 15163, 15208 |
| PDCoV/Swine/Thailand/S5011/2015 spike protein (1-1160) | 14939 | 15074, 15119 | 15164, 15209 |
| PDCoV/Swine/Thailand/S5011/2015 spike protein (1-1160)((E855P_V856P) | 14940 | 15075, 15120 | 15165, 15210 |
| PDCoV/Swine/Thailand/S5011/2015 spike protein (variant) | 14941 | 15076, 15121 | 15166, 15211 |
| PDCoV/Swine/Thailand/S5011/2015 spike protein (variant) | 14942 | 15077, 15122 | 15167, 15212 |
| PEDV/NPL-PEDv/2013/P10 spike protein (1-1386) | 14943 | 15078, 15123 | 15168, 15213 |
| PEDV/NPL-PEDv/2013/P10 spike protein (1-1386)(I1076P_L1077P) | 14944 | 15079, 15124 | 15169, 15214 |
| PEDV/NPL-PEDv/2013/P10 spike protein (variant) | 14945 | 15080, 15125 | 15170, 15215 |
| PEDV/NPL-PEDv/2013/P10 spike protein (variant) | 14946 | 15081, 15126 | 15171, 15216 |
| MHV/S spike protein (1-1361) | 14947 | 15082, 15127 | 15172, 15217 |
| MHV/S spike protein (1-1361)(A1073P_L1074P) | 14948 | 15083, 15128 | 15173, 15218 |
| MHV/S spike protein (variant) | 14949 | 15084, 15129 | 15174, 15219 |
| MHV/S spike protein (variant) | 14950 | 15085, 15130 | 15175, 15220 |

In preferred embodiments, the (additional) nucleic acid, preferably the RNA, comprises or consists of a nucleic acid sequence encoding a SARS-associated virus spike protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 15041-15220 or a fragment or variant of any of these sequences. Further information regarding respective nucleic acid sequences is provided under <223> identifier of the respective SEQ ID NO in the sequence listing, and in Table 5 (see in particular Column C and D). Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides.

Nucleic Acid Encoding MERS-CoV Spike Protein

In a particularly preferred embodiment, the Coronavirus spike protein (S) is selected or derived from at least one MERS-associated virus spike protein, preferably a MERS-CoV spike protein (S1, S2, or S1 and S2), or an immunogenic fragment or immunogenic variant thereof.

In a particularly preferred embodiment, the Coronavirus spike protein (S) is selected from a MERS-CoV virus.

It has to be understood that generic embodiments and features that have been described paragraph “Nucleic acid encoding a Coronavirus spike protein” may also apply to a nucleic acid encoding a MERS-CoV spike protein.

Suitable antigenic peptide or protein sequences that are provided by the (additional) nucleic acid are disclosed in Table 6, rows 1 to 16, Column A and B. In addition, further information regarding said suitable antigenic peptide or protein sequences selected or derived from MERS-associated virus, preferably a MERS-CoV are provided under <223> identifier of the ST25 sequence listing.

In particularly preferred embodiments, the encoded at least one antigenic peptide or protein comprises or consists of a MERS-associated virus spike protein (S), preferably a MERS-CoV spike protein (S), wherein the spike protein (S) comprises or consists of a spike protein fragment S1, or an immunogenic fragment or immunogenic variant thereof.

In particularly preferred embodiments, the encoded at least one antigenic peptide or protein comprises or consists of a full-length MERS-associated virus spike protein (S), preferably a MERS-CoV spike protein (S) or an immunogenic fragment or immunogenic variant of any of these.

In particularly preferred embodiments, the MERS-associated virus spike protein (S), preferably a MERS-CoV spike protein (S) that is provided by the nucleic acid is designed or adapted to stabilize the antigen in pre-fusion conformation. A pre-fusion conformation is particularly advantageous in the context of an efficient Coronavirus vaccine, as several potential epitopes for neutralizing antibodies may merely be accessible in said pre-fusion protein conformation. Furthermore, remaining of the protein in the pre-fusion conformation is aimed to avoid immunopathological effects, like e.g. enhanced disease and/or antibody dependent enhancement (ADE).

Accordingly, in preferred embodiments, the (additional) nucleic acid of comprises at least one coding sequence encoding at least one antigenic peptide or protein that is selected or derived from an MERS-associated virus spike protein (S), preferably a MERS-CoV-1 spike protein (S), wherein the spike protein (S) is a pre-fusion stabilized spike protein (S_stab). Suitably, said pre-fusion stabilized spike protein comprises at least one pre-fusion stabilizing mutation.

Stabilization of the MERS-CoV spike protein may be obtained by substituting at least one amino acids at position V1060 and/or L1061 with amino acids that stabilize the spike protein in a perfusion conformation (amino acid positions according to reference SEQ ID NO: 14794).

In embodiments, the pre-fusion stabilizing mutation of MERS-CoV spike protein comprises an amino acid substitution at position V1060, wherein the amino acids V1060 is substituted with one selected from A, I, L, M, F, V, G, or P (amino acid positions according to reference SEQ ID NO: 14794), preferably wherein the amino acids V1060 is substituted with P. In embodiments, the pre-fusion stabilizing mutation comprises an amino acid substitution at position L1061, wherein the amino acids L1061 is substituted with one selected from A, I, L, M, F, V, G, or P (amino acid positions according to reference SEQ ID NO: 14794), preferably wherein the amino acids L1061 is substituted with P.

Accordingly, in preferred embodiments, the pre-fusion stabilized spike protein (S_stab) of MERS-CoV comprises at least one pre-fusion stabilizing mutation, wherein the at least one pre-fusion stabilizing mutation comprises the following amino acid substitutions: V1060P and L1061P (amino acid positions according to reference SEQ ID NO: 14794).

Any MERS-CoV spike protein or fragments or variants thereof can be chosen by the skilled person to introduce such amino acid changes, preferably amino acid substitutions: V1060P and L1061P (amino acid positions according to reference SEQ ID NO: 14794).

Any MERS-CoV spike protein or fragments or variants thereof can be chosen by the skilled person to introduce such amino acid changes (e.g. such a double Proline mutation). Examples comprise MERS-CoV/MERS-CoV-Jeddah-human-1 spike protein (1-1353)(V1060P_L1061P), MERS-CoV/AI-Hasa_4_2013 spike protein (1-1353)(V1060P_L1061P), MERS-CoV/Riyadh_14_2013 spike protein (1-1353)(V1060P_L1061P), MERS-CoV/England 1 spike protein (1-1353)(V1060P_L1061P).

In embodiments, the at least one pre-fusion stabilizing mutation of MERS-CoV spike protein comprises a cavity filling mutation. In some embodiments, the cavity filling substitutions to stabilize the MERS-CoV S ectodomain the prefusion conformation, may be selected from the following amino acid substitutions: N1072F and A1083I; N1072F and L1086F; N1072F and V1087I; N1072F and E1090I; T1076F and A1083I; T1076F and L1086F; T1076F and V1087I; T1076F and EI 0901; T1076I and A1083I; T1076I and L1086F; T1076I and V1087I; T1076I and E10901; A1018V; or A1018I.

In embodiments, the at least one pre-fusion stabilizing mutation of MERS-CoV spike protein comprises a mutated protonation site (R1020Q).

In embodiments, the at least one pre-fusion stabilizing mutation of MERS-CoV spike protein comprises a repacking substitution to stabilize the S ectodomain the prefusion conformation, such as one of: E793M and K1102F; E793M, K1102F, and H1138F; D1068M and R1069W; A1083L; A1083L and V1087I; A1083L, V1087, and E1090L; A834L and Q1084M; Q1066M; S454F; R921W; S612F and G1052F; or P476V, T477A, and R1057W.

In embodiments, the at least one pre-fusion stabilizing mutation of the MERS-CoV spike protein comprises an artificial intramolecular disulfide bond. Such an artificial intramolecular disulfide bond can be introduced to further stabilize the membrane distal portion of the MERS-CoV spike protein (including the N-terminal region) in the pre-fusion conformation; that is, in a conformation that specifically binds to one or more pre-fusion specification antibodies, and/or presents a suitable antigenic site that is present on the pre-fusion conformation but not in the post fusion conformation of the MERS-CoV spike protein.

In some embodiments, the disulfide bond substitutions to stabilize the MERS-CoV S ectodomain the prefusion conformation, may be selected from the following amino acid substitutions: T63C and V631C; T63C and Q638C; Q733C and D940C; S676C and D910C; V1087C (which forms a disulfide bond with a cysteine present in the native sequence); A432C and L1058C; or A432C and D1059C to stabilize the S ectodomain the prefusion conformation.

Preferred antigenic peptide or proteins selected or derived from a MERS-CoV as defined above are provided in Table 6 (rows 1 to 16). Therein, each row 1 to 16 corresponds to a suitable MERS-CoV S constructs. Column A of Table 6 provides a short description of suitable MERS-CoV S antigen constructs. Column B of Table 6 provides protein (amino acid) SEQ ID NOs of respective MERS-CoV S constructs. Column D of Table 6 provides SEQ ID NO of the corresponding G/C optimized nucleic acid coding sequences (opt1, gc). Column E of Table 6 provides SEQ ID NO of the corresponding human codon usage adapted nucleic acid coding sequences (opt 3, human).

Notably, the description of the invention explicitly includes the information provided under <223> identifier of the ST25 sequence listing of the present application. Preferred nucleic acid constructs comprising coding sequences of Table 6, e.g. mRNA sequences comprising the coding sequences of Table 6 are provided in Table 7.

TABLE 6
Preferred MERS-COV constructs (amino acid sequences and nucleic acid coding sequences);

| row | A | B | D | E |
|---|---|---|---|---|
| 1 | MERS-COV/MERS-COV-Jeddah-human-1 spike protein (1-1353) | 14794 | 14810 | 14826 |
| 2 | MERS-COV/MERS-COV-Jeddah-human-1 | 14795 | 14811 | 14827 |
| | spike protein (1-1353)(V1060P_L1061P) | | | |
| 3 | MERS-COV/Al-Hasa_4_2013 spike protein (1-1353) | 14796 | 14812 | 14828 |
| 4 | MERS-COV/Al-Hasa_4_2013 spike protein (1-1353)(V1060P_L1061P) | 14797 | 14813 | 14829 |
| 5 | MERS-COV/Riyadh_14_2013 spike protein (1-1353) | 14798 | 14814 | 14830 |
| 6 | MERS-COV/Riyadh_14_2013 spike protein (1-1353)(V1060P_L1061P) | 14799 | 14815 | 14831 |
| 7 | MERS-COV/Riyadh_14_2013 spike protein (1-1353)(R1020Q) | 14800 | 14816 | 14832 |
| 8 | MERS-COV/Riyadh_14_2013 spike protein (1-17_726-1296)(R1020Q) | 14801 | 14817 | 14833 |
| 9 | MERS-COV/Riyadh_14_2013 spike protein (1-17_726- | 14802 | 14818 | 14834 |
| | 1296)(R1020Q)_LeucineZipper | | | |
| 10 | MERS-COV/England 1 spike protein (1-1353) | 14803 | 14819 | 14835 |
| 11 | MERS-COV/England 1 spike protein (1-1353)(V1060P_L1061P) | 14804 | 14820 | 14836 |
| 12 | MERS-COV/England 1 spike protein (variant) | 14805 | 14821 | 14837 |
| 13 | MERS-COV/England 1 spike protein (variant) | 14806 | 14822 | 14838 |
| 14 | MERS-COV/England 1 spike protein (variant) | 14807 | 14823 | 14839 |
| 15 | MERS-COV/England 1 spike protein (variant) | 14808 | 14824 | 14840 |
| 16 | MERS-COV/England 1 spike protein (variant) | 14809 | 14825 | 14841 |

In preferred embodiments, the at least one antigenic peptide or protein selected or derived from MERS-CoV spike protein encoded by the at least one nucleic acid comprises or consists of at least one of the amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 14794-14809 or an immunogenic fragment or immunogenic variant of any of these. Further information regarding said amino acid sequences is also provided in Table 6 (see rows 1 to 16 of Column A and B), and under <223> identifier of the ST25 sequence listing of respective sequence SEQ ID NOs.

In further embodiments, the at least one antigenic peptide or protein selected or derived from at least one MERS-CoV spike comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 1-152 or 1448 to 1548 of published PCT application WO2018115527, or an immunogenic fragment or immunogenic variant of any of these. Accordingly, SEQ ID NOs: 1-152 or 1448 to 1548 of WO2018115527 and the corresponding disclosure relating thereto (e.g. information in the respective sequence listing, column 1 or 2 of Tables 1-4 and Table 7) are herewith incorporated by reference. In further embodiments, the at least one antigenic peptide or protein selected or derived from at least one MERS-CoV spike comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 2-4, 28-29 of published PCT application WO2018081318, or an immunogenic fragment or immunogenic variant of any of these. Accordingly, SEQ ID NOs: 2-4, 28-29 of WO2018081318 and the corresponding disclosure relating thereto are herewith incorporated by reference. In further embodiments, the at least one antigenic peptide or protein selected or derived from at least one MERS-CoV spike comprises or consists of at least one of the amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NO: 24-28 or 33 of published PCT application WO2017070626, or an immunogenic fragment or immunogenic variant of any of these. Accordingly, SEQ ID NO: 24-28 or 33 of WO2017070626 and the corresponding disclosure relating thereto are herewith incorporated by reference. Further suitable antigenic peptide or proteins selected or derived from MERS-CoV spike can be selected or derived from Table 12 of WO2017070626. Accordingly, the full content of Table 12 of WO2017070626 herewith incorporated by reference.

In preferred embodiments, the at least one antigenic peptide or protein (pre-fusion stabilized spike protein (S_stab)) selected or derived from MERS-CoV encoded by the at least one (additional) nucleic acid comprises or consists of at least one of the amino acid sequences being identical or at least 50%, 60%, 70%, 80%, 85%, 86%, 87%, 88%, 89%, 90%, 91%, 92%, 93%, 94%, 95%, 96%, 97%, 98%, or 99% identical to any one of SEQ ID NOs: 14795, 14797, 14799-14802, 14804 or an immunogenic fragment or immunogenic variant of any of these. Further information regarding said amino acid sequences is also provided in Table 6 (Column A and B), and under <223> identifier of the ST25 sequence listing of respective sequence SEQ ID NOs.

According to preferred embodiments, the (additional) nucleic acid comprises at least one coding sequence encoding at least one antigenic peptide or protein derived from MERS-CoV as defined above, or fragments and variants thereof. In that context, any coding sequence encoding at least one MERS-CoV antigenic protein as defined herein, or fragments and variants thereof may be understood as suitable coding sequence and may therefore be comprised in the nucleic acid.

In preferred embodiments, the (additional) nucleic acid comprises or consists of at least one coding sequence encoding at least one antigenic peptide or protein selected or derived from a MERS-CoV as defined herein, preferably encoding any one of SEQ ID NOs: 14794-14809; SEQ ID NOs: 1-152, 1448-1548 of WO2018115527; SEQ ID NOs: 2-4, 28-29 of WO2018081318; SEQ ID NO: 24-28 or 33 of WO2017070626, or fragments of variants thereof. It has to be understood that, on nucleic acid level, any sequence (DNA or RNA sequence) which encodes an amino acid sequences being identical or at least 70%, 80%, 85%, 86%, 87%, 88%, 89%, 90%, 91%, 92%, 93%, 94%, 95%, 96%, 97%, 98%, or 99% identical to any one of SEQ ID NOs: 14794-14809; SEQ ID NOs: 1-152, 1448-1548 of WO2018115527; SEQ ID NOs: 2-4, 28-29 of WO2018081318; SEQ ID NO: 24-28 or 33 of WO2017070626, or fragments or variants thereof, may be selected and may accordingly be understood as suitable coding sequence of the invention. Further information regarding said amino acid sequences is also provided in Table 6, Table 7, and under <223> identifier of the ST25 sequence listing of respective sequence SEQ ID NOs.

In preferred embodiments, the (additional) nucleic acid comprises a coding sequence that comprises at least one of the nucleic acid sequences encoding a MERS-CoV spike antigen being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences SEQ ID NOs: 14810-14905 or a fragment or a fragment or variant of any of these sequences. Further information regarding said nucleic acid sequences is also provided in Table 6, Table 7, and under <223> identifier of the ST25 sequence listing of respective sequence SEQ ID NOs.

In further embodiments, the at least one coding sequence of the at least one (additional) nucleic acid comprises or consists at least one nucleic acid sequence encoding a MERS-CoV spike antigen being identical or at least 70%, 80%, 85%, 86%, 87%, 88%, 89%, 90%, 91%, 92%, 93%⁰, 94%, 95%, 96%, 97%, 98%, or 99% identical to any one of the nucleic acid sequences SEQ ID NO: 153-304 or 1549-1649, 305-1368,1650-2356, 2365, 2366, 2373-2378 of published PCT application WO2018115527, or an immunogenic fragment or immunogenic variant of any of these. Notably, SEQ ID NO: 153-304 or 1549-1649, 305-1368, 1650-2356, 2365, 2366, 2373-2378 of WO2018115527 and the corresponding disclosure relating thereto (e.g. information in the respective sequence listing, column 4 or 4 of Tables 1-4 and Table 7) are herewith incorporated by reference. In further embodiments, the at least one coding sequence of the at least one (additional) nucleic acid comprises or consists at least one nucleic acid sequence encoding a MERS-CoV spike antigen being identical or at least 70%, 80%, 85%, 86%, 87%, 88%, 89%, 90%, 91%, 92%, 93%, 94%, 95%, 96%, 97%, 98%, or 99% identical to any one of the nucleic acid sequences SEQ ID NO: 20-23, 65-68 of published PCT application WO2017070626, or an immunogenic fragment or immunogenic variant of any of these. Notably, SEQ ID NO: 20-23, 65-68 of WO2017070626 and the corresponding disclosure relating thereto (e.g. information in the respective sequence listing, Tables 1-4 and Table 7) are herewith incorporated by reference.

In preferred embodiments, the at least one coding sequence of the (additional) nucleic acid is a codon modified coding sequence as defined herein, wherein the amino acid sequence, that is the MERS-CoV peptide or protein, encoded by the at least one codon modified coding sequence, is preferably not being modified compared to the amino acid sequence encoded by the corresponding wild type or reference coding sequence.

In particularly preferred embodiments, the at least one coding sequence of the (additional) nucleic acid is a codon modified coding sequence, wherein the codon modified coding sequence is selected a G/C optimized coding sequence, a human codon usage adapted coding sequence, or a G/C modified coding sequence.

Preferred nucleic acid sequences, including particularly preferred mRNA sequences, are provided in Table 7 (column C and D). Therein, each row represents a specific suitable MERS-CoV spike construct of the invention (compare with Table 6), wherein the description of the MERS-CoV spike construct is indicated in column A of Table 7 and the SEQ ID NOs of the amino acid sequence of the respective MERS-CoV spike construct is provided in column B. The corresponding SEQ ID NOs of the coding sequences encoding the respective MERS-CoV spike constructs are provided in in Table 6. Further information is provided under <223> identifier of the respective SEQ ID NOs in the sequence listing.

TABLE 7
Nucleic acid, preferably mRNA constructs encoding MERS-COV spike

| A | B | C | D |
|---|---|---|---|
| MERS-COV/MERS-COV-Jeddah-human-1 spike protein (1-1353) | 14794 | 14842, 14858 | 14874, 14890 |
| MERS-COV/MERS-COV-Jeddah-human-1 spike protein (1- | 14795 | 14843, 14859 | 14875, 14891 |
| 1353)(V1060P_L1061P) | | | |
| MERS-COV/AI-Hasa_4_2013 spike protein (1-1353) | 14796 | 14844, 14860 | 14876, 14892 |
| MERS-COV/Al-Hasa_4_2013 spike protein (1-1353)(V1060P_L1061P) | 14797 | 14845, 14861 | 14877, 14893 |
| MERS-COV/Riyadh_14_2013 spike protein (1-1353) | 14798 | 14846, 14862 | 14878, 14894 |
| MERS-COV/Riyadh_14_2013 spike protein (1-1353)(V1060P_L1061P) | 14799 | 14847, 14863 | 14879, 14895 |
| MERS-COV/Riyadh_14_2013 spike protein (1-1353)(R1020Q) | 14800 | 14848, 14864 | 14880, 14896 |
| MERS-COV/Riyadh_14_2013 spike protein (1-17_726-1296)(R1020Q) | 14801 | 14849, 14865 | 14881, 14897 |
| MERS-COV/Riyadh_14_2013 spike protein (1-17_726- | 14802 | 14850, 14866 | 14882, 14898 |
| 1296)(R1020Q)_LeucineZipper | | | |
| MERS-COV/England 1 spike protein (1-1353) | 14803 | 14851, 14867 | 14883, 14899 |
| MERS-COV/England 1 spike protein (1-1353)(V1060P_L1061P) | 14804 | 14852, 14868 | 14884, 14900 |
| MERS-COV/England 1 spike protein (variant) | 14805 | 14853, 14869 | 14885, 14901 |
| MERS-COV/England 1 spike protein (variant) | 14806 | 14854, 14870 | 14886, 14902 |
| MERS-COV/England 1 spike protein (variant) | 14807 | 14855, 14871 | 14887, 14903 |
| MERS-COV/England 1 spike protein (variant) | 14808 | 14856, 14872 | 14888, 14904 |
| MERS-COV/England 1 spike protein (variant) | 14809 | 14857, 14873 | 14889, 14905 |

In preferred embodiments, the (additional) nucleic acid, preferably the RNA, comprises or consists of a nucleic acid sequence encoding a MERS-CoV spike protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence seceded from SEQ ID NOs: 14842-14905 or a fragment or variant of any of these sequences. Further information regarding respective nucleic acid sequences is provided under <223> identifier of the respective SEQ ID NO in the sequence listing, and in Table 7 (see in particular Column C and D). Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides

In further embodiments, the (additional) nucleic acid, preferably the RNA, comprises or consists of a nucleic acid sequence encoding a MERS-CoV spike protein which is identical or at least 70%, 80%, 85%, 86%, 87%, 88%, 89%, 90%, 91%, 92%, 93%, 94%, 95%, 96%, 97%, 98%, or 99% identical to a nucleic acid sequence selected from SEQ ID NOs: 2373-2378 of WO2018115527 and SEQ ID NOs: 65-68 of WO2017070626, or a fragment or variant of any of these sequences. Further information regarding respective nucleic acid sequences is provided under <223> identifier of the respective SEQ ID NOs in the sequence listing of WO2018115527 or WO2017070626, and in Tables 1-4 and Table 7 of WO2018115527. Optionally, said nucleic acid sequences comprise a cap1 structure as defined herein, and/or at least one, preferably all uracil nucleotides in said RNA sequences are replaced by pseudouridine (ψ) nucleotides and/or N1-methylpseudouridine (m1ψ) nucleotides.

Combinations of M, N, NSP3, NSP4, NSP6, NSP13, NSP14, ORF3A, ORF8, E and/or S (Multivalent Composition)

In preferred embodiments, the pharmaceutical composition comprises at least two nucleic acids, wherein the at least two nucleic acids are selected from (a) at least one nucleic acid encoding Coronavirus M, N, M, N, NSP3, NSP4, NSP6, NSP13, NSP14, ORF3A, ORF8, E (as defined herein), and (b) at least one (additional) nucleic acid encoding Coronavirus S.

Accordingly, in embodiments, the pharmaceutical composition comprises 2, 3, 4, 5, 6, 7, or more of M, N, NSP3, NSP4, NSP6, NSP13, NSP14, ORF3A, ORF8, E, additionally comprising S, wherein the antigenic peptide or proteins are selected or derived from the same Coronavirus, preferably from SARS-CoV-2 or a SARS-Cov-2 variant.

In embodiments, the pharmaceutical composition comprises 2, 3, 4, 5, 6, 7, or more of M, N, NSP3, NSP4, NSP6, NSP13, NSP14, ORF3A, ORF8, E, additionally comprising S, wherein the antigenic peptide or proteins are selected or derived from different Coronaviruses, preferably different pandemic Coronaviruses, e.g. SARS-CoV-2, SARS-CoV-1, and/or MERS-CoV or more preferably derived from different SARS-CoV-2 and SARS-CoV-2 variants.

Suitable combinations of the above mentioned nucleic acids encoding Coronavirus antigens, preferably SARS-CoV-2 or SARS-CoV2 variant antigens, are provided in the following.

In preferred embodiments, the pharmaceutical composition comprises at least two nucleic acid sequences (encoding M, N, NSP3, NSP4, NSP6, NSP13, NSP14, ORF3A, ORF8, E, and/or S) according to the following combinations:
M and S; N and S; NSP3 and S; NSP4 and S; NSP6 and S; NSP14 and S; NSP14 and S, ORF3A and S; or ORF8 and S.

In preferred embodiments, the pharmaceutical composition comprises at least three nucleic acid sequences (encoding M, N, NSP3, NSP4, NSP6, NSP13, NSP14, ORF3A, ORF8, and/or S) according to the following combinations:
M and N and S; M and NSP3 and S; M and NSP4 and S; M and NSP6 and S; M and SNP13 and S; M and SNSP14 and S; M and ORF3A and S; M and ORF8 and S; N and NSP3 and S; N and NSP4 and S; N and NSP6 and S; N and NSP13 and S; N and NSP14 and S; N and ORF3A and S; N and ORF8 and S; NSP3 and NSP4 and S; NSP3 and NSP6 and S; NSP3 and NSP13 and S; NSP3 and NSP14 and S; NSP3 and ORF3A and S; NSP3 and ORF8 and S; NSP4 and NSP6 and S; NSP4 and NSP13 and S; NSP4 and NSP14 and S; NSP4 and ORF3A and S; NSP4 and ORF8 and S; NSP6 and NSP13 and S; NSP6 and NSP14 and S; NSP6 and ORF3A and S; NSP6 and ORF8 and S; NSP13 and NSP14 and S; NSP13 and ORF3A and S; NSP13 and ORF8 and S; NSP14 and ORF3A and S; NSP14 and ORF8 and S; or ORF3A and ORF8 and S.

In preferred embodiments, the pharmaceutical composition comprises at least four nucleic acid sequences (encoding M, N, NSP3, NSP4, NSP6, NSP13 and NSP14, ORF3A, ORF8, and/or S) according to the following combinations:
M and N and NSP3 and S; M and N and NSP4 and S; M and N and NSP6 and S; M and N and NSP13 and S; M and N and NSP14 and S; M and N and ORF3A and S; M and N and ORF8 and S; M and NSP3 and NSP4 and S; M and NSP3 and NSP6 and S; M and NSP3 and NSP13 and S; M and NSP3 and NSP14 and S; M and NSP3 and ORF3A and S; M and NSP3 and ORF8 and S; M and NSP4 and NSP6 and S; M and NSP4 and SNP13 and S; M and NSP4 and NSP14 and S; M and NSP4 and ORF3A and S; M and NSP4 and ORF8 and S; M and NSP6 and NSP13 and S; M and NSP6 and NSP14 and S; M and NSP6 and ORF3A and S; M and NSP6 and ORF8 and S; M and NSP13 and NSP14 and S; M and NSP13 and ORF3a and S; M and NSP13 and ORF8 and S; M and NSP14 and ORF3A and S; M and NSP14 and ORF8 and S; M and ORF3A and ORF8 and S; N and NSP3 and NSP4 and S; N and NSP3 and NSP6 and S; N and NSP3 and NSP13 and S; N and NSP3 and NSP14 and S; N and NSP3 and ORF3A and S; N and NSP3 and ORF8 and S; N and NSP4 and NSP6 and S; N and NSP4 and NSP13 and S; N and NSP4 and NSP14 and S; N and NSP4 and ORF3A and S; N and NSP4 and ORF8 and S; N and NSP6 and ORF3A and S; N and NSP6 and ORF8 and S; N and NSP6 and NSP13 and S; N and NSP6 and NSP14 and S; N and NSP13 and NSP14 and S; N and NSP13 and ORF3A and S; N and NSP13 and ORF8 and S; N and NSP14 and ORF3a and S; N and NSP14 and ORF8 and S; N and ORF3A and ORF8 and S; NSP3 and NSP4 and NSP6 and S; NSP3 and NSP4 and NSP13 and S; NSP3 and NSP4 and NSP14 and S; NSP3 and NSP4 and ORF3A and S; NSP3 and NSP4 and ORF8 and S; NSP3 and NSP6 and NSP13 and S; NSP3 and NSP6 and NSP14 and S; NSP3 and NSP6 and ORF3A and S; NSP3 and NSP6 and ORF8 and S; NSP3 and NSP13 and NSP14 and S; NSP3 and NSP13 and ORF3A and S; NSP3 and NSP13 and ORF8 and S; NSP3 and ORF3A and ORF8 and S; NSP4 and NSP6 and NSP13 and S; NSP4 and NSP6 and NSP14 and S; NSP4 and NSP6 and ORF3A and S; NSP4 and NSP6 and ORF8 and S; NSP4 and NSP13 and NSP14 and S; NSP4 and NSP13 and ORF3A and S; NSP4 and NSP13 an ORF8 and S; NSP4 and NSP14 and ORF3A and S; NSP4 and NSP14 and ORF8 and S; NSP4 and ORF3A and ORF8 and S; NSP6 and NSP13 and NSP14 and S; NSP6 and NSP13 and ORF3A and S; NSP6 and NSP13 and ORF8 and S; NSP6 and NSP14 and ORF3A and S; NSP6 and NSP14 and ORF8 and S; NSP6 and ORF3A and ORF8 and S; NSP13 and NSP14 and ORF3A and S; NSP13 and NSP14 and ORF8 and S; NSP13 and ORF3A and ORF8 and S; NSP14 and ORF3A and ORF8 and S.

In preferred embodiments, the pharmaceutical composition comprises at least five nucleic acid sequences (encoding M, N, NSP3, NSP4, NSP6, NSP13, NSP14, ORF3A, ORF8, and/or S) according to the following combinations:
NSP3 and NSP4 and NSP6 and M and S; NSP3 and NSP4 and ORF3A and M and S;
NSP3 and NSP4 and ORF8 and M and S; NSP3 and NSP6 and ORF3A and M and S;
NSP3 and NSP6 and ORF8 and M and S; NSP3 and ORF3A and ORF8 and M and S;
NSP4 and NSP6 and ORF3A and M and S; NSP4 and NSP6 and ORF8 and M and S;
NSP4 and ORF3A and ORF8 and M and S; NSP6 and ORF3A and ORF8 and M and S;
NSP3 and NSP4 and NSP6 and N and S; NSP3 and NSP4 and ORF3A and N and S;
NSP3 and NSP4 and ORF8 and N and S; NSP3 and NSP6 and ORF3A and N and S;
NSP3 and NSP6 and ORF8 and N and S; NSP3 and ORF3A and ORF8 and N and S;
NSP4 and NSP6 and ORF3A and N and S; NSP4 and NSP6 and ORF8 and N and S; or
NSP4 and ORF3A and ORF8 and N and S; NSP6 and ORF3A and ORF8 and N and S.

In preferred embodiments, the pharmaceutical composition comprises at least six nucleic acid sequences (encoding M, N, NSP3, NSP4, NSP6, ORF3A, ORF8, and/or S) according to the following combinations:
NSP3 and NSP4 and NSP6 and M and N and S; NSP3 and NSP4 and ORF3A and M and N and S;
NSP3 and NSP4 and ORF8 and M and N and S; NSP3 and NSP6 and ORF3A and M and N and S;
NSP3 and NSP6 and ORF8 and M and N and S; NSP3 and ORF3A and ORF8 and M and N and S;
NSP4 and NSP6 and ORF3A and M and N and S; NSP4 and NSP6 and ORF8 and M and N and S; or
NSP4 and ORF3A and ORF8 and M and N and S; NSP6 and ORF3A and ORF8 and M and N and S.

Nucleic Acid Features and Embodiments:

In the following, suitable features and embodiments relating to nucleic acid comprised in the pharmaceutical composition are further specified. It has to be understood that suitable features and embodiments provided herein may relate to any of the at least one nucleic acid comprising at least one coding sequence encoding at least one antigenic peptide or protein from at least one Coronavirus M, N, non-structural protein, and/or accessory protein as defined herein. Further, suitable features and embodiments provided herein may relate to any of the at least one (additional) nucleic acid comprising at least one coding sequence encoding at least one antigenic peptide or protein selected or derived from at least one Coronavirus spike protein (S) as defined herein.

In preferred embodiments, the at least one nucleic acid is an artificial nucleic acid, e.g. an artificial DNA or an artificial RNA.

The term “artificial nucleic acid” as used herein is intended to refer to a nucleic acid that does not occur naturally. In other words, an artificial nucleic acid may be understood as a non-natural nucleic acid molecule. Such nucleic acid molecules may be non-natural due to its individual sequence (e.g. G/C content modified coding sequence, UTRs) and/or due to other modifications, e.g. structural modifications of nucleotides. Typically, artificial nucleic acid may be designed and/or generated by genetic engineering to correspond to a desired artificial sequence of nucleotides. In this context, an artificial nucleic acid is a sequence that may not occur naturally, i.e. a sequence that differs from the wild type sequence/the naturally occurring sequence or reference sequence by at least one nucleotide (via e.g. codon modification as further specified below). The term “artificial nucleic acid” is not restricted to mean “one single molecule” but is understood to comprise an ensemble of essentially identical nucleic acid molecules. Accordingly, it may relate to a plurality of essentially identical nucleic acid molecules. The term “artificial nucleic acid” as used herein may for example relate to an artificial DNA or, preferably, to an artificial RNA.

In preferred embodiments, at least one nucleic acid, e.g. the DNA or RNA, is a modified and/or stabilized nucleic acid, preferably a modified and/or stabilized artificial nucleic acid.

According to preferred embodiments, at least one nucleic acid may thus be provided as a “stabilized artificial nucleic acid” or “stabilized coding nucleic acid” that is to say a nucleic acid showing improved resistance to in vivo degradation and/or a nucleic acid showing improved stability in vivo, and/or a nucleic acid showing improved translatability in vivo. In the following, specific suitable modifications/adaptations in this context are described which are suitably to “stabilize” the nucleic acid. Preferably, the nucleic acid of the present invention may be provided as a “stabilized RNA”, “stabilized coding RNA”, “stabilized DNA” or “stabilized coding DNA”.

In the following, suitable modifications are described that are capable of “stabilizing” the at least one nucleic acid.

In preferred embodiments, the at least one nucleic acid, e.g. the RNA or DNA, comprises at least one codon modified coding sequence.

In preferred embodiments, the at least one coding sequence of the at least one nucleic acid is a codon modified coding sequence. Suitably, the amino acid sequence encoded by the at least one codon modified coding sequence is not being modified compared to the amino acid sequence encoded by the corresponding wild type coding sequence or reference coding sequence.

The term “codon modified coding sequence” relates to coding sequences that differ in at least one codon (triplets of nucleotides coding for one amino acid) compared to the corresponding wild type or reference coding sequence. Suitably, a codon modified coding sequence in the context of the invention may show improved resistance to in vivo degradation and/or improved stability in vivo, and/or improved translatability in vivo. Codon modifications in the broadest sense make use of the degeneracy of the genetic code wherein multiple codons may encode the same amino acid and may be used interchangeably (cf. Table 8 or Table 1 of WO2020002525) to optimize/modify the coding sequence for in vivo applications.

In preferred embodiments, the at least one coding sequence of the at least one nucleic acid is a codon modified coding sequence, wherein the codon modified coding sequence is selected from C maximized coding sequence, CAI maximized coding sequence, human codon usage adapted coding sequence, G/C content modified coding sequence, and G/C optimized coding sequence, or any combination thereof.

In preferred embodiments, the at least one coding sequence of the at least one nucleic acid has a G/C content of at least about 50%, 55%, or 60%. In particular embodiments, the at least one coding sequence of the nucleic acid of has a G/C content of at least about 50%, 51%, 52%, 53%, 54%, 55%, 56%, 57%, 58%, 59%, 60%, 61%, 62%, 63%, 64%, 65%, 66%, 67%, 68%, 69%, or 70%.

When transfected into mammalian host cells, the at least one nucleic acid comprising a codon modified coding sequence has a stability of between 12-18 hours, or greater than 18 hours, e.g., 24, 36, 48, 60, 72, or greater than 72 hours and are capable of being expressed by the mammalian host cell (e.g. a muscle cell).

When transfected into mammalian host cells, the at least one nucleic acid comprising a codon modified coding sequence is translated into protein, wherein the amount of protein is at least comparable to, or preferably at least 10% more than, or at least 20% more than, or at least 30% more than, or at least 40% more than, or at least 50% more than, or at least 100% more than, or at least 200% or more than the amount of protein obtained by a naturally occurring or wild type or reference coding sequence transfected into mammalian host cells.

In embodiments, the at least one nucleic acid may be modified, wherein the C content of the at least one coding sequence may be increased, preferably maximized, compared to the C content of the corresponding wild type or reference coding sequence (herein referred to as “C maximized coding sequence”). The amino acid sequence encoded by the C maximized coding sequence of the nucleic acid is preferably not modified compared to the amino acid sequence encoded by the respective wild type or reference coding sequence. The generation of a C maximized nucleic acid sequences may suitably be carried out using a modification method according to WO2015062738. In this context, the disclosure of WO2015062738 is included herewith by reference.

In preferred embodiments, the at least one nucleic acid may be modified, wherein the G/C content of the at least one coding sequence may be optimized compared to the G/C content of the corresponding wild type or reference coding sequence (herein referred to as “G/C content optimized coding sequence”). “Optimized” in that context refers to a coding sequence wherein the G/C content is preferably increased to the essentially highest possible G/C content. The amino acid sequence encoded by the G/C content optimized coding sequence of the nucleic acid is preferably not modified as compared to the amino acid sequence encoded by the respective wild type or reference coding sequence. The generation of a G/C content optimized nucleic acid sequence (RNA or DNA) may be carried out using a method according to WO2002098443. In this context, the disclosure of WO2002098443 is included in its full scope in the present invention. Throughout the description, including the <223> identifier of the sequence listing, G/C optimized coding sequences are indicated by the abbreviations “opt1” or “gc”.

In preferred embodiments, the at least one nucleic acid may be modified, wherein the codons in the at least one coding sequence may be adapted to human codon usage (herein referred to as “human codon usage adapted coding sequence”). Codons encoding the same amino acid occur at different frequencies in humans. Accordingly, the coding sequence of the nucleic acid is preferably modified such that the frequency of the codons encoding the same amino acid corresponds to the naturally occurring frequency of that codon according to the human codon usage. For example, in the case of the amino acid Ala, the wild type or reference coding sequence is preferably adapted in a way that the codon “GCC” is used with a frequency of 0.40, the codon “GCT” is used with a frequency of 0.28, the codon “GCA” is used with a frequency of 0.22 and the codon “GCG” is used with a frequency of 0.10 etc. (see Table 8). Accordingly, such a procedure (as exemplified for Ala) is applied for each amino acid encoded by the coding sequence of the nucleic acid to obtain sequences adapted to human codon usage. Throughout the description, including the <223> identifier of the sequence listing, human codon usage adapted coding sequences are indicated by the abbreviation “opt3” or “human”.

TABLE 8
Human codon usage table with frequencies indicated for each amino acid

| Amino acid | codon | frequency | Amino acid | codon | frequency |
|---|---|---|---|---|---|
| Ala | GCG | 0.10 | Pro | CCG | 0.11 |
| Ala | GCA | 0.22 | Pro | CCA | 0.27 |
| Ala | GCT | 0.28 | Pro | CCT | 0.29 |
| Ala | GCC* | 0.40 | Pro | CCC* | 0.33 |
| Cys | TGT | 0.42 | Gln | CAG* | 0.73 |
| Cys | TGC* | 0.58 | Gln | CAA | 0.27 |
| Asp | GAT | 0.44 | Arg | AGG | 0.22 |
| Asp | GAC* | 0.56 | Arg | AGA* | 0.21 |
| Glu | GAG* | 0.59 | Arg | CGG | 0.19 |
| Glu | GAA | 0.41 | Arg | CGA | 0.10 |
| Phe | TTT | 0.43 | Arg | CGT | 0.09 |
| Phe | TTC* | 0.57 | Arg | CGC | 0.19 |
| Gly | GGG | 0.23 | Ser | AGT | 0.14 |
| Gly | GGA | 0.26 | Ser | AGC* | 0.25 |
| Gly | GGT | 0.18 | Ser | TCG | 0.06 |
| Gly | GGC* | 0.33 | Ser | TCA | 0.15 |
| His | CAT | 0.41 | Ser | TCT | 0.18 |
| His | CAC* | 0.59 | Ser | TCC | 0.23 |
| Ile | ATA | 0.14 | Thr | ACG | 0.12 |
| Ile | ATT | 0.35 | Thr | ACA | 0.27 |
| Ile | ATC* | 0.52 | Thr | ACT | 0.23 |
| Lys | AAG* | 0.60 | Thr | ACC* | 0.38 |
| Lys | AAA | 0.40 | Val | GTG* | 0.48 |
| Leu | TTG | 0.12 | Val | GTA | 0.10 |
| Leu | TTA | 0.06 | Val | GTT | 0.17 |
| Leu | CTG* | 0.43 | Val | GTC | 0.25 |
| Leu | CTA | 0.07 | Trp | TGG* | 1 |
| Leu | CTT | 0.12 | Tyr | TAT | 0.42 |
| Leu | CTC | 0.20 | Tyr | TAC* | 0.58 |
| Met | ATG* | 1 | Stop | TGA* | 0.61 |
| Asn | AAT | 0.44 | Stop | TAG | 0.17 |
| Asn | AAC* | 0.56 | Stop | TAA | 0.22 |

*most frequent human codon

In embodiments, the at least one nucleic acid may be modified, wherein the G/C content of the at least one coding sequence may be modified compared to the 010 content of the corresponding wild type or reference coding sequence (herein referred to as “G/C content modified coding sequence”). In this context, the terms “G/C optimization” or “G/C content modification” relate to a nucleic acid that comprises a modified, preferably an increased number of guanosine and/or cytosine nucleotides as compared to the corresponding wild type or reference coding sequence. Such an increased number may be generated by substitution of codons containing adenosine or thymidine nucleotides by codons containing guanosine or cytosine nucleotides. Advantageously, nucleic acid sequences having an increased G/C content are more stable or show a better expression than sequences having an increased A/U. The amino acid sequence encoded by the G/C content modified coding sequence of the at least one nucleic acid is preferably not modified as compared to the amino acid sequence encoded by the respective wild type or reference sequence. Preferably, the G/C content of the coding sequence of the at least one nucleic acid is increased by at least 10%, 20%, 30%, preferably by at least 40% compared to the G/C content of the coding sequence of the corresponding wild type or reference nucleic acid sequence (herein referred to “opt 10” or “gc mod”)

In some embodiments, the at least one nucleic acid may be modified, wherein the codon adaptation index (CAI) may be increased or preferably maximised in the at least one coding sequence (herein referred to as “CAI maximized coding sequence”). It is preferred that all codons of the wild type or reference nucleic acid sequence that are relatively rare in e.g. a human are exchanged for a respective codon that is frequent in the e.g. a human, wherein the frequent codon encodes the same amino acid as the relatively rare codon. Suitably, the most frequent codons are used for each amino acid of the encoded protein (see Table 8, most frequent human codons are marked with asterisks). Suitably, the nucleic acid comprises at least one coding sequence, wherein the codon adaptation index (CAI) of the at least one coding sequence is at least 0.5, at least 0.8, at least 0.9 or at least 0.95. Most preferably, the codon adaptation index (CAI) of the at least one coding sequence is 1 (CAI=1). For example, in the case of the amino acid Ala, the wild type or reference coding sequence may be adapted in a way that the most frequent human codon “GCC” is always used for said amino acid. Accordingly, such a procedure (as exemplified for Ala) may be applied for each amino acid encoded by the coding sequence of the nucleic acid to obtain CAI maximized coding sequences.

In embodiments, the at least one nucleic acid may be modified by altering the number of A and/or U nucleotides in the nucleic acid sequence with respect to the number of A and/or U nucleotides in the original nucleic acid sequence (e.g. the wild type or reference sequence). Preferably, such an AU alteration is performed to modify the retention time of the individual nucleic acids in a composition, to (i) allow co-purification using a HPLC method, and/or to allow analysis of the obtained nucleic acid composition. Such a method is described in detail in published PCT application WO2019092153A1. Claims 1 to 70 of WO2019092153A1 herewith incorporated by reference.

In particularly preferred embodiments, the at least one coding sequence of the at least one nucleic acid is a codon modified coding sequence, wherein the codon modified coding sequence is selected from a G/C optimized coding sequence, a human codon usage adapted coding sequence, or a G/C modified coding sequence, preferably a G/C optimized coding sequence.

In preferred embodiments, the at least one nucleic acid comprises at least one heterologous untranslated region (UTR).

The term “untranslated region” or “UTR” or “UTR element” will be recognized and understood by the person of ordinary skill in the art, and are e.g. intended to refer to a part of a nucleic acid molecule typically located 5′ or 3′ of a coding sequence. An UTR is not translated into protein. An UTR may be part of a nucleic acid, e.g. a DNA or an RNA. An UTR may comprise elements for controlling gene expression, also called regulatory elements. Such regulatory elements may be, e.g., ribosomal binding sites, miRNA binding sites, promotor elements etc.

In preferred embodiments, the at least one nucleic acid comprises a protein-coding region (“coding sequence” or “ods”), and 5′-UTR and/or 3′-UTR. Notably, UTRs may harbor regulatory sequence elements that determine nucleic acid, e.g. RNA turnover, stability, and localization. Moreover, UTRs may harbor sequence elements that enhance translation. In medical application of nucleic acid sequences (including DNA and RNA), translation of the nucleic acid into at least one peptide or protein is of paramount importance to therapeutic efficacy. Certain combinations of 3′-UTRs and/or 5′-UTRs may enhance the expression of operably linked coding sequences encoding peptides or proteins of the invention. Nucleic acid molecules harboring said UTR combinations advantageously enable rapid and transient expression of antigenic peptides or proteins after administration to a subject, preferably after intramuscular administration. Accordingly, the at least one nucleic acid comprising certain combinations of 3′-UTRs and/or 5′-UTRs as provided herein is particularly suitable for administration as a vaccine, in particular, suitable for administration into the muscle, the dermis, or the epidermis of a subject.

Suitably, the at least one nucleic acid comprises at least one heterologous 5′-UTR and/or at least one heterologous 3′-UTR. Said heterologous 5′-UTRs or 3′-UTRs may be derived from naturally occurring genes or may be synthetically engineered. In preferred embodiments, the nucleic acid, preferably the RNA comprises at least one coding sequence as defined herein operably linked to at least one (heterologous) 3′-UTR and/or at least one (heterologous) 5′-UTR.

In preferred embodiments, the at least one nucleic acid, e.g. the RNA or DNA, comprises at least one heterologous 3′-UTR.

Preferably, the RNA comprises a 3′-UTR, which may be derivable from a gene that relates to an RNA with enhanced half-life (i.e. that provides a stable RNA).

The term “3′-untranslated region” or “3′-UTR” or “3′-UTR element” will be recognized and understood by the person of ordinary skill in the art, and are e.g. intended to refer to a part of a nucleic acid molecule located 3′ (i.e. downstream) of a coding sequence and which is not translated into protein. A 3′-UTR may be part of a nucleic acid, e.g. a DNA or an RNA, located between a coding sequence and an (optional) terminal poly(A) sequence. A 3′-UTR may comprise elements for controlling gene expression, also called regulatory elements. Such regulatory elements may be, e.g., ribosomal binding sites, miRNA binding sites etc.

Preferably, the at least one nucleic acid comprises a 3′-UTR, which may be derivable from a gene that relates to an RNA with enhanced half-life (i.e. that provides a stable RNA).

In some embodiments, a 3′-UTR comprises one or more of a polyadenylation signal, a binding site for proteins that affect a nucleic acid stability of location in a cell, or one or more miRNA or binding sites for miRNAs.

MicroRNAs (or miRNA) are 19-25 nucleotide long noncoding RNAs that bind to the 3′-UTR of nucleic acid molecules and down-regulate gene expression either by reducing nucleic acid molecule stability or by inhibiting translation. E.g., microRNAs are known to regulate RNA, and thereby protein expression, e.g. in liver (miR-122), heart (miR-Id, miR-149), endothelial cells (miR-17-92, miR-126), adipose tissue (let-7, miR-30c), kidney (miR-192, miR-194, miR-204), myeloid cells (miR-142-3p, miR-142-5p, miR-16, miR-21, miR-223, miR-24, miR-27), muscle (miR-133, miR-206, miR-208), and lung epithelial cells (let-7, miR-133, miR-126). The RNA may comprise one or more microRNA target sequences, microRNA sequences, or microRNA seeds. Such sequences may e.g. correspond to any known microRNA such as those taught in US2005/0261218 and US2005/0059005.

Accordingly, miRNA, or binding sites for miRNAs as defined above may be removed from the 3′-UTR or may be introduced into the 3′-UTR in order to tailor the expression of the nucleic acid, e.g. the DNA or RNA to desired cell types or tissues (e.g. muscle cells).

In preferred embodiments, the at least one nucleic acid comprises at least one heterologous 3′-UTR, wherein the at least one heterologous 3′-UTR comprises a nucleic acid sequence is derived or selected from a 3′-UTR of a gene selected from PSMB3, ALB7, alpha-globin (referred to as “muag”), CASP1, COX6B1, GNAS, NDUFA1 and RPS9, or from a homolog, a fragment or variant of any one of these genes, preferably according to nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 253-268, 22902-22905, 22892-22895 or a fragment or a variant of any of these. Particularly preferred nucleic acid sequences in that context can be derived from published PCT application WO2019077001A1, in particular, claim 9 of WO2019077001A1. The corresponding 3′-UTR sequences of claim 9 of WO2019077001A1 are herewith incorporated by reference (e.g., SEQ ID NOs: 23-34 of WO2019077001A1, or fragments or variants thereof).

In preferred embodiments, the at least one nucleic acid comprises a 3′-UTR derived from an alpha-globin gene. Said 3′-UTR derived from a alpha-globin gene (“muag”) may comprise or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 267, 268, 22896-22901, 22906-22911 or a fragment or a variant thereof.

In further embodiments, the at least one nucleic acid comprises a 3-UTR derived from a RPS9 gene. Said 3-UTR derived from a RPS9 gene may comprise or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 263, 264, 22894, 22895, 22904, or 22905 or a fragment or a variant thereof.

In other embodiments, the nucleic acid comprise a 3′-UTR which comprise or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 22876-22891 or a fragment or a variant thereof.

In preferred embodiments, the at least one nucleic acid comprises a 3′-UTR derived from a PSMB3 gene. Said 3′-UTR derived from a PSMB3 gene may comprise or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 253 or 254 or a fragment or a variant thereof.

In other embodiments, the at least one nucleic acid may comprise a 3′-UTR as described in WO2016107877, the disclosure of WO2016107877 relating to 3′-UTR sequences herewith incorporated by reference. Suitable 3′-UTRs are SEQ ID NOs: 1-24 and SEQ ID NOs: 49-318 of WO2016107877, or fragments or variants of these sequences. In other embodiments, the nucleic acid comprises a 3′-UTR as described in WO2017036580, the disclosure of WO2017036580 relating to 3′-UTR sequences herewith incorporated by reference. Suitable 3′-UTRs are SEQ ID NOs: 152-204 of WO2017036580, or fragments or variants of these sequences. In other embodiments, the nucleic acid comprises a 3′-UTR as described in WO2016022914, the disclosure of WO2016022914 relating to 3′-UTR sequences herewith incorporated by reference. Particularly preferred 3′-UTRs are nucleic acid sequences according to SEQ ID NOs: 20-36 of WO2016022914, or fragments or variants of these sequences.

In preferred embodiments, the at least one nucleic acid, e.g. the RNA or DNA, comprises at least one heterologous 5′-UTR.

The terms “5-untranslated region” or “5′-UTR” or “5′-UTR element” will be recognized and understood by the person of ordinary skill in the art, and are e.g. intended to refer to a part of a nucleic acid molecule located 5′ (i.e. “upstream”) of a coding sequence and which is not translated into protein. A 5′-UTR may be part of a nucleic acid located 5′ of the coding sequence. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, miRNA binding sites etc. The 5′-UTR may be post-transcriptionally modified, e.g. by enzymatic or post-transcriptional addition of a 5′-cap structure (e.g. for mRNA as defined below).

Preferably, the at least one nucleic acid comprises a 5′-UTR, which may be derivable from a gene that relates to an RNA with enhanced half-life (i.e. that provides a stable RNA).

In some embodiments, a 5′-UTR comprises one or more of a binding site for proteins that affect an RNA stability or RNA location in a cell, or one or more miRNA or binding sites for miRNAs (as defined above).

Accordingly, miRNA or binding sites for miRNAs as defined above may be removed from the 5′-UTR or introduced into the 5′-UTR in order to tailor the expression of the nucleic acid to desired cell types or tissues (e.g. muscle cells).

In preferred embodiments, the at least one nucleic acid comprises at least one heterologous 5′-UTR, wherein the at least one heterologous 5′-UTR comprises a nucleic acid sequence is derived or selected from a 5′-UTR of gene selected from HSD17B4, RPL32, ASAH1, ATP5A1, MP68, NDUFA4, NOSIP, RPL31, SLC7A3, TUBB4B, and UBQLN2, or from a homolog, a fragment or variant of any one of these genes according to nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 231-252, 22870-22875 or a fragment or a variant of any of these. Particularly preferred nucleic acid sequences in that context can be selected from published PCT application WO2019077001A1, in particular, claim 9 of WO2019077001A1. The corresponding 5′-UTR sequences of claim 9 of WO2019077001A1 are herewith incorporated by reference (e.g., SEQ ID NOs: 1-20 of WO2019077001A1, or fragments or variants thereof).

In preferred embodiments, the at least one nucleic acid comprises comprises a 5′-UTR derived from a RPL31 gene, wherein said 5′-UTR derived from a RPL31 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 243, 244, 22872, 22873 or a fragment or a variant thereof.

In embodiments, the at least one nucleic acid comprises may comprise a 5′-UTR derived from a SLC7A3 gene, wherein said 5′-UTR derived from a SLC7A3 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 245, 246, 22874, 22875 or a fragment or a variant thereof.

In particularly preferred embodiments, the at least one nucleic acid comprises a 5′-UTR derived or selected from a HSD17B4 gene, wherein said 5′-UTR derived from a HSD17B4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 231, 232, 22870, 22871 or a fragment or a variant thereof.

In other embodiments, the at least one nucleic acid comprises a 5′-UTR which comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NO: 22848-22869 or a fragment or a variant thereof.

In other embodiments, the at least one nucleic acid may comprise a 5′-UTR as described in WO2013143700, the disclosure of WO2013143700 relating to 5′-UTR sequences herewith incorporated by reference. Particularly preferred 5′-UTRs are nucleic acid sequences derived from SEQ ID NOs: 1-1363, SEQ ID NO: 1395, SEQ ID NO: 1421 and SEQ ID NO: 1422 of WO2013143700, or fragments or variants of these sequences. In other embodiments, the nucleic acid comprises a 5′-UTR as described in WO2016107877, the disclosure of WO2016107877 relating to 5′-UTR sequences herewith incorporated by reference. Particularly preferred 5′-UTRs are nucleic acid sequences according to SEQ ID NOs: 25-30 and SEQ ID NOs: 319-382 of WO2016107877, or fragments or variants of these sequences. In other embodiments, the nucleic acid comprises a 5′-UTR as described in WO2017036580, the disclosure of WO2017036580 relating to 5′-UTR sequences herewith incorporated by reference. Particularly preferred 5′-UTRs are nucleic acid sequences according to SEQ ID NOs: 1-151 of WO2017036580, or fragments or variants of these sequences. In other embodiments, the nucleic acid comprises a 5′-UTR as described in WO2016022914, the disclosure of WO2016022914 relating to 5′-UTR sequences herewith incorporated by reference. Particularly preferred 5′-UTRs are nucleic acid sequences according to SEQ ID NOs: 3-19 of WO2016022914, or fragments or variants of these sequences.

In preferred embodiments, the at least one nucleic acid comprises at least one coding sequence as specified herein encoding at least one antigenic protein as defined herein, operably linked to a 3′-UTR and/or a 5′-UTR selected from the following 5′UTR/3′UTR combinations (“also referred to UTR designs”):
a-1 (HSD17B4/PSMB3), a-2 (NDUFA4/PSMB3), a-3 (SLC7A3/PSMB3), a-4 (NOSIP/PSMB3), a-5 (MP68/PSMB3), b-1 (UBQLN2/RPS9), b-2 (ASAH1/RPS9), b-3 (HSD17B4/RPS9), b-4 (HSD17B4/CASP1), b-5 (NOSIP/COX6B1), c-1 (NDUFA4/RPS9), c-2 (NOSIP/NDUFA1), c-3 (NDUFA4/COX6B1), c-4 (NDUFA4/NDUFA1), c-5 (ATP5A1/PSMB3), d-1 (Rpl31/PSMB3), d-2 (ATP5A1/CASP1), d-3 (SLC7A3/GNAS), d-4 (HSD17B4/NDUFA1), d-5 (Slc7a3/Ndufa1), e-1 (TUBB4B/RPS9), e-2 (RPL31/RPS9), e-3 (MP68/RPS9), e-4 (NOSIP/RPS9), e-5 (ATP5A1/RPS9), e-6 (ATP5A1/COX6B1), f-1 (ATP5A1/GNAS), f-2 (ATP5A1/NDUFA1), f-3 (HSD17B4/COX6B1), f-4 (HSD17B4/GNAS), f-5 (MP68/COX6B1), g-1 (MP68/NDUFA1), g-2 (NDUFA4/CASP1), g-3 (NDUFA4/GNAS), g-4 (NOSIP/CASP1), g-5 (RPL31/CASP1), h-1 (RPL31/COX6B1), h-2 (RPL31/GNAS), h-3 (RPL31/NDUFA1), h-4 (Slc7a3/CASP1), h-5 (SLC7A3/COX6B1), i-1 (SLC7A3/RPS9), i-2 (RPL32/ALB7), i-2 (RPL32/ALB7), or i-3 (alpha-globin gene).

In particularly preferred embodiments, the at least one nucleic acid comprises at least one coding sequence as defined herein encoding at least one antigenic protein as defined herein, wherein said coding sequence is operably linked to a HSD17B4 5′-UTR and a PSMB3 3′-UTR (HSD17B4/PSMB3 (UTR design a-1)).

In further preferred embodiments, the at least one nucleic acid comprises at least one coding sequence as specified herein encoding at least one antigenic protein as defined herein, preferably derived from SARS-CoV-2 (nCoV-2019) coronavirus, wherein said coding sequence is operably linked to a SLC7A3 5′-UTR and a PSMB3 3′-UTR (SLC7A3/PSMB3 (UTR design a-3)).

In further preferred embodiments, the at least one nucleic acid comprises at least one coding sequence as specified herein encoding at least one antigenic protein as defined herein, preferably derived from SARS-CoV-2 (nCoV-2019) coronavirus, wherein said coding sequence is operably linked to a RPL31 5′-UTR and a RPS9 3′-UTR (RPL31/RPS9 (UTR design e-2)).

In particularly preferred embodiments, the at least one nucleic acid comprises at least one coding sequence as defined herein encoding at least one antigenic protein as defined herein, wherein said coding sequence is operably linked to an alpha-globin (“muag”) 3′-UTR.

In embodiments, the at least one nucleic acid, e.g. the DNA or RNA may be monocistronic, bicistronic, or multicistronic.

The term “monocistronic” will be recognized and understood by the person of ordinary skill in the art, and is e.g. intended to refer to a nucleic acid that comprises only one coding sequence. The terms “bicistronic”, or “multicistronic” as used herein will be recognized and understood by the person of ordinary skill in the art, and are e.g. intended to refer to a nucleic acid that may comprise two (bicistronic) or more (multicistronic) coding sequences.

In preferred embodiments, the at least one nucleic acid is monocistronic.

In embodiments, the at least one nucleic acid is monocistronic and the coding sequence of said nucleic acid encodes at least two different antigenic peptides or proteins. Accordingly, said coding sequence may encode at least two, three, four, five, six, seven, eight and more antigenic peptides or proteins, linked with or without an amino acid linker sequence, wherein said linker sequence can comprise rigid linkers, flexible linkers, cleavable linkers, or a combination thereof. Such constructs are herein referred to as “multi-antigen-constructs”.

In embodiments, the nucleic acid of at least one nucleic acid may be bicistronic or multicistronic and comprises at least two coding sequences, wherein the at least two coding sequences encode two or more different antigenic peptides or proteins as specified herein. Accordingly, the coding sequences in a bicistronic or multicistronic nucleic acid suitably encodes distinct antigenic proteins or peptides as defined herein or immunogenic fragments or immunogenic variants thereof. Preferably, the coding sequences in said bicistronic or multicistronic constructs may be separated by at least one IRES (internal ribosomal entry site) sequence. Thus, the term “encoding two or more antigenic peptides or proteins” may mean, without being limited thereto, that the bicistronic or multicistronic nucleic acid encodes e.g. at least two, three, four, five, six or more (preferably different) antigenic peptides or proteins of virus isolates. Alternatively, the bicistronic or multicistronic nucleic acid may encode e.g. at least two, three, four, five, six or more (preferably different) antigenic peptides or proteins derived from the same virus. In that context, suitable IRES sequences may be selected from the list of nucleic acid sequences according to SEQ ID NOs: 1566-1662 of the patent application WO2017081082, or fragments or variants of these sequences. In this context, the disclosure of WO2017081082 relating to IRES sequences is herewith incorporated by reference.

It has to be understood that, in the context of the invention, certain combinations of coding sequences may be generated by any combination of monocistronic, bicistronic and multicistronic DNA and/or RNA constructs and/or multi-antigen-constructs to obtain a nucleic acid set encoding multiple antigenic peptides or proteins as defined herein.

In embodiments, the A/U (A/T) content in the environment of the ribosome binding site of the at least one nucleic acid may be increased compared to the A/U (A/T) content in the environment of the ribosome binding site of its respective wild type or reference nucleic acid. This modification (an increased A/U (A/T) content around the ribosome binding site) increases the efficiency of ribosome binding to the nucleic acid, e.g. to an RNA. An effective binding of the ribosomes to the ribosome binding site in turn has the effect of an efficient translation the nucleic acid.

Accordingly, in a particularly preferred embodiment, the at least one nucleic acid comprises a ribosome binding site, also referred to as “Kozak sequence” identical to or at least 80%, 85%, 90%, 95% identical to any one of the sequences SEQ ID NOs: 180, 181, 22845-22847 or fragments or variants thereof.

In preferred embodiments, the at least one nucleic acid comprises at least one poly(N) sequence, e.g. at least one poly(A) sequence, at least one poly(U) sequence, at least one poly(C) sequence, or combinations thereof.

In preferred embodiments, the at least one nucleic acid, preferably the RNA comprises at least one poly(A) sequence.

The terms “poly(A) sequence”, “poly(A) tail” or “3′-poly(A) tail” as used herein will be recognized and understood by the person of ordinary skill in the art, and are e.g. intended to be a sequence of adenosine nucleotides, typically located at the 3′-end of a linear RNA (or in a circular RNA), of up to about 1000 adenosine nucleotides. Preferably, said poly(A) sequence is essentially homopolymeric, e.g. a poly(A) sequence of e.g. 100 adenosine nucleotides has essentially the length of 100 nucleotides. In other embodiments, the poly(A) sequence may be interrupted by at least one nucleotide different from an adenosine nucleotide, e.g. a poly(A) sequence of e.g. 100 adenosine nucleotides may have a length of more than 100 nucleotides (comprising 100 adenosine nucleotides and in addition said at least one nucleotide—or a stretch of nucleotides—different from an adenosine nucleotide). It has to be understood that “poly(A) sequence” as defined herein typically relates to RNA—however in the context of the invention, the term likewise relates to corresponding sequences in a DNA molecule (e.g. a “poly(T) sequence”).

The poly(A) sequence may comprise about 10 to about 500 adenosine nucleotides, about 10 to about 200 adenosine nucleotides, about 40 to about 200 adenosine nucleotides, or about 40 to about 150 adenosine nucleotides. Suitably, the length of the poly(A) sequence may be at least about or even more than about 10, 50, 64, 75, 100, 200, 300, 400, or 500 adenosine nucleotides. In certain embodiments the RNA comprises at least one poly(A) sequence comprising 30 to 200 adenosine nucleotides, wherein the 3′ terminal nucleotide of said RNA is an adenosine.

In preferred embodiments, the at least one nucleic acid comprises at least one poly(A) sequence comprising about 30 to about 200 adenosine nucleotides. In particularly preferred embodiments, the poly(A) sequence comprises about 64 adenosine nucleotides (A64). In other particularly preferred embodiments, the poly(A) sequence comprises about 100 adenosine nucleotides (A100). In other embodiments, the poly(A) sequence comprises about 150 adenosine nucleotides.

In further embodiments, the at least one nucleic acid comprises at least one poly(A) sequence comprising about 100 adenosine nucleotides, wherein the poly(A) sequence is interrupted by non-adenosine nucleotides, preferably by 10 non-adenosine nucleotides (A30-N10-A70).

The poly(A) sequence as defined herein may be located directly at the 3′ terminus of the at least one nucleic acid, preferably directly located at the 3′ terminus of an RNA.

In preferred embodiments, the 3-terminal nucleotide (that is the last 3′-terminal nucleotide in the polynucleotide chain) is the 3-terminal A nucleotide of the at least one poly(A) sequence. The term “directly located at the 3′ terminus” has to be understood as being located exactly at the 3′ terminus—in other words, the 3′ terminus of the nucleic acid consists of a poly(A) sequence terminating with an A nucleotide.

In a particularly preferred embodiment the nucleic acid sequence, preferably the RNA comprises a poly(A) sequence of at least 70 adenosine nucleotides, preferably consecutive at least 70 adenosine nucleotides, wherein the 3′-terminal nucleotide is an adenosine nucleotide.

In this context it has been shown that ending on an adenosine nucleotide decreases the induction of IFNalpha by the RNA vaccine. This is particularly important as the induction of IFNalpha is thought to be the main factor for induction of fever in vaccinated subjects, which of course has to be avoided.

In embodiments where the at least one nucleic acid is an RNA, the poly(A) sequence of the nucleic acid is preferably obtained from a DNA template during RNA in vitro transcription. In other embodiments, the poly(A) sequence is obtained in vitro by common methods of chemical synthesis without being necessarily transcribed from a DNA template. In other embodiments, poly(A) sequences are generated by enzymatic polyadenylation of the RNA (after RNA in vitro transcription) using commercially available polyadenylation kits and corresponding protocols known in the art, or alternatively, by using immobilized poly(A)polymerases e.g. using a methods and means as described in WO2016174271.

The at least one nucleic acid may comprise a poly(A) sequence obtained by enzymatic polyadenylation, wherein the majority of nucleic acid molecules comprise about 100 (+/−20) to about 500 (+/−50), preferably about 250 (+/−20) adenosine nucleotides.

In embodiments, the at least one nucleic acid comprises a poly(A) sequence derived from a template DNA and, optionally, additionally comprises at least one additional poly(A) sequence generated by enzymatic polyadenylation, e.g. as described in WO2016091391.

In embodiments, the at least one nucleic acid comprises at least one polyadenylation signal.

In embodiments, the at least one nucleic acid comprises at least one poly(C) sequence.

The term “poly(C) sequence” as used herein is intended to be a sequence of cytosine nucleotides of up to about 200 cytosine nucleotides. In preferred embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In a particularly preferred embodiment, the poly(C) sequence comprises about 30 cytosine nucleotides.

In preferred embodiments, the at least one nucleic acid comprises at least one histone stem-loop (hSL) or histone stem loop structure.

The term “histone stem-loop” (abbreviated as “hSL” in e.g. the sequence listing) is intended to refer to nucleic acid sequences that form a stem-loop secondary structure predominantly found in histone mRNAs.

Histone stem-loop sequences/structures may suitably be selected from histone stem-loop sequences as disclosed in WO2012019780, the disclosure relating to histone stem-loop sequences/histone stem-loop structures incorporated herewith by reference. A histone stem-loop sequence that may be used within the present invention may preferably be derived from formulae (I) or (II) of WO2012019780. According to a further preferred embodiment, the at least one nucleic acid comprises at least one histone stem-loop sequence derived from at least one of the specific formulae (Ia) or (IIa) of the patent application WO2012019780.

In preferred embodiments, the at least one nucleic acid comprises at least one histone stem-loop, wherein said histone stem-loop (hSL) comprises or consists a nucleic acid sequence identical or at least 70%, 80%, 90%, 91%, 92%, 93%, 94%, 95%, 96%, 97%, 98%, or 99% identical to SEQ ID NOs: 178 or 179, or fragments or variants thereof.

In other embodiments, the at least one nucleic acid does not comprise a histone stem-loop as defined herein

In embodiments, in particular in embodiments that relate to RNA, the at least one nucleic acid comprises a 3′-terminal sequence element. Said 3-terminal sequence element comprises a poly(A) sequence and a histone-stem-loop sequence. Accordingly, the at least one nucleic acid comprises at least one 3′-terminal sequence element comprising or consisting of a nucleic acid sequence being identical or at least 70%, 80%, 90%, 91%, 92%, 93%, 94%, 95%, 96%, 97%, 98%, or 99% identical to SEQ ID NOs: 182-230, 22912, 22913 or a fragment or variant thereof.

In embodiments, in particular in embodiments that relate to RNA, the at least one nucleic acid comprises a 3-terminal sequence element. Said 3′-terminal sequence element comprises a poly(A). Accordingly, the at least one nucleic acid comprises at least one 3-terminal sequence element comprising or consisting of a nucleic acid sequence being identical or at least 70%, 80%, 90%, 91%, 92%, 93%, 94%, 95%, 96%, 97%, 98%, or 99% identical to SEQ ID NOs: 182-230, 22912, 22913 or a fragment or variant thereof.

In preferred embodiments, in particular in embodiments that relate to RNA, the nucleic acid comprises a 3′-terminal sequence element. Said 3-terminal sequence element may comprise a poly(A) sequence and optionally a histone-stem-loop sequence. Accordingly, the nucleic acid of the invention comprises at least one 3-terminal sequence element comprising or consisting of a nucleic acid sequence being identical or at least 70%, 80%, 90%, 91%, 92%, 93%, 94%, 95%, 96%, 97%, 98%, or 99% identical to SEQ ID NOs: 182, 187, 189, 192, 199, 207, or a fragment or variant thereof.

In various embodiments, in particular in embodiments that relate to RNA, the at least one nucleic acid may comprise a 5′-terminal sequence element according to SEQ ID NOs: 176, 177, 22840-22844, or a fragment or variant thereof. Such a 5′-terminal sequence element comprises e.g. a binding site for T7 RNA polymerase. Further, the first nucleotide of said 5′-terminal start sequence may preferably comprise a 2′O methylation, e.g. 2′O methylated guanosine or a 2′O methylated adenosine.

In preferred embodiments, the nucleic acid comprises at least one heterologous 5′-UTR that comprises or consists of a nucleic acid sequence derived from a 5′-UTR from HSD17B4 and at least one heterologous 3′-UTR comprises or consists of a nucleic acid sequence derived from a 3′-UTR of PSMB3. In certain embodiments, the 5′-UTR from HSD17B4 is at least about 95%, 96%, 97%, 98% to 99% identical to SEQ ID NO: 232. In some embodiments, the 3′-UTR of PSMB3 is at least about 95%, 96%, 97%, 98% to 99% identical to SEQ ID NO: 254. In especially preferred embodiments the nucleic acid, preferably the RNA comprises, from 5′ to 3′: i) 5′-cap1 structure; ii) 5′-UTR derived from a 5′-UTR of a HSD17B4 gene, preferably according to SEQ ID NO: 232; iii) the at least one coding sequence (encoding a Coroanavirus antigen according to the invention); iv) 3′-UTR derived from a 3′-UTR of a PSMB3 gene, preferably according to SEQ ID NO: 254; v) optionally, a histone stem-loop sequence; and vi) poly(A) sequence comprising about 100 A nucleotides, wherein the 3′ terminal nucleotide of said RNA is an adenosine.

Preferably, the at least one nucleic acid, e.g. the RNA or DNA, typically comprises about 50 to about 20000 nucleotides, or about 500 to about 10000 nucleotides, or about 1000 to about 10000 nucleotides, or preferably about 1000 to about 5000 nucleotides, or even more preferably about 2000 to about 5000 nucleotides.

In embodiments, the at least one nucleic acid is a DNA or an RNA.

In embodiments, the DNA is a plasmid DNA or a linear coding DNA construct, wherein the DNA comprises or consists of the nucleic acid elements as defined herein (e.g. including coding sequences, UTRs, poly(A/T), polyadenylation signal, a promoter).

In embodiments, the at least one nucleic acid is a DNA expression vector. Such a DNA expression vector may be selected from the group consisting of a bacterial plasmid, an Adenovirus, a Poxvirus, a Parapoxivirus (orf virus), a Vaccinia virus, a Fowlpox virus, a Herpes virus, an Adeno-associated virus (AAV), an Alphavirus, a Lentivirus, a Lambda phage, a Lymphocytic choriomeningitis virus, a Listeria sp and Salmonella sp.

Suitably, the DNA may also comprise a promoter that is operably linked to the respective antigen coding sequence of the at least one nucleic acid. The promoter operably linked to the antigen coding sequence can be e.g. a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter can also be a promoter from a human gene such as human actin, human myosin, human hemoglobin, human muscle creatine, or human metalothionein. The promoter can also be a tissue specific promoter, such as a muscle or skin specific promoter, natural or synthetic. Examples of such promoters are described in US patent application publication no. US20040175727. In preferred embodiments, the vector can be pVAX, pcDNA3.0, or provax, or any other expression vector capable of expressing DNA encoding the coronavirus antigen and enabling a cell to translate the sequence to an antigen that is recognized by the immune system.

Further suitable plasmid DNA may be generated to allow efficient production of the encoded antigens in cell lines, e.g. in insect cell lines, for example using vectors as described in WO2009150222A2 and as defined in PCT claims 1 to 33, the disclosure relating to claim 1 to 33 of WO2009150222A2 herewith incorporated by reference.

In embodiments, the at least one nucleic acid is an adenovirus based vector. Such an adenovirus based vector may comprise at least one coding sequence encoding at least one antigenic peptide or protein as defined herein (e.g. at least one Coronavirus antigen).

In the context of the invention, any suitable adenovirus based vector may be used such as those described in WO2005071093 or WO2006048215. Suitably, the adenovirus based vector used is a simian adenovirus, thereby avoiding dampening of the immune response after vaccination by pre-existing antibodies to common human entities such as AdHu5. Suitable simian adenovirus vectors include AdCh63 (see WO2005071093) or AdCh68 but others may also be used. Suitably the adenovirus vector will have the E1 region deleted, rendering it replication-deficient in human cells. Other regions of the adenovirus such as E3 and E4 may also be deleted.

In embodiments, the at least one nucleic acid is an orf virus based vector. Such an orf virus based vector may comprise at least one coding sequence encoding at least one antigenic peptide or protein as defined.

In particularly preferred embodiments of the invention, the at least one nucleic acid is an RNA.

Preferably, the RNA typically comprises about 50 to about 20000 nucleotides, or about 500 to about 10000 nucleotides, or about 1000 to about 10000 nucleotides, or preferably about 1000 to about 5000 nucleotides, or even more preferably about 2000 to about 5000 nucleotides.

According to preferred embodiments, the at least one nucleic acid is an RNA, preferably a coding RNA.

In preferred embodiments, the (coding) RNA is selected from an mRNA, a (coding) self-replicating RNA, a (coding) circular RNA, a (coding) viral RNA, or a (coding) replicon RNA.

In embodiments, the RNA is a circular RNA. As used herein, “circular RNA” or “circRNAs” have to be understood as a circular polynucleotide constructs that encode at least one antigenic peptide or protein as defined herein. Preferably, such a circRNA is a single stranded RNA molecule. In preferred embodiments, said circRNA comprises at least one coding sequence encoding at least one antigenic protein as defined herein, or an immunogenic fragment or an immunogenic variant thereof.

In embodiments, the RNA is a replicon RNA. The term “replicon RNA” will be recognized and understood by the person of ordinary skill in the art, and is e.g. intended to be an optimized self-replicating RNA. Such constructs may include replicase elements derived from e.g. alphaviruses (e.g. SFV, SIN, VEE, or RRV) and the substitution of the structural virus proteins with the nucleic acid of interest (that is, the coding sequence encoding an antigenic peptide or protein as defined herein). Alternatively, the replicase may be provided on an independent coding RNA construct or a coding DNA construct. Downstream of the replicase may be a sub-genomic promoter that controls replication of the replicon RNA.

In particularly preferred embodiments, the at least one nucleic acid is not a replicon RNA or a self-replicating RNA.

In particularly preferred embodiments, the at least one nucleic acid is an mRNA.

Preferably, the mRNA does not comprise a replicase element (e.g. a nucleic acid encoding a replicase).

The terms “RNA” and “mRNA” will be recognized and understood by the person of ordinary skill in the art, and are e.g. intended to be a ribonucleic acid molecule, i.e. a polymer consisting of nucleotides. These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e. ribose, of a first and a phosphate moiety of a second, adjacent monomer. The specific succession of the monomers is called the RNA-sequence. The mRNA (messenger RNA) provides the nucleotide coding sequence that may be translated into an amino-acid sequence of a particular peptide or protein.

In the context of the invention, the RNA, preferably the mRNA, provides at least one coding sequence encoding an antigenic protein as defined herein that is translated into a (functional) antigen after administration (e.g. after administration to a subject, e.g. a human subject).

Accordingly, the RNA, preferably the mRNA, is suitable for a vaccine, preferably a multivalent vaccine of the invention.

In preferred embodiments, the RNA, preferably the mRNA is suitable for a SARS-CoV-2 vaccine, preferably a SARS-CoV-2 vaccine against at least one of the following SARS-CoV-2 isolates: B.1.351 (South Africa), B.1.1.7 (UK), P.1 (Brazil), B.1.429 (California), B.1.525 (Nigeria), B.1.258 (Czech republic), B.1.526 (New York), A.23.1 (Uganda), B.1.617.1 (India), B.1.617.2 (India), B.1.617.3 (India), P.2 (Brazil), C37.1 (Peru).

In particularly preferred embodiments, the RNA, preferably the mRNA is suitable for a SARS-CoV-2 vaccine, preferably a SARS-CoV-2 vaccine against B.1.351 (South Africa).

In particularly preferred embodiments, the RNA, preferably the mRNA is suitable for a SARS-CoV-2 vaccine, 40 preferably a SARS-CoV-2 vaccine against B.1.617.1 (India), B.1.617.2 (India), and/or B.1.617.3 (India). In further particularly preferred embodiments, the RNA, preferably the mRNA is suitable for a SARS-CoV-2 vaccine, preferably a SARS-CoV-2 vaccine against C37.1 (Peru).

Suitably, the RNA may be modified by the addition of a 5′-cap structure, which preferably stabilizes the RNA and/or 45 enhances expression of the encoded antigen and/or reduces the stimulation of the innate immune system (after administration to a subject). A 5′-cap structure is of particular importance in embodiments where the nucleic acid is an RNA, in particular a linear coding RNA, e.g. a linear mRNA or a linear coding replicon RNA.

Accordingly, in preferred embodiments, the at least one nucleic acid comprises a 5′-cap structure, preferably m7G, cap0, cap1, cap2, a modified cap0 or a modified cap1 structure.

The term “5′-cap structure” as used herein will be recognized and understood by the person of ordinary skill in the art, and is e.g. intended to refer to a 5′ modified nucleotide, particularly a guanine nucleotide, positioned at the 5-end of an RNA, e.g. an mRNA. Preferably, the 5′-cap structure is connected via a 5′-5′-triphosphate linkage to the RNA.

5′-cap structures which may be suitable in the context of the present invention are cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

A 5′-cap (cap0 or cap1) structure may be formed in chemical RNA synthesis or in RNA in vitro transcription (co-transcriptional capping) using cap analogues.

The term “cap analogue” as used herein will be recognized and understood by the person of ordinary skill in the art, and is e.g. intended to refer to a non-polymerizable di-nucleotide or tri-nucleotide that has cap functionality in that it facilitates translation or localization, and/or prevents degradation of a nucleic acid molecule, particularly of an RNA molecule, when incorporated at the 5′-end of the nucleic acid molecule. Non-polymerizable means that the cap analogue will be incorporated only at the 5-terminus because it does not have a 5′ triphosphate and therefore cannot be extended in the 3′-direction by a template-dependent polymerase, particularly, by template-dependent RNA polymerase. Examples of cap analogues include, but are not limited to, a chemical structure selected from the group consisting of m7GpppG, m7GpppA, m7GpppC; unmethylated cap analogues (e.g. GpppG); dimethylated cap analogue (e.g. m2,7GpppG), trimethylated cap analogue (e.g. m2,2,7GpppG), dimethylated symmetrical cap analogues (e.g. m7Gpppm7G), or anti reverse cap analogues (e.g. ARCA; m7,2′OmeGpppG, m7,2′dGpppG, m7,3′OmeGpppG, m7,3′dGpppG and their tetraphosphate derivatives). Further cap analogues have been described previously (WO2008016473, WO2008157688, WO2009149253, WO2011015347, and WO2013059475). Further suitable cap analogues in that context are described in WO2017066793, WO2017066781, WO2017066791, WO2017066789, WO2017053297, WO2017066782, WO2018075827 and WO2017066797 wherein the disclosures referring to cap analogues are incorporated herewith by reference.

In embodiments, a modified cap1 structure is generated using tri-nucleotide cap analogue as disclosed in WO2017053297, WO2017066793, WO2017066781, WO2017066791, WO2017066789, WO2017066782, WO2018075827 and WO2017066797. In particular, any cap structures derivable from the structure disclosed in claim 1-5 of WO2017053297 may be suitably used to co-transcriptionally generate a modified cap1 structure. Further, any cap structures derivable from the structure defined in claim 1 or claim 21 of WO2018075827 may be suitably used to co-transcriptionally generate a modified cap1 structure.

In preferred embodiments, the RNA, in particular the mRNA comprises a cap1 structure.

In preferred embodiments, the 5′-cap structure may suitably be added co-transcriptionally using tri-nucleotide cap analogue as defined herein, preferably in an RNA in vitro transcription reaction as defined herein.

In preferred embodiments, the cap1 structure of the RNA is formed using co-transcriptional capping using tri-nucleotide cap analogues m7G(5′)ppp(5′)(2′OMeA)pG or m7G(5′)ppp(5′)(2′OMeG)pG. A preferred cap1 analogues in that context is m7G(5′)ppp(5′)(2′OMeA)pG.

In other preferred embodiments, the cap1 structure of the RNA of the invention is formed using co-transcriptional capping using tri-nucleotide cap analogue 3′OMe-m7G(5′)ppp(5′)(2′OMeA)pG.

In other embodiments, a cap0 structure of the RNA of the invention is formed using co-transcriptional capping using cap analogue 3′OMe-m7G(5′)ppp(5′)G.

In other embodiments, the 5′-cap structure is formed via enzymatic capping using capping enzymes (e.g. vaccinia virus capping enzymes and/or cap-dependent 2′-0 methyltransferases) to generate cap0 or cap1 or cap2 structures. The 5-cap structure (cap0 or cap1) may be added using immobilized capping enzymes and/or cap-dependent 2′-0 methyltransferases using methods and means disclosed in WO2016193226.

In preferred embodiments, about 70%, 75%, 80%, 85%, 90%, 95% of the RNA (species) comprises a cap1 structure as determined using a capping assay. In preferred embodiments, less than about 20%, 15%, 10%, 5%, 4%, 3%, 2%, 1% of the RNA (species) does not comprises a cap1 structure as determined using a capping assay. In other preferred embodiments, about 70%, 75%, 80%, 85%, 90%, 95% of the RNA (species) comprises a cap0 structure as determined using a capping assay. In preferred embodiments, less than about 20%, 15%, 10%, 5%, 4%, 3%, 2%, 1% of the RNA (species) does not comprises a cap0 structure as determined using a capping assay.

In the context of the invention, the term “RNA species” is not restricted to mean “one single molecule” but is understood to comprise an ensemble of essentially identical RNA molecules. Accordingly, it may relate to a plurality of essentially identical (coding) RNA molecules.

For determining the presence/absence of a cap0 or a cap1 structure, a capping assays as described in published PCT application WO2015101416, in particular, as described in claims 27 to 46 of published PCT application WO2015101416 can be used. Other capping assays that may be used to determine the presence/absence of a cap0 or a cap1 structure of an RNA are described in PCT/EP201808667, or published PCT applications WO2014152673 and WO2014152659.

In preferred embodiments, the RNA comprises an m7G(5′)ppp(5′)(2′OMeA) cap structure. In such embodiments, the RNA comprises a 5-terminal m7G cap, and an additional methylation of the ribose of the adjacent nucleotide of m7GpppN, in that case, a 2′O methylated Adenosine. Preferably, about 70%, 75%, 80%, 85%, 90%, 95% of the RNA (species) comprises such a cap1 structure as determined using a capping assay.

In other preferred embodiments, the RNA comprises an m7G(5′)ppp(5′)(2′OMeG) cap structure. In such embodiments, the RNA comprises a 5-terminal m7G cap, and an additional methylation of the ribose of the adjacent nucleotide, in that case, a 2′O methylated guanosine. Preferably, about 70%, 75%, 80%, 85%, 90%, 95% of the coding RNA (species) comprises such a cap1 structure as determined using a capping assay.

Accordingly, the first nucleotide of said RNA or mRNA sequence, that is, the nucleotide downstream of the m7G(5′)ppp structure, may be a 2′O methylated guanosine or a 2′O methylated adenosine.

According to embodiments, the RNA is a modified RNA, wherein the modification refers to chemical modifications comprising backbone modifications as well as sugar modifications or base modifications.

A modified RNA may comprise nucleotide analogues/modifications, e.g. backbone modifications, sugar modifications or base modifications. A backbone modification in the context of the invention is a modification, in which phosphates of the backbone of the nucleotides of the RNA are chemically modified. A sugar modification in the context of the invention is a chemical modification of the sugar of the nucleotides of the RNA. Furthermore, a base modification in the context of the invention is a chemical modification of the base moiety of the nucleotides of the RNA. In this context, nucleotide analogues or modifications are preferably selected from nucleotide analogues which are applicable for transcription and/or translation.

In particularly preferred embodiments, the nucleotide analogues/modifications which may be incorporated into a modified RNA are preferably selected from 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5-triphosphate, 2-thiouridine-5-triphosphate, 2′-Fluorothymidine-5′-triphosphate, 2′-O-Methyl-inosine-5′-triphosphate 4-thiouridine-5-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-iodo-2′-deoxycytidine-5′-triphosphate, 5-iodouridine-5-triphosphate, 5-iodo-2′-deoxyuridine-5′-triphosphate, 5-methylcytidine-5′-triphosphate, 5-methyluridine-5-triphosphate, 5-Propynyl-2′-deoxycytidine-5-triphosphate, 5-Propynyl-2′-deoxyuridine-5′-triphosphate, 6-azacytidine-5-triphosphate, 6-azauridine-5-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphate)-uidine, 5′-O-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, 7-deaza-adenosine.

In some embodiments, the at least one modified nucleotide is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

Particularly preferred in that context are pseudouridine (ψ), N1-methylpseudouridine (m1ψ), 5-methylcytosine, and 5-methoxyuridine.

Accordingly, in embodiments, the at least one nucleic acid comprises at least one modified nucleotide.

In some embodiments, essentially all, e.g. essentially 100% of the uracil in the coding sequence of the at least one nucleic acid have a chemical modification, preferably a chemical modification is in the 5-position of the uracil.

Incorporating modified nucleotides such as e.g. pseudouridine (ψ), N1-methylpseudouridine (m1ψ), 5-methylcytosine, and/or 5-methoxyuridine into the coding sequence may be advantageous as unwanted innate immune responses (upon administration of the coding RNA or the vaccine) may be adjusted or reduced (if required).

In embodiments, the RNA comprises at least one coding sequence encoding at least one antigenic protein as defined herein, wherein said coding sequence comprises at least one modified nucleotide selected from pseudouridine (ψ) and N1-methylpseudouridine (m1ψ), preferably wherein all uracil nucleotides are replaced by pseudouridine (ψ) nucleotides and/or N1-methylpseudouridine (m1ψ) nucleotides, optionally wherein all uracil nucleotides are replaced by pseudouridine (ψ) nucleotides and/or N1-methylpseudouridine (m1ψ) nucleotides.

In preferred embodiments, the RNA does not comprise N1-methylpseudouridine (m1ψ) substituted positions. In further embodiments, the RNA does not comprise pseudouridine (ψ), N1-methylpseudouridine (m1ψ), 5-methylcytosine, and 5-methoxyuridine substituted position.

In preferred embodiments, the RNA comprises a coding sequence that consists only of G, C, A and U nucleotides and therefore does not comprise modified nucleotides (except of the 5′ terminal cap structure (cap0, cap1, cap2)).

In embodiments, the at least one nucleic acid is an RNA, wherein the RNA may be prepared using any method known in the art, including chemical synthesis such as e.g. solid phase RNA synthesis, as well as in vitro methods, such as RNA in vitro transcription reactions. Accordingly, in a preferred embodiment, the RNA is obtained by RNA in vitro transcription.

Accordingly, in preferred embodiments, the at least one nucleic acid is preferably an in vitro transcribed RNA.

The terms “RNA in vitro transcription” or “in vitro transcription” relate to a process wherein RNA is synthesized in a cell-free system (in vitro). RNA may be obtained by DNA-dependent in vitro transcription of an appropriate DNA template, which according to the present invention is a linearized plasmid DNA template or a PCR-amplified DNA template. The promoter for controlling RNA in vitro transcription can be any promoter for any DNA-dependent RNA polymerase. Particular examples of DNA-dependent RNA polymerases are the T7, T3, SP6, or Syn5 RNA polymerases. In a preferred embodiment of the present invention the DNA template is linearized with a suitable restriction enzyme, before it is subjected to RNA in vitro transcription.

Reagents used in RNA in vitro transcription typically include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); optionally, a cap analogue as defined herein; optionally, further modified nucleotides as defined herein; a DNA-dependent RNA polymerase capable of binding to the promoter sequence within the DNA template (e.g. T7, T3, SP6, or Syn5 RNA polymerase); optionally, a ribonuclease (RNase) inhibitor to inactivate any potentially contaminating RNase; optionally, a pyrophosphatase to degrade pyrophosphate, which may inhibit RNA in vitro transcription; MgCl₂, which supplies Mg2+ ions as a co-factor for the polymerase; a buffer (TRIS or HEPES) to maintain a suitable pH value, which can also contain antioxidants (e.g. DTT), and/or polyamines such as spermidine at optimal concentrations, e.g. a buffer system comprising TRIS-Citrate as disclosed in WO2017109161.

In preferred embodiments, the cap1 structure of the RNA is formed using co-transcriptional capping using tri-nucleotide cap analogues m7G(5′)ppp(5′)(2′OMeA)pG or m7G(5′)ppp(5′)(2′OMeG)pG. A preferred cap1 analogue that may suitably be used in manufacturing the coding RNA of the invention is m7G(5′)ppp(5′)(2′OMeA)pG.

In other preferred embodiments, the cap1 structure of the RNA of the invention is formed using co-transcriptional capping using tri-nucleotide cap analogue 3′OMe-m7G(5′)ppp(5′)(2′OMeA)pG.

In other embodiments, a cap0 structure of the RNA of the invention is formed using co-transcriptional capping using cap analogue 3′OMe-m7G(5′)ppp(5′)G.

In embodiments, the nucleotide mixture used in RNA in vitro transcription may additionally comprise modified nucleotides as defined herein. In that context, preferred modified nucleotides may be selected from pseudouridine (ψ), N1-methylpseudouridine (m1ψ), 5-methylcytosine, and 5-methoxyuridine. In particular embodiments, uracil nucleotides in the nucleotide mixture are replaced (either partially or completely) by pseudouridine (ψ) and/or N1-methylpseudouridine (m1ψ) to obtain a modified RNA.

In preferred embodiments, the nucleotide mixture used in RNA in vitro transcription does not comprise modified nucleotides as defined herein. In preferred embodiments, the nucleotide mixture used in RNA in vitro transcription does only comprise G, C, A and U nucleotides, and, optionally, a cap analog as defined herein.

In preferred embodiments, the nucleotide mixture (i.e. the fraction of each nucleotide in the mixture) used for RNA in vitro transcription reactions may be optimized for the given RNA sequence, preferably as described WO2015188933.

In this context, the in vitro transcription has been performed in the presence of a sequence optimized nucleotide mixture and optionally a cap analog, preferably wherein the sequence optimized nucleotide mixture does not comprise chemically modified nucleotides.

In this context a sequence-optimized nucleoside triphosphate (NTP) mix is a mixture of nucleoside triphosphates (NTPs) for use in an in vitro transcription reaction of an RNA molecule of a given sequence comprising the four nucleoside triphosphates (NTPs) GTP, ATP, CTP and UTP, wherein the fraction of each of the four nucleoside triphosphates (NTPs) in the sequence-optimized nucleoside triphosphate (NTP) mix corresponds to the fraction of the respective nucleotide in said RNA molecule. If a ribonucleotide is not present in the RNA molecule, the corresponding nucleoside triphosphate is also not present in the sequence-optimized nucleoside triphosphate (NTP) mix. In embodiment where more than one different RNA as defined herein have to be produced, e.g. where 2, 3, 4, 5, 6, 7, 8, 9, 10 or even more different RNAs have to be produced, procedures as described in WO2017109134 may suitably be used.

In the context of nucleic acid-based vaccine production, it may be required to provide GMP-grade nucleic acid, e.g. a GMP grade RNA or DNA. GMP-grade RNA or DNA may be produced using a manufacturing process approved by regulatory authorities. Accordingly, in a particularly preferred embodiment, RNA production is performed under current good manufacturing practice (GMP), implementing various quality control steps on DNA and RNA level, preferably according to WO2016180430. In preferred embodiments, the RNA of the invention is a GMP-grade RNA, particularly a GMP-grade mRNA. Accordingly, an RNA for a vaccine is preferably a GMP grade RNA.

The obtained RNA products are preferably purified using PureMessenger® (CureVac, Tubingen, Germany; RP-HPLC according to WO2008077592) and/or tangential flow filtration (as described in WO2016193206) and/or oligo d(T) purification (see WO2016180430).

Preferably, the RNA according to the invention is purified using RP-HPLC, preferably using Reversed-Phase High pressure liquid chromatography (RP-HPLC) with a macroporous styrene/divinylbenzene column (e.g. particle size 30 μm, pore size 4000Å and additionally using a filter cassette with a cellulose based membrane with a molecular weight cutoff of about 100 kDa.

In a further preferred embodiment, the at least one nucleic acid, preferably the RNA, is lyophilized (e.g. according to WO2016165831 or WO2011069586) to yield a temperature stable dried nucleic acid (powder) as defined herein (e.g. RNA or DNA). The nucleic acid of the invention, particularly the RNA may also be dried using spray-drying or spray-freeze drying (e.g. according to WO2016184575 or WO2016184576) to yield a temperature stable RNA (powder) as defined herein. Accordingly, in the context of manufacturing and purifying nucleic acid, in particular RNA, the disclosures of WO2017109161, WO2015188933, WO2016180430, WO2008077592, WO2016193206, WO2016165831, WO2011069586, WO2016184575, and WO2016184576 are incorporated herewith by reference.

Accordingly, in preferred embodiments, the at least one nucleic acid is a dried nucleic acid, particularly a dried RNA.

The term “dried RNA” as used herein has to be understood as RNA that has been lyophilized, or spray-dried, or spray-freeze dried as defined above to obtain a temperature stable dried RNA (powder).

In preferred embodiments, the at least one nucleic acid is a purified nucleic acid, particularly a purified RNA.

The term “purified nucleic acid” as used herein has to be understood as nucleic acid which has a higher purity after certain purification steps than the starting material. Typical impurities that are essentially not present in purified nucleic acid comprise peptides or proteins, spermidine, BSA, abortive nucleic acid sequences, nucleic acid fragments, free nucleotides, bacterial impurities, or impurities derived from purification procedures. Accordingly, it is desirable in this regard for the “degree of nucleic acid purity” to be as close as possible to 100%. It is also desirable for the degree of nucleic acid purity that the amount of full-length nucleic acid is as close as possible to 100%. Accordingly “purified nucleic acid” as used herein has a degree of purity of more than 75%, 80%, 85%, very particularly 90%, 91%, 92%, 93%, 94%, 95%, 96%, 97%, 98% and most favorably 99% or more. The degree of purity may for example be determined by an analytical HPLC, wherein the percentages provided above correspond to the ratio between the area of the peak for the target nucleic acid and the total area of all peaks representing the by-products. Alternatively, the degree of purity may for example be determined by an analytical agarose gel electrophoresis or capillary gel electrophoresis.

In preferred embodiments, the at least one nucleic acid is a purified RNA, preferably a purified mRNA.

The term “purified RNA” or “purified mRNA” as used herein has to be understood as RNA which has a higher purity after certain purification steps (e.g. HPLC, TFF, Oligo d(T) purification, precipitation steps) than the starting material (e.g. in vitro transcribed RNA). Typical impurities that are essentially not present in purified RNA comprise peptides or proteins (e.g. enzymes derived from DNA dependent RNA in vitro transcription, e.g. RNA polymerases, RNases, pyrophosphatase, restriction endonuclease, DNase), spermidine, BSA, abortive RNA sequences, RNA fragments (short double stranded RNA fragments, abortive sequences etc.), free nucleotides (modified nucleotides, conventional NTPs, cap analogue), template DNA fragments, buffer components (HEPES, TRIS, MgCl₂) etc. Other potential impurities that may be derived from e.g. fermentation procedures comprise bacterial impurities (bioburden, bacterial DNA) or impurities derived from purification procedures (organic solvents etc.). Accordingly, it is desirable in this regard for the “degree of RNA purity” to be as close as possible to 100%. It is also desirable for the degree of RNA purity that the amount of full-length RNA transcripts is as close as possible to 100%. Accordingly, “purified RNA” as used herein has a degree of purity of more than 75%, 80%, 85%, very particularly 90%, 91%, 92%, 93%, 94%, 95%, 96%, 97%, 98% and most favorably 99% or more. The degree of purity may for example be determined by an analytical HPLC, wherein the percentages provided above correspond to the ratio between the area of the peak for the target RNA and the total area of all peaks representing the by-products. Alternatively, the degree of purity may for example be determined by an analytical agarose gel electrophoresis or capillary gel electrophoresis.

In particularly preferred embodiments where the nucleic acid is an RNA, the RNA has been purified by RP-HPLC and/or TFF to remove double-stranded RNA, non-capped RNA and/or RNA fragments.

The formation of double stranded RNA as side products during e.g. RNA in vitro transcription can lead to an induction of the innate immune response, particularly IFNalpha which is the main factor of inducing fever in vaccinated subjects, which is of course an unwanted side effect. Current techniques for immunoblotting of dsRNA (via dot Blot, serological specific electron microscopy (SSEM) or ELISA for example) are used for detecting and sizing dsRNA species from a mixture of nucleic acids.

Suitably, the RNA of the invention has been purified by RP-HPLC and/or TFF as described herein to reduce the amount of dsRNA.

Preferably, the RNA according to the invention is purified using RP-HPLC, preferably using Reversed-Phase High pressure liquid chromatography (RP-HPLC) with a macroporous styrene/divinylbenzene column (e.g. particle size 30 μm, pore size 4000A and additionally using a filter cassette with a cellulose based membrane with a molecular weight cutoff of about 100 kDa.

In this context it is particularly preferred that the purified RNA has been purified by RP-HPLC and/or TFF which results in about 5%, 10%, or 20% less double stranded RNA side products as in RNA that has not been purified with RP-HPLC and/or TFF. Accordingly, the RNA of the invention comprises about 5%, 10%, or 20% less double stranded RNA side products as an RNA that has not been purified with RP-HPLC and/or TFF.

Alternatively, the purified RNA that has been purified by RP-HPLC and/or TFF comprises about 5%, 10%, or 20% less double stranded RNA side products as an RNA that has been purified with Oligo dT purification, precipitation, filtration and/or anion exchange chromatography. Accordingly, the RP-HPLC and/or TFF purified RNA of the invention comprises about 5%, 10%, or 20% less double stranded RNA side products as an RNA that has been purified with Oligo dT purification, precipitation, filtration and/or AEX.

It has to be understood that “dried RNA” as defined herein and “purified RNA” as defined herein or “GMP-grade RNA” as defined herein may have superior stability characteristics (in vitro, in vivo) and improved efficiency (e.g. better translatability of the mRNA in vivo) and are therefore particularly suitable for a medical purpose, e.g. a vaccine.

In embodiments, RNA of a composition has an RNA integrity ranging from about 40% to about 100%.

The term “RNA integrity” generally describes whether the complete RNA sequence is present in the composition. Low RNA integrity could be due to, amongst others, RNA degradation, RNA cleavage, incorrect or incomplete chemical synthesis of the RNA, incorrect base pairing, integration of modified nucleotides or the modification of already integrated nucleotides, lack of capping or incomplete capping, lack of polyadenylation or incomplete polyadenylation, or incomplete RNA in vitro transcription. RNA is a fragile molecule that can easily degrade, which may be caused e.g. by temperature, ribonucleases, pH or other factors (e.g. nucleophilic attacks, hydrolysis etc.), which may reduce the RNA integrity and, consequently, the functionality of the RNA.

In preferred embodiments, the RNA of a composition has an RNA integrity of at least about 50%, preferably of at least about 60%, more preferably of at least about 70%, most preferably of at least about 80% or about 90%. RNA is suitably determined using analytical HPLC, preferably analytical RP-HPLC.

The skilled person can choose from a variety of different chromatographic or electrophoretic methods for determining an RNA integrity. Chromatographic and electrophoretic methods are well-known in the art. In case chromatography is used (e.g. RP-HPLC), the analysis of the integrity of the RNA may be based on determining the peak area (or “area under the peak”) of the full-length RNA in a corresponding chromatogram. The peak area may be determined by any suitable software which evaluates the signals of the detector system. The process of determining the peak area is also referred to as integration. The peak area representing the full-length RNA is typically set in relation to the peak area of the total RNA in a respective sample. The RNA integrity may be expressed in % RNA integrity.

In the context of aspects of the invention, RNA integrity may be determined using analytical (RP)HPLC. Typically, a test sample of the composition comprising lipid based carrier encapsulating RNA may be treated with a detergent (e.g. about 2% Triton X100) to dissociate the lipid based carrier and to release the encapsulated RNA. The released RNA may be captured using suitable binding compounds, e.g. Agencourt AMPure XP beads (Beckman Coulter, Brea, CA, USA) essentially according to the manufacturer's instructions. Following preparation of the RNA sample, analytical (RP)HPLC may be performed to determine the integrity of RNA. Typically, for determining RNA integrity, 45 the RNA samples may be diluted to a concentration of 0.1 g/l using e.g. water for injection (WFI). About 10 μl of the diluted RNA sample may be injected into an HPLC column (e.g. a monolithic poly(styrene-divinylbenzene) matrix). Analytical (RP)HPLC may be performed using standard conditions, for example: Gradient 1: Buffer A (0.1M TEAA (pH 7.0)); Buffer B (0.1M TEAA (pH 7.0) containing 25% acetonitrile). Starting at 30% buffer B the gradient extended to 32% buffer B in 2 min, followed by an extension to 55% buffer B over 15 minutes at a flow rate of 1 ml/min. HPLC chromatograms are typically recorded at a wavelength of 260 nm. The obtained chromatograms may be evaluated using a software and the relative peak area may be determined in percent (%) as commonly known in the art. The relative peak area indicates the amount of RNA that has 100% RNA integrity. Since the amount of the RNA injected into the HPLC is typically known, the analysis of the relative peak area provides information on the integrity of the RNA. Thus, if e.g. 100 ng RNA have been injected in total, and 100 ng are determined as the relative peak area, the RNA integrity would be 100%. If, for example, the relative peak area would correspond to 80 ng, the RNA integrity would be 80%. Accordingly, RNA integrity in the context of the invention is determined using analytical HPLC, preferably analytical RP-HPLC.

In embodiments, RNA of a composition has an RNA integrity ranging from about 40% to about 100%. In embodiments, the RNA has an RNA integrity ranging from about 50% to about 100%. In embodiments, the RNA has an RNA integrity ranging from about 60% to about 100%. In embodiments, the RNA has an RNA integrity ranging from about 70% to about 100%. In embodiments, the RNA integrity is for example about 50%, about 60%, about 70%, about 80%, or about 90%. RNA is suitably determined using analytical HPLC, preferably analytical RP-HPLC.

In preferred embodiments, the RNA of a composition has an RNA integrity of at least about 50%, preferably of at least about 60%, more preferably of at least about 70%, most preferably of at least about 80% or about 90%. RNA integrity is suitably determined using analytical HPLC, preferably analytical RP-HPLC.

Following co-transcriptional capping as defined herein, and following purification as defined herein, the capping degree of the obtained RNA may be determined using capping assays as described in published PCT application WO2015101416, in particular, as described in Claims 27 to 46 of published PCT application WO2015101416 can be used. Alternatively, a capping assays described in PCT/EP2018/08667 may be used.

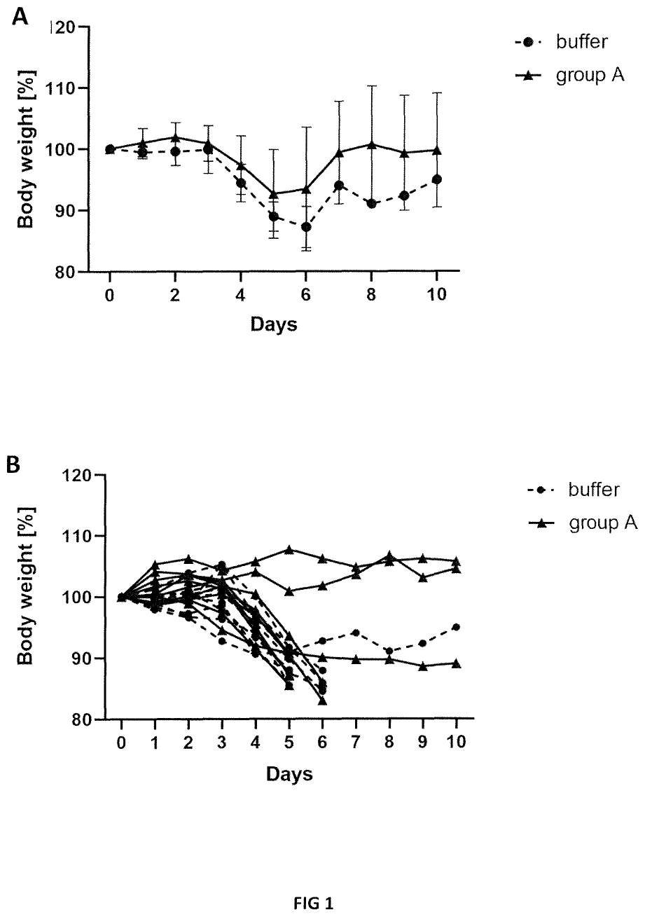

In embodiments, an automated device for performing RNA in vitro transcription may be used to produce and purify the at least one nucleic acid. Such a device may also be used to produce the composition or the vaccine. Preferably, a device as described in WO2020002598, in particular, a device as described in claims 1 to 59 and/or 68 to 76 of WO2020002598 (and FIGS. 1-18) may suitably be used.

In a further aspect, the present invention provides a method of stabilizing a pharmaceutical composition or a combination vaccine comprising lyophilizing or spray-drying the composition or vaccine to produce a stabilized composition or vaccine. Preferably, the stabilized composition or vaccine has a water content of less than about 10%, preferably a water content of between about 0.5% and 5.0%. Accordingly, the invention provides a stabilized, lyophilized composition or vaccine produced by a method of stabilizing as defined herein.

The methods described herein may preferably applied to a method of producing a pharmaceutical composition or vaccine as described in further detail below.

In various embodiments the at least one nucleic acid, preferably the mRNA comprises, preferably in 5′- to 3′-direction, the following elements:
A) 5-cap structure, preferably as specified herein;
B) 5-terminal start element, preferably as specified herein;
C) optionally, a 5′-UTR, preferably as specified herein;
D) a ribosome binding site, preferably as specified herein;
E) at least one coding sequence, preferably as specified herein;
F) 3′-UTR, preferably as specified herein;
G) optionally, poly(A) sequence, preferably as specified herein;
H) optionally, poly(C) sequence, preferably as specified herein;
I) optionally, histone stem-loop preferably as specified herein;
J) optionally, 3-terminal sequence element, preferably as specified herein.

In preferred embodiments the at least one nucleic acid, preferably the mRNA, comprises the following elements preferably in 5′- to 3-direction:
A) 5′-cap structure selected from m7G(5′), m7G(5′)ppp(5′)(2′OMeA), or m7G(5′)ppp(5′)(2′OMeG);
B) 5-terminal start element selected from SEQ ID NOs: 176 or 177 or fragments or variants thereof;
C) optionally, a 5′-UTR derived from a HSD17B4 gene;
D) a ribosome binding site selected from SEQ ID NOs: 180, 181, 22845-22847 or fragments or variants thereof;
E) at least one coding sequence as specified herein, preferably a codon optimized coding sequence;
F) 3′-UTR derived from a 3′-UTR of a PSMB3 gene or an alpha-globin gene (“muag”);
G) optionally, poly(A) sequence comprising about 30 to about 500 adenosines;
H) optionally, poly(C) sequence comprising about 10 to about 100 cytosines;
I) optionally, histone stem-loop selected from SEQ ID NOs: 178 or 179;
J) optionally, 3-terminal sequence element selected from SEQ ID NOs: 182-230.

In particularly preferred embodiments the at least one nucleic acid, preferably the mRNA, comprises the following elements in 5′- to 3-direction:
A) cap1 structure as defined herein;
B) coding sequence as specified herein, preferably a codon optimized coding sequence;
C) 3′-UTR derived from a 3′-UTR of a muag gene as defined herein, preferably according to SEQ ID NO: 267, 268, 22896-22901, 22906-22911;
D) poly(A) sequence comprising about 64 A nucleotides.
E) poly(C) sequence comprising about 10 to about 100 cytosines;
F) histone stem-loop selected from SEQ ID NOs: 178 or 179;

In preferred embodiments the at least one nucleic acid, preferably the mRNA, comprises the following elements in 5′- to 3′-direction:
A) cap1 structure as defined herein, preferably a codon optimized coding sequence;
B) 5′-UTR derived from a HSD17B4 gene as defined herein, preferably according to SEQ ID NO: 231 or 232;
C) coding sequence as specified herein;
D) 3-UTR derived from a 3-UTR of a PSMB3 gene as defined herein, preferably according to SEQ ID NO: 253 or 254;
E) poly(A) sequence comprising about 64 A nucleotides.
F) optionally a poly(C) sequence comprising about 10 to about 100 cytosines;
G) histone stem-loop selected from SEQ ID NOs: 178 or 179;
H) optionally, 3-terminal sequence element SEQ ID NOs: 182-230.

In particularly preferred embodiments the at least one nucleic acid, preferably the mRNA, comprises the following elements in 5′- to 3-direction:
A) cap1 structure as defined herein;
B) 5′-UTR derived from a HSD17B4 gene as defined herein, preferably according to SEQ ID NO: 231 or 232;
C) coding sequence selected as specified herein, preferably a codon optimized coding sequence;
D) 3′-UTR derived from a 3′-UTR of a PSMB3 gene as defined herein, preferably according to SEQ ID NO: 253 or 254;
E) a histone stem-loop selected from SEQ ID NOs: 178 or 179;
F) poly(A) sequence comprising about 100 A nucleotides, preferably representing the 3′ terminus.

In even more particularly preferred embodiments, the at least one nucleic acid, preferably the mRNA, comprises the following elements in 5′- to 3′-direction:
A) cap1 structure as defined herein;
B) 5′-UTR derived from a HSD17B4 gene as defined herein, preferably according to SEQ ID NO: 231 or 232;
C) coding sequence selected as specified herein, preferably a codon optimized coding sequence;
D) 3′-UTR derived from a 3′-UTR of a PSMB3 gene as defined herein, preferably according to SEQ ID NO: 253 or 254;
E) poly(A) sequence comprising about 100 A nucleotides, preferably representing the 3′ terminus.

In further preferred embodiments, the at least one nucleic acid, preferably the mRNA, comprises the following elements in 5′- to 3′-direction:
A) cap1 structure as defined herein;
B) 5′-UTR derived from a SLC7A3 gene as defined herein, preferably according to SEQ ID NO: 245 or 246;
C) coding sequence selected as specified herein, preferably a codon optimized coding sequence;
D) 3′-UTR derived from a 3′-UTR of a PSMB3 gene as defined herein, preferably according to SEQ ID NO: 253 or 254;
E) optionally, a histone stem-loop selected from SEQ ID NOs: 178 or 179;
F) poly(A) sequence comprising about 100 A nucleotides, preferably representing the 3′ terminus.

In further preferred embodiments, the at least one nucleic acid, preferably the, comprises the following elements in 5′- to 3′-direction:
A) cap1 structure as defined herein;
B) 5′-UTR derived from a RPL31 gene as defined herein, preferably according to SEQ ID NO: 243 or 243;
C) coding sequence selected as specified herein, preferably a codon optimized coding sequence;
D) 3′-UTR derived from a 3′-UTR of a RPS9 gene as defined herein, preferably according to SEQ ID NO: 263 or 264;
E) optionally, a histone stem-loop selected from SEQ ID NOs: 178 or 179;
F) poly(A) sequence comprising about 100 A nucleotides, preferably representing the 3′ terminus.

In the following Table 9, suitable constructs selected or derived from SARS-CoV2 are provided, with the encoded antigenic protein indicated therein. Further indicated are suitable codon optimized cds sequences (CDS opt), the used UTR design (5′-UTR/3′-UTR), the used 3′ end of the construct (3′ end), and the respective protein, cds and mRNA sequences.

TABLE 9
Overview of preferred mRNA constructs

| | | | 5′-UTR/3′- | | SEQ ID | SEQ | SEQ |
|---|---|---|---|---|---|---|---|
| | | CDS | UTR; UTR | | NO: | ID NO: | ID NO: |
| RNA ID | Name | opt. | Design | 3′-end | Protein | CDS | mRNA |
| R9922 | SARS-CoV-2/Wuhan-Hu-1 | opt1 | —/muag; i-3 | A64-N5-C30- | 15235 | 17112 | 20866 |
| | membrane protein | (gc) | | hSL-N5 | | | |
| R9924 | SARS-CoV-2/Wuhan-Hu-1 | opt1 | HDS17B4/ | hSL-A100 | 15235 | 17112 | 18989 |
| | membrane protein | (gc) | PSMB3; a-1 | | | | |
| R9923 | SARS-CoV-2/Wuhan-Hu-1 | opt1 | —/muag; i-3 | A64-N5-C30- | 15310 | 17187 | 20941 |
| | nucleocapsid protein | (gc) | | hSL-N5 | | | |
| R9925 | SARS-CoV-2/Wuhan-Hu-1 | opt1 | HDS17B4/ | hSL-A100 | 15310 | 17187 | 19064 |
| | nucleocapsid protein | (gc) | PSMB3; a-1 | | | | |
| R9941 | SARS-CoV-2/Wuhan-Hu-1 | opt1 | —/muag; i-3 | A64-N5-C30- | 15733 | 17610 | 21364 |
| | non-structural protein 3 | (gc) | | hSL-N5 | | | |
| R9946 | SARS-CoV-2/Wuhan-Hu-1 | opt1 | HDS17B4/ | hSL-A100 | 15733 | 17610 | 19487 |
| | non-structural protein 3 | (gc) | PSMB3; a-1 | | | | |
| R9942 | SARS-CoV-2/Wuhan-Hu-1 | opt1 | —/muag; i-3 | A64-N5-C30- | 16415 | 18292 | 22046 |
| | non-structural protein 4 | (gc) | | hSL-N5 | | | |
| R9947 | SARS-CoV-2/Wuhan-Hu-1 | opt1 | HDS17B4/ | hSL-A100 | 16415 | 18292 | 20169 |
| | non-structural protein 4 | (gc) | PSMB3; a-1 | | | | |
| R9943 | SARS-CoV-2/Wuhan-Hu-1 | opt1 | —/muag; i-3 | A64-N5-C30- | 16582 | 18459 | 22213 |
| | non-structural protein 6 | (gc) | | hSL-N5 | | | |
| R9948 | SARS-CoV-2/Wuhan-Hu-1 | opt1 | HDS17B4/ | hSL-A100 | 16582 | 18459 | 20336 |
| | non-structural protein 6 | (gc) | PSMB3; a-1 | | | | |
| R9944 | SARS-CoV-2/Wuhan-Hu-1 | opt1 | —/muag; i-3 | A64-N5-C30- | 16684 | 18561 | 22315 |
| | ORF3a protein | (gc) | | hSL-N5 | | | |
| R9949 | SARS-CoV-2/Wuhan-Hu-1 | opt1 | HDS17B4/ | hSL-A100 | 16684 | 18561 | 20438 |
| | ORF3a protein | (gc) | PSMB3; a-1 | | | | |
| R9945 | SARS-CoV-2/Wuhan-Hu-1 | opt1 | —/muag; i-3 | A64-N5-C30- | 16997 | 18874 | 22628 |
| | ORF8 protein | (gc) | | hSL-N5 | | | |
| R9950 | SARS-CoV-2/Wuhan-Hu-1 | opt1 | HDS17B4/ | hSL-A100 | 16997 | 18874 | 20751 |
| | ORF8 protein | (gc) | PSMB3; a-1 | | | | |
| R9515 | SARS-CoV-2/Wuhan-Hu-1 or SARS- | opt1 | —/muag; i-3 | A64-N5-C30- | 10, 341 | 137, 11798 | 163, 12810 |
| | CoV-2/Wuhan/WIV05/2019 | (gc) | | hSL-N5 | | | |
| | S_stab_PP (K986P_V987P) | | | | | | |
| R9709 | SARS-CoV-2/Wuhan-Hu-1 or SARS- | opt1 | HDS17B4/ | hSL-A100 | 10 | 137 | 149 |
| | CoV-2/Wuhan/WIV05/2019 | (gc) | PSMB3; a-1 | | | | |
| | S_stab_PP (K986P_V987P) | | | | | | |
| R10384 | SARS-CoV-2/Wuhan-Hu-1 or SARS- | opt1 | HDS17B4/ | hSL-A100 | 22961 | 23091 | 23531 |
| | CoV-2/Wuhan/WIV05/2019 | (gc) | PSMB3; a-1 | | | | |
| | S_stab_PP (K986P_V987P_L18F_{—} | | | | | | |
| | D80A_D215G_L242del_A243del_{—} | | | | | | |
| | L244del_R246I_K417N_E484K_{—} | | | | | | |
| | N501Y_D614G_A701V) | | | | | | |
| R10385 | SARS-CoV-2/Wuhan-Hu-1 or SARS- | opt1 | HDS17B4/ | hSL-A100 | 22963 | 23093 | 23533 |
| | CoV-2/Wuhan/WIV05/2019 | (gc) | PSMB3; a-1 | | | | |
| | S_stab_PP (K986P_V987P_L18F_{—} | | | | | | |
| | T20N_P26S_D138Y_R190S_K417T_{—} | | | | | | |
| | E484K_N501Y_D614G_H655Y_{—} | | | | | | |
| | T1027I) | | | | | | |
| R10354 | SARS-CoV-2/Wuhan-Hu-1 non- | opt1 | —/muag; i-3 | A64-N5-C30- | 27908 | 28029 | 28401 |
| | structural protein 13 protein | (gc) | | hSL-N5 | | | |
| R10355 | SARS-CoV-2/Wuhan-Hu-1 non- | opt1 | —/muag; i-3 | A64-N5-C30- | 27909 | 28030 | 28402 |
| | structural protein 14 protein | (gc) | | hSL-N5 | | | |
| R10382 | SARS-CoV-2/Wuhan-Hu-1 non- | opt1 | HDS17B4/ | hSL-A100 | 27908 | 28029 | 28150 |
| | structural protein 13 protein | (gc) | PSMB3; a-1 | | | | |
| R10383 | SARS-CoV-2/Wuhan-Hu-1 non- | opt1 | HDS17B4/ | hSL-A100 | 27909 | 28030 | 28151 |
| | structural protein 14 protein | (gc) | PSMB3; a-1 | | | | |

Formulation and Complexation:

In the following, suitable features and embodiments relating to the formulation and/or complexation of the pharmaceutical composition are further specified. In particular, suitable features and embodiments relating to formulation and/or complexation of nucleic acid molecules of the composition are further specified.

It has to be understood that suitable formulations/complexations provided herein may relate to any of the at least one nucleic acid comprising at least one coding sequence encoding at least one antigenic peptide or protein from at least one Coronavirus M, N, non-structural protein, and/or accessory protein as defined herein. Further, suitable features and embodiments provided herein may relate to any of the at least one (additional) nucleic acid comprising at least one coding sequence encoding at least one antigenic peptide or protein selected or derived from at least one Coronavirus spike protein (S) as defined herein.

In preferred embodiments, the pharmaceutical composition comprises at least one pharmaceutically acceptable carrier or excipient.

The term “pharmaceutically acceptable carrier” or “pharmaceutically acceptable excipient” as used herein preferably includes the liquid or non-liquid basis of the composition for administration. If the composition is provided in liquid form, the carrier may be water, e.g. pyrogen-free water; isotonic saline or buffered (aqueous) solutions, e.g. phosphate, citrate etc. buffered solutions. Water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, preferably at least 3 mM of a potassium salt. According to preferred embodiments, the sodium, calcium and, optionally, potassium salts may occur in the form of their halogenides, e.g. chlorides, iodides, or bromides, in the form of their hydroxides, carbonates, hydrogen carbonates, or sulfates, etc. Examples of sodium salts include NaCl, NaI, NaBr, Na₂CO₃, NaHCO₃, Na₂SO₄, examples of the optional potassium salts include KCl, KI, KBr, K₂CO₃, KHCO₃, K₂SO₄, and examples of calcium salts include CaCl₂), CaI₂, CaBr₂, CaCO₃, CaSO₄, Ca(OH)₂.

Furthermore, organic anions of the aforementioned cations may be in the buffer. Accordingly, in embodiments, the pharmaceutical composition may comprise pharmaceutically acceptable carriers or excipients using one or more pharmaceutically acceptable carriers or excipients to e.g. increase stability, increase cell transfection, permit the sustained or delayed, increase the translation of encoded antigenic peptides or proteins in vivo, and/or alter the release profile of encoded antigenic peptides or proteins protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with polynucleotides, hyaluronidase, nanoparticle mimics and combinations thereof. In embodiments, one or more compatible solid or liquid fillers or diluents or encapsulating compounds may be used as well, which are suitable for administration to a subject. The term “compatible” as used herein means that the constituents of the composition are capable of being mixed with the at least one nucleic acid and, optionally, a plurality of nucleic acids of the composition, in such a manner that no interaction occurs, which would substantially reduce the biological activity or the pharmaceutical effectiveness of the composition under typical use conditions (e.g., intramuscular or intradermal administration). Pharmaceutically acceptable carriers or excipients must have sufficiently high purity and sufficiently low toxicity to make them suitable for administration to a subject to be treated. Compounds which may be used as pharmaceutically acceptable carriers or excipients may be sugars, such as, for example, lactose, glucose, trehalose, mannose, and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

The at least one pharmaceutically acceptable carrier or excipient of the pharmaceutical composition may preferably be selected to be suitable for intramuscular or intradermal delivery/administration of said pharmaceutical composition. The pharmaceutical composition is preferably a composition suitable for intramuscular administration to a subject.

Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Pharmaceutical compositions of the present invention may suitably be sterile and/or pyrogen-free.

In a preferred embodiments, the at least one nucleic acid is complexed or associated with further compound to obtain a formulated composition. A formulation in that context may have the function of a transfection agent. A formulation in that context may also have the function of protecting the nucleic acid from degradation.

In embodiments where at least two nucleic acid species are in the composition, the at least two nucleic acid species are formulated separately.

In preferred embodiments where at least two nucleic acid species are in the composition, the at least two nucleic acid species are co-formulated.

In a preferred embodiment, the at least one nucleic acid, preferably the at least one RNA, is complexed or associated with, or at least partially complexed or partially associated with one or more cationic or polycationic compound.

In preferred where at least two nucleic acid species, the least two nucleic acid species are complexed or associated with, or at least partially complexed or partially associated with one or more cationic or polycationic compound, wherein the at least two nucleic acid species are formulated separately or are co-formulated.

In preferred embodiments, the one or more cationic or polycationic compound is selected from a cationic or polycationic polymer, cationic or polycationic polysaccharide, cationic or polycationic lipid, cationic or polycationic protein, cationic or polycationic peptide, or any combinations thereof.

The term “cationic or polycationic compound” as used herein will be recognized and understood by the person of ordinary skill in the art, and is for example intended to refer to a charged molecule, which is positively charged at a pH value ranging from about 1 to 9, at a pH value ranging from about 3 to 8, at a pH value ranging from about 4 to 8, at a pH value ranging from about 5 to 8, more preferably at a pH value ranging from about 6 to 8, even more preferably at a pH value ranging from about 7 to 8, most preferably at a physiological pH, e.g. ranging from about 7.2 to about 7.5. Accordingly, a cationic component, e.g. a cationic peptide, cationic protein, cationic polymer, cationic polysaccharide, cationic lipid (including lipidoids) may be any positively charged compound or polymer which is positively charged under physiological conditions. A “cationic or polycationic peptide or protein” may contain at least one positively charged amino acid, or more than one positively charged amino acid, e.g. selected from Arg, His, Lys or Orn. Accordingly, “polycationic” components are also within the scope exhibiting more than one positive charge under the given conditions.

Cationic or polycationic compounds, being particularly preferred in this context may be selected from the following list of cationic or polycationic peptides or proteins of fragments thereof: protamine, nucleoline, spermine or spermidine, or other cationic peptides or proteins, such as poly-L-lysine (PLL), poly-arginine, basic polypeptides, cell penetrating peptides (CPPs), including HIV-binding peptides, HIV-1 Tat (HIV), Tat-derived peptides, Penetratin, VP22 derived or analog peptides, HSV VP22 (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides, pAntp, pIsI, FGF, Lactoferrin, Transportan, Buforin-2, Bac715-24, SynB, SynB(1), pVEC, hCT-derived peptides, SAP, or histones. More preferably, the nucleic acid (e.g. DNA or RNA), e.g. the coding RNA, preferably the mRNA, is complexed with one or more polycations, preferably with protamine or oligofectamine, most preferably with protamine. In preferred embodiment, the at least one nucleic (e.g. DNA or RNA), preferably the at least one RNA, is complexed with protamine.

Further preferred cationic or polycationic compounds, which can be used as transfection or complexation agent may include cationic polysaccharides, for example chitosan, polybrene etc.; cationic lipids, e.g. DOTMA, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS, DIMRI, DOTAP, DC-6-14, CLIP1, CLIP6, CLIP9, oligofectamine; or cationic or polycationic polymers, e.g. modified polyaminoacids, such as beta-aminoacid-polymers or reversed polyamides, etc., modified polyethylenes, such as PVP etc., modified acrylates, such as pDMAEMA etc., modified amidoamines such as pAMAM etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI, poly(propyleneimine), etc., polyallylamine, sugar backbone based polymers, such as cyclodextrin based polymers, dextran based polymers, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole); etc.

In this context it is particularly preferred that the at least one nucleic acid, preferably the at least one RNA, is complexed or at least partially complexed with a cationic or polycationic compound and/or a polymeric carrier, preferably cationic proteins or peptides. In this context, the disclosure of WO2010037539 and WO2012113513 is incorporated herewith by reference. Partially means that only a part of the nucleic acid is complexed with a cationic compound and that the rest of the nucleic acid is in uncomplexed form (“free”).

In embodiments, the pharmaceutical composition comprises at least one nucleic acid, preferably at least one RNA, complexed with one or more cationic or polycationic compounds, preferably protamine, and at least one free (non-complexed) nucleic acid.

In this context it is particularly preferred that the at least one nucleic acid (e.g. DNA or RNA), preferably the at least one RNA is complexed, or at least partially complexed with protamine. Preferably, the molar ratio of the nucleic acid, particularly the RNA of the protamine-complexed RNA to the free RNA may be selected from a molar ratio of about 0.001:1 to about 1:0.001, including a ratio of about 1:1. Suitably, the complexed RNA is complexed with protamine by addition of protamine-trehalose solution to the RNA sample at a RNA:protamine weight to weight ratio (w/w) of 2:1.

Further preferred cationic or polycationic proteins or peptides that may be used for complexation of the nucleic acid can be derived from formula (Arg)l;(Lys)m;(His)n;(Om)o;(Xaa)x of the patent application WO2009030481 or WO2011026641, the disclosure of WO2009030481 or WO2011026641 relating thereto incorporated herewith by reference.

In preferred embodiments, the at least one nucleic acid (e.g. DNA or RNA), preferably the at least one RNA is complexed, or at least partially complexed, with at least one cationic or polycationic proteins or peptides preferably selected from SEQ ID NOs: 269 to 273, or any combinations thereof.

According to various embodiments, the composition of the present invention comprises at least one nucleic acid (e.g. DNA or RNA), preferably at least one RNA as defined herein, and a polymeric carrier.

The term “polymeric carrier” as used herein will be recognized and understood by the person of ordinary skill in the art, and are e.g. intended to refer to a compound that facilitates transport and/or complexation of another compound (e.g. cargo nucleic acid). A polymeric carrier is typically a carrier that is formed of a polymer. A polymeric carrier may be associated to its cargo (e.g. DNA, or RNA) by covalent or non-covalent interaction. A polymer may be based on different subunits, such as a copolymer.

Suitable polymeric carriers in that context may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PEGylated PLL and polyethylenimine (PEI), dithiobis(succinimidylpropionate) (DSP), Dimethyl-3,3′-dithiobispropionimidate (DTBP), poly(ethylene imine) biscarbamate (PEIC), poly(L-lysine) (PLL), histidine modified PLL, poly(N-vinylpyrrolidone) (PVP), poly(propylenimine (PPI), poly(amidoamine) (PAMAM), poly(amido ethylenimine) (SS-PAEI), triehtylenetetramine (TETA), poly(β-aminoester), poly(4-hydroxy-L-proine ester) (PHP), poly(allylamine), poly(α-[4-aminobutyl]-L-glycolic acid (PAGA), Poly(D,L-lactic-co-glycolid acid (PLGA), Poly(N-ethyl-4-vinylpyridinium bromide), poly(phosphazene)s (PPZ), poly(phosphoester)s (PPE), poly(phosphoramidate)s (PPA), poly(N-2-hydroxypropylmethacrylamide) (pHPMA), poly(2-(dimethylamino)ethyl methacrylate) (pDMAEMA), poly(2-aminoethyl propylene phosphate) PPE_EA), galactosylated chitosan, N-dodecylated chitosan, histone, collagen and dextran-spermine. In one embodiment, the polymer may be an inert polymer such as, but not limited to, PEG. In one embodiment, the polymer may be a cationic polymer such as, but not limited to, PEI, PLL, TETA, poly(allylamine), Poly(N-ethyl-4-vinylpyridinium bromide), pHPMA and pDMAEMA. In one embodiment, the polymer may be a biodegradable PEI such as, but not limited to, DSP, DTBP and PEIC. In one embodiment, the polymer may be biodegradable such as, but not limited to, histine modified PLL, SS-PAEI, poly(β-aminoester), PHP, PAGA, PLGA, PPZ, PPE, PPA and PPE-EA.

A suitable polymeric carrier may be a polymeric carrier formed by disulfide-crosslinked cationic compounds. The disulfide-crosslinked cationic compounds may be the same or different from each other. The polymeric carrier can also contain further components. The polymeric carrier used according to the present invention may comprise mixtures of cationic peptides, proteins or polymers and optionally further components as defined herein, which are crosslinked by disulfide bonds (via —SH groups).

In this context, polymeric carriers according to formula {(Arg)l;(Lys)m;(His)n;(Orn)o;(Xaa)x(Cys)y} and formula Cys,{(Arg)l;(Lys)m;(His)n;(Om)o;(Xaa)x}Cys2 of the patent application WO2012013326 are preferred, the disclosure of WO2012013326 relating thereto incorporated herewith by reference.

In embodiments, the polymeric carrier used to complex the at least one nucleic acid (e.g. DNA or RNA), preferably the at least one RNA, may be derived from a polymeric carrier molecule according formula (L-P¹-S-[S-P²-S]ₙ—S—P³-L) of the patent application WO2011026641, the disclosure of WO2011026641 relating thereto incorporated herewith by reference.

In embodiments, the polymeric carrier compound is formed by, or comprises or consists of the peptide elements CysArg12Cys (SEQ ID NO: 269) or CysArg12 (SEQ ID NO: 270) or TrpArg12Cys (SEQ ID NO: 271). In particularly preferred embodiments, the polymeric carrier compound consists of a (R₁₂C)-(R₁₂C) dimer, a (WR₁₂C)-(WR₁₂C) dimer, or a (CR₁₂)-(CR₁₂C)-(CR₁₂) trimer, wherein the individual peptide elements in the dimer (e.g. (WR₁₂C)), or the trimer (e.g. (CR₁₂)), are connected via —SH groups.

In a embodiments, at least one nucleic acid (e.g. DNA or RNA), preferably the at least one RNA is complexed or associated with a polyethylene glycol/peptide polymer comprising HO-PEG5000-S-(S-CHHHHHHRRRRHHHHHHC-S-)7-S-PEG5000-OH (SEQ ID NO: 272 as peptide monomer), HO-PEG5000-S-(S-CHHHHHHRRRRHHHHHHC-S-)4-S-PEG5000-OH (SEQ ID NO: 272 as peptide monomer), HO-PEG5000-S-(S-CGHHHHHRRRRHHHHHGC-S-)7-S-PEG5000-OH (SEQ ID NO: 273 as peptide monomer) and/or a polyethylene glycol/peptide polymer comprising HO-PEG5000-S-(S-CGHHHHHRRRRHHHHHGC-S-)4-S-PEG5000-OH (SEQ ID NO: 273 of the peptide monomer).

In other embodiments, the composition comprises at least one nucleic acid (e.g. DNA or RNA), wherein the at least one nucleic acid, preferably the at least one RNA is complexed or associated with polymeric carriers and, optionally, with at least one lipid component as described in WO2017212008A1, WO2017212006A1, WO2017212007A1, and WO2017212009A1. In this context, the disclosures of WO2017212008A1, WO2017212006A1, WO2017212007A1, and WO2017212009A1 are herewith incorporated by reference.

In preferred embodiments, the polymeric carrier is a peptide polymer, preferably a polyethylene glycol/peptide polymer as defined above, and a lipid component, preferably a lipidoid component.

A lipidoid (or lipidoit) is a lipid-like compound, i.e. an amphiphilic compound with lipid-like physical properties. The lipidoid is preferably a compound, which comprises two or more cationic nitrogen atoms and at least two lipophilic tails. In contrast to many conventional cationic lipids, the lipidoid may be free of a hydrolysable linking group, in particular linking groups comprising hydrolysable ester, amide or carbamate groups. The cationic nitrogen atoms of the lipidoid may be cationisable or permanently cationic, or both types of cationic nitrogens may be present in the compound. In the context of the present invention, the term lipid is considered to also encompass lipidoids.

In some embodiments of the inventions, the lipidoid may comprise a PEG moiety.

In preferred embodiments, the at least one nucleic acid (e.g. DNA or RNA), preferably the at least one RNA, is complexed or associated with a polymeric carrier, preferably with a polyethylene glycol/peptide polymer as defined above, and a lipidoid component.

In embodiments where at least two nucleic acid species are in the composition, the at least two nucleic acid species are complexed or associated with, or at least partially complexed or partially associated with polymer as defined above, and a lipid or lipidoid component as defined above, wherein the at least two nucleic acid species are formulated separately or are co-formulated.

Suitably, the lipidoid is cationic, which means that it is cationisable or permanently cationic. In one embodiment, the lipidoid is cationisable, i.e. it comprises one or more cationisable nitrogen atoms, but no permanently cationic nitrogen atoms. In another embodiment, at least one of the cationic nitrogen atoms of the lipidoid is permanently cationic. Optionally, the lipidoid comprises two permanently cationic nitrogen atoms, three permanently cationic nitrogen atoms, or even four or more permanently cationic nitrogen atoms.

In a preferred embodiment, the lipidoid component may be any one selected from the lipidoids of the lipidoids provided in the table of page 50-54 of published PCT patent application WO2017212009A1, the specific lipidoids provided in said table, and the specific disclosure relating thereto herewith incorporated by reference.

In preferred embodiments, the lipidoid component may be any one selected from 3-C12-OH, 3-C12-OH-cat, 3-C12-amide, 3-C12-amide monomethyl, 3-C12-amide dimethyl, RevPEG(10)-3-C12-OH, RevPEG(10)-DLin-pAbenzoic, 3C12amide-TMA cat, 3C12amide-DMA, 3C12amide-NH2, 3C12amide-OH, 3C12Ester-OH, 3C12 Ester-amin, 3C12Ester-DMA, 2C12Amid-DMA, 3C12-lin-amid-DMA, 2C12-sperm-amid-DMA, or 3C12-sperm-amid-DMA (see table of published PCT patent application WO2017212009A1 (pages 50-54)). Particularly preferred are 3-C12-OH or 3-C12-OH-cat.

In preferred embodiments, the polyethylene glycol/peptide polymer comprising a lipidoid as specified above (e.g. 3-C12-OH or 3-C12-OH-cat), is used to complex the at least one nucleic acid to form complexes having an N/P ratio from about 0.1 to about 20, or from about 0.2 to about 15, or from about 2 to about 15, or from about 2 to about 12, wherein the N/P ratio is defined as the mole ratio of the nitrogen atoms of the basic groups of the cationic peptide or polymer to the phosphate groups of the nucleic acid. In that context, the disclosure of published PCT patent application WO2017212009A1, in particular claims 1 to 10 of WO2017212009A1, and the specific disclosure relating thereto is herewith incorporated by reference.

Further suitable lipidoids may be derived from published PCT patent application WO2010053572. In particular, lipidoids derivable from claims 1 to 297 of published PCT patent application WO2010053572 may be used in the context of the invention, e.g. incorporated into the peptide polymer as described herein, or e.g. incorporated into the lipid nanoparticle (as described below). Accordingly, claims 1 to 297 of published PCT patent application WO2010053572, and the specific disclosure relating thereto, is herewith incorporated by reference.

Encapsulation/Complexation in Liposomes or LNPs:

In preferred embodiments, the at least one nucleic acid (e.g. DNA or RNA), preferably the at least one RNA is complexed, encapsulated, partially encapsulated, or associated with one or more lipids (e.g. cationic lipids and/or neutral lipids), thereby forming lipid-based carriers such as liposomes, lipid nanoparticles (LNPs), lipoplexes, and/or nanoliposomes, preferably lipid nanoparticles.

In embodiments where at least two nucleic acid species are in the composition, the at least two nucleic acid species are formulated separately (in any formulation or complexation agent defined herein), preferably wherein the at least two nucleic acid species are formulated in separate liposomes, lipid nanoparticles (LNP), lipoplexes, and/or nanoliposomes

In embodiments where at least two nucleic acid species are in the composition, the at least two nucleic acid species are co-formulated (in any formulation or complexation agent defined herein), preferably wherein the at least two nucleic acid species are co-formulated in liposomes, lipid nanoparticles (LNP), lipoplexes, and/or nanoliposomes

The liposomes, lipid nanoparticles (LNPs), lipoplexes, and/or nanoliposomes—incorporated nucleic acid (e.g. DNA or RNA) may be completely or partially located in the interior space of the liposomes, lipid nanoparticles (LNPs), lipoplexes, and/or nanoliposomes, within the lipid layer/membrane, or associated with the exterior surface of the lipid layer/membrane. The incorporation of a nucleic acid into liposomes/LNPs is also referred to herein as “encapsulation” wherein the nucleic acid, e.g. the RNA is entirely contained within the interior space of the liposomes, lipid nanoparticles (LNPs), lipoplexes, and/or nanoliposomes. The purpose of incorporating nucleic acid into liposomes, lipid nanoparticles (LNPs), lipoplexes, and/or nanoliposomes is to protect the nucleic acid, preferably RNA from an environment which may contain enzymes or chemicals or conditions that degrade nucleic acid and/or systems or receptors that cause the rapid excretion of the nucleic acid. Moreover, incorporating nucleic acid, preferably RNA into liposomes, lipid nanoparticles (LNPs), lipoplexes, and/or nanoliposomes may promote the uptake of the nucleic acid, and hence, may enhance the therapeutic effect of the nucleic acid, e.g. the nucleic acid encoding at least one antigenic peptide or protein. Accordingly, incorporating a nucleic acid, e.g. RNA or DNA, into liposomes, lipid nanoparticles (LNPs), lipoplexes, and/or nanoliposomes may be particularly suitable for a vaccine of the invention, e.g. for intramuscular and/or intradermal administration. In this context, the terms “complexed” or “associated” refer to the essentially stable combination of nucleic acid with one or more lipids into larger complexes or assemblies without covalent binding.

The term “lipid nanoparticle”, also referred to as “LNP”, is not restricted to any particular morphology, and include any morphology generated when a cationic lipid and optionally one or more further lipids are combined, e.g. in an aqueous environment and/or in the presence of a nucleic acid, e.g. an RNA. For example, a liposome, a lipid complex, a lipoplex and the like are within the scope of a lipid nanoparticle (LNP).

Liposomes, lipid nanoparticles (LNPs), lipoplexes, and/or nanoliposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter.

LNPs of the invention are suitably characterized as microscopic vesicles having an interior aqua space sequestered from an outer medium by a membrane of one or more bilayers. Bilayer membranes of LNPs are typically formed by amphiphilic molecules, such as lipids of synthetic or natural origin that comprise spatially separated hydrophilic and hydrophobic domains. Bilayer membranes of the liposomes can also be formed by amphophilic polymers and surfactants (e.g., polymerosomes, niosomes, etc.). In the context of the present invention, an LNP typically serves to transport the at least one nucleic acid, preferably the at least one RNA, to a target tissue.

Accordingly, in preferred embodiments, the at least one nucleic acid, preferably the at least one RNA, is complexed with one or more lipids thereby forming lipid nanoparticles (LNP), liposomes, nanoliposomes, lipoplexes, preferably LNPs. Preferably, LNPs (liposomes, nanoliposomes, lipoplexes) are particularly suitable for intramuscular and/or intradermal administration.

In embodiments, at least about 80%, 85%, 90%, 95% of lipid-based carriers, preferably the LNPs, have a spherical morphology, preferably comprising a solid core or partially solid core.

LNPs (or liposomes, nanoliposomes, lipoplexes) typically comprise a cationic lipid and one or more excipients selected from neutral lipids, charged lipids, steroids and polymer conjugated lipids (e.g. PEGylated lipid). The nucleic acid (e.g. RNA, DNA) may be encapsulated in the lipid portion of the LNP or an aqueous space enveloped by some or the entire lipid portion of the LNP. The nucleic acid (e.g. RNA, DNA) or a portion thereof may also be associated and complexed with the LNP. An LNP may comprise any lipid capable of forming a particle to which the nucleic acids are attached, or in which the one or more nucleic acids are encapsulated. Preferably, the LNP comprising nucleic acids comprises one or more cationic lipids, and one or more stabilizing lipids. Stabilizing lipids include neutral lipids and PEGylated lipids.

Preferably, the LNP (or liposomes, nanoliposomes, lipoplexes) comprises
(i) at least one cationic lipid;
(ii) at least one neutral lipid;
(iii) at least one steroid or steroid analogue, preferably cholesterol; and
(iv) at least one polymer conjugated lipid, preferably a PEG-lipid;
wherein (i) to (iv) are in a molar ratio of about 20-60% cationic lipid, 5-25% neutral lipid, 25-55% sterol, and 0.5-15% polymer conjugated lipid.

The cationic lipid of an LNP (or liposomes, nanoliposomes, lipoplexes) may be cationisable, i.e. it becomes protonated as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

Such lipids (for liposomes, lipid nanoparticles (LNP), lipoplexes, and/or nanoliposomes) include, but are not limited to, DSDMA, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), ckk-E12, ckk, 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (y-DLenDMA), 98N12-5, 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA·Cl), ICE (Imidazol-based), HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, XTC (2,2-Dilinoleyl-4-dimethylaminoethyl-[1,3]-dioxolane) HGT4003, 1,2-Dilinoleoyl-3-trimethylaminopropane chloride salt (DLin-TAP·Cl), 1,2-Dilinoleyloxy-3-(N-methylpiperazino)propane (DLin-MPZ), or 3-(N,N-Dilinoleyamino)-1, 2-propanediol (DLinAP), 3-(N,N-Dioleylamino)-1.2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate (MC3), ALNY-100 ((3aR,5s,6aS)-N,N-dimethyl-2,2-di((9Z,12Z)-octadeca-9,12-dienyl)tetrahydro-3aH-cyclopenta[d][1,3]dioxol-5-amine)), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), NC98-5 (4,7, 13-tris(3-oxo-3-(undecylamino)propyl)-N,N 16-diundecyl-4,7, 10,13-tetraazahexadecane-1,16-diamide), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic lipids for use in the compositions and methods of the invention include those described in international patent publications WO2010053572 (and particularly, CI 2-200 described at paragraph [00225]) and WO2012170930, both of which are incorporated herein by reference, HGT4003, HGT5000, HGTS001, HGT5001, HGT5002 (see US20150140070A1).

In embodiments, the cationic lipid of the liposomes, lipid nanoparticles (LNP), lipoplexes, and/or nanoliposomes may be an amino lipid.

Representative amino lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA·Cl), 1,2-dilinoleoyl-3-trimethylaminopropane chloride salt (DLin-TAP·Cl), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20100324120).

In embodiments, the cationic lipid of the liposomes, lipid nanoparticles (LNP), lipoplexes, and/or nanoliposomes may an aminoalcohol lipidoid.

Aminoalcohol lipidoids which may be used in the present invention may be prepared by the methods described in U.S. Pat. No. 8,450,298, herein incorporated by reference in its entirety. Suitable (ionizable) lipids can also be the compounds as disclosed in Tables 1, 2 and 3 and as defined in claims 1-24 of WO2017075531A1, hereby incorporated by reference.

In another embodiment, suitable lipids can also be the compounds as disclosed in WO2015074085A1 (i.e. ATX-001 to ATX-032 or the compounds as specified in claims 1-26), U.S. Appl. Nos. 61/905,724 and 15/614,499 or U.S. Pat. Nos. 9,593,077 and 9,567,296 hereby incorporated by reference in their entirety.

In other embodiments, suitable cationic lipids can also be the compounds as disclosed in WO2017117530A1 (i.e. lipids 13, 14, 15, 16, 17, 18, 19, 20, or the compounds as specified in the claims), hereby incorporated by reference in its entirety.

In preferred embodiments, ionizable or cationic lipids may also be selected from the lipids disclosed in WO2018078053A1 (i.e. lipids derived from formula I, II, and III of WO2018078053A1, or lipids as specified in Claims 1 to 12 of WO2018078053A1), the disclosure of WO2018078053A1 hereby incorporated by reference in its entirety. In that context, lipids disclosed in Table 7 of WO2018078053A1 (e.g. lipids derived from formula I-1 to I-41) and lipids disclosed in Table 8 of WO2018078053A1 (e.g. lipids derived from formula II-1 to II-36) may be suitably used in the context of the invention. Accordingly, formula I-1 to formula I-41 and formula II-1 to formula II-36 of WO2018078053A1, and the specific disclosure relating thereto, are herewith incorporated by reference.

In preferred embodiments, cationic lipids may be derived from formula III of published PCT patent application WO2018078053A1. Accordingly, formula III of WO2018078053A1, and the specific disclosure relating thereto, are herewith incorporated by reference.

In particularly preferred embodiments, the at least one nucleic acid (e.g. DNA or RNA), preferably the at least one RNA, is complexed with one or more lipids thereby forming LNPs (or liposomes, nanoliposomes, lipoplexes), wherein the cationic lipid of the LNP is selected from structures III-1 to III-36 of Table 9 of published PCT patent application WO2018078053A1. Accordingly, formula III-1 to III-36 of WO2018078053A1, and the specific disclosure relating thereto, are herewith incorporated by reference.

In particularly preferred embodiments, the at least one nucleic acid (e.g. DNA or RNA), preferably the at least one RNA is complexed with one or more lipids thereby forming liposomes, lipid nanoparticles (LNP), lipoplexes, and/or nanoliposomes, preferably LNPs, wherein the liposomes, lipid nanoparticles (LNP), lipoplexes, and/or nanoliposomes, preferably the LNPs comprise a cationic lipid according to formula III-3:

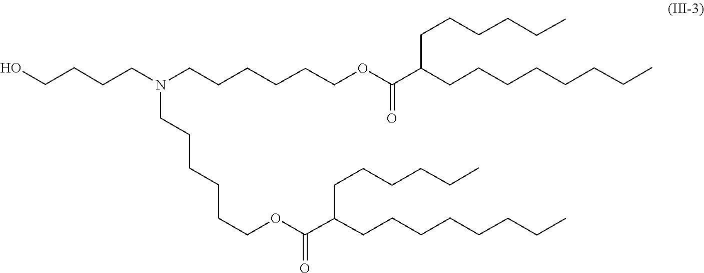 [SMILES: CCCCCCCCC(CCCCCC)C(=O)OCCCCCCN(CCCCO)CCCCCCOC(=O)C(CCCCCC)CCCCCCCC]

The lipid of formula III-3 as suitably used herein has the chemical term ((4-hydroxybutyl)azanediyl)bis(hexane-6,1-diyl)bis(2-hexyldecanoate), also referred to as ALC-0315.

In certain embodiments, the cationic lipid as defined herein, more preferably cationic lipid compound III-3, is present in the LNP (or liposomes, nanoliposomes, lipoplexes) in an amount from about 30 to about 95 mole percent, relative to the total lipid content of the LNP. If more than one cationic lipid is incorporated within the LNP, such percentages apply to the combined cationic lipids.

In embodiments, the cationic lipid is present in the LNP (or liposomes, nanoliposomes, lipoplexes) in an amount from about 30 to about 70 mole percent. In one embodiment, the cationic lipid is present in the LNP (or liposomes, nanoliposomes, lipoplexes) in an amount from about 40 to about 60 mole percent, such as about 40, 41, 42, 43, 44, 45, 46, 47, 48, 49, 50, 51, 52, 53, 54, 55, 56, 57, 58, 59 or 60 mole percent, respectively. In embodiments, the cationic lipid is present in the LNP (or liposomes, nanoliposomes, lipoplexes) in an amount from about 47 to about 48 mole percent, such as about 47.0, 47.1, 47.2, 47.3, 47.4, 47.5, 47.6, 47.7, 47.8, 47.9, 50.0 mole percent, respectively, wherein 47.7 mole percent are particularly preferred.

In some embodiments, the cationic lipid is present in a ratio of from about 20 mol % to about 70 or 75 mol % or from about 45 to about 65 mol % or about 20, 25, 30, 35, 40, 45, 50, 55, 60, 65, or about 70 mol % of the total lipid present in the LNP (or liposomes, nanoliposomes, lipoplexes). In further embodiments, the LNPs (or liposomes, nanoliposomes, lipoplexes) comprise from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the lipid nanoparticle). In some embodiments, the ratio of cationic lipid to nucleic acid (e.g. coding RNA or DNA) is from about 3 to about 15, such as from about 5 to about 13 or from about 7 to about 11.

Other suitable (cationic or ionizable) lipids are disclosed in WO2009086558, WO2009127060, WO2010048536, WO2010054406, WO2010088537, WO2010129709, WO2011153493, WO 2013063468, US20110256175, US20120128760, US20120027803, U.S. Pat. No. 8,158,601, WO2016118724, WO2016118725, WO2017070613, WO2017070620, WO2017099823, WO2012040184, WO2011153120, WO2011149733, WO2011090965, WO2011043913, WO2011022460, WO2012061259, WO2012054365, WO2012044638, WO2010080724, WO201021865, WO2008103276, WO2013086373, WO2013086354, U.S. Pat. Nos. 7,893,302, 7,404,969, 8,283,333, 8,466,122 and 8,569,256 and US Patent Publication No. US20100036115, US20120202871, US20130064894, US20130129785, US20130150625, US20130178541, US20130225836, US20140039032 and WO2017112865. In that context, the disclosures of WO2009086558, WO2009127060, WO2010048536, WO2010054406, WO2010088537, WO2010129709, WO2011153493, WO 2013063468, US20110256175, US20120128760, US20120027803, U.S. Pat. No. 8,158,601, WO2016118724, WO2016118725, WO2017070613, WO2017070620, WO2017099823, WO2012040184, WO2011153120, WO2011149733, WO2011090965, WO2011043913, WO2011022460, WO2012061259, WO2012054365, WO2012044638, WO2010080724, WO201021865, WO2008103276, WO2013086373, WO2013086354, U.S. Pat. Nos. 7,893,302, 7,404,969, 8,283,333, 8,466,122 and 8,569,256 and US Patent Publication No. US20100036115, US20120202871, US20130064894, US20130129785, US20130150625, US20130178541, US20130225836 and US20140039032 and WO2017112865 specifically relating to (cationic) lipids suitable for LNPs (or liposomes, nanoliposomes, lipoplexes) are incorporated herewith by reference.

In embodiments, amino or cationic lipids as defined herein have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of lipids have to be present in the charged or neutral form. Lipids having more than one protonatable or deprotonatable group, or which are zwitterionic, are not excluded and may likewise suitable in the context of the present invention. In some embodiments, the protonatable lipids have a pKa of the protonatable group in the range of about 4 to about 11, e.g., a pKa of about 5 to about 7.

LNPs (or liposomes, nanoliposomes, lipoplexes) can comprise two or more (different) cationic lipids as defined herein. Cationic lipids may be selected to contribute to different advantageous properties. For example, cationic lipids that differ in properties such as amine pKa, chemical stability, half-life in circulation, half-life in tissue, net accumulation in tissue, or toxicity can be used in the LNP (or liposomes, nanoliposomes, lipoplexes). In particular, the cationic lipids can be chosen so that the properties of the mixed-LNP are more desirable than the properties of a single-LNP of individual lipids.

The amount of the permanently cationic lipid or lipidoid may be selected taking the amount of the nucleic acid cargo into account. In one embodiment, these amounts are selected such as to result in an N/P ratio of the nanoparticle(s) or of the composition in the range from about 0.1 to about 20. In this context, the N/P ratio is defined as the mole ratio of the nitrogen atoms (“N”) of the basic nitrogen-containing groups of the lipid or lipidoid to the phosphate groups (“P”) of the nucleic acid which is used as cargo. The N/P ratio may be calculated on the basis that, for example, 1 ug RNA typically contains about 3nmol phosphate residues, provided that the RNA exhibits a statistical distribution of bases. The “N”-value of the lipid or lipidoid may be calculated on the basis of its molecular weight and the relative content of permanently cationic and—if present—cationisable groups.

In vivo characteristics and behavior of LNPs (or liposomes, nanoliposomes, lipoplexes) can be modified by addition of a hydrophilic polymer coating, e.g. polyethylene glycol (PEG), to the LNP surface to confer steric stabilization. Furthermore, LNPs (or liposomes, nanoliposomes, lipoplexes) can be used for specific targeting by attaching ligands (e.g. antibodies, peptides, and carbohydrates) to its surface or to the terminal end of the attached PEG chains (e.g. via PEGylated lipids or PEGylated cholesterol).

In some embodiments, the LNPs (or liposomes, nanoliposomes, lipoplexes) comprise a polymer conjugated lipid. The term “polymer conjugated lipid” refers to a molecule comprising both a lipid portion and a polymer portion. An example of a polymer conjugated lipid is a PEGylated lipid. The term “PEGylated lipid” refers to a molecule comprising both a lipid portion and a polyethylene glycol portion. PEGylated lipids are known in the art and include 1-(monomethoxy-polyethyleneglycol)-2,3-dimyristoylglycerol (PEG-s-DMG) and the like.

A polymer conjugated lipid as defined herein may serve as an aggregation reducing lipid.

In certain embodiments, the LNP (or liposomes, nanoliposomes, lipoplexes) comprises a stabilizing-lipid which is a polyethylene glycol-lipid (PEGylated lipid). Suitable polyethylene glycol-lipids include PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyl]-1,2-dimyristyloxlpropyl-3-amine (PEG-c-DMA). In a preferred embodiment, the polyethylene glycol-lipid is PEG-2000-DMG. In one embodiment, the polyethylene glycol-lipid is PEG-c-DOMG). In other embodiments, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a PEGylated ceramide (PEG-cer), or a PEG dialkoxypropylcarbamate such as ω-methoxy(polyethoxy)ethyl-N-(2,3di(tetradecanoxy)propyl)carbamate or 2,3-di(tetradecanoxy)propyl-N-(ω-methoxy(polyethoxy)ethyl)carbamate.

In preferred embodiments, the PEGylated lipid is preferably derived from formula (IV) of published PCT patent application WO2018078053A1. Accordingly, PEGylated lipids derived from formula (IV) of published PCT patent application WO2018078053A1, and the respective disclosure relating thereto, are herewith incorporated by reference.

In a preferred embodiments, the at least one nucleic acid is complexed with one or more lipids thereby forming LNPs (or liposomes, nanoliposomes, lipoplexes), wherein the LNP comprises a polymer conjugated lipid, preferably a PEGylated lipid, wherein the PEG lipid is preferably derived from formula (IVa) of published PCT patent application WO2018078053A1. Accordingly, PEGylated lipid derived from formula (IVa) of published PCT patent application WO2018078053A1, and the respective disclosure relating thereto, is herewith incorporated by reference.

In a preferred embodiment, the at least one nucleic acid, preferably the at least one RNA, is complexed with one or more lipids thereby forming lipid nanoparticles (or liposomes, nanoliposomes, lipoplexes), wherein the LNP (or liposomes, nanoliposomes, lipoplexes) comprises a polymer conjugated lipid, preferably a PEGylated lipid/PEG lipid. In preferred embodiments, said PEG lipid or PEGylated lipid is of formula (IVa):

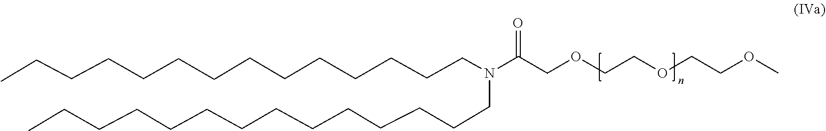

wherein n has a mean value ranging from 30 to 60, such as about 30±2, 32±2, 34±2, 36±2, 38±2, 40±2, 42±2, 44±2, 46±2, 48±2, 50±2, 52±2, 54±2, 56±2, 58±2, or 60±2. In a most preferred embodiment n is about 49. In another preferred embodiment n is about 45. In further preferred embodiments, said PEG lipid is of formula (IVa) wherein n is an integer selected such that the average molecular weight of the PEG lipid is about 2000 g/mol to about 3000 g/mol or about 2300 g/mol to about 2700 g/mol, even more preferably about 2500 g/mol.

The lipid of formula IVa as suitably used herein has the chemical term 2[(polyethylene glycol)-2000]-N,N-ditetradecylacetamide, also referred to as ALC-0159.

Further examples of PEG-lipids suitable in that context are provided in US20150376115A1 and WO2015199952, each of which is incorporated by reference in its entirety.

In some embodiments, LNPs (or liposomes, nanoliposomes, lipoplexes) include less than about 3, 2, or 1 mole percent of PEG or PEG-modified lipid, based on the total moles of lipid in the LNP. In further embodiments, LNPs (or liposomes, nanoliposomes, lipoplexes) comprise from about 0.1% to about 20% of the PEG-modified lipid on a molar basis, e.g., about 0.5 to about 10%, about 0.5 to about 5%, about 10%, about 5%, about 3.5%, about 3%, about 2.5%, about 2%, about 1.5%, about 1%, about 0.5%, or about 0.3% on a molar basis (based on 100% total moles of lipids in the LNP). In preferred embodiments, LNPs (or liposomes, nanoliposomes, lipoplexes) comprise from about 1.0% to about 2.0% of the PEG-modified lipid on a molar basis, e.g., about 1.2 to about 1.9%, about 1.2 to about 1.8%, about 1.3 to about 1.8%, about 1.4 to about 1.8%, about 1.5 to about 1.8%, about 1.6 to about 1.8%, in particular about 1.4%, about 1.5%, about 1.6%, about 1.7%, about 1.8%, about 1.9%, most preferably 1.7% (based on 100% total moles of lipids in the LNP). In various embodiments, the molar ratio of the cationic lipid to the PEGylated lipid ranges from about 100:1 to about 25:1.

In preferred embodiments, the LNP (or liposomes, nanoliposomes, lipoplexes) comprises one or more additional lipids, which stabilize the formation of particles during their formulation or during the manufacturing process (e.g. neutral lipid and/or one or more steroid or steroid analogue).

In preferred embodiments, the at least one nucleic acid, preferably the at least one RNA is complexed with one or more lipids thereby forming lipid nanoparticles (or liposomes, nanoliposomes, lipoplexes), wherein the LNP (or liposomes, nanoliposomes, lipoplexes) comprises one or more neutral lipid and/or one or more steroid or steroid analogue.

Suitable stabilizing lipids include neutral lipids and anionic lipids. The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

In embodiments, the LNP (or liposome, nanoliposome, lipoplexe) comprises one or more neutral lipids, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE), or mixtures thereof.

In some embodiments, the LNPs (or liposomes, nanoliposomes, lipoplexes) comprise a neutral lipid selected from DSPC, DPPC, DMPC, DOPC, POPC, DOPE and SM. In various embodiments, the molar ratio of the cationic lipid to the neutral lipid ranges from about 2:1 to about 8:1.

In preferred embodiments, the neutral lipid is 1,2-distearoyl-sn-glycero-3-phosphocholine (DSPC). Suitably, the molar ratio of the cationic lipid to DSPC may be in the range from about 2:1 to about 8:1.

In preferred embodiments, the steroid is cholesterol. Suitably, the molar ratio of the cationic lipid to cholesterol may be in the range from about 2:1 to about 1:1. In some embodiments, the cholesterol may be PEGylated.

The sterol can be about 10 mol % to about 60 mol % or about 25 mol % to about 40 mol % of the lipid particle. In one embodiment, the sterol is about 10, 15, 20, 25, 30, 35, 40, 45, 50, 55, or about 60 mol % of the total lipid present in the lipid particle. In another embodiment, the LNPs include from about 5% to about 50% on a molar basis of the sterol, e.g., about 15% to about 45%, about 20% to about 40%, about 48%, about 40%, about 38.5%, about 35%, about 34.4%, about 31.5% or about 31% on a molar basis (based upon 100% total moles of lipid in the lipid nanoparticle).

Preferably, lipid LNPs (or liposomes, nanoliposomes, lipoplexes) comprise:
(a) the at least one nucleic acid, preferably the at least one RNA, (b) a cationic lipid, (c) an aggregation reducing agent (such as polyethylene glycol (PEG) lipid or PEG-modified lipid), (d) optionally a non-cationic lipid (such as a neutral lipid), and (e) optionally, a sterol.

In some embodiments, the cationic lipids (as defined above), non-cationic lipids (as defined above), cholesterol (as defined above), and/or PEG-modified lipids (as defined above) may be combined at various relative molar ratios. For example, the ratio of cationic lipid to non-cationic lipid to cholesterol-based lipid to PEGylated lipid may be between about 30-60:20-35:20-30:1-15, or at a ratio of about 40:30:25:5, 50:25:20:5, 50:27:20:3, 40:30:20:10, 40:32:20:8, 40:32:25:3 or 40:33:25:2, or at a ratio of about 50:25:20:5, 50:20:25:5, 50:27:20:3 40:30:20:10, 40:30:25:5 or 40:32:20:8, 40:32:25:3 or 40:33:25:2, respectively.

In some embodiments, the LNPs (or liposomes, nanoliposomes, lipoplexes) comprise a lipid of formula (III), at least one nucleic acid, preferably the at least one RNA as defined herein, a neutral lipid, a steroid and a PEGylated lipid. In preferred embodiments, the lipid of formula (III) is lipid compound 111-3 (ALC-0315), the neutral lipid is DSPC, the steroid is cholesterol, and the PEGylated lipid is the compound of formula (IVa).

In a preferred embodiment, the LNP (or liposomes, nanoliposomes, lipoplexes) consists essentially of (i) at least one cationic lipid; (ii) a neutral lipid; (iii) a sterol, e.g., cholesterol; and (iv) a PEG-lipid, e.g. PEG-DMG or PEG-cDMA, in a molar ratio of about 20-60% cationic lipid: 5-25% neutral lipid: 25-55% sterol; 0.5-15% PEG-lipid.

In particularly preferred embodiments, the at least one nucleic acid is complexed with one or more lipids thereby forming lipid nanoparticles (or liposomes, nanoliposomes, lipoplexes), wherein the LNP (or liposome, nanoliposome, lipoplex) comprises
(i) at least one cationic lipid as defined herein, preferably a lipid of formula (III), more preferably lipid III-3 (ALC-0315);
(ii) at least one neutral lipid as defined herein, preferably 1,2-distearoyl-sn-glycero-3-phosphocholine (DSPC);
(iii) at least one steroid or steroid analogue as defined herein, preferably cholesterol; and
(iv) at least one polymer conjugated lipid, preferably a PEG-lipid as defined herein, e.g. PEG-DMG or PEG-cDMA, preferably a PEGylated lipid that is or is derived from formula (Iva; ALC-0159).

In particularly preferred embodiments, the at least one nucleic acid is complexed with one or more lipids thereby forming lipid nanoparticles (LNP), wherein the LNP comprises (i) to (iv) in a molar ratio of about 20-60% cationic lipid: 5-25% neutral lipid: 25-55% sterol; 0.5-15% polymer conjugated lipid, preferably PEG-lipid.

In one preferred embodiment, the lipid nanoparticle (or liposome, nanoliposome, lipoplexe) comprises: a cationic lipid with formula (III) and/or PEG lipid with formula (IV), optionally a neutral lipid, preferably 1,2-distearoyl-sn-glycero-3-phosphocholine (DSPC) and optionally a steroid, preferably cholesterol, wherein the molar ratio of the cationic lipid to DSPC is optionally in the range from about 2:1 to 8:1, wherein the molar ratio of the cationic lipid to cholesterol is optionally in the range from about 2:1 to 1:1.

In a particular preferred embodiment, the composition comprises lipid nanoparticles (LNPs), which have a molar ratio of approximately 50:10:38.5:1.5, preferably 47.5:10:40.8:1.7 or more preferably 47.4:10:40.9:1.7 (i.e. proportion (mol %) of cationic lipid (preferably lipid III-3 (ALC-0315)), DSPC, cholesterol and polymer conjugated lipid, preferably PEG-lipid (preferably PEG-lipid of formula (IVa) with n=49, even more preferably PEG-lipid of formula (IVa) with n=45; ALC-0159); solubilized in ethanol).

In embodiments where the composition is a multivalent composition as defined above, the RNA species, preferably mRNA species of the multivalent composition may be formulated separately, preferably formulated separately in liposomes or LNPs. Suitably, the RNA species of the multivalent composition are separately formulated in LNPs which have a molar ratio of approximately 50:10:38.5:1.5, preferably 47.5:10:40.8:1.7 or more preferably 47.4:10:40.9:1.7 proportion (mol %) of cationic lipid III-3 (ALC-0315), DSPC, cholesterol and PEG-lipid of formula (IVa) (with n=49 or with n=45). Nucleic acid species for multivalent compositions are preferably selected as defined above (see section “Combinations of M, N, NSP3, NSP4, NSP6, NSP13, NSP14, ORF3A, ORF8, E, and/or S”).

The total amount of nucleic acid in the lipid nanoparticles may vary and is defined depending on the e.g. nucleic acid to total lipid w/w ratio. In one embodiment of the invention the nucleic acid, in particular the RNA to total lipid ratio is less than 0.06 w/w, preferably between 0.03 w/w and 0.04 w/w.

In preferred embodiments, the wt/wt ratio of lipid to nucleic acid is from about 10:1 to about 60:1, preferably from about 20:1 to about 30:1, for example about 25:1.

In preferred embodiments, the n/p ratio of the liposome, lipid nanoparticle (LNP), lipoplex, and/or nanoliposome encapsulating the nucleic acid is in a range from about 1 to about 10, preferably in a range from about 5 to about 7, more preferably about 6.

In some embodiments, the lipid nanoparticles (or liposomes, nanoliposomes, lipoplexes) are composed of only three lipid components, namely imidazole cholesterol ester (ICE), 1,2-dioleoyl-sn-glycero-3-phosphoethanolamine (DOPE), and 1,2-dimyristoyl-sn-glycerol, methoxypolyethylene glycol (DMG-PEG-2K)

In one embodiment, the lipid nanoparticle (or liposomes, nanoliposomes, lipoplexes) of the composition comprises a cationic lipid, a steroid, a neutral lipid, and a polymer conjugated lipid, preferably a pegylated lipid. Preferably, the polymer conjugated lipid is a pegylated lipid or PEG-lipid. In a specific embodiment, lipid nanoparticles comprise a cationic lipid resembled by the cationic lipid COATSOME® SS-EC (former name: SS-33/4PE-15; NOF Corporation, Tokyo, Japan), in accordance with the following formula

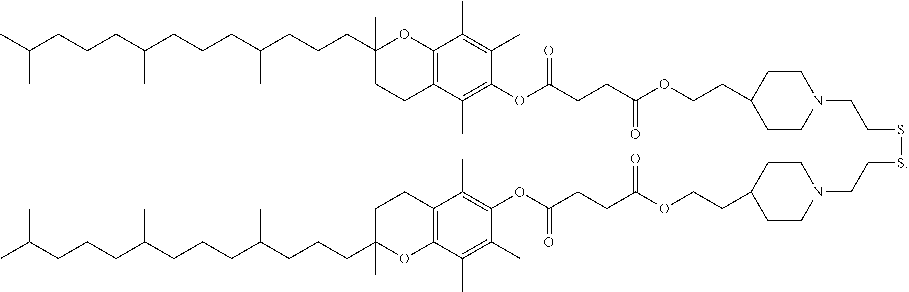 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

As described further below, those lipid nanoparticles are termed “GN01”.

Furthermore, in a specific embodiment, the GN01 lipid nanoparticles comprise a neutral lipid being resembled by the structure 1,2-diphytanoyl-sn-glycero-3-phosphoethanolamine (DPhyPE):

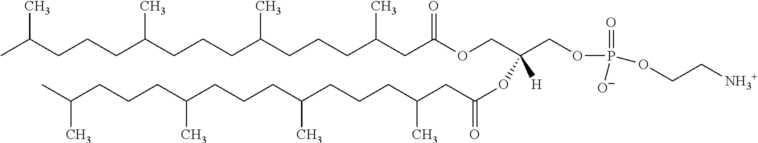 [SMILES: CC(C)CCCC(C)CCCC(C)CCCC(C)CC(=O)OC[C@H](COP(=O)([O-])OCC[NH3+])OC(=O)CC(C)CCCC(C)CCCC(C)CCCC(C)C]

Furthermore, in a specific embodiment, the GN01 lipid nanoparticles comprise a polymer conjugated lipid, preferably a pegylated lipid, being 1,2-dimyristoyl-rac-glycero-3-methoxypolyethylene glycol 2000 (DMG-PEG 2000) having the following structure:

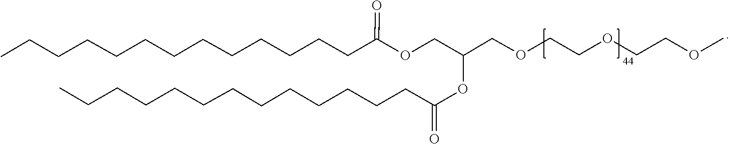

As used in the art, “DMG-PEG 2000” is considered a mixture of 1,2-DMG PEG2000 and 1,3-DMG PEG2000 in ˜97:3 ratio.

Accordingly, GN01 lipid nanoparticles (GN01-LNPs) according to one of the preferred embodiments comprise a SS-EC cationic lipid, neutral lipid DPhyPE, cholesterol, and the polymer conjugated lipid (pegylated lipid) 1,2-dimyristoyl-rac-glycero-3-methoxypolyethylene glycol (PEG-DMG).

In a preferred embodiment, the GN01 LNPs comprise:
(a) cationic lipid SS-EC (former name: SS-33/4PE-15; NOF Corporation, Tokyo, Japan) at an amount of 45-65 mol %;
(b) cholesterol at an amount of 25-45 mol %;
(c) DPhyPE at an amount of 8-12 mol %; and
(d) PEG-DMG 2000 at an amount of 1-3 mol %;
   each amount being relative to the total molar amount of all lipidic excipients of the GN01 lipid nanoparticles.

In a further preferred embodiment, the GN01 lipid nanoparticles as described herein comprises 59 mol % cationic lipid, 10 mol % neutral lipid, 29.3 mol % steroid and 1.7 mol % polymer conjugated lipid, preferably pegylated lipid. In a most preferred embodiment, the GN01 lipid nanoparticles as described herein comprise 59 mol % cationic lipid SS-EC, 10 mol % DPhyPE, 29.3 mol % cholesterol and 1.7 mol % DMG-PEG 2000.

The amount of the cationic lipid relative to that of the nucleic acid in the GN01 lipid nanoparticle may also be expressed as a weight ratio (abbreviated e.g. “m/m”). For example, the GN01 lipid nanoparticles comprise the at least one nucleic acid, preferably the at least one RNA at an amount such as to achieve a lipid to RNA weight ratio in the range of about 20 to about 60, or about 10 to about 50. In other embodiments, the ratio of cationic lipid to nucleic acid or RNA is from about 3 to about 15, such as from about 5 to about 13, from about 4 to about 8 or from about 7 to about 11. In a very preferred embodiment of the present invention, the total lipid/RNA mass ratio is about 40 or 40, i.e. about 40 or 40 times mass excess to ensure RNA encapsulation. Another preferred RNA/lipid ratio is between about 1 and about 10, about 2 and about 5, about 2 and about 4, or preferably about 3.

Further, the amount of the cationic lipid may be selected taking the amount of the nucleic acid cargo such as the RNA compound into account. In one embodiment, the N/P ratio can be in the range of about 1 to about 50. In another embodiment, the range is about 1 to about 20, about 1 to about 10, about 1 to about 5. In one preferred embodiment, these amounts are selected such as to result in an N/P ratio of the GN01 lipid nanoparticles or of the composition in the range from about 10 to about 20. In a further very preferred embodiment, the N/P is 14 (i.e. 14 times mol excess of positive charge to ensure nucleic acid encapsulation).

In a preferred embodiment, GN01 lipid nanoparticles comprise 59 mol % cationic lipid COATSOME® SS-EC (former name: SS-33/4PE-15 as apparent from the examples section; NOF Corporation, Tokyo, Japan), 29.3 mol % cholesterol as steroid, 10 mol % DPhyPE as neutral lipid/phospholipid and 1.7 mol % DMG-PEG 2000 as polymer conjugated lipid. A further inventive advantage connected with the use of DPhyPE is the high capacity for fusogenicity due to its bulky tails, whereby it is able to fuse at a high level with endosomal lipids. For “GN01”, N/P (lipid to nucleic acid, e.g. RNA mol ratio) preferably is 14 and total lipid/RNA mass ratio preferably is 40 (m/m).

In other embodiments, the at least one nucleic acid, preferably the at least one RNA, is complexed with one or more lipids thereby forming lipid nanoparticles (or liposomes, nanoliposomes, lipoplexes), wherein the LNP (or liposomes, nanoliposomes, lipoplexes) comprises
1. at least one cationic lipid;
Ii. at least one neutral lipid;
Iii. at least one steroid or steroid analogue; and
Iiii. at least one PEG-lipid as defined herein,
   wherein the cationic lipid is DLin-KC2-DMA (50 mol %) or DLin-MC3-DMA (50 mol %), the neutral lipid is DSPC (10 mol %), the PEG lipid is PEG-DOMG (1.5 mol %) and the structural lipid is cholesterol (38.5 mol %).

In other embodiments, the at least one nucleic acid (e.g. DNA or RNA), preferably the at least one RNA, is complexed with one or more lipids thereby forming lipid nanoparticles (LNP), wherein the LNP comprises SS15/Chol/DOPE (or DOPC)/DSG-5000 at mol % 50/38.5/10/1.5.

In other embodiments, the at least one nucleic acid may be formulated in liposomes, e.g. in liposomes as described in WO2019222424, WO2019226925, WO2019232095, WO2019232097, or WO2019232208, the disclosure of WO2019222424, WO2019226925, WO2019232095, WO2019232097, or WO2019232208 relating to liposomes or lipid-based carrier molecules herewith incorporated by reference.

In various embodiments, LNPs that suitably encapsulates the at least one nucleic acid have a mean diameter of from about 50 nm to about 200 nm, from about 60 nm to about 200 nm, from about 70 nm to about 200 nm, from about 80 nm to about 200 nm, from about 90 nm to about 200 nm, from about 90 nm to about 190 nm, from about 90 nm to about 180 nm, from about 90 nm to about 170 nm, from about 90 nm to about 160 nm, from about 90 nm to about 150 nm, from about 90 nm to about 140 nm, from about 90 nm to about 130 nm, from about 90 nm to about 120 nm, from about 90 nm to about 100 nm, from about 70 nm to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 180 nm, 190 nm, or 200 nm and are substantially non-toxic. As used herein, the mean diameter may be represented by the z-average as determined by dynamic light scattering as commonly known in the art.

In another preferred embodiment of the invention the lipid nanoparticles have a hydrodynamic diameter in the range from about 50 nm to about 300 nm, or from about 60 nm to about 250 nm, from about 60 nm to about 150 nm, or from about 60 nm to about 120 nm, respectively.

In another preferred embodiment of the invention the lipid nanoparticles have a hydrodynamic diameter in the range from about 50 nm to about 300 nm, or from about 60 nm to about 250 nm, from about 60 nm to about 150 nm, or from about 60 nm to about 120 nm, respectively.

In another preferred embodiments, the lipid nanoparticles have a Z-average size in a range of about 60 nm to about 120 nm, preferably less than about 120 nm, more preferably less than about 100 nm, most preferably less than about 80 nm.

Suitably, LNPs comprise less than about 10%, 9%, 8%, 7%, 6%, 5%, 4%, 3%, 2%, 1% LNPs that have a particle size exceeding about 500 nm. Suitably, the LNPs comprise less than about 10%, 9%, 8%, 7%, 6%, 5%, 4%, 3%, 2%, 1% LNPs that have a particle size smaller than about 20 nm.

The polydispersity index (PDI) of the nanoparticles (e.g. LNPs) is typically in the range of 0.1 to 0.5. In a particular embodiment, a PDI is below 0.2. Typically, the PDI is determined by dynamic light scattering.

In preferred embodiments, the composition has a polydispersity index (PDI) value of less than about 0.4, preferably of less than about 0.3, more preferably of less than about 0.2, most preferably of less than about 0.1.

In various embodiments, nucleic acid, e.g., RNA of the pharmaceutical composition does not exceed a certain proportion of free RNA.

In this context, the term “free RNA” or “non-complexed RNA” or “non-encapsulated RNA” comprise the RNA molecules that are not encapsulated in the lipid-based carriers as defined herein. During formulation of the liquid composition (e.g. during encapsulation of the RNA into the lipid-based carriers), free RNA may represent a contamination or an impurity.

The skilled person can choose from a variety of different methods for determining the amount and/or the proportion of free nucleic acid of free RNA in the liquid composition. Free RNA in the liquid composition may be determined by chromatographic methods (e.g. AEX, SEC) or by using probes (e.g. dyes) that bind to free RNA in the composition. In the context of the invention, the amount of free RNA or non-encapsulated RNA may be determined using a dye based assay. Suitable dyes that may be used to determine the amount and/or the proportion of free RNA comprise RiboGreen®, PicoGreen® dye, OliGreen® dye, QuantiFluor® RNA dye, Qubit® RNA dye, Quant-iT™ RNA dye, TOTO®-1 dye, YOYO®-1 dye. Such dyes are suitable to discriminate between free RNA and encapsulated RNA. Reference standards consisting of defined amounts of free RNA or encapsulated RNA may be used and mixed with the respective reagent (e.g. RiboGreen® reagent (Excitation 500 nm/Emission 525 nm)) as recommended by the supplier's instructions. Typically, the free RNA of the liquid composition is quantitated using the Quant-iT RiboGreen RNA Reagent according to the manufacturer's instructions. The proportion of free RNA in the context of the invention is typically determined using a RiboGreen assay.

In embodiments, a composition comprises free nucleic acid, such as free RNA ranging from about 30% to about 0%. In embodiments, the composition comprises about 20% free RNA (and about 80% encapsulated RNA), about 15% free RNA (and about 85% encapsulated RNA), about 10% free RNA (and about 90% encapsulated RNA), or about 5% free RNA (and about 95% encapsulated RNA). In preferred embodiments, the composition comprises less than about 20% free RNA, preferably less than about 15% free RNA, more preferably less than about 10% free RNA, most preferably less than about 5% free RNA.

In aspects comprising RNA nucleic acids, the term “encapsulated RNA” comprise the RNA molecules that are encapsulated in the lipid-based carriers as defined herein. The proportion of encapsulated RNA in the context of the invention is typically determined using a RiboGreen assay.

Accordingly, in embodiments, about 70% to about 100% of the RNA in the composition is encapsulated in the lipid-based carriers. In embodiments, the liquid composition comprises about 80% encapsulated RNA (and about 20% free RNA), about 85% encapsulated RNA (and about 15% free RNA), about 90% encapsulated RNA (and about 10% free RNA), or about 95% encapsulated RNA (and 5% about free RNA).

In preferred embodiments, 80% of the nucleic acid (e.g., RNA) comprised in the composition is encapsulated, preferably 85% of the RNA comprised in the composition is encapsulated, more preferably 90% of the RNA comprised in the composition is encapsulated, most preferably 95% of the RNA comprised in the composition is encapsulated.

In embodiments, the lipid-based carriers encapsulating the RNA has been purified by at least one purification step, preferably by at least one step of TFF and/or at least one step of clarification and/or at least one step of filtration. Suitably, the purified composition w comprises less than about 500ppM ethanol, preferably less than about 50ppM ethanol, more preferably less than about 5ppM ethanol.

In various embodiments, the pharmaceutical composition of the first aspect comprises a sugar in a concentration of about 50 mM to about 300 mM, preferably sucrose in a concentration of about 150 mM.

In embodiments where more than one or a plurality, e.g. 2, 3, 4, 5, 6, 7, 8, 9, 10, 11, 12, 13, 14, 15 of nucleic acid species of the invention are comprised in the composition, said more than one or said plurality e.g. 2, 3, 4, 5, 6, 7, 8, 9, 10, 11, 12, 13, 14, 15 of nucleic acid species may be complexed within one or more lipids thereby forming LNPs comprising more than one or a plurality, e.g. 2, 3, 4, 5, 6, 7, 8, 9, 10, 11, 12, 13, 14, 15 of different nucleic acid species.

In embodiments, the nucleic acid formulation (e.g. Polymers, LNPs, liposomes, nanoliposomes, lipoplexes) described herein may be lyophilized in order to improve storage stability of the formulation and/or the at least one nucleic acid. In embodiments, the nucleic acid formulation (e.g. Polymers, LNPs, liposomes, nanoliposomes, lipoplexes) described herein may be spray dried in order to improve storage stability of the formulation and/or the at least one nucleic acid. Lyoprotectants for lyophilization and or spray drying may be selected from trehalose, sucrose, mannose, dextran and inulin.

A preferred lyoprotectant is sucrose, optionally comprising a further lyoprotectant. A further preferred lyoprotectant is trehalose, optionally comprising a further lyoprotectant.

Suitably, the pharmaceutical composition, e.g. the composition comprising LNPs, is lyophilized (e.g. according to WO2016165831 or WO2011069586) to yield a temperature stable dried nucleic acid (powder) composition as defined herein (e.g. RNA or DNA). The composition, e.g. the composition comprising LNPs, may also be dried using spray-drying or spray-freeze drying (e.g. according to WO2016184575 or WO2016184576) to yield a temperature stable composition (powder) as defined herein.

Accordingly, in preferred embodiments, the pharmaceutical composition is a dried composition.

The term “dried composition” as used herein has to be understood as composition that has been lyophilized, or spray-dried, or spray-freeze dried as defined above to obtain a temperature stable dried composition (powder) e.g. comprising LNP complexed RNA (as defined above).

In embodiments, lyophilized or spray-dried composition has a water content of less than about 10%.

In preferred embodiments, lyophilized or spray-dried composition has a water content of between about 0.5% and 5%.

In preferred embodiments, the lyophilized or spray-dried composition is stable for at least 2 months after storage at about 5° C., preferably for at least 3 months, 4 months, 5 months, 6 months.

According to further embodiments, the pharmaceutical composition may comprise at least one adjuvant.

Suitably, the adjuvant is preferably added to enhance the immunostimulatory properties of the composition.

The term “adjuvant” as used herein will be recognized and understood by the person of ordinary skill in the art, and is for example intended to refer to a pharmacological and/or immunological agent that may modify, e.g. enhance, the effect of other agents or that may be suitable to support administration and delivery of the composition. The term “adjuvant” refers to a broad spectrum of substances. Typically, these substances are able to increase the immunogenicity of antigens. For example, adjuvants may be recognized by the innate immune systems and, e.g., may elicit an innate immune response (that is, a non-specific immune response). “Adjuvants” typically do not elicit an adaptive immune response. In the context of the invention, adjuvants may enhance the effect of the antigenic peptide or protein provided by the nucleic acid. In that context, the at least one adjuvant may be selected from any adjuvant known to a skilled person and suitable for the present case, i.e. supporting the induction of an immune response in a subject, e.g. in a human subject.

Accordingly, the pharmaceutical composition may comprise at least one adjuvant, wherein the at least one adjuvant may be suitably selected from any adjuvant provided in WO2016203025. Adjuvants disclosed in any of the claims 2 to 17 of WO2016203025, preferably adjuvants disclosed in claim 17 of WO2016203025 are particularly suitable, the specific content relating thereto herewith incorporated by reference. Adjuvants may suitably used and comprised in the composition of the first aspect, or the vaccine of the second aspect, to e.g. reduce the amount of nucleic acid required for a sufficient immune response against the encoded protein and/or the improve the efficacy of the composition/the vaccine for treatment/vaccination, in particular of the elderly. A suitable adjuvant in the context of a coronavirus composition or vaccine (in particular for compositions comprising a polypeptide of the third aspect) may be a Toll-like receptor 9 (TLR9) agonist adjuvant, CpG 1018TM.

The pharmaceutical composition may comprise, besides the components specified herein, at least one further component which may be selected from the group consisting of further antigens (e.g. in the form of a peptide or protein, preferably derived from a coronavirus as specified herein and/or a further virus as specified herein) or further antigen-encoding nucleic acids; a further immunotherapeutic agent; one or more auxiliary substances (cytokines, such as monokines, lymphokines, interleukins or chemokines); or any further compound, which is known to be immune stimulating due to its binding affinity (as ligands) to human Toll-like receptors; and/or an adjuvant nucleic acid, preferably an immunostimulatory RNA (isRNA), e.g. CpG-RNA etc.

In embodiments, the pharmaceutical composition comprises at least one antagonist of at least one RNA sensing pattern recognition receptor. Such an antagonist may preferably be co-formulated in lipid-based carriers as defined herein.

Suitable antagonist of at least one RNA sensing pattern recognition receptor are disclosed in PCT patent application PCT/EP2020/072516, the full disclosure herewith incorporated by reference. In particular, the disclosure relating to suitable antagonist of at least one RNA sensing pattern recognition receptors as defined in any one of the claims 1 to 94 of PCT/EP2020/072516 are incorporated.

In preferred embodiments, the composition comprises at least one antagonist of at least one RNA sensing pattern recognition receptor selected from a Toll-like receptor, preferably TLR7 and/or TLR8.

In embodiments, the at least one antagonist of at least one RNA sensing pattern recognition receptor is selected from a nucleotide, a nucleotide analog, a nucleic acid, a peptide, a protein, a small molecule, a lipid, or a fragment, variant or derivative of any of these.

In preferred embodiments, the at least one antagonist of at least one RNA sensing pattern recognition receptor is a single stranded oligonucleotide, preferably a single stranded RNA Oligonucleotide.

In embodiments, the antagonist of at least one RNA sensing pattern recognition receptor is a single stranded oligonucleotide that comprises or consists of a nucleic acid sequence identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 85-212 of PCT/EP2020/072516, or fragments of any of these sequences.

In preferred embodiments, the antagonist of at least one RNA sensing pattern recognition receptor is a single stranded oligonucleotide that comprises or consists of a nucleic acid sequence identical or at least 70%, 80%, 85%, 86%, 87%, 88%, 89%, 90%, 91%, 92%, 93%, 94%, 95%, 96%, 97%, 98%, or 99% identical to a nucleic acid sequence selected from the group consisting of SEQ ID NOs: 85-87, 149-212 of PCT/EP2020/072516, or fragments of any of these sequences.

A particularly preferred antagonist of at least one RNA sensing pattern recognition receptor in the context of the invention is 5′-GAG CGmG CCA-3′ (SEQ ID NO: 85 of PCT/EP2020/072516), or a fragment thereof.

In embodiments, the molar ratio of the at least one antagonist of at least one RNA sensing pattern recognition receptor as defined herein to the at least one nucleic acid, preferably RNA encoding an antigenic peptide or protein as defined herein suitably ranges from about 1:1, to about 100:1, or ranges from about 20:1, to about 80:1.

In embodiments, the wherein the weight to weight ratio of the at least one antagonist of at least one RNA sensing pattern recognition receptor as defined herein to the at least one nucleic acid, preferably RNA encoding an antigenic peptide or protein as defined herein suitably ranges from about 1:1, to about 1:30, or ranges from about 1:2, to about 1:10.

In preferred embodiments, administration of the pharmaceutical composition as defined herein induces an antigen-specific humoral immune responses against the peptide or protein encoded by the at least one nucleic acid in said subject. Suitably, administration of the pharmaceutical composition as defined herein induces an antigen-specific humoral immune responses against all Coronavirus peptide or proteins (e.g., M, N, S) encoded by the at least one nucleic acid in said subject.

In preferred embodiments, administration of the pharmaceutical composition as defined herein induces antigen-specific T-cell responses against the peptide or protein encoded by the at least one nucleic acid in said subject. Suitably, administration of the pharmaceutical composition as defined herein induces an antigen-specific T-cell responses against all Coronavirus peptide or proteins (e.g., M, N, S) encoded by the at least one nucleic acid in said subject. In preferred embodiments, the T cell immune response comprises a CD4+ T cell immune response and/or a CD8+ T cell immune response.

In preferred embodiments, administration of the pharmaceutical composition as defined herein induces antigen-specific B-cell memory response against the peptide or protein encoded by the at least one nucleic acid in said subject. Suitably, administration of the pharmaceutical composition as defined herein induces an antigen-specific B-cell memory response against all Coronavirus peptide or proteins (e.g., M, N, S) encoded by the at least one nucleic acid in said subject.

In preferred embodiments, administration of the pharmaceutical composition as defined herein induces Coronavirus neutralizing antibodies induced in said subject.

In preferred embodiments, administration of the pharmaceutical composition as defined herein more Coronavirus neutralizing antibodies are induced in said subject than compared to Coronavirus neutralizing antibodies induced upon administration of a reference composition.

A reference composition (or vaccine) may be a corresponding peptide/protein-based vaccine or a nucleic-acid-based vaccine that comprises a nucleic acid encoding S (and lacking other nucleic acids encoding membrane protein (M), nucleocapsid protein (N), non-structural protein, and/or accessory protein or an immunogenic fragment or immunogenic variant thereof).

Vaccine, Multivalent Coronavirus Vaccine

In a second aspect, the invention relates to a nucleic acid based Coronavirus vaccine, preferably a multivalent Coronavirus vaccine.

It has to be noted that specific features and embodiments that are described in the context of the first aspect of the invention, that is the pharmaceutical composition of the invention, are likewise applicable to the second aspect (vaccine), the third aspect (kit or kit of parts of the invention), or further aspects including e.g. medical uses (first and second medical uses) and e.g. method of treatments.

In embodiments, the nucleic acid-based vaccine comprises one or more of M, N, NSP3, NSP4, NSP6, NSP13, NSP14, ORF3A, ORF8, and/or E, wherein the antigenic peptide or proteins are selected or derived from the same Coronavirus, preferably SARS-CoV-2. In embodiments, the pharmaceutical composition comprises one or more of M, N, NSP3, NSP4, NSP6, NSP13, NSP14, ORF3A, ORF8, and/or E, wherein the antigenic peptide or proteins are selected or derived from different Coronaviruses, preferably different pandemic Coronaviruses, e.g. SARS-CoV-2, SARS-CoV-1, and/or MERS-CoV. (suitable nucleic acid sequences encoding M, N, NSP3, NSP4, NSP6, NSP13, NSP14, ORF3A, ORF8, and/or E are defined in the first aspect).

In embodiments, the pharmaceutical composition comprises 2, 3, 4, 5, 6, 7, or more of M, N, NSP3, NSP4, NSP6, NSP13, NSP14, ORF3A, ORF8, and/or E wherein the antigenic peptide or proteins are selected or derived from the same Coronavirus, preferably SARS-CoV-2. In embodiments, the 2, 3, 4, 5, 6, 7 or more antigenic peptide or proteins are selected or derived from different Coronaviruses, preferably different pandemic Coronaviruses, e.g. SARS-CoV-2, SARS-CoV-1, and/or MERS-CoV. (suitable nucleic acid sequences encoding M, N, NSP3, NSP4, NSP6, NSP13, NSP14, ORF3A, ORF8, and/or E are defined in the first aspect).

In preferred embodiments, the pharmaceutical composition comprises 2, 3, 4, 5, 6, 7, or more of M, N, NSP3, NSP4, NSP6, NSP13, NSP14, ORF3A, ORF8, and/or E additionally comprising S, wherein the antigenic peptide or proteins are selected or derived from the same Coronavirus, preferably from SARS-CoV-2. In embodiments, the pharmaceutical composition comprises 2, 3, 4, 5, 6, 7, or more of M, N, NSP3, NSP4, NSP6, NSP13, NSP14, ORF3A, ORF8, and/or E additionally comprising S, wherein the antigenic peptide or proteins are selected or derived from different Coronaviruses, preferably different pandemic Coronaviruses, e.g. SARS-CoV-2, SARS-CoV-1, and/or MERS-CoV (suitable nucleic acid sequences encoding M, N, NSP3, NSP4, NSP6, NSP13, NSP14, ORF3A, ORF8, and/or E are defined in the first aspect).

Accordingly, in preferred embodiments, the vaccine is a multivalent Coronavirus vaccine, preferably a multivalent SARS-CoV-2 vaccine.

Further suitable combinations of antigens (provided nucleic acids) are defined in the context of the first aspect.

In embodiments, the vaccine is against multiple different Coronaviruses.

In embodiments, the vaccine is against at least one SARS-CoV-2, and at least one further (pandemic) Coronavirus.

According to a preferred embodiment, the vaccine as defined herein may further comprise a pharmaceutically acceptable carrier or excipient and optionally at least one adjuvant as specified in the context of the first aspect.

The vaccine typically comprises a safe and effective amount of nucleic acid (e.g. DNA or RNA), preferably RNA of the first aspect. As used herein, “safe and effective amount” means an amount of nucleic acid component sufficient to significantly induce a positive modification of a disease or disorder related to an infection with a virus as specified herein. At the same time, a “safe and effective amount” is small enough to avoid serious side-effects. In relation to the nucleic acid, composition, or vaccine of the present invention, the expression “safe and effective amount” preferably means an amount of nucleic acid, composition, or vaccine that is suitable for stimulating the adaptive immune system against a virus as specified herein in such a manner that no excessive or damaging immune reactions (e.g. innate immune responses) are achieved.

A “safe and effective amount” of the nucleic acid, composition, or vaccine as defined herein will vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the skilled person. Moreover, the “safe and effective amount” of the nucleic acid, the composition, or vaccine may depend from application/delivery route (intradermal, intramuscular, intranasal), application device (jet injection, needle injection, microneedle patch, electroporation device) and/or complexation/formulation (protamine complexation or LNP encapsulation, DNA or RNA). Moreover, the “safe and effective amount” of the nucleic acid, the composition, or the vaccine may depend from the physical condition of the treated subject (infant, pregnant women, immunocompromised human subject etc.).

The pharmaceutically acceptable carrier as used herein preferably includes the liquid or non-liquid basis of the inventive coronavirus vaccine. If the inventive vaccine is provided in liquid form, the carrier will be water, typically pyrogen-free water; isotonic saline or buffered (aqueous) solutions, e.g. phosphate, citrate etc. buffered solutions. Preferably, Ringer-Lactate solution is used as a liquid basis for the vaccine or the composition according to the invention as described in WO2006122828, the disclosure relating to suitable buffered solutions incorporated herewith by reference. Other preferred solutions used as a liquid basis for the vaccine or the composition, in particular for compositions/vaccines comprising LNPs, comprise sucrose and/or trehalose.

The choice of a pharmaceutically acceptable carrier or excipient as defined herein is determined, in principle, by the manner, in which the pharmaceutical composition(s) or vaccine according to the invention is administered. The Coronavirus vaccine is preferably administered locally. Routes for local administration in general include, for example, topical administration routes but also intradermal, transdermal, subcutaneous, or intramuscular injections or intralesional, intracranial, intrapulmonal, intracardial, intraarticular and sublingual injections. More preferably, composition or vaccines according to the present invention may be administered by an intradermal, subcutaneous, or intramuscular route, preferably by injection, which may be needle-free and/or needle injection. Preferred in the context of the invention is intramuscular injection. Compositions/vaccines are therefore preferably formulated in liquid or solid form. The suitable amount of the vaccine or composition according to the invention to be administered can be determined by routine experiments, e.g. by using animal models. Such models include, without implying any limitation, rabbit, sheep, mouse, rat, dog and non-human primate models. Preferred unit dose forms for injection include sterile solutions of water, physiological saline or mixtures thereof. The pH of such solutions should be adjusted to about 7.4.

In embodiments, the Coronavirus vaccine is provided in lyophilized or spray-dried form. Such a lyophilized or spray-dried vaccine may comprise trehalose and/or sucrose and may be re-constituted in a suitable liquid buffer before administration to a subject. In some embodiments, a lyophilized vaccine comprises RNA complexed with LNPs. In some embodiments, a lyophilized composition has a water content of less than about 10%. For example, a lyophilized composition can have a water content of about 0.1% to 10%, 0.1% to 7.5%, or 0.5% to 7.5%, preferably the lyophilized composition has a water content of about 0.5% to about 5.0%.

The vaccine can be used according to the invention for human medical purposes and also for veterinary medical purposes (mammals, vertebrates, or avian species).

In preferred embodiments, the composition or vaccine elicits an adaptive immune response against at least one Coronavirus, preferably against a pandemic Coronavirus, e.g. against SARS-CoV-2, SARS-CoV-1, or MERS-CoV.

In preferred embodiments, the vaccine elicits neutralizing antibody titers against at least one Coronavirus, preferably against a pandemic Coronavirus, e.g. against SARS-CoV-2, SARS-CoV-1, or MERS-CoV.

In some embodiments, the neutralizing antibody titer is at least 100 neutralizing units per milliliter (NU/mL), at least 500NU/mL, or at least 1000NU/mL.

In some embodiments, detectable levels of the respective antigen are produced in a subject at about 1 to about 72 hours post administration of the composition or the vaccine.

In some embodiments, a neutralizing antibody titer (against Coronavirus) of at least 100NU/ml, at least 500NU/ml, or at least 1000NU/ml is produced in the serum of the subject at about 1 days to about 72 days post administration of the composition or the vaccine.

In particularly preferred embodiments, the vaccine or the composition elicits functional antibodies that can effectively neutralize a Coronavirus (e.g. SARS-CoV-2 coronavirus).

In further preferred embodiments, the vaccine or the composition elicits mucosal IgA immunity by inducing of mucosal IgA antibodies.

In particularly preferred embodiments, the vaccine or the composition elicits functional antibodies that can effectively neutralize the Coronavirus (e.g. SARS-CoV-2 coronavirus).

In further particularly preferred embodiments, the vaccine or the composition induces broad, functional cellular T-cell responses against the Coronavirus (e.g. SARS-CoV-2 coronavirus).

In further particularly preferred embodiments, the vaccine or the composition induces a well-balanced B cell and T cell response against the Coronavirus (e.g. SARS-CoV-2 coronavirus).

In some embodiments, the neutralizing antibody titer is sufficient to reduce Coronavirus virus infection by at least 50% relative to a neutralizing antibody titer of an unvaccinated control subject or relative to a neutralizing antibody titer of a subject vaccinated with a live attenuated viral vaccine, an inactivated viral vaccine, or a protein sub unit viral vaccine.

In some embodiments, the neutralizing antibody titer and/or a T cell immune response is sufficient to reduce the rate of asymptomatic viral infection relative to the neutralizing antibody titer of unvaccinated control subjects.

In some embodiments, the neutralizing antibody titer and/or a T cell immune response is sufficient to prevent viral latency in the subject.

In some embodiments, the neutralizing antibody titer is sufficient to block fusion of Coronavirus with epithelial cells of the subject.

In preferred embodiments, the neutralizing antibody titer is induced following a single 1 μg-100 μg dose of the composition, or the vaccine. In further preferred embodiments, the neutralizing antibody titer is induced following a single 2 μg-20 μg dose of the composition, or the vaccine.

In some embodiments, the neutralizing antibody titer is induced within 20 days following a single 1 μg-100 μg dose of the composition or the vaccine, or within 40 days following a second 1 μg-100 μg dose of the nucleic acid, the composition or the vaccine.

In preferred embodiments, administration of a therapeutically effective amount of the composition or the vaccine to a subject induces a T cell immune response against Coronavirus in the subject. In preferred embodiments, the T cell immune response comprises a CD4+ T cell immune response and/or a CD8+ T cell immune response.

In preferred embodiments, the vaccine elicits antigen-specific immune responses comprising T-cell responses and/or B-cell responses against all encoded antigens provided by the composition (e.g., M, N, S).

In preferred embodiments, the vaccine elicits antigen-specific immune responses comprising T-cell responses and/or B-cell responses against all encoded antigens (e.g., M, N, S) upon administration of a low dose of the vaccine. In preferred embodiments, a low dose comprises less that about 100 μg, preferably less than about 50 μg, more preferably less than about 20 μg of nucleic acid, even more preferably less than about 10 μg.

In preferred embodiments, the vaccine elicits antigen-specific immune responses in a subject that has an age of about 5 years old or younger. Accordingly, the nucleic acid based Coronavirus vaccines are particularly suitable for infants.

In preferred embodiments, the vaccine elicits antigen-specific immune responses in a subject that has an age of about 60 years old or older. Accordingly, the nucleic acid based Coronavirus vaccines are particularly suitable for the elderly.

Kit or Kit of Parts, Application, Medical Uses, Method of Treatment:

In a third aspect, the present invention provides a kit or kit of parts suitable for treating or preventing Coronavirus infections. Preferably, said kit or kit of parts is suitable for treating or preventing a Coronavirus infection, preferably a SARS-CoV-2 infection.

Notably, embodiments relating to the pharmaceutical composition of the first aspect and the vaccine of the second aspect may likewise be read on and be understood as suitable embodiments of the kit or kit of parts of the third aspect of the invention.

In preferred embodiments, the kit or kit of parts comprises at least one pharmaceutical composition of the first aspect, and/or at least one vaccine of the second aspect.

In addition, the kit or kit of parts may comprise a liquid vehicle for solubilising or diluting, and/or technical instructions providing information on administration and dosage of the components.

The technical instructions of said kit may contain information about administration and dosage and patient groups. Such kits, preferably kits of parts, may be applied e.g. for any of the applications or uses mentioned herein, preferably for the use of the pharmaceutical composition of the first aspect or the vaccine of the second aspect, for the treatment or prophylaxis of an infection or diseases caused by a Coronavirus, preferably SARS-CoV-2 coronavirus.

Preferably, the pharmaceutical composition or the vaccine is provided in a separate part of the kit, wherein the pharmaceutical composition or the vaccine is optionally lyophilised or spray-dried or spray-freeze dried. The kit may further contain as a part a vehicle (e.g. buffer solution) for solubilising the dried or lyophilized nucleic composition or the vaccine.

In preferred embodiments, the kit or kit of parts as defined herein comprises a multi-dose container for administration of the composition/the vaccine and/or an administration device (e.g. an injector for intradermal injection or a syringe for intramuscular injection).

Any of the above kits may be used in a treatment or prophylaxis as defined herein.

First and Second/Further Medical Use:

A further aspect relates to the first medical use of the pharmaceutical composition of the first aspect, the vaccine of the second aspect, and the kit or kit of parts of the third aspect.

Notably, embodiments relating to the composition of the first aspect, the vaccine of the second aspect, or the kit or kit of parts of the third aspect may likewise be read on and be understood as suitable embodiments of medical uses of the invention.

Accordingly, the invention provides a pharmaceutical composition as defined in the first aspect for use as a medicament, the vaccine as defined in the second aspect for use as a medicament, and the kit or kit of parts as defined in the third aspect for use as a medicament.

The present invention furthermore provides several applications and uses of the composition, vaccine, or kit.

In particular, pharmaceutical compositions, vaccine, or kit may be used for human medical purposes and also for veterinary medical purposes, preferably for human medical purposes.

In particular, the pharmaceutical composition, the vaccine, or kit or kit of parts is for use as a medicament for human medical purposes, wherein said composition, vaccine, or kit or kit of parts may be suitable for young infants, newborns, immunocompromised recipients, as well as pregnant and breast-feeding women and elderly people. In particular, the pharmaceutical composition, vaccine, or kit or kit of parts is for use as a medicament for human medical purposes, wherein said composition, vaccine, or kit or kit of parts is particularly suitable for elderly human subjects.

Said pharmaceutical composition, vaccine, or kit is for use as a medicament for human medical purposes, wherein said composition, vaccine, or the kit or kit of parts is particularly suitable for intramuscular injection or intradermal injection.

In yet another aspect, the invention relates to the second medical use of the provided pharmaceutical composition, vaccine, or kit.

In an aspect, the invention relates to a pharmaceutical composition of the first aspect, a vaccine of the second aspect, a kit or kit of parts of the third aspect, for use in the treatment or prophylaxis of an infection with a Coronavirus, or a disorder related to such an infection.

In a preferred embodiment, the invention relates to a pharmaceutical composition of the first aspect, a vaccine of the second aspect, a kit or kit of parts of the third aspect, for use in the treatment or prophylaxis of an infection with SARS-CoV-2, or a disorder related to such an infection.

In an aspect, the invention relates to a pharmaceutical composition of the first aspect, a vaccine of the second aspect, a kit or kit of parts of the third aspect, for use in the treatment or prophylaxis of an infection with a Coronavirus, preferably SARS-CoV-2 (or a disorder related to such an infection) and in the treatment or prophylaxis of an infection with at least one further Coronavirus, e.g. a pandemic Coronavirus such as SARS-CoV-2 or MERS-CoV.

Suitably, the pharmaceutical composition of the first aspect, the vaccine of the second aspect, or the kit or kit of parts of the third aspect may be used in a method of prophylactic (pre-exposure prophylaxis or post-exposure prophylaxis) and/or therapeutic treatment of infections caused by at least one Coronavirus, preferably SARS-CoV-2 coronavirus.

The pharmaceutical composition or vaccine may preferably be administered locally. In particular, composition or vaccines may be administered by an intradermal, subcutaneous, intranasal, or intramuscular route. In embodiments, the inventive nucleic acid, composition, polypeptide, vaccine may be administered by conventional needle injection or needle-free jet injection. Preferred in that context is intramuscular injection.

In embodiments where plasmid DNA is used and comprised in the pharmaceutical composition or vaccine, the composition/vaccine may be administered by a device, e.g. an electroporation device, preferably an electroporation device for intradermal or intramuscular delivery. Suitably, a device as described in U.S. Pat. No. 7,245,963B2 may be used, in particular a device as defined by claims 1 to 68 of U.S. Pat. No. 7,245,963B2.

In embodiments where adenovirus DNA is used and comprised in the pharmaceutical composition or vaccine, the composition/vaccine may be administered by intranasal administration.

In embodiments, the nucleic acid as comprised in a composition or vaccine as defined herein is provided in an amount of about 100 ng to about 500 ug, in an amount of about 1 ug to about 200 ug, in an amount of about 1 ug to about 100 ug, in an amount of about 5 ug to about 100 ug, preferably in an amount of about 10 ug to about 50 ug, specifically, in an amount of about 1 ug, 2 ug, 3 ug, 4 ug, 5 ug, 6 ug, 7 ug, 8 ug, 9 ug, 10 ug, 11 ug, 12 ug, 13 ug, 14 ug, 15 ug, 20 ug, 25 ug, 30 ug, 35 ug, 40 ug, 45 ug, 50 ug, 55 ug, 60 ug, 65 ug, 70 ug, 75 ug, 80 ug, 85 ug, 90 ug, 95 ug or 100 ug. Notably, the amount relates to the total amount of nucleic acid comprised in the composition or vaccine.

In some embodiments, the vaccine comprising the nucleic acid, or the composition comprising the nucleic acid is formulated in an effective amount to produce an antigen specific immune response in a subject. In some embodiments, the effective amount is a total dose of 1 ug to 200 ug, 1 ug to 100 ug, or 5 ug to 100 ug. Notably, the effective amount relates to the total amount of nucleic acid comprised in the composition or vaccine.

In embodiments where the nucleic acid is provided in a lipid-based carrier, e.g. an LNP, the amount of PEG-lipid as defined herein comprised in one dose is lower than about 50 μg PEG lipid, preferably lower than about 45 μg PEG lipid, more preferably lower than about 40 μg PEG lipid.

Having a low amount of PEG lipid in one dose may reduce the risk of adverse effects (e.g. allergies).

In particularly preferred embodiments, the amount of PEG-lipid comprised in one dose is in a range from about 3.5 μg PEG lipid to about 35 μg PEG lipid.

In embodiments where the nucleic acid is provided in a lipid-based carrier, e.g. an LNP, the amount of cationic lipid as defined herein comprised in one dose is lower than about 400 μg cationic lipid, preferably lower than about 350 μg cationic lipid, more preferably lower than about 300 μg cationic lipid.

Having a low amount of cationic lipid in one dose may reduce the risk of adverse effects (e.g. fewer).

In particularly preferred embodiments, the amount of cationic-lipid comprised in one dose is in a range from about 30 μg PEG lipid to about 300 μg PEG lipid.

In one embodiment, the immunization protocol for the treatment or prophylaxis of a subject against at least one Coronavirus, preferably SARS-CoV-2 comprises one single dose of the pharmaceutical composition or the vaccine.

In some embodiments, the effective amount is a dose of 1 ug administered to the subject in one vaccination. In some embodiments, the effective amount is a dose of 2 ug administered to the subject in one vaccination. In some embodiments, the effective amount is a dose of 3 ug administered to the subject in one vaccination. In some embodiments, the effective amount is a dose of 4 ug administered to the subject in one vaccination. In some embodiments, the effective amount is a dose of 5 ug administered to the subject in one vaccination. In some embodiments, the effective amount is a dose of 6 ug administered to the subject in one vaccination. In some embodiments, the effective amount is a dose of 7 ug administered to the subject in one vaccination. In some embodiments, the effective amount is a dose of 8 ug administered to the subject in one vaccination. In some embodiments, the effective amount is a dose of 9 ug administered to the subject in one vaccination. In some embodiments, the effective amount is a dose of 10 ug administered to the subject in one vaccination. In some embodiments, the effective amount is a dose of 1 1 ug administered to the subject in one vaccination. In some embodiments, the effective amount is a dose of 12 ug administered to the subject in one vaccination. In some embodiments, the effective amount is a dose of 13 ug administered to the subject in one vaccination. In some embodiments, the effective amount is a dose of 14 ug administered to the subject in one vaccination. In some embodiments, the effective amount is a dose of 15 ug administered to the subject in one vaccination. In some embodiments, the effective amount is a dose of 20 ug administered to the subject in one vaccination. In some embodiments, the effective amount is a dose of 25 ug administered to the subject in one vaccination. In some embodiments, the effective amount is a dose of 30 ug administered to the subject in one vaccination. In some embodiments, the effective amount is a dose of 40 ug administered to the subject in one vaccination. In some embodiments, the effective amount is a dose of 50 ug administered to the subject in one vaccination. In some embodiments, the effective amount is a dose of 100 ug administered to the subject in one vaccination. In some embodiments, the effective amount is a dose of 200 ug administered to the subject in one vaccination. Notably, the effective amount relates to the total amount of nucleic acid comprised in the composition or vaccine.

In preferred embodiments, the immunization protocol for the treatment or prophylaxis of at least one Coronavirus infection comprises a series of single doses or dosages of the pharmaceutical composition or the vaccine. A single dosage, as used herein, refers to the initial/first dose, a second dose or any further doses, respectively, which are preferably administered in order to “boost” the immune reaction.

In some embodiments, the effective amount is a dose of 1 ug administered to the subject a total of two times. In some embodiments, the effective amount is a dose of 2 ug administered to the subject a total of two times. In some embodiments, the effective amount is a dose of 3 ug administered to the subject a total of two times. In some embodiments, the effective amount is a dose of 4 ug administered to the subject a total of two times. In some embodiments, the effective amount is a dose of 5 ug administered to the subject a total of two times. In some embodiments, the effective amount is a dose of 6 ug administered to the subject a total of two times. In some embodiments, the effective amount is a dose of 7 ug administered to the subject a total of two times. In some embodiments, the effective amount is a dose of 8 ug administered to the subject a total of two times. In some embodiments, the effective amount is a dose of 9 ug administered to the subject a total of two times. In some embodiments, the effective amount is a dose of 10 ug administered to the subject a total of two times. In some embodiments, the effective amount is a dose of 11 ug administered to the subject a total of two times. In some embodiments, the effective amount is a dose of 12 ug administered to the subject a total of two times. In some embodiments, the effective amount is a dose of 13 ug administered to the subject a total of two times. In some embodiments, the effective amount is a dose of 14 ug administered to the subject a total of two times. In some embodiments, the effective amount is a dose of 15 ug administered to the subject a total of two times. In some embodiments, the effective amount is a dose of 20 ug administered to the subject a total of two times. In some embodiments, the effective amount is a dose of 25 ug administered to the subject a total of two times. In some embodiments, the effective amount is a dose of 30 ug administered to the subject a total of two times. In some embodiments, the effective amount is a dose of 40 ug administered to the subject a total of two times. In some embodiments, the effective amount is a dose of 50 ug administered to the subject a total of two times. In some embodiments, the effective amount is a dose of 100 ug administered to the subject a total of two times. In some embodiments, the effective amount is a dose of 200 ug administered to the subject a total of two times. Notably, the effective amount relates to the total amount of nucleic acid comprised in the composition or vaccine.

In preferred embodiments, the vaccine/composition immunizes the subject against a Coronavirus infection (upon administration as defined herein) for at least 1 year, preferably at least 2 years. In preferred embodiments, the vaccine/composition immunizes the subject against a Coronavirus infection for more than 2 years, more preferably for more than 3 years, even more preferably for more than 4 years, even more preferably for more than 5-10 years.

Method of Treatment and Use, Diagnostic Method and Use:

In another aspect, the present invention relates to a method of treating or preventing a disorder.

Notably, embodiments relating to the pharmaceutical composition of the first aspect, the vaccine of the second aspect, the kit or kit of parts of the third aspect, or medical uses may likewise be read on and be understood as suitable embodiments of methods of treatments as provided herein. Furthermore, specific features and embodiments relating to method of treatments as provided herein may also apply for medical uses of the invention.

Preventing (Inhibiting) or treating a disease, in particular a virus infection relates to inhibiting the full development of a disease or condition, for example, in a subject who is at risk for a disease such as a virus infection. “Treatment” refers to a therapeutic intervention that ameliorates a sign or symptom of a disease or pathological condition after it has begun to develop. The term “ameliorating”, with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

In preferred embodiments, the present invention relates to a method of treating or preventing a disorder, wherein the method comprises applying or administering to a subject in need thereof at least one pharmaceutical composition of the first aspect, the vaccine of the second aspect, or the kit or kit of parts of the third aspect.

In preferred embodiments, the disorder is an infection with a Coronavirus, or a disorder related to such infections, in particular an infection with SARS-CoV-2, or a disorder related to such infections (e.g. COVID-19).

In preferred embodiments, the present invention relates to a method of treating or preventing a disorder as defined above, wherein the method comprises applying or administering to a subject in need thereof at least pharmaceutical composition of the first aspect, the vaccine of the second aspect, or the kit or kit of parts of the third aspect, wherein the subject in need is preferably a mammalian subject.

In particularly preferred embodiments, the subject in need is a mammalian subject, preferably a human subject, e.g. new-born, pregnant, immunocompromised, and/or elderly.

In particularly preferred embodiments, the human subject is an elderly human subject.

In certain embodiments, a method of treating or preventing disease by applying or administering to a subject in need thereof at least pharmaceutical composition of the first aspect, the vaccine of the second aspect, or the kit or kit of parts of the third aspect is further defined as a method of reducing disease burden in the subject. For example, the method preferably reduces the severity and/or duration of one or more symptom of COVID-19 disease. In some aspects, a method reduces the probability that a subject will require hospital admission, intensive care unit admission, treatment with supplemental oxygen and/or treatment with a ventilator. In further aspects, the method reduces the probability that a subject will develop a fever, breathing difficulties; loss of smell and/or loss of taste. In preferred aspects, the method reduces the probability that a subject will develop severe or moderate COVID-19 disease. In certain aspects, a method of the embodiments prevents severe or moderate COVID-19 disease in the subject between about 2 weeks and 1 month, 2 months, 3 months, 4 months, 5 months, 6 months, 1 year or 2 years after the subject is administered a composition of the embodiments. In preferred aspects, a method of the embodiments prevents symptomatic COVID-19 disease. In further aspects, a method of the embodiment prevents detectable levels of SARS-CoV-2 nucleic acid in the subject between about 2 weeks and 1 month, 2 months, 3 months, 4 months, 5 months, 6 months, 1 year or 2 years after the subject is administered a composition of the embodiments. In further aspects, a method of the embodiments is defined as a method for providing protective immunity to a coronavirus infection (e.g., SARS-CoV-2 infection) in the subject. In still further aspects, a method of the embodiments prevents moderate and severe COVID-19 disease in at least 80%, 85%, 90% or 95% of treated subjects. In yet further aspects, a method of the embodiments prevents moderate and severe COVID-19 disease in at least 80%, 85%, 90% or 95% of treated subjects from about 2 weeks to about 1 year after administering the second or subsequent immunogenic composition (e.g., a booster administration). In yet further aspects, a method of the embodiments prevents moderate and severe COVID-19 disease in at least 80%, 85%, 90% or 95% of treated subjects from about 2 weeks to about 3 month, 6 months, 9 months, 1 year, 1.5 years, 2 years or 3 years after administering the second or subsequent composition.

As used herein severe COVID-19 disease is defined as a subject experiencing one or more of the following:
Clinical signs at rest indicative of severe systemic illness (respiratory rate ≥30 breaths per minute, heart rate ≥125 per minute, SpO2≤93% on room air at sea level or PaO2/FIO2<300 mm Hg (adjusted according to altitude))
Respiratory failure (defined as needing high flow-oxygen, noninvasive ventilation, mechanical ventilation or ECMO)
Evidence of shock (SBP<90 mm Hg, DBP<60 mmHg, or requiring vasopressors)
Significant renal, hepatic, or neurologic dysfunction
Admission to ICU
Death

As used herein moderate COVID-19 disease is defined as a subject experiencing one or more of the following:
Shortness of breath or difficulty breathing
Respiratory rate ≥20 breaths per minute
Abnormal SpO2 but still >93% on room air at sea level (adjusted according to altitude)
Clinical or radiographic evidence of lower respiratory tract disease
Radiologic evidence of deep vein thrombosis (DVT)

As used herein mild COVID-19 disease is defined as a subject experiencing all of the following:
Symptomatic AND
No shortness of breath or difficulty breathing AND
No hypoxemia (adjusted according to altitude) AND
Does not meet the case definition of moderate or severe COVID-19 disease

In particularly preferred embodiments, the subject in need is a mammalian subject, preferably a human subject, e.g. newborn, pregnant, immunocompromised, and/or elderly. In some embodiments, the subject between the ages of 6 months and 100 years, 6 months and 80 years, 1 year and 80 years, 1 year and 70 years, 2 years and 80 years or 2 years and 60 years. In other embodiments the subject is a newborn or infant of an age of not more than 3 years, of not more than 2 years, of not more than 1.5 years, of not more than 1 year (12 months), of not more than 9 months, 6 months or 3 months. In certain embodiments, the human subject is an elderly human subject. In some other embodiments the subject is an elderly subject of an age of at least 50, 60, 65, or 70 years. In further aspects, a subject for treatment according to the embodiments is 61 years of age or older. In still further aspects, the subject is 18 years old to 60 years old.

In further embodiments, the mammalian subject is a human subject is 60 years of age or less. In certain embodiments the human subject is human subject is 55, 50, 45 or 40 years of age or less. Thus, in some embodiments, is the human subject is between about 12 and 60; 12 and 55; 12 and 50; 12 and 45; or 12 and 40 years of age. In further embodiments the human subject is between about 18 and 60; 18 and 55; 18 and 50; 18 and 45; or 18 and 40 years of age. In some embodiments the human subject is 18 to 50 or 18 to 40 years of age.

In certain embodiments, a subject for treatment according to the embodiments is a pregnant subject, such a pregnant human. In some aspects, the subject has been pregnant for more than about one month, two months, three months, four months, five months, six months, seven months or eight months.

In certain aspects, a subject for treatment according to the embodiments has native American, African, Asian or European heritage. In some aspects, the subject has at least about 10%, 15%, 20%, 25%, 30%, 35%, 40%, 45%, 50%, 55%, 60%, 65%, 70%, 75%, 80%, 85% or 90% native American, African, Asian or European heritage. In certain aspects, the subject has native American heritage, such as at least about 10%, 25% or 50% native American heritage. In further aspects, the subject is an elderly subject having native American heritage, e.g., a subject who is at least 55, 60, 65 or 70 years of age.

In further aspects, a subject for treatment according to the embodiments has a disease or is immune compromised. In some aspects, the subject has liver disease, kidney disease diabetes, hypertension, heart disease or lung disease. In further aspects, a subject for treatment according to the embodiments is a subject with history of allergic reaction, such a subject having food allergies. In some aspect, the subject has had a previous allergic reaction to a vaccine, such as an anaphylactic reaction. In still further aspects, a subject for treatment according to the methods is a subject having detectable anti-PEG antibodies, such as detectable anti-PEG IgE in the serum.

In further aspects, a subject for treatment according to the embodiments has at least one co-morbidity selected from:
(i) Chronic kidney disease: Kidney function will be ascertained from the serum creatinine measurement within the last 3-6 months, converted into estimated glomerular filtration rate (eGFR) using the Chronic Kidney Disease Epidemiology Collaboration (CKD-EPI) equation, with impaired kidney function defined as eGFR <60 mL/min/1.73 m².
   Mild chronic kidney disease is defined as an eGFR between 60-89 mL/min/1.73 m².
   Moderate chronic kidney disease is defined as an eGFR between 31-59 mL/min/1.73 m² with stable therapy and good maintenance over at least 6 months (modified from Clinical Practice Clinical Guidelines for Chronic Kidney Disease: Am J Kidney Dis, 2002).
(ii) COPD (including emphysema and chronic bronchitis).
   Mild COPD with or without cough or sputum production is defined as forced expiratory volume in 1 second/forced vital capacity (FEV1/FVC)<0.7 and FEV1 ≥80% predicted.
   Moderate COPD with or without cough or sputum production is defined as FEV1/FVC <0.7 and FEV1 ≥50%, but <80% predicted with stable treatment (GOLD Criteria for COPD severity).
(iii) Obesity with body mass index (BMI) of >32 kg/m²—any extreme morbid obesity will also be included.
(iv) Chronic cardiovascular conditions (heart failure, coronary artery disease, cardiomyopathies, arterial hypertension), including the following:
   Class I heart failure with potential high risk for developing heart failure in future with no functional or structural heart disorder.
   Class II heart failure: subjects with cardiac disease resulting in slight limitation of physical activity. Comfortable at rest.
   Ordinary physical activity results in fatigue, palpitation, dyspnea, or anginal pain.
   Class III heart failure with marked limitation of physical activity, but comfortable at rest but less than ordinary activity results in symptoms.
   A structural heart disorder without symptoms at any stage.
   Mild left ventricular systolic or diastolic dysfunction, usually with not much produced clinical signs.
   Moderate left ventricular failure with exertional dyspnea or orthopnea or paroxysmal nocturnal dyspnea as per New York Heart Association (NYHA), stable with medication (Class II-III).
   Coronary artery disease of 2 and above metabolic equivalent threshold (MET) up to moderate, stable with medication. (MET is defined as the amount of oxygen consumed while sitting at rest and is equal to 3.5 ml 02 per kg body weight×min; 4 Normal, can climb a flight of stairs or walk up a hill and can participate in other strenuous activities; 1 can take care of him/herself and may not maintain themselves and gets constraints on exertion.)
   Cardiomyopathies of non-infective and metabolic origin of 2-3 MET with medication.
   Stage 1 hypertension or Stage 2 hypertension stable and controlled with medications.
(v) Chronic HIV infection with stable aviraemia (<50 copies/mL) and CD4 count >350/mL as documented by blood samples taken within 12 months before enrolment. (Viral load <50 copies/mL with transient changes of 50-350 copies/mL is allowed.)
(vi) Type 2 diabetes mellitus, either controlled with medication [hemoglobin A1c (HbA1c)<58 mmol/mol (7.45%)] or uncontrolled with recent HbA1c of >58 mmol/mol (7.45%); [(HbA1c in %−2.15)×10.929=HbA1c in mmol/mol]; in uncontrolled DM HbA1c should be within <10% variation and should not have any history of diabetic ketoacidosis or episode of severe symptomatic hypoglycemia within prior 3 months.
(vii) Subjects who underwent renal transplant at least a year ago under stable conditions for at least 6 months with medications, categorized as low risk of rejection.

In still further aspects, a subject for treatment according to the embodiments has not been treated with an immunosuppressant drug for more than 14 days in the last 6 months. In some aspects, a subject for treatment according to the embodiments has not received a live vaccine for at least 28 days prior to the administration and/or has not received an inactivated vaccine for at least 14 days prior to the administration. In further aspects a subject for treatment according to the embodiments has NOT:
Had virologically-confirmed COVID-19 illness;
For females: experienced pregnancy or lactation with-in a month prior to administration of the composition of the embodiments;
had treatment with an investigational or non-registered product (e.g., vaccine or drug) within 28 days preceding the administration of the composition of the embodiments;
received a licensed vaccines within 28 days (for live vaccines) or 14 days (for inactivated vaccines) prior to the administration of the composition of the embodiments;
been previously or concurrently treated with any investigational SARS-CoV-2 vaccine or another coronavirus (SARS-CoV, MERS-CoV) vaccine;
been treated with immunosuppressants or other immune-modifying drugs (e.g., corticosteroids, biologicals and methotrexate) for >14 days total within 6 months preceding the administration of the composition of the embodiments;
had any medically diagnosed or suspected immunosuppressive or immunodeficient condition based on medical history and physical examination including known infection with human immunodeficiency virus (HIV), hepatitis B virus (HBV) or hepatitis C virus (HCV); current diagnosis of or treatment for cancer including leukemia, lymphoma, Hodgkin disease, multiple myeloma, or generalized malignancy; chronic renal failure or nephrotic syndrome; and receipt of an organ or bone marrow transplant.
had a history of angioedema (hereditary or idiopathic), or history of any anaphylactic reaction or pIMD.
a history of allergy to any component of CVnCoV vaccine.
been administered of immunoglobulins or any blood products within 3 months prior to the administration of the composition of the embodiments;
experienced a significant acute or chronic medical or psychiatric illness; and/or
experienced severe and/or uncontrolled cardiovascular disease, gastrointestinal disease, liver disease, renal disease, respiratory disease, endocrine disorder, and neurological and psychiatric illnesses.

In certain aspects, a subject for treatment according to the methods of the embodiments does not have any potential immune-mediated disease (pIMD). In further aspects, a treatment method of the embodiments does not induce any pIMD in a treated subject. As used herein pIMDs are defined as Celiac disease; Crohn's disease; Ulcerative colitis; Ulcerative proctitis; Autoimmune cholangitis; Autoimmune hepatitis; Primary biliary cirrhosis; Primary sclerosing cholangitis; Addison's disease; Autoimmune thyroiditis (including Hashimoto thyroiditis; Diabetes mellitus type I; Grave's or Basedow's disease; Antisynthetase syndrome; Dermatomyositis; Juvenile chronic arthritis (including Still's disease); Mixed connective tissue disorder; Polymyalgia rheumatic; Polymyositis; Psoriatic arthropathy; Relapsing polychondritis; Rheumatoid arthritis; Scleroderma, (e.g., including diffuse systemic form and CREST syndrome); Spondyloarthritis, (e.g., including ankylosing spondylitis, reactive arthritis (Reiter's Syndrome) and undifferentiated spondyloarthritis); Systemic lupus erythematosus; Systemic sclerosis; Acute disseminated encephalomyelitis, (including site specific variants (e.g., non-infectious encephalitis, encephalomyelitis, myelitis, myeloradiculomyelitis)); Cranial nerve disorders, (e.g., including paralyses/paresis (e.g., Bell's palsy)); Guillain-Barré syndrome, (e.g., including Miller Fisher syndrome and other variants); Immune-mediated peripheral neuropathies, Parsonage-Turner syndrome and plexopathies, (e.g., including chronic inflammatory demyelinating polyneuropathy, multifocal motor neuropathy, and polyneuropathies associated with monoclonal gammopathy); Multiple sclerosis; Narcolepsy; Optic neuritis; Transverse Myelitis; Alopecia areata; Autoimmune bullous skin diseases, including pemphigus, pemphigoid and dermatitis herpetiformis; Cutaneous lupus erythematosus; Erythema nodosum; Morphoea; Lichen planus; Psoriasis; Sweet's syndrome; Vitiligo; Large vessels vasculitis (e.g., including: giant cell arteritis such as Takayasu's arteritis and temporal arteritis); Medium sized and/or small vessels vasculitis (e.g., including: polyarteritis nodosa, Kawasaki's disease, microscopic polyangiitis, Wegener's granulomatosis, Churg-Strauss syndrome (allergic granulomatous angiitis), Buerger's disease thromboangiitis obliterans, necrotizing vasculitis and anti-neutrophil cytoplasmic antibody (ANCA) positive vasculitis (type unspecified), Henoch-Schonlein purpura, Behcet's syndrome, leukocytoclastic vasculitis); Antiphospholipid syndrome; Autoimmune hemolytic anemia; Autoimmune glomerulonephritis (including IgA nephropathy, glomerulonephritis rapidly progressive, membranous glomerulonephritis, membranoproliferative glomerulonephritis, and mesangioproliferative glomerulonephritis); Autoimmune myocarditis/cardiomyopathy; Autoimmune thrombocytopenia; Goodpasture syndrome; Idiopathic pulmonary fibrosis; Pernicious anemia; Raynaudvs phenomenon; Sarcoidosis; Sjögren's syndrome; Stevens-Johnson syndrome; Uveitis).

In certain aspects, a vaccination method of the embodiments does not result in a subject experiencing any adverse events of special interest (AESIs). As used herein AESIs are defined as a pIMD listed above; Anaphylaxis; Vasculitides; Enhanced disease following immunization; Multisystem inflammatory syndrome in children; Acute Respiratory Distress Syndrome; COVID-19 disease; Acute cardiac injury; Microangiopathy; Heart failure and cardiogenic shock; Stress cardiomyopathy; Coronary artery disease; Arrhythmia; Myocarditis, pericarditis; Thrombocytopenia; Deep vein thrombosis; Pulmonary embolus; Cerebrovascular stroke; Limb ischemia; Hemorrhagic disease; Acute kidney injury; Liver injury; Generalized convulsion; Guillain-Barré Syndrome; Acute disseminated encephalomyelitis; Anosmia, ageusia; Meningoencephalitis; Chilblain-like lesions; Single organ cutaneous vasculitis; Erythema multiforme; Serious local/systemic AR following immunization.

In a further aspects, a method of the embodiments comprises (i) obtaining a composition (e.g., a vaccine composition) of the embodiments, wherein the composition is lyophilized; (ii) solubilizing the lyophilized composition in a pharmaceutically acceptable liquid carrier to produce a liquid composition; and (iii) administering an effective amount of the liquid composition to the subject. In some aspects, the lyophilized composition comprises less than about 10% water content. For example, the lyophilized composition can preferably comprise about 0.1% to about 10%, 0.5% to 7.5% or 0.5% to 5.0% water.

In particular, such the method of treatment may comprise the steps of
a) providing at least one pharmaceutical composition of the first aspect, at least one vaccine of the second aspect, or the kit or kit of parts of the third aspect;
b) applying or administering said composition, vaccine, or kit or kit of parts to a subject as a first dose
c) optionally, applying or administering said composition, vaccine, or kit or kit of parts to a subject as a second dose or a further dose, preferably at least 1, 2, 3, 4, 5, 6, 7, 8, 9, 10, 11, 12, months after the first dose.

The first dosage, as used herein, refers to the initial/first dose, a second dose or any further doses, respectively, which are preferably administered in order to “boost” the immune reaction.

In certain aspects, the vaccine/composition is administered to a subject one, two three, four or more times. In some aspects, the vaccine/composition is administered to the subject at least first and a second time (e.g., a prime and boost). In some aspects, the send administration is at least 10 days, 14 days, 21 days, 28 days, 35 days, 42 days, 49 days or 56 days after the first administration. In some aspects, the time between the first administration and the second administration is between about 7 days and about 56 days; about 14 days and about 56 days; about 21 days and about 56 days; or about 28 days and about 56 days. In further aspects, the vaccine/composition is administered to a subject three or more times. In certain aspects, there is at least 10 days, 14 days, 21 days, 28 days, 35 days, 42 days, 49 days or 56 days between each administration of the vaccine/composition.

In some aspects, a subject for treatment according to the embodiments was previously infected with SARS-CoV-2 or was previously treated with at least a first SARS-CoV-2 vaccine composition. In some aspects, the subject was treated with one, two, three or more doses of a first SARS-CoV-2 vaccine composition. In some aspects, the composition of the embodiments used to treat a subject is a different type of vaccine composition than the composition previously used to treat the subject. In some aspects, the subject was previously treated with a mRNA vaccine, such as BNT162 or mRNA-1273. In further aspects, the subject was previously treated with a protein subunit vaccine, such as spike protein based vaccine, e.g., NVX-CoV2373 or COVAX. In further aspects, the subject was previously treated with a viral vector vaccine, such as an adenovirus vector based vaccine, e.g., ADZ1222 orAd26.COV-2.S. In further aspects, a subject previously treated with a vaccine composition has detectable SARS-CoV-2 binding antibodies, such as SARS-CoV-2 S protein-binding antibodies or SARS-CoV-2 N protein-binding antibodies. In further aspects, a subject for treatment according the embodiments was treated with a first SARS-CoV-2 vaccine composition at least about 3 month, 6 months, 9 months, 1 year, 1.5 years, 2 years or 3 years ago. In still further aspects, a subject for treatment according the embodiments was treated with a first SARS-CoV-2 vaccine composition between about 3 months and 2 years ago or between about 6 months and 2 years ago. In some aspects, a subjects treated with a further vaccine composition of the embodiments are protected from moderate and severe COVID-19 disease in at least 80%, 85%, 90% or 95% of treated subjects. For example, the treated subjects can be protected from moderate and severe COVID-19 disease in at least 80%, 85%, 90% or 95% of treated subjects from about 2 weeks to about 1 year after administration of the further composition. In still further aspects, administering the further vaccine composition of the embodiments prevents moderate and severe COVID-19 disease in at least 80%, 85%, 90% or 95% of treated subjects from about 2 weeks to about 3 month, 6 months, 9 months, 1 year, 1.5 years, 2 years or 3 years after said administration.

In certain aspects, a method of the embodiments is further defined as a method of stimulating an antibody or CD8+ T-cell response in a subject. In some aspects, the method is defined as a method of stimulating a neutralizing antibody response in a subject. In further aspects, the method is defined as a method of stimulating a protective immune response in a subject. In yet further aspects, the method is defined as a method of stimulating TH2 directed immune response in a subject.

In further aspects, administration of a vaccine/composition of the embodiments stimulates an antibody response that produces between about 10 and about 500 coronavirus spike protein-binding antibodies for every coronavirus neutralizing antibody in the subject. For example, the administration can stimulate an antibody response that produces no more than about 200 spike protein-binding antibodies for every coronavirus neutralizing antibody. In further aspects, the administration stimulates an antibody response that produces between about 10 and about 300; about 20 and about 300; about 20 and about 200; about 30 and about 100; or about 30 and about 80 coronavirus spike protein-binding antibodies for every coronavirus neutralizing antibody. In still further aspects, administration of composition of the embodiments stimulates an antibody response in a subject that includes a ratio of spike protein-binding antibodies to coronavirus neutralizing antibodies that is with 20%, 15%, 10% or 5% of the ratio of spike protein-binding antibodies to coronavirus neutralizing antibodies found in average convalescent patient serum (from a subject who has recovered from coronavirus infection).

In yet further aspects, administration of a composition of the embodiments stimulates an antibody response that produces between about 1 and about 500 coronavirus spike protein receptor binding domain (RBD)-binding antibodies for every coronavirus neutralizing antibody in the subject. In further aspects, the administration stimulates an antibody response that produces no more than about 50 spike protein RBD-binding antibodies for every coronavirus neutralizing antibody. In still further aspects, administration stimulates an antibody response that produces between about 1 and about 200; about 2 and about 100; about 3 and about 200; about 5 and about 100; about 5 and about 50; or about 5 and about 20 spike protein RBD-binding antibodies for every coronavirus neutralizing antibody. In still further aspects, administration of composition of the embodiments stimulates an antibody response in a subject that includes a ratio of spike protein RBD-binding antibodies to coronavirus neutralizing antibodies that is with 20%, 15%, 10% or 5% of the ratio of spike protein RBD-binding antibodies to coronavirus neutralizing antibodies found in average convalescent patient serum (from a subject who has recovered from coronavirus infection).

In still further aspects, administration of a vaccine/composition of the embodiments induces essentially no increase in IL-4, IL-13, TNF and/or IL-1β in the subject. In further aspects, the administration of a vaccine/composition of the embodiments induces essentially no increase in serum IL-4, IL-13, TNF and/or IL-1β in the subject. In some aspects, the administration of a vaccine/composition of the embodiments induces essentially no increase in IL-4, IL-13, TNF and/or IL-1β at the injection site (e.g., an intramuscular injection site) in the subject.

In still further aspects, a method of the embodiments comprises administration of a vaccine/composition of the embodiments to a human subject having a disease. In certain aspects, the subject has cardiovascular disease, kidney disease, lung disease or an autoimmune disease. In some aspects, a vaccine/composition of the embodiments is administered to a subject who is receiving anti-coagulation therapy.

In embodiments, the subject was previously treated with at least a first SARS-CoV-2 vaccine composition.

In embodiments, the first SARS-CoV-2 vaccine composition was a mRNA vaccine.

In embodiments, the first SARS-CoV-2 vaccine composition was BNT162 or mRNA-1273.

In embodiments, the first SARS-CoV-2 vaccine composition was a protein subunit vaccine.

In embodiments, the first SARS-CoV-2 vaccine composition was NVX-CoV2373 or COVAX.

In embodiments, the first SARS-CoV-2 vaccine composition was an adenovirus vector vaccine.

In embodiments, the first SARS-CoV-2 vaccine composition was ADZ1222 or Ad26.COV-2.S.

In embodiments, the subject has detectable SARS-CoV-2 binding antibodies after receiving the treatment as defined herein. In embodiments, the subject has detectable SARS-CoV-2 M protein-binding antibodies after receiving the treatment as defined herein. In embodiments, the subject has detectable SARS-CoV-2 N protein-binding antibodies after receiving the treatment as defined herein. In embodiments, the subject has detectable SARS-CoV-2 NSP protein-binding antibodies after receiving the treatment as defined herein. In embodiments, the subject has detectable SARS-CoV-2 E protein-binding antibodies after receiving the treatment as defined herein. In embodiments, the subject has detectable SARS-CoV-2 S protein-binding antibodies after receiving the treatment as defined herein.

According to a further aspect, the present invention also provides a method of manufacturing a composition or a vaccine, comprising the steps of:
a) optionally, adaptation of AU count of the respective coding sequences according to WO2019092153A1 (claims 1 to 70), thereby modifying the retention time of the nucleic acid on HPLC, allowing co-purification of the nucleic acid composition in step c);
b) RNA in vitro transcription step using a DNA template in the presence of a cap analogue to obtain capped mRNA, optionally wherein 2, 3, 4, 5, 6, 7, 8, 9, 10 or even more different RNAs are simultaneously produced in the RNA in vitro transcription step using 2, 3, 4, 5, 6, 7, 8, 9, 10 different DNA templates
c) Purifying the obtained capped RNA of step b) using RP-HPLC, and/or TFF, and/or Oligo(dT) purification and/or AEX, preferably using RP-HPLC and TFF;
d) Providing a first liquid composition comprising the purified capped RNA of step c);
e) Providing a second liquid composition comprising at least one cationic lipid as defined herein, a neutral lipid as defined herein, a steroid or steroid analogue as defined herein, and a PEG-lipid as defined herein;
f) Introducing the first liquid composition and the second liquid composition into at least one mixing means to allow the formation of LNPs comprising capped RNA;
g) Purifying the obtained LNPs comprising capped RNA;
h) optionally, lyophilizing the purified LNPs comprising capped RNA.

Preferably, the mixing means of step f) is a T-piece connector or a microfluidic mixing device. Preferably, the purifying step g) comprises at least one step selected from precipitation step, dialysis step, filtration step, TFF step. Optionally, an enzymatic polyadenylation step may be performed after step b). Optionally, further purification steps may be implemented to e.g. remove residual DNA, buffers, small RNA by-products etc. Optionally, RNA in vitro transcription is performed in the absence of a cap analog, and an enzymatic capping step is performed after RNA vitro transcription. Optionally, RNA in vitro transcription is performed in the presence of at least one modified nucleotide as defined herein.

Abbreviations
MERS-CoV: Middle East respiratory syndrome-related coronavirus
SARS-CoV: Severe acute respiratory syndrome-related coronavirus
SARS-CoV-2: Severe acute respiratory syndrome-related coronavirus 2
HCoV: Human coronavirus
Bat SARS-like CoV: Bat SARS-like coronavirus
BatCoV: Rousettus bat coronavirus
PDCoV: Porcine deltacoronavirus
PEDV: Porcine epidemic diarrhea virus
MHV: Murine hepatitis virus

### EXAMPLES

In the following, particular examples illustrating various embodiments and aspects of nucleic acid-based multivalent Coronavirus vaccines of the invention are presented, wherein the encoded antigens are selected or derived from SARS-CoV-2. However, the present invention shall not to be limited in scope by the specific embodiments relating to SARS-CoV-2 presented herein, and should rather be understood as being applicable to other Coronaviruses as defined in the specification, in particular to other pandemic Coronaviruses, e.g. MERS-CoV and/or SARS-CoV-1.

Accordingly, the following preparations and examples are given to enable those skilled in the art to more clearly understand and to practice the present invention. The present invention, is not limited in scope by the exemplified embodiments, which are intended as illustrations of single aspects of the invention only, and methods, which are functionally equivalent are within the scope of the invention. Indeed, various modifications of the invention in addition to those described herein will become readily apparent to those skilled in the art from the foregoing description and the examples below. All such modifications fall within the scope of the appended claims.

### Example 1: Preparation of DNA and RNA Constructs, Compositions, and Vaccines

The present example provides methods of obtaining the RNA of the invention as well as methods of generating a composition or a vaccine of the invention.

1.1. Preparation of DNA and RNA Constructs:

DNA sequences encoding different Coronavirus antigens were prepared and used for subsequent RNA in vitro transcription reactions. Said DNA sequences were prepared by modifying the wild type or reference encoding DNA sequences by introducing a G/C optimized or modified coding sequence (e.g., “cds opt1”) for stabilization and expression optimization. Sequences were introduced into a pUC derived DNA vector to comprise stabilizing 3′-UTR sequences and, optionally, 5′-UTR sequences, additionally comprising a stretch of adenosines (e.g. A64 or A100), and optionally a histone-stem-loop (hSL) structure, and optionally a stretch of 30 cytosines (e.g. C30) (see Table 10). The obtained plasmid DNA constructs were transformed and propagated in bacteria using common protocols known in the art. Eventually, the plasmid DNA constructs were extracted, purified, and used for subsequent RNA in vitro transcription.

TABLE 10
mRNA used in Examples

| | | SEQ ID | SEQ ID | SEQ |
|---|---|---|---|---|
| | | NO: | NO: CDS | ID NO: |
| RNA ID | Name | Protein | opt1 (gc) | mRNA |
| R9922 | SARS-COV-2/Wuhan-Hu-1 membrane protein | 15235 | 17112 | 20866 |
| R9924 | SARS-COV-2/Wuhan-Hu-1 membrane protein (A100) | 15235 | 17112 | 18989 |
| R9923 | SARS-COV-2/Wuhan-Hu-1 nucleocapsid protein | 15310 | 17187 | 20941 |
| R9925 | SARS-COV-2/Wuhan-Hu-1 nucleocapsid protein (A100) | 15310 | 17187 | 19064 |
| R9941 | SARS-COV-2/Wuhan-Hu-1 non-structural protein 3 | 15733 | 17610 | 21364 |
| R9946 | SARS-COV-2/Wuhan-Hu-1 non-structural protein 3 (A100) | 15733 | 17610 | 19487 |
| R9942 | SARS-COV-2/Wuhan-Hu-1 non-structural protein 4 | 16415 | 18292 | 22046 |
| R9947 | SARS-COV-2/Wuhan-Hu-1 non-structural protein 4 (A100) | 16415 | 18292 | 20169 |
| R9943 | SARS-COV-2/Wuhan-Hu-1 non-structural protein 6 | 16582 | 18459 | 22213 |
| R9948 | SARS-COV-2/Wuhan-Hu-1 non-structural protein 6 (A100) | 16582 | 18459 | 20336 |
| R10354 | SARS-COV-2/Wuhan-Hu-1 non-structural protein 13 protein | 27908 | 28029 | 28401 |
| R10355 | SARS-COV-2/Wuhan-Hu-1 non-structural protein 14 protein | 27909 | 28030 | 28402 |
| R10382 | SARS-COV-2/Wuhan-Hu-1 non-structural protein 13 protein (A100) | 27908 | 28029 | 28150 |
| R10383 | SARS-COV-2/Wuhan-Hu-1 non-structural protein 14 protein (A100) | 27909 | 28030 | 28151 |
| R9944 | SARS-COV-2/Wuhan-Hu-1 ORF3a protein | 16684 | 18561 | 22315 |
| R9949 | SARS-COV-2/Wuhan-Hu-1 ORF3a protein (A100) | 16684 | 18561 | 20438 |
| R9945 | SARS-COV-2/Wuhan-Hu-1 ORF8 protein | 16997 | 18874 | 22628 |
| R9950 | SARS-COV-2/Wuhan-Hu-1 ORF8 protein (A100) | 16997 | 18874 | 20751 |
| R9515 | SARS-COV-2/Wuhan-Hu-1 or SARS-COV-2/Wuhan/WIV05/2019 | 10 | 137 | 163 |
| | S_stab_PP (K986P_V987P) | | | |
| R9707 | SARS-COV-2/Wuhan-Hu-1 or SARS-COV-2/Wuhan/WIV05/2019 | 10 | 137 | 149 |
| | S_stab_PP (K986P_V987P) (A100) | | | |
| R10384 | SARS-COV-2/Wuhan-Hu-1 or SARS-COV-2/Wuhan/WIV05/2019 | 22961 | 23091 | 23531 |
| | S_stab_PP | | | |
| | (K986P_V987P_L18F_D80A_D215G_L242del_A243del_L244del_ | | | |
| | R24 61_K417N_E484K_N501Y_D614G_A701V) | | | |
| R10385 | SARS-COV-2/Wuhan-Hu-1 or SARS-COV-2/Wuhan/WIV05/2019 | 22963 | 23093 | 23533 |
| | S_stab_PP | | | |
| | (K986P_V987P_L18F_T20N_P26S_D138Y_R190S_K417T_E484K_ | | | |
| | N 501Y_D614G_H655Y_T1027I) | | | |

1.2. RNA In Vitro Transcription from Plasmid DNA Templates:

DNA plasmids prepared according to section 1.1 were enzymatically linearized using a restriction enzyme and used for DNA dependent RNA in vitro transcription using T7 RNA polymerase in the presence of a nucleotide mixture (ATP/GTP/CTP/UTP) and cap analog (e.g. m7GpppG, m7G(5′)ppp(5′)(2′OMeA)pG or m7G(5′)ppp(5′)(2′OMeG)pG)) under suitable buffer conditions. The obtained RNA constructs were purified using RP-HPLC (PureMessenger®, CureVac AG, Tubingen, Germany; WO2008077592) and used for in vitro and in vivo experiments. The generated RNA sequences/constructs are provided in Table 10 with the encoded antigenic protein indicated therein. Further indicated are the used codon optimization (CDS opt), the used UTR design (5′-UTR/3′-UTR), the used 3′ end of the construct (3′ end), and the respective protein, cds and mRNA sequences.

Generation of Chemically Modified RNA (Prophetic):

To obtain chemically modified mRNA, RNA in vitro transcription is performed in the presence of a modified nucleotide mixture comprising pseudouridine or N1-methylpseudouridine (m1ψ) instead of uracil. The obtained chemically modified RNA is purified using RP-HPLC (PureMessenger®, CureVac AG, Tubingen, Germany; WO2008077592) and used for further experiments.

Generation of Capped RNA Using Enzymatic Capping (Prophetic):

Some RNA constructs are in vitro transcribed in the absence of a cap analog. The cap-structure (cap0 or cap1) is then added enzymatically using capping enzymes as commonly known in the art. In short, in vitro transcribed RNA is capped using a capping kit to obtain cap0-RNA. cap0-RNA is additionally modified using cap specific 2′-O-methyltransferase to obtain cap1-RNA. cap1-RNA is purified e.g. as explained above and used for further experiments.

RNA for clinical development is produced under current good manufacturing practice e.g. according to WO2016180430, implementing various quality control steps on DNA and RNA level.

The RNA Constructs of the Examples:

The generated RNA sequences/constructs are provided in Table 10 with the encoded antigenic protein and the respective UTR elements. If not indicated otherwise, the RNA sequences/constructs of Table 4 have been produced using RNA in vitro transcription in the presence of a m7GpppG, m7G(5′)ppp(5′)(2′OMeA)pG; accordingly, the RNA sequences/constructs comprise a 5′ cap1 structure. If not indicated otherwise, the RNA sequences/constructs of Table 10 have been produced in the absence of chemically modified nucleotides (e.g. pseudouridine (ψ) or N1-methylpseudouridine (m1ψ).

1.3. RNA In Vitro Transcription from PCR Amplified DNA Templates (Prophetic):

Purified PCR amplified DNA templates prepared according to paragraph 1.1 is transcribed in vitro using DNA dependent T7 RNA polymerase in the presence of a nucleotide mixture (ATP/GTP/CTP/UTP) and cap analog (m7GpppG or 3′-O-Me-m7G(5′)ppp(5′)G)) under suitable buffer conditions. Alternatively, PCR amplified DNA is transcribed in vitro using DNA dependent T7 RNA polymerase in the presence of a modified nucleotide mixture (ATP, GTP, CTP, N1-methylpseudouridine (m1ψ) or pseudouridine (ψ) and cap analogue (m7GpppG, m7G(5′)ppp(5′)(2′OMeA)pG or m7G(5′)ppp(5′)(2′OMeG)pG) under suitable buffer conditions. Some RNA constructs are in vitro transcribed in the absence of a cap analog and the cap-structure (cap0 or cap1) is added enzymatically using capping enzymes as commonly known in the art. The obtained RNA is purified e.g. as explained above and used for further experiments. The obtained mRNAs are purified e.g. using RP-HPLC (PureMessenger®, CureVac AG, Tubingen, Germany; WO2008077592) and used for in vitro and in vivo experiments.

1.4. Preparation of an LNP Formulated mRNA Composition:

LNPs were prepared using cationic lipids, structural lipids, a PEG-lipids, and cholesterol. Lipid solution (in ethanol) was mixed with RNA solution (aqueous buffer) using T-piece formulation as outlined below. Obtained LNPs were re-buffered in a carbohydrate buffer via dialysis, and optionally up-concentrated to a target concentration using ultracentrifugation. LNP-formulated mRNA was stored at −80° C. prior to use in in vitro or in vivo experiments.

In short, lipid nanoparticles were prepared and tested according to the general procedures described in PCT Pub. Nos. WO2015199952, WO2017004143 and WO2017075531, the full disclosures of which are incorporated herein by reference. Lipid nanoparticle (LNP)-formulated mRNA was prepared using an ionizable amino lipid (cationic lipid), phospholipid, cholesterol and a PEGylated lipid (lipids see Table A). LNPs were prepared as follows. Cationic lipid according to formula III-3 (ALC-0315), DSPC, cholesterol and PEG-lipid according to formula IVa (ALC-0159) were solubilized in ethanol at a molar ratio of approximately 47.5:10:40.8:1.7. Lipid nanoparticles (LNP) comprising compound Ill-3 (ALC-0315) were prepared at a ratio of mRNA to total Lipid of 0.03-0.04 w/w. Briefly, the mRNA was diluted to 0.05 to 0.2 mg/mL in 10 to 50 mM citrate buffer, pH 4. Syringe pumps were used to mix the ethanolic lipid solution with the mRNA aqueous solution at a ratio of about 1:5 to 1:3 (vol/vol) with total flow rates above 15 ml/min. The ethanol was then removed and the external buffer replaced with PBS by dialysis. Finally, the lipid nanoparticles were filtered through a 0.2 μm pore sterile filter. Lipid nanoparticle particle diameter size was 60-90 nm as determined by quasi-elastic light scattering using a Malvern Zetasizer Nano (Malvern, UK).

TABLE A
Lipid-based carrier composition of the examples

| | | Ratio | | |
|---|---|---|---|---|
| | Compounds | (mol %) | Structure | Mass |
| 1 | Cholesterol | 40.9 | 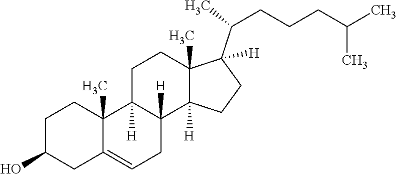 | 386.4 |
| 2 | 1,2-distearoyl- sn-glycero-3- phosphocholine (DSPC) | 10 | 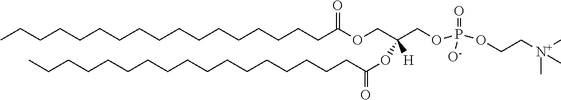 [SMILES: CCCCCCCCCCCCCCCCCC(=O)OC[C@H](COP(=O)([O-])OCC[N+](C)(C)C)OC(=O)CCCCCCCCCCCCCCCCC] | 789.6 |
| 3 | Cationic Lipid | 47.4 | 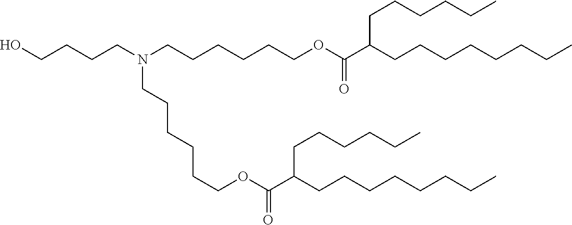 [SMILES: CCCCCCCCC(CCCCCC)C(=O)OCCCCCCN(CCCCO)CCCCCCOC(=O)C(CCCCCC)CCCCCCCC] | 765.7 |
| 4 | PEG Lipid | 1.7 | 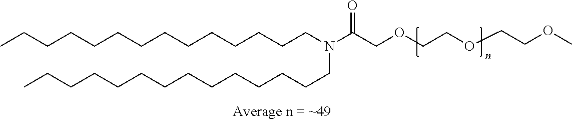 | 2010.1 |

1.5. Preparation of Combination mRNA Vaccines Comprising Antigen Combinations (Multivalent Vaccine Compositions):

Combination mRNA vaccines were formulated with LNPs either in a separate or co-formulated way. For separately mixed or formulated mRNA vaccines, each mRNA component was prepared and separately LNP formulated as described in Example 1.4, followed by mixing of the different LNP-formulated components. For co-formulated mRNA vaccine, the different mRNA components are firstly mixed together, followed by a co-formulation in LNPs as described in Example 1.4.

### Example 2: Expression Analysis of mRNA Constructs Encoding SARS-CoV-2 Proteins

2.1. Expression of SARS-CoV-2 Proteins in an In Vitro Translation Assay

To determine in vitro protein expression of the mRNA constructs, each of the mRNA constructs prepared according to Example 1 and listed in Table 10 are mixed with components of Promega Rabbit Reticulocyte Lysate System according to manufacture protocol. The lysate contains the cellular components necessary for protein synthesis (tRNA, ribosomes, amino acids, initiation, elongation and termination factors). As positive control, Luciferase RNA from Lysate System Kit is used. The translation result is analyzed by SIDS-Page and Western Blot analysis (IRDye 800GW streptavidin antibody (1:2000)).

2.2. Expression of SARS-CoV-2 Proteins in HeLa Cells and Analysis by FAGS

To determine in vitro protein expression of the mRNA constructs, HeLa cells are transiently transfected with each of the mRNA encoding SARS-CoV-2 proteins and stained using suitable antibodies (raised in mouse), counterstained with a FITC-coupled secondary antibody. HeLa cells are seeded in a 6-well plate at a density of 400,000 cells/well in cell culture medium (RPMI, 10% FCS, 1% L-Glutamine, 1% Pen/Strep), 24h prior to transfection. HeLa cells are transfected with 2 μg unformulated mRNA using Lipofectamine 2000 (Invitrogen). The mRNA constructs prepared according to Example 1 and listed in Table 10 are used in the experiment, including a negative control (water for injection). 24 hours post transfection, HeLa cells are stained with suitable antibodies (raised in mouse; 1:250) and anti-mouse FITC labelled secondary antibody (1:500) and are subsequently analyzed by flow cytometry (FACS) on a BD FACS Canto II using the FACS Diva software. Quantitative analysis of the fluorescent FITC signal is performed using the FlowJo software package (Tree Star, Inc.).

2.3. Expression Analysis of SARS-CoV-2 Proteins Using Western Blot

For the analysis of SARS-CoV-2 protein expression, HeLa cells are transfected with unformulated mRNA using Lipofectamine as the transfection agent. HeLa cells are seeded in a 6-well plate at a density of 300,000 cells/well. HeLa cells are transfected with 2 μg unformulated mRNA using Lipofectamine 2000 (Invitrogen). The mRNA constructs prepared according to Example 1 and listed in Table 10 are used in the experiment, including a negative control (water for injection). 24h post transfection, HeLa cells are detached by trypsin-free/EDTA buffer, harvested, and cell lysates are prepared. Cell lysates are subjected to SDS-PAGE followed by western blot detection. Western blot analysis is performed using respective antibodies used in combination with a suitable secondary antibody.

### Example 3: Vaccination of Mice with mRNA Encoding SARS-CoV-2 Proteins

3.1. Preparation of LNP Formulated mRNA Vaccine:

mRNA constructs are prepared as described in Example 1 (RNA in vitro transcription). HPLC purified mRNA is formulated with LNPs according to Example 1.4 prior to use in in vivo vaccination experiments. In the experiment, individual antigens are used and tested to assess the induction of immune responses of individual components of the nucleic acid-based multivalent Coronavirus vaccine.

3.2. Immunization:

Female BALB/c mice (6-8 weeks old) are injected intramuscularly (i.m.) with mRNA vaccine compositions and doses as indicated in Table 11. As a negative control, one group of mice is vaccinated with buffer. All animals are vaccinated on day 0 and 21. Blood samples are collected on day 21 (post prime) and 42 (post boost) for the determination of antibody titers.

TABLE 11
Vaccination regimen (Example 3):

| Group | Antigen | mRNA ID | Formulation | Dose |
|---|---|---|---|---|
| 1 | M | R9922 | LNP | 1-5 μg |
| 2 | M | R9923 | LNP | 1-5 μg |
| 3 | N | R9923 | LNP | 1-5 μg |
| 4 | N | R9925 | LNP | 1-5 μg |
| 5 | NSP3 | R9941 | LNP | 1-5 μg |
| 6 | NSP4 | R9942 | LNP | 1-5 μg |
| 7 | NSP6 | R9943 | LNP | 1-5 μg |
| 8 | ORF3a | R9944 | LNP | 1-5 μg |
| 9 | ORF8 | R9945 | LNP | 1-5 μg |
| 10 | NSP3 | R9946 | LNP | 1-5 μg |
| 11 | NSP4 | R9947 | LNP | 1-5 μg |
| 12 | NSP6 | R9948 | LNP | 1-5 μg |
| 13 | ORF3a | R9949 | LNP | 1-5 μg |
| 14 | ORF8 | R9950 | LNP | 1-5 μg |
| 15 | S | R9151 | LNP | 5 μg |
| 16 | | buffer | | |

3.3. Determination of IgG1 and IgG2 Antibody Titers Using ELISA:

ELISA is performed using recombinant SARS-CoV-2 protein for coating. Coated plates are incubated using respective serum dilutions, and binding of specific antibodies to the SARS-CoV-2 proteins are detected using biotinylated isotype specific anti-mouse antibodies followed by streptavidin-HRP (horse radish peroxidase) with Amplex as substrate. Endpoint titers of antibodies (IgG1, IgG2a) are measured by ELISA on day 21, and 42 post vaccinations.

3.4. Detection of SARS-CoV-2 Protein-Specific Immune Responses:

Hela cells are transfected with 2 μg of mRNA encoding SARS-CoV-2 proteins using lipofectamine. The cells are harvested 20h post transfection, and seeded at 1×10⁵ per well into a 96 well plate. The cells are incubated with serum samples of vaccinated mice (diluted 1:50) followed by a FITC-conjugated anti-mouse IgG antibody. Cells were acquired on BD FACS Canto II using DIVA software and analyzed by FlowJo.

3.5. Intracellular Cytokine Staining:

Splenocytes from vaccinated mice are isolated according to a standard protocol known in the art. Briefly, isolated spleens are grinded through a cell strainer and washed in PBS/1% FBS followed by red blood cell lysis. After an extensive washing step with PBS/1% FBS, splenocytes are seeded into 96-well plates (2×10⁶ cells per well). Cells are stimulated with a mixture of SARS-CoV-2 protein specific peptide epitopes (5 μg/ml of each peptide) in the presence of 2.5 μg/ml of an anti-CD28 antibody (BD Biosciences) for 6 hours at 37° C. in the presence of a protein transport inhibitor. After stimulation, cells are washed and stained for intracellular cytokines using the Cytofix/Cytoperm reagent (BD Biosciences) according to the manufacturer's instructions. The following antibodies are used for staining: Thy1.2-FITC (1:200), CD8-APC-H7 (1:100), TNF-PE (1:100), IFNγ-APC (1:100) (eBioscience), CD4-BD Horizon V450 (1:200) (BD Biosciences) and incubated with Fcγ-block diluted 1:100. Aqua Dye is used to distinguish live/dead cells (Invitrogen), Cells are acquired using a Canto II flow cytometer (Beckton Dickinson). Flow cytometry data is analyzed using FlowJo software package (Tree Star, Inc.)

3.6. Determination of Virus Neutralization Titers:

Serum is collected for determination of SARS-CoV-2 neutralization titers (VNTs) detected via CPE (cytopathic effect) or via a pseudo typed particle-based assay.

### Example 4: Vaccination of Mice with Nucleic Acid Based Multivalent Vaccines

4.1. Preparation of LNP Formulated mRNA Vaccine:

mRNA constructs for a multivalent SARS-CoV-2 vaccine are prepared as described in Example 1 (RNA in vitro transcription). HPLC purified mRNA is formulated with LNPs according to Example 1.4 prior to use in in vivo vaccination experiments.

4.2. Immunization:

Female BALB/c mice (6-8 weeks old) are injected intramuscularly (i.m.) with mRNA vaccine (respective RNA identifiers and constructs can be derived from Table 10) with doses as indicated in Table 12. As a negative control, one group of mice is vaccinated with buffer. All animals are vaccinated on day 0 and 21. Blood samples are collected on day 21 (post prime) and 42 (post boost) for the determination of antibody titers. After vaccination experiments, the efficiency of the vaccines is determined.

TABLE 12
Overview of the nucleic acid based vaccines and vaccination scheme

| | Antigen combination | Formulation | Dose per antigen |
|---|---|---|---|
| 1 | M and N | LNP | 0.1-5 μg |
| 2 | M and NSP3 | LNP | 0.1-5 μg |
| 3 | M and NSP4 | LNP | 0.1-5 μg |
| 4 | M and NSP6 | LNP | 0.1-5 μg |
| 5 | M and ORF3A | LNP | 0.1-5 μg |
| 6 | M and ORF8 | LNP | 0.1-5 μg |
| 7 | N and NSP3 | LNP | 0.1-5 μg |
| 8 | N and NSP4 | LNP | 0.1-5 μg |
| 9 | N and NSP6 | LNP | 0.1-5 μg |
| 10 | N and ORF3A | LNP | 0.1-5 μg |
| 11 | N and ORF8 | LNP | 0.1-5 μg |
| 12 | M and N and S | LNP | 0.1-5 μg |
| 13 | M and NSP3 and S | LNP | 0.1-5 μg |
| 14 | M and NSP4 and S | LNP | 0.1-5 μg |
| 15 | M and NSP6 and S | LNP | 0.1-5 μg |
| 16 | M and ORF3A and S | LNP | 0.1-5 μg |
| 17 | M and ORF8 and S | LNP | 0.1-5 μg |
| 18 | N and NSP3 and S | LNP | 0.1-5 μg |
| 19 | N and NSP4 and S | LNP | 0.1-5 μg |
| 20 | N and NSP6 and S | LNP | 0.1-5 μg |
| 21 | N and ORF3A and S | LNP | 0.1-5 μg |
| 22 | N and ORF8 and S | LNP | 0.1-5 μg |
| 23 | N and NSP3 and NSP4 and ORF8 | LNP | 0.1-5 μg |
| 24 | N and NSP3 and NSP4 and ORF8 and S | LNP | 0.1-5 μg |
| | Buffer control | | |

4.3. ELISA, FACS, ICS:

ELISA is performed essentially as described in Example 3. FAGS analysis is performed essentially as described in Example 3. Intracellular cytokine staining is performed essentially as described in Example 3.

4.4. Determination of Virus Neutralization Titers:

Serum is collected for determination of neutralization titers (VNTs) detected via CPE (cytopathic effect) or via a pseudo typed particle-based assay.

### Example 5: Vaccination of Rats with mRNA Encoding SARS-CoV-2 Antigen

Preparation of LNP Formulated mRNA Vaccine:

mRNA constructs for a multivalent SARS-CoV-2 vaccine are prepared as described in Example 1 (RNA in vitro transcription). HPLC purified mRNA is formulated with LNPs according to Example 1.4 prior to use in in vivo vaccination experiments.

Immunization:

Rats were injected intramuscularly (i.m.) with mRNA vaccine compositions as indicated in Table 12 (see Example 4) in doses ranging from 0.5 μg to 80 μg. As a negative control, one group of rats was vaccinated with buffer. All animals were vaccinated on day 0 and day 21. Blood samples were collected on day 21 (post prime) and 42 (post boost) for the determination of antibody titers.

Determination of IgG1 and IgG2 Antibody Titers Using ELISA:

ELISA is performed using recombinant SARS-CoV-2 protein for coating. Coated plates were incubated using respective serum dilutions, and binding of specific antibodies to SARS-CoV-2 S were detected directly with labeled HRP antibody instead of a secondary HRP antibody used for mouse ELISA. The lack of signal amplification in rat ELISA might account for lower titers, therefore ELISA titers between rat and mouse studies are currently not comparable.

### Example 6: Challenge Study of Hamster with SARS-CoV-2

Preparation of LNP Formulated mRNA Vaccine:

mRNA constructs for a multivalent SARS-CoV-2 vaccine are prepared as described in Example 1 (RNA in vitro transcription). HPLC purified mRNA is formulated with LNPs according to Example 1.4 prior to use in in vivo vaccination experiments.

Immunization and Challenge:

Female hamsters are injected intramuscularly (i.m.) with mRNA vaccine compositions as indicated in Table 12, administered as a 0.4 μg, 2 μg, and 10 μg dose. As a negative control, one group of hamsters is vaccinated with buffer. Group 2 is infected intranasal with SARS-CoV-2 virus. All further animals are vaccinated on day 0 and 28. Blood samples were collected on day 28 (post prime) and 42 (post boost) for the determination of antibody titers. The animals are challenged with SARS-CoV-2 virus at day 56. Protection from challenge infection may be analyzed by viral load in lungs and upper respiratory tract, affected lung tissue, and histology of lungs, liver and heart (4 days after challenge or 7 days after challenge). Alternatively, the animals are challenged at a later time point to show an advantage effect for long-term immunogenicity mediated through e.g. T-cell induced immune responses.

### Example 7: Challenge Study in K18-hACE2 Transgenic Mice

7.1 Preparation of LNP Formulated mRNA Vaccine:

mRNA constructs for a SARS-CoV-2 vaccine are prepared as described in Example 1 (RNA in vitro transcription). HPLC purified mRNA is formulated with LNPs according to Example 1.4 prior to use in in vivo vaccination experiments.

7.2. Immunization and Challenge:

K18-hACE2 transgenic mice are injected intramuscularly (i.m.) with mRNA vaccine compositions as indicated in Table 11 or 12, with doses ranging from 0.1-4 μg per group. As a negative control, one group of mice is vaccinated with buffer. All animals are vaccinated on day 0 and day 28. Blood samples were collected on day 0, day 28 (post prime), and day 56 (post boost) for the determination of antibody titers. The animals are challenged with SARS-CoV-2 virus at day 56 and monitored for 10 days for changes in body weight and survival, which indicates protection from challenge. Alternatively, the animals are challenged at a later time point to show an advantage effect for long-term immunogenicity mediated through e.g. T-cell induced immune responses. Additional parameters of protection include reduced viral loads in lungs and other organs and reduced pathology of the lung.

### Example 8: Challenge Study in K18-hACE2 Transgenic Mice

Generally, mice are not susceptible to infection with SARS-CoV-2, but a genetically engineered mouse model has been developed that expresses the human receptor ACE2 (hACE2), required for entry of the virus into the host cell under the K18 promoter. The model was originally developed to investigate the causative agent of SARS (SARS-CoV) (MCCRAY, Paul B., et al. Lethal infection of K18-hACE2 mice infected with severe acute respiratory syndrome coronavirus. Journal of virology, 2007, 81. Jg., Nr. 2, S. 813-821) but is now also used as a suitable small animal model for COVID-19. Previously, hACE2 mice have been shown to be susceptible to SARS-CoV-2 and to exhibit a disease course with weight loss, pulmonary pathology, and symptoms similar to those in humans (e.g. BAO, Linlin, et al. The pathogenicity of SARS-CoV-2 in hACE2 transgenic mice. Nature, 2020, 583. Jg., Nr. 7818, S. 830-833, or YINDA, Claude Kwe, et al. K18-hACE2 mice develop respiratory disease resembling severe COVID-19. PLoS pathogens, 2021, 17. Jg., Nr. 1, S. e1009195; DE ALWIS, Ruklanthi M., et al. A Single Dose of Self-Transcribing and Replicating RNA Based SARS-CoV-2 Vaccine Produces Protective Adaptive Immunity In Mice. BioRxiv, 2020.). In principle, the K18-hACE2 mouse is suitable for vaccine studies to investigate the prevention of infection with SARS-CoV-2 or the reduction of viral load, and at the same time to investigate the correlates and causes of a protective effect of an mRNA vaccine against COVID-19 with well-established immunological methods, which are generally available for mouse models.

The present example shows that multivalent SARS-CoV-2 S mRNA vaccines are able to protect K18-hACE2 mice from SARS-CoV-2 viral challenge, which can be shown e.g. by measuring the viral loads of infected animals, by monitoring the disease progression with weight loss, pulmonary pathology and other symptoms, or by histopathology and survival.

8.1 Preparation of LNP Formulated mRNA Vaccine:

mRNA constructs for a multivalent SARS-CoV-2 vaccine were prepared as described in Example 1 (RNA in vitro transcription). HPLC purified mRNA was formulated with LNPs according to Example 1.4 and Example 1.5 (separately mixed or formulated mRNA vaccines) prior to use in in vivo vaccination experiments.

8.2. Immunization and Challenge:

K18-hACE2 transgenic mice (female, n=10) were injected intramuscularly (i.m.) with a mRNA vaccine composition as indicated in Table 13 (group A). As a negative control, one group of mice was treated with buffer. The animals were vaccinated on day 0 and day 28 with indicated doses at a volume of 20 μl. Blood samples were collected on day 0, day 28 (post prime), and day 56 (post boost) for the determination of antibody titers. The animals were challenged/infected with SARS-CoV-2 virus (10⁵ TCID50 SARS-CoV-2 BavPat1) at day 56 and monitored for 10 days for changes in body weight, general health and survival, which indicates protection from challenge. Additional parameters of protection include reduced viral loads in lungs and other organs and reduced pathology of the lung. RNA extraction and RT-qPCR and sgRNA RT-PCR can be performed as described in Hoffmann et al 2021 (Hoffmann, D., Corleis, B., Rauch, S. et al. CVnCoV and CV2CoV protect human ACE2 transgenic mice from ancestral B BavPat1 and emerging B.1.351 SARS-CoV-2. Nat Commun 12, 4048 (2021)).

TABLE 13
Vaccination regimen (Example 8):

| | | | 5′-UTR/ | | SEQ ID | SEQ ID | |
|---|---|---|---|---|---|---|---|
| | | mRNA | 3′-UTR; | | NO: | NO: | |
| Group | Vaccine composition | ID | UTR Design | 3′-end | Protein | RNA | Dose |
| A | mRNA encoding | | | | | | |
| | N | R9925, | HSD17B4/ | hSL-A100 | 15310 | 17187 | 4 μg |
| | nsp3 | R9946, | PSMB3; a-1 | | 15733 | 17610 | 4 μg |
| | nsp4 | R9947, | | | 16415 | 18292 | 4 μg |
| | Orf8 | R9950 | | | 16997 | 18874 | 4 μg |
| | formulated in LNPs | | | | | | (in total 16 μg) |
| | NaCl buffer | | | | | | |

RBD Antibody Enzyme-Linked Immunosorbent Assay (ELISA)

Sera were analyzed using an indirect multi-species ELISA based on the RBD of SARS-CoV-234. For this, ELISA plates (Greiner Bio-One GmbH) were coated with 100 ng/well the RBD overnight at 4° C. in 0.1M carbonate buffer (1.59 g Na₂CO₃ and 2.93 g NaHCO₃, ad. 1 L aqua dest., pH9.6) or were treated with the coating buffer only. Afterwards, the plates were blocked for 1h at 37° C. using 5% skim milk in PBS. Sera were pre-diluted 1/100 in TBS-Tween (TBST) and incubated on the coated and uncoated wells for 1h at RT. A multi-species conjugate (SBVMILK; obtained from ID Screen® Schmallenberg virus Milk Indirect ELISA; IDvet) was diluted 1/80 and then added for 1h at RT. Following the addition of tetramethylbenzidine substrate (IDEXX), the ELISA readings were taken at a wavelength of 450 nm on a Tecan Spectra Mini instrument (Tecan Group Ltd.). Between each step, the plates were washed three times with TBST. The absorbance was calculated by subtracting the optical density measured on the uncoated wells from the values obtained from the protein-coated wells for the respective sample. Of note, the ELISA determines relative abundance of anti-RBD Ig levels and therefore does not allow a direct comparison between different studies.

Results:

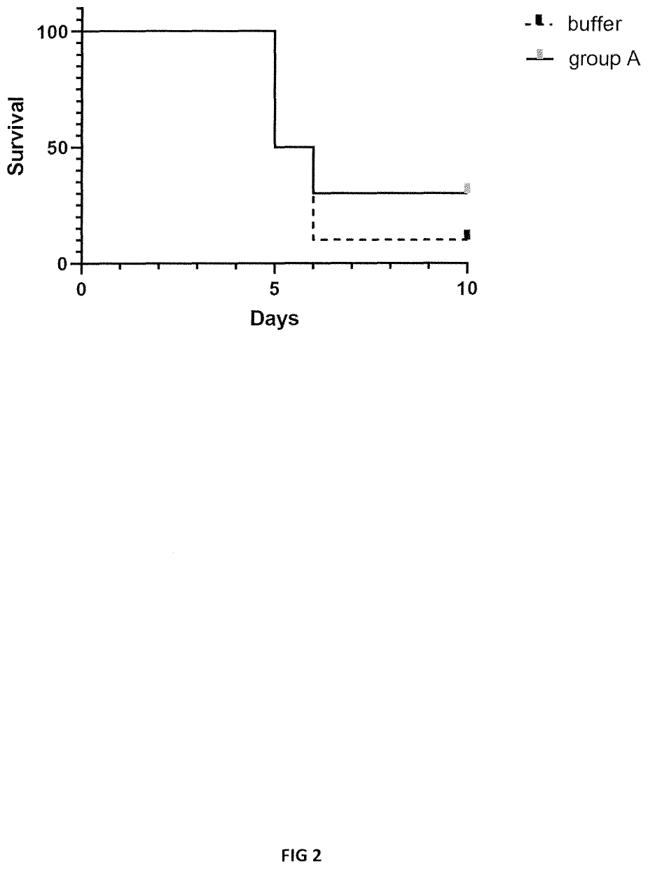

As shown in FIGS. 1A,1B, and 2 mice of group A benefit from treatment with multivalent vaccine composition comprising mRNA encoding SARS-CoV-2 antigens N, NSP3, NSP4 and ORF8. FIG. 1 demonstrates the percentage body weight changes in days post infection/challenge (FIG. 1A: mean percentage body weight, FIG. 1B: percentage body weight per mouse). FIG. 2 demonstrates survival of challenged mice in days post infection/challenge. The multivalent vaccine composition comprising mRNA encoding SARS-CoV-2 antigens N, NSP3, NSP4 and ORF8 protects 30% of challenged mice.

### Example 9: Challenge Study in K18-hACE2 Transgenic Mice (Prophetic)

The present example analyses multivalent SARS-CoV-2 S mRNA vaccines (including optionally Spike S as an additional antigen) in the described K18-hACE2 mice challenge model. Readouts are e.g. measuring the viral loads of infected animals, by monitoring the disease progression with weight loss, pulmonary pathology and other symptoms, or by histopathology and survival.

8.1 Preparation of LNP Formulated mRNA Vaccine:

mRNA constructs for a multivalent SARS-CoV-2 vaccine are prepared as described in Example 1 (RNA in vitro transcription). HPLC purified mRNA are formulated with LNPs according to Example 1.4 and Example 1.5 (separately mixed or formulated mRNA vaccines) prior to use in in vivo vaccination experiments.

8.2. Immunization and Challenge:

K18-hACE2 transgenic mice (female, n=10) are injected intramuscularly (i.m.) with mRNA vaccine compositions as indicated in Table 14 and described in Example 8. The multivalent vaccine compositions are chosen exemplary and can be changed according to disclosed multivalent composition/combinations of M, N, NSP3, NSP4, NSP6, NSP13, NSP14, ORF3A, ORF8, and/or E and optionally with additional S.

TABLE 14
Vaccination regimen (Example 9):

| | | | 5′-UTR/3′- | | SEQ ID | SEQ ID | | |
|---|---|---|---|---|---|---|---|---|
| | Vaccine | mRNA | UTR; UTR | | NO: | NO: | | |
| Group | composition | ID | Design | 3′-end | Protein | RNA | Dose | |
| A | mRNA encoding | R9707 | HSD17B4/ | hSL-A100 | 10 | 149 | 0.5 | μg |
| | S_stab formulated | | PSMB3; a-1 | | | | | |
| | in LNPs | | | | | | | |
| B | mRNA encoding | | | | | | 0.5 | μg |
| | S_stab | R9707, | HSD17B4/ | hSL-A100 | 10 | 149 | 4 | μg |
| | N | R9925, | PSMB3; a-1 | | 15310 | 17187 | 4 | μg |
| | nsp3 | R9946, | | | 15733 | 17610 | 4 | μg |
| | nsp4 | R9947, | | | 16415 | 18292 | 4 | μg |
| | Orf8 | R9950 | | | 16997 | 18874 | | |
| | formulated in LNPs | | | | | | (in total 16.5 μg) | |
| C | mRNA encoding | | | | | | | |
| | N | R9925, | HSD17B4/ | hSL-A100 | 15310 | 17187 | 4 | μg |
| | nsp3 | R9946, | PSMB3; a-1 | | 15733 | 17610 | 4 | μg |
| | nsp4 | R9947, | | | 16415 | 18292 | 4 | μg |
| | Orf8 | R9950 | | | 16997 | 18874 | 4 | μg |
| | formulated in LNPs | | | | | | (in total 16 μg) | |
| D | NaCl buffer | | | | | | | |

Measurements to determine humoral or cellular immune responses can be performed as follows:

RBD Antibody Enzyme-Linked Immunosorbent Assay (ELISA):

Sera are analyzed using an indirect multi-species ELISA based on the RBD of SARS-CoV-234. For this, ELISA plates (Greiner Bio-One GmbH) are coated with 100 ng/well the RBD overnight at 40C in 0.1 M carbonate buffer (1.59 g Na₂CO₃ and 2.93 g NaHCO₃, ad. 1 L aqua dest., pH9.6) or are treated with the coating buffer only. Afterwards, the plates are blocked for 1 h at 3700 using 5% skim milk in PBS. Sera are pre-diluted 1/100 in TBS-Tween (TBST) and incubated on the coated and uncoated wells for 1 h at RT. A multi-species conjugate (SBVMILK; obtained from ID Screen® Schmallenberg virus Milk Indirect ELISA; IDvet) is diluted 1/80 and then added for 1h at RT. Following the addition of tetramethylbenzidine substrate (IDEXX), the ELISA readings are taken at a wavelength of 450 nm on a Tecan Spectra Mini instrument (Tecan Group Ltd.). Between each step, the plates are washed three times with TBST. The absorbance is calculated by subtracting the optical density measured on the uncoated wells from the values obtained from the protein-coated wells for the respective sample. Of note, the ELISA determines relative abundance of anti-RBD Ig levels and therefore does not allow a direct comparison between different studies.

Determination of IgG1 and IgG2 Antibody Titers Using ELISA:

Alternatively, ELISA is performed using recombinant SARS-CoV-2 S (extracellular domain) protein for coating. Coated plates are incubated using respective serum dilutions, and binding of specific antibodies to SARS-CoV-2 S are detected using biotinylated isotype specific anti-mouse antibodies followed by streptavidin-HRP (horse radish peroxidase) with Amplex as substrate. Endpoint titers of antibodies (IgG1, IgG2a) are measured by ELISA on day 21, and 42 post prime vaccination. To determine SARS-CoV-2 specific antibody titers to e.g. Nucleoprotein N, plates are coated with recombinant N protein.

Determination of Virus Neutralization Titers (VNTs):

A Virus neutralization test is performed to evaluate specifically the presence of virus neutralizing antibodies in serum samples (pre-challenge), Therefore, sera are pre-diluted 1/16 or 1/32 with Dulbecco's modified Eagle's medium (DMEM) in a 96-well deep well master plate. Three times 100 μl, representing three technical replicates, of this pre-dilution are transferred into a 96-well plate. A log 2 dilution is conducted by passaging 50 μl of the serum dilution in 50 μl DMEM, leaving 50 μl of sera dilution in each well. Subsequently, 50 μl of the respective SARS-CoV-2 (BavPat1) virus dilution (100 TCID50/well) is added to each well and incubated for 1h at 37° C. Lastly, 100 μl of trypsinated VeroE6 cells (cells of one confluent TC175 flask per 100 ml) in DMEM with 1% penicillin/streptomycin supplementation is added to each well. After 72h incubation at 37° C., the cells are evaluated by light microscopy for a specific CPE.

A serum dilution is counted as neutralizing in the case no specific CPE was visible. The virus titer is confirmed by virus titration; positive and negative serum samples were included.

Intracellular Cytokine Staining:

Splenocytes from vaccinated mice are isolated according to a standard protocol known in the art. Briefly, isolated spleens are grinded through a cell strainer and washed in PBS/1% FBS followed by red blood cell lysis. After an extensive washing step with PBS/1% FBS, splenocytes are seeded into 96-well plates (2×10⁶ cells per well). Cells are stimulated with a mixture of SARS-CoV-2 S protein specific peptide epitopes (5 μg/ml of each peptide) in the presence of 2.5 μg/ml of an anti-CD28 antibody (BD Biosciences) for 6 hours at 37° C. in the presence of a protein transport inhibitor. After stimulation, cells are washed and stained for intracellular cytokines using the Cytofix/Cytoperm reagent (BD Biosciences) according to the manufacturer's instructions. The following antibodies are used for staining: Thy1.2-FITC (1:200), CD8-APC-H7 (1:100), TNF-PE (1:100), IFNγ-APC (1:100) (eBioscience), CD4-BD Horizon V450 (1:200) (BD Biosciences) and incubated with Fcγ-block diluted 1:100. Aqua Dye is used to distinguish live/dead cells (Invitrogen). Cells are acquired using a Canto II flow cytometer (Beckton Dickinson). Flow cytometry data is analyzed using FlowJo software package (Tree Star, Inc.)

The immunization and challenge of K18-hACE2 mice as described in the present Example can be used to determine the protective efficacy of further inventive mRNA constructs and compositions. Furthermore, by using mutated virus variants or isolates of SARS-CoV-2 (e.g. B.1.351, see also Table 15), it can be shown, that the inventive mRNA vaccine compositions are effective in addition against these virus variants or isolates. Emerging SARS-CoV-2 variants or isolates for analysis are listed in List 1C. Further variants may arise or exist and can be tested as well.

1. A pharmaceutical composition comprising at least one nucleic acid comprising at least one RNA coding sequence encoding at least one antigenic peptide or protein from at least one Coronavirus, wherein the at least one antigenic peptide or protein is a nucleocapsid protein (N) having a sequence at least 90% identical to the sequence according to SEQ ID No.: 15310.

2-11. (canceled)

12. The pharmaceutical composition of claim 1, wherein the at least one RNA coding sequence comprises or consists at least one nucleic acid sequence encoding a nucleocapsid protein (N) being identical or at least 85% identical to any one of the nucleic acid sequences selected from SEQ ID NOs: 17187, 19064 and 20941.

13-43. (canceled)

44. The pharmaceutical composition of claim 1, comprising the at least one further RNA coding sequence encoding at least one further antigenic peptide or protein from a Coronavirus.

45. The pharmaceutical composition of claim 44, comprising at least two RNA nucleic acid sequences according to the following combinations:
M and N; N and NSP4; N and NSP6; N and NSP13; N and NSP 14; N and ORF3A; or N and ORF8.

46. (canceled)

47. The pharmaceutical composition of claim 44, comprising at least four RNA coding sequences encoding:
NSP3 and NSP4 and NSP6 and N; NSP3 and NSP4 and ORF3A and N;
NSP3 and NSP4 and ORF8 and N; NSP3 and NSP6 and ORF3A and N;
NSP3 and NSP6 and ORF8 and N; NSP3 and ORF3A and ORF8 and N;
NSP4 and NSP6 and ORF3A and N; NSP4 and NSP6 and ORF8 and N; or
NSP4 and ORF3A and ORF8 and N; NSP6 and ORF3A and ORF8 and N.

48. The pharmaceutical composition of claim 44, comprising at least five RNA coding sequences encoding:
NSP3 and NSP4 and NSP6 and M and N; NSP3 and NSP4 and ORF3A and M and N;
NSP3 and NSP4 and ORF8 and M and N; NSP3 and NSP6 and ORF3A and M and N;
NSP3 and NSP6 and ORF8 and M and N; NSP3 and ORF3A and ORF8 and M and N;
NSP4 and NSP6 and ORF3A and M and N; NSP4 and NSP6 and ORF8 and M and N; or
NSP4 and ORF3A and ORF8 and M and N; NSP6 and ORF3A and ORF8 and M and N.

49. The pharmaceutical composition of claim 1, additionally comprising at least one RNA comprising at least one coding sequence encoding at least one antigenic peptide or protein selected or derived from at least one Coronavirus spike protein (S), or an immunogenic fragment thereof.

50-53. (canceled)

54. The pharmaceutical composition of claim 49, wherein the Coronavirus spike protein (S) is a SARS-CoV-2 spike protein (S1, S2, or S1 and S2), or an immunogenic fragment thereof

55. The pharmaceutical composition of claim 54, wherein the spike protein is a pre-fusion stabilized spike protein (S_stab) comprising at least one pre-fusion stabilizing mutation.

56. The pharmaceutical composition of claim 55, wherein the at least one pre-fusion stabilizing mutation comprises the following amino acid substitutions: K986P and V987P.

57-96. (canceled)

97. The pharmaceutical composition of claim 96, wherein the at least one RNA coding sequence is a codon modified coding sequence having an increased G/C content.

98. The pharmaceutical composition of claim 97, wherein the at least one RNA coding sequence has a G/C content of at least about 50%, 55%, or 60%.

99. The pharmaceutical composition of claim 1, wherein the at least one nucleic acid comprises at least one heterologous untranslated region selected from at least one heterologous 5′-UTR and/or at least one heterologous 3′-UTR.

100-106. (canceled)

107. The pharmaceutical composition of claim 1, wherein the at least one nucleic acid is a e N mRNA, a self-replicating RNA, a circular RNA, or a replicon RNA.

108. The pharmaceutical composition of claim 107, wherein the at least one nucleic acid is an mRNA.

109-110. (canceled)

111. The pharmaceutical composition of claim 107, wherein all uracil nucleotides are replaced by pseudouridine (Ψ) nucleotides and/or N1-methylpseudouridine (m1Ψ) nucleotides.

112-115. (canceled)

116. The pharmaceutical composition of claim 1, wherein the at least one nucleic acid is complexed or associated with one or more lipids thereby forming lipid nanoparticles (LNPs).

117-146. (canceled)

147. A Kit or kit of parts, comprising at least one pharmaceutical composition of claim 1 and technical instructions providing information on administration and dosage of the kit components.

148-149. (canceled)

150. A method of treating or preventing a disorder, wherein the method comprises applying or administering to a subject in need thereof the pharmaceutical composition of claim 1.

151-156. (canceled)

157. A method of stabilizing a pharmaceutical composition of claim 1 comprising lyophilizing the composition or vaccine to a produce a stabilized composition or vaccine.

158-160. (canceled)
